SEQUENCE LISTING (20 sequences)

SEQ ID NO: 1 - protein, 588 aa, Felis silvestris catus
MPGLLSPPALVLSVMGALLMAGDPGEEVSSTPALPGGPATGTGGLIFHPDWDWQPPGSPQDPLCLVTLDRGGNGSGSPLRVVGALRGYEHAFLEAVRRARWGPHGLATFGVCTPRDRQAAPFSLRQLQAWLGEPGGRRLVVLHLEEVTWEPTPSLKFQEPPPGGAGPLELAMLVLYPGPGPEVTVTGAGLPGTQSLCQSRDTRYLVLAVDHPEGAWRSPGLTLTLQPRRDGAPLSTAQLQELLFGPDPRCFTRMTPALLLLPGPAPAPLPARGLLDQVPLPPPRPSQEQAPEEPRSSADPFLETLTRLVRALRGPPAQASPARLALDPGALAGFPQGLVNLSDPAAQERLLNGGDEPLLLLLLPPATPTAAAAAAGDPAPPRGPASAPWAAGLARRVAAELQAAAAELRGLPGLPPAATPLLARLLALCPGDSGDSGDPGAPPGGPGGPLRALLLLKALQGLRAEWRGREQAGPARAQRSAGAGAADGPCALRELSVDLRAERSVLIPETYQANNCQGACGWPQSDRNPRYGNHVVLLLKMQARGAALARPPCCVPTAYAGKLLISLSEERISAHHVPNMVATECGCR

SEQ ID NO: 2 - protein, 572 aa, Canis lupus familiaris
MGALALWPLALALSGMGPLLGAEAPGGEVSGTPASPGEPATGTGGLLFQPDWDWPPSAPQDPLCLVTLDKGGNGSSPPLRVAGALRGYEHTFLEAVRRARWGPHDLATFGACAASDGRTTQLSLRQLQAWLGAPGGRRLVVLHLEEVTWEPALSLKFQEPPPGGASPLELALLVLYPGPGPEVAVTGAGLPGTQNLCRSRNTRYLVLALDHPVGAWHSPRVTLTVHARGDGAPLSTPQLQELLFGPDARCFTRMTPALLVLRLPGPTAVPARGLLDLVPFPPPRPSREPAEPPPSADPFLETLTRLVRALRGPPTPASPPRLALDPGALAGFPQGLLNLSDPATQERLLGGEEPLLLLLPPPTAAAGPPAPPPRPASAPWAAGLALRVAAELRAAAAELRGLPGLPPAAAPLLERLLALCPGGSGGSGGSGDPLRALLLLKALQGLRAEWRGRERGGPPRAQRSAGAGAADGPCALRELSVDLRAERSVLIPETYQANNCQGACGWPQSDRNPRYGNHVVLLLKMQARGAALARPPCCVPTAYGGKLLISLSEERISAHHVPNMVATECGCR

SEQ ID NO: 3 - protein, 575 aa, Artificial sequence
  Exemplary chimeric feline MIS amino acid sequence
MPGLLSPPALVLSVMGALLMAGDPGEEVSSTPALPGGPATGTGGLIFHPDWDWQPPGSPQDPLCLVTLDRGGNGSGSPLRVVGALRGYEHAFLEAVRRARWGPHGLATFGVCTPRDRQAAPFSLRQLQAWLGEPGGRRLVVLHLEEVTWEPTPSLKFQEPPPGGAGPLELAMLVLYPGPGPEVTVTGAGLPGTQSLCQSRDTRYLVLAVDHPEGAWRSPGLTLTLQPRRDGAPLSTAQLQELLFGPDPRCFTRMTPALLLLPGPAPAPLPARGLLDQVPLPPPRPSQEQAPEEPRSSADPFLETLTRLVRALRGPPAQASPARLALDPGALAGFPQGLVNLSDPAAQERLLNGGDEPLLLLLPPPTAAAGPPAPPPRPASAPWAAGLALRVAAELRAAAAELRGLPGLPPATAPLLERLLALCPGGSGGSGGSGDPLRALLLLKALQGLRAEWRGRERGGPPRAQRSAGAGAADGPCALRELSVDLRAERSVLIPETYQANNCQGACGWPQSDRNPRYGNHVVLLLKMQARGAALARPPCCVPTAYAGKLLISLSEERISAHHVPNMVATECGCR

SEQ ID NO: 4 - protein, 560 aa, Homo sapiens
MRDLPLTSLALVLSALGALLGTEALRAEEPAVGTSGLIFREDLDWPPGSPQEPLCLVALGGDSNGSSSPLRVVGALSAYEQAFLGAVQRARWGPRDLATFGVCNTGDRQAALPSLRRLGAWLRDPGGQRLVVLHLEEVTWEPTPSLRFQEPPPGGAGPPELALLVLYPGPGPEVTVTRAGLPGAQSLCPSRDTRYLVLAVDRPAGAWRGSGLALTLQPRGEDSRLSTARLQALLFGDDHRCFTRMTPALLLLPRSEPAPLPAHGQLDTVPFPPPRPSAELEESPPSADPFLETLTRLVRALRVPPARASAPRLALDPDALAGFPQGLVNLSDPAALERLLDGEEPLLLLLRPTAATTGDPAPLHDPTSAPWATALARRVAAELQAAAAELRSLPGLPPATAPLLARLLALCPGGPGGLGDPLRALLLLKALQGLRVEWRGRDPRGPGRAQRSAGATAADGPCALRELSVDLRAERSVLIPETYQANNCQGVCGWPQSDRNPRYGNHVVLLLKMQVRGAALARPPCCVPTAYAGKLLISLSEERISAHHVPNMVATECGCR

SEQ ID NO: 5 - DNA, 1767 bp, Felis silvestris catus
atgcctggcctgctgtcccctcccgccctggtgctgtccgtcatgggtgccctgctgatggctggagaccctggtgaagaagtgtcaagcaccccagccctgcctggaggacctgcaaccggcaccggaggcctgatcttccacccagactgggattggcagccacctggctctccacaggatccactgtgcctggtgaccctggacagaggaggaaacggatctggctccccactgagagtggtgggcgccctgaggggatacgagcacgcctttctggaggccgtgaggagagcaagatggggacctcacggcctggccaccttcggcgtgtgcacccctagggatagacaggccgccccattttccctgaggcagctgcaggcatggctgggagagccaggaggccggcgcctggtggtgctgcacctggaggaggtgacctgggagccaacccccagcctgaagttccaggagccaccacctggaggagcaggacccctggagctggccatgctggtgctgtaccctggaccaggaccagaggtgaccgtgaccggagcaggcctgcctggcacccagtctctgtgccagtccagggataccagatacctggtgctggccgtggaccacccagagggagcatggcgctcccctggcctgaccctgaccctgcagcctaggagagatggagcaccactgagcaccgcacagctgcaggagctgctgttcggacctgacccacgctgttttaccaggatgacccccgccctgctgctgctgccaggacctgcaccagcaccactgcctgccagaggcctgctggatcaggtgcccctgccaccacctagacctagccaggagcaggcacctgaggagccacggtccagcgccgaccctttcctggaaaccctgaccaggctggtgcgcgccctgaggggaccaccagcacaggcctctccagccagactggccctggatccaggcgccctggccggatttcctcagggcctggtgaacctgtccgatccagcagcacaggagcggctgctgaatggaggcgacgagccactgctgctgctgctgctgcctccagcaaccccaaccgccgccgcagcagcagcaggcgaccctgccccccctcggggcccagcctctgccccctgggccgccggcctggcccggcgcgtggccgccgagctgcaggccgccgccgccgagctgagaggcctgccaggcctgccacccgccgccacccccctgctggcccggctgctggccctgtgccctggcgactccggcgatagcggcgacccaggagcacctccaggaggaccaggaggccctctgcgcgccctgctgctgctgaaggccctgcagggcctgagggccgagtggcggggccgcgagcaggccggccctgccagagcacagcggtctgccggagcaggagcagcagatggcccatgtgcactgagggagctgagcgtggacctgagggcagagaggtctgtgctgatccccgaaacctaccaggccaacaattgccagggagcatgtggatggccacagtccgacagaaacccccggtacggcaatcacgtggtgctgctgctgaagatgcaggcaaggggagccgccctggccaggcccccttgctgcgtgccaaccgcatacgcaggcaagctgctgatcagcctgtctgaagaaagaatctctgctcatcacgtccccaatatggtcgccactgaatgcggttgtcggtga

SEQ ID NO: 6 - DNA, 1728 bp, Artificial sequence
  Nucleic acid sequence encoding an exemplary chimeric feline MIS
atgcctgggctgctgtctccacccgctctcgtcctgtccgtgatgggggctctcctgatggctggcgaccctggggaagaagtgtcctctacccccgccctgcctggaggaccagcaaccggcaccggaggcctgatcttccacccagactgggattggcagccacctggcagcccccaggaccctctgtgcctggtgaccctggataggggaggaaacggatctggcagcccactgagggtggtgggcgccctgagaggatacgagcacgccttcctggaggccgtgaggagagcaagatggggacctcacggcctggccaccttcggcgtgtgcaccccacgggaccgccaggcagcacctttctccctgagacagctgcaggcatggctgggagagccaggaggccggcgcctggtggtgctgcacctggaggaggtgacctgggagccaaccccttctctgaagttccaggagccaccacctggaggagcaggacccctggagctggcaatgctggtgctgtacccaggaccaggacctgaggtgaccgtgaccggagcaggcctgcctggcacccagagcctgtgccagtccagggacaccagatacctggtgctggcagtggatcacccagagggagcatggagatcccccggcctgaccctgaccctgcagccaaggagagacggagcacctctgtctaccgcacagctgcaggagctgctgttcggaccagatccaaggtgtttcaccaggatgacccccgccctgctgctgctgcctggaccagcaccagcacctctgccagcaaggggcctgctggaccaggtgccactgccaccacctcggccctctcaggagcaggcaccagaggagccacgcagctccgccgatcccttcctggaaaccctgaccagactggtgcgggccctgaggggaccaccagcacaggccagccctgcccggctggccctggacccaggcgccctggccggcttcccacagggcctggtgaacctgtccgacccagcagcacaggagcgcctgctgaacggaggcgatgagccactgctgctgctgctgcctccaccaaccgcagcagcaggacctccagcaccacctccaaggcctgccagcgccccatgggccgccggcctggccctgcgggtggccgccgagctgcgcgccgccgccgccgagctgcggggcctgcctggcctgccccctgccaccgccccactgctggagcgcctgctggccctgtgccccggcggcagcggcggctccggcggctctggcgaccctctgagggccctgctgctgctgaaggccctgcagggcctgagagcagagtggaggggaagagagcggggcggcccacccagggcccagagaagcgccggagcaggagcagcagacggaccttgtgccctgagagagctgtctgtggatctgagggcagagcgcagcgtgctgatcccagaaacctaccaggccaacaactgccagggagcatgtggatggcctcagtccgataggaacccaagatacggcaaccacgtggtgctgctgctgaagatgcaggcaaggggagccgccctggccagacctccatgctgcgtgccaaccgcatacgcaggcaagctgctgatctccctgtctgaggaaagaatctctgctcatcacgtcccaaacatggtcgcaaccgagtgtggctgtcgctga

SEQ ID NO: 7 - protein, 554 aa, Artificial sequence
  Exemplary recombinant modified human MIS (LRhMIS) protein
MKWVTFISLLFLFSSAYSLRAEEPAVGTSGLIFREDLDWPPGSPQEPLCLVALGGDSNGSSSPLRVVGALSAYEQAFLGAVQRARWGPRDLATFGVCNTGDRQAALPSLRRLGAWLRDPGGQRLVVLHLEEVTWEPTPSLRFQEPPPGGAGPPELALLVLYPGPGPEVTVTRAGLPGAQSLCPSRDTRYLVLAVDRPAGAWRGSGLALTLQPRGEDSRLSTARLQALLFGDDHRCFTRMTPALLLLPRSEPAPLPAHGQLDTVPFPPPRPSAELEESPPSADPFLETLTRLVRALRVPPARASAPRLALDPDALAGFPQGLVNLSDPAALERLLDGEEPLLLLLRPTAATTGDPAPLHDPTSAPWATALARRVAAELQAAAAELRSLPGLPPATAPLLARLLALCPGGPGGLGDPLRALLLLKALQGLRVEWRGRDPRGPGRARRSAGATAADGPCALRELSVDLRAERSVLIPETYQANNCQGVCGWPQSDRNPRYGNHVVLLLKMQVRGAALARPPCCVPTAYAGKLLISLSEERISAHHVPNMVATECGCR

SEQ ID NO: 8 - DNA, 1680 bp, Artificial sequence
  Nucleic acid encoding an exemplary recombinant modified human (hMIS) protein of SEQ ID NO 7
atgaagtgggtgagcttcatcagcctgctgttcctgttcagcagcgcttactcccgcggtgtgttccgccgcagagcagaggagccagctgtgggcaccagtggcctcatcttccgagaagacttggactggcctccaggcagcccacaagagcctctgtgcctggtggcactgggcggggacagcaatggcagcagctcccccctgcgggtggtgggggctctaagcgcctatgagcaggccttcctgggggccgtgcagagggcccgctggggcccccgagacctggccaccttcggggtctgcaacaccggtgacaggcaggctgccttgccctctctacggcggctgggggcctggctgcgggaccctggggggcagcgcctggtggtcctacacctggaggaagtgacctgggagccaacaccctcgctgaggttccaggagcccccgcctggaggagctggccccccagagctggcgctgctggtgctgtaccctgggcctggccctgaggtcactgtgacgagggctgggctgccgggtgcccagagcctctgcccctcccgagacacccgctacctggtgttagcggtggaccgccctgcgggggcctggcgcggctccgggctggccttgaccctgcagccccgcggagaggactcccggctgagtaccgcccggctgcaggcactgctgttcggcgacgaccaccgctgcttcacacggatgaccccggccctgctcctgctgccgcggtccgagcccgcgccgctgcctgcgcacggccagctggacaccgtgcccttcccgccgcccaggccatccgcggaactcgaggagtcgccacccagcgcagaccccttcctggagacgctcacgcgcctggtgcgggcgctgcgggtccccccggcccgggcctccgcgccgcgcctggccctggatccggacgcgctggccggcttcccgcagggcctagtcaacctgtcggaccccgcggcgctggagcgcctactcgacggcgaggagccgctgctgctgctgctgaggcccactgcggccaccaccggggatcctgcgcccctgcacgaccccacgtcggcgccgtgggccacggccctggcgcgccgcgtggctgctgaactgcaagcggcggctgccgagctgcgaagcctcccgggtctgcctccggccacagccccgctgctggcgcgcctgctcgcgctctgcccaggtggccccggcggcctcggcgatcccctgcgagcgctgctgctcctgaaggcgctgcagggcctgcgcgtggagtggcgcgggcgggatccgcgcgggccgggtcgggcacggcgcagcgcgggggccaccgccgccgacgggccgtgcgcgctgcgcgagctcagcgtagacctccgcgccgagcgctccgtactcatccccgagacctaccaggccaacaattgccagggcgtgtgcggctggcctcagtccgaccgcaacccgcgctacggcaaccacgtggtgctgctgctgaagatgcaggcccgtggggccgccctggcgcgcccaccctgctgcgtgcccaccgcctacgcgggcaagctgctcatcagcctgtcggaggagcgcatcagcgcgcaccacgtgcccaacatggtggccaccgagtgtggctgccggtga

SEQ ID NO: 9 - protein, 19 aa, Artificial sequence
  Exemplary leader sequence
MTRLTVLALLAGLLASSRA

SEQ ID NO: 10 - protein, 18 aa, Artificial sequence
  Exemplary leader sequence
MKWVTFISLLFLFSSAYS

SEQ ID NO: 11 - protein, 24 aa, Artificial sequence
  Exemplary leader sequence
MKWVTFISLLFLFSSAYSRGVFRR

SEQ ID NO: 12 - protein, 24 aa, Artificial sequence
  Exemplary leader sequence
MKWVTFISLLFLFSSAYSRGVFRR

SEQ ID NO: 13 - protein, 18 aa, Artificial sequence
  Exemplary leader sequence
MKWVSFISLLFLFSSAYS

SEQ ID NO: 14 - protein, 585 aa, Artificial sequence
  Exemplary LR-fcMISv2 amino acid sequence
MKWVTFISLLLLFSSAYSGDPGEEVSSTPALPGGPATGTGGLIFHPDWDWQPPGSPQDPLCLVTLDRGGNGSGSPLRVVGALRGYEHAFLEAVRRARWGPHGLATFGVCTPRDRQAAPFSLRQLQAWLGEPGGRRLVVLHLEEVTWEPTPSLKFQEPPPGGAGPLELAMLVLYPGPGPEVTVTGAGLPGTQSLCQSRDTRYLVLAVDHPEGAWRSPGLTLTLQPRRDGAPLSTAQLQELLFGPDPRCFTRMTPALLLLPGPAPAPLPARGLLDQVPLPPPRPSQEQAPEEPRSSADPFLETLTRLVRALRGPPAQASPARLALDPGALAGFPQGLVNLSDPAAQERLLNGGDEPLLLLLLPPATPTAAAAAAGDPAPPRGPASAPWAAGLARRVAAELQAAAAELRGLPGLPPAATPLLARLLALCPGDSGDSGDPGAPPGGPGGPLRALLLLKALQGLRAEWRGREQAGPARARRSAGAGAADGPCALRELSVDLRAERSVLIPETYQANNCQGACGWPQSDRNPRYGNHVVLLLKMQARGAALARPPCCVPTAYAGKLLISLSEERISAHHVPNMVATECGCR

SEQ ID NO: 15 - protein, 568 aa, Artificial sequence
  Exemplary LR-clMIS
MKWVTFISLFFLFSSAYSEAPGGEVSGTPASPGEPATGTGGLLFQPDWDWPPSAPQDPLCLVTLDKGGNGSSPPLRVAGALRGYEHTFLEAVRRARWGPHDLATFGACAASDGRTTQLSLRQLQAWLGAPGGRRLVVLHLEEVTWEPALSLKFQEPPPGGASPLELALLVLYPGPGPEVAVTGAGLPGTQNLCRSRNTRYLVLALDHPVGAWHSPRVTLTVHARGDGAPLSTPQLQELLFGPDARCFTRMTPALLVLRLPGPTAVPARGLLDLVPFPPPRPSREPAEPPPSADPFLETLTRLVRALRGPPTPASPPRLALDPGALAGFPQGLLNLSDPATQERLLGGEEPLLLLLPPPTAAAGPPAPPPRPASAPWAAGLALRVAAELRAAAAELRGLPGLPPAAAPLLERLLALCPGGSGGSGGSGDPLRALLLLKALQGLRAEWRGRERGGPPRARRSAGAGAADGPCALRELSVDLRAERSVLIPETYQANNCQGACGWPQSDRNPRYGNHVVLLLKMQARGAALARPPCCVPTAYGGKLLISLSEERISAHHVPNMVATECGCR

SEQ ID NO: 16 - DNA, 1713 bp, Artificial sequence
  Exemplary nucleic acid encoding LR-clMIS
atgaagtgggtgaccttcatcagcctattcttcctgttcagcagcgcctacagcgaagcacctggcggcgaggtttccggaacacccgcttcacctggcgaaccagcaactggaacaggaggcctactattccagccagactgggattggcccccttcagccccccaagaccctctatgcctagtaaccctagataaaggaggcaacggctcttccccacccctacgagtggcaggagccctacgaggctacgaacacacattcctagaggccgtacgacgagctcgatggggaccccacgacctagcaacctttggcgcctgcgccgcttctgatggacgaacaacccagctatccctacgacagctacaagcttggctaggagcacctggaggccgacgactagtggtactacacctagaagaggtgacatgggaacccgccctatctctaaagtttcaggagcctccaccaggaggagcctcccctctagaactagctctactagtactataccctggaccaggaccagaggtggcagtaaccggagctggcctaccaggcactcaaaacctatgccgatctcgaaacacccgatacctcgtgctcgctctagaccaccctgtaggagcatggcactcaccacgagtgaccctaactgtacacgcacgaggagacggcgccccactatctaccccccagctacaagagctactattcggccccgatgcccgatgctttactcgaatgacacctgctctactagtgctacgactacctggcccaactgctgtacctgcacgaggactactagacctagtgccattccctccaccccgaccttcccgagaaccagcagagcctccaccctcagccgatccatttctagaaacactaacccgactagtgcgagccctacgaggacctccaacacccgcttccccccctcgactagcactagaccccggagctctagcaggcttccctcagggactactaaacctatcagatccagctactcaagagcgactactaggaggcgaagagcccctactactactactaccaccccctacagcagccgctggaccaccagctcctccaccccgacccgcctccgctccttgggcagccggactagcactacgagtagctgcagaactacgagccgctgcagccgagctacgaggactaccaggcctacctccagctgcagcccccctactagaacgactactagccctatgccctggaggatccggaggatcaggaggatctggcgacccactacgagccctactactactaaaagctctacagggcctacgagcagaatggcgaggacgagagagaggaggaccacctcgagcacggcgatctgcaggagcaggagctgcagacggaccttgcgcactacgagagctatcagtggatctacgagccgaacgatctgtactaatcccagagacttaccaggccaacaactgccaaggagcttgcggctggccacagtccgatcgaaacccccgatatggcaaccacgtggtactactactaaaaatgcaagcacgaggagccgctctagcccgaccaccatgctgcgtgcctaccgcatatggaggcaagctactaatttcactatctgaagaacggatctccgcacatcacgtacctaatatggtagctactgagtgtggttgtagatgaggtacc

SEQ ID NO: 17 - DNA, 1725 bp, Canis lupus familiaris
atgggcgcattagcactttggcctttagccttagcactatcaggaatgggaccactactgggagcagaagcacctggcggcgaggtttccggaacacccgcttcacctggcgaaccagcaactggaacaggaggcctactattccagccagactgggattggcccccttcagccccccaagaccctctatgcctagtaaccctagataaaggaggcaacggctcttccccacccctacgagtggcaggagccctacgaggctacgaacacacattcctagaggccgtacgacgagctcgatggggaccccacgacctagcaacctttggcgcctgcgccgcttctgatggacgaacaacccagctatccctacgacagctacaagcttggctaggagcacctggaggccgacgactagtggtactacacctagaagaggtgacatgggaacccgccctatctctaaagtttcaggagcctccaccaggaggagcctcccctctagaactagctctactagtactataccctggaccaggaccagaggtggcagtaaccggagctggcctaccaggcactcaaaacctatgccgatctcgaaacacccgatacctcgtgctcgctctagaccaccctgtaggagcatggcactcaccacgagtgaccctaactgtacacgcacgaggagacggcgccccactatctaccccccagctacaagagctactattcggccccgatgcccgatgctttactcgaatgacacctgctctactagtgctacgactacctggcccaactgctgtacctgcacgaggactactagacctagtgccattccctccaccccgaccttcccgagaaccagcagagcctccaccctcagccgatccatttctagaaacactaacccgactagtgcgagccctacgaggacctccaacacccgcttccccccctcgactagcactagaccccggagctctagcaggcttccctcagggactactaaacctatcagatccagctactcaagagcgactactaggaggcgaagagcccctactactactactaccaccccctacagcagccgctggaccaccagctcctccaccccgacccgcctccgctccttgggcagccggactagcactacgagtagctgcagaactacgagccgctgcagccgagctacgaggactaccaggcctacctccagctgcagcccccctactagaacgactactagccctatgccctggaggatccggaggatcaggaggatctggcgacccactacgagccctactactactaaaagctctacagggcctacgagcagaatggcgaggacgagagagaggaggaccacctcgagcacaacgatctgcaggagcaggagctgcagacggaccttgcgcactacgagagctatcagtggatctacgagccgaacgatctgtactaatcccagagacttaccaggccaacaactgccaaggagcttgcggctggccacagtccgatcgaaacccccgatatggcaaccacgtggtactactactaaaaatgcaagcacgaggagccgctctagcccgaccaccatgctgcgtgcctaccgcatatggaggcaagctactaatttcactatctgaagaacggatctccgcacatcacgtacctaatatggtagctactgagtgtggttgtagatgaggtacc

SEQ ID NO: 18 - protein, 589 aa, Felis silvestris catus
MPGLLSPPALVLSVMGALLMAGDPGEEVSSTPALPGGPATGTGGLIFHPDWDWQPPGSPQDPLCLVTLDRGGNGSGSPLRVVGALRGYEHAFLEAVRRARWGPHGLATFGVCTPRDRQAAPFSLRQLQAWLGEPGGRRLVVLHLEEVTWEPTPSLKFQEPPPGGAGPLELAMLVLYPGPGPEVTVTGAGLPGTQSLCQSRDTRYLVLAVDHPEGAWRSPGLTLTLQPRRDGAPLSTAQLQELLFGPDPRCFTRMTPALLLLPGPAPAPLPARGLLDQVPLPPPRPSQEQAPEEPRSSADPFLETLTRLVRALRGPPAQASPARLALDPGALAGFPQGLVNLSDPAAQERLLNGGDEPLLLLLLPPATPTAAAAAAAGDPAPPRGPASAPWAAGLARRVAAELQAAAAELRGLPGLPPAATPLLARLLALCPGDSGDSGDPGAPPGGPGGPLRALLLLKALQGLRAEWRGREQGGPARAQRSAGAGAADGPCALRELSVDLRAERSVLIPETYQANNCQGACGWPQSDRNPRYGNHVVLLLKMQARGAALARPPCCVPTAYAGKLLISLSEERISAHHVPNMVATECGCR

SEQ ID NO: 19 - DNA, 3423 bp, Felis silvestris catus
cggtgtccctgtgtcctcccaggatgccggtgggtgaaggacagacccaggcagtcagcagcgggtctgggctctcttctgcggccgctcaccctccttggggtctccagccaaatggctgtgcttctgcagctccagggtgccaagaggcagtgtgagcgagcacttggggagccccagcttcgctgggagaccaccctctggccaagcccacgtgcacccagcggtctgaacaccaagatctccggtccccatcagagccgggtgccggaggcctccagggcgcctgcccccctgcccccattccagagctgttgatcgccggtctggttcccactccctcctgcagagggggaaaacctcatcaaggacagtcgttggaccaactgggcacgggcggcactctgtatcacgggtaggaagataggcggtcaggctggaacagaagaggctttgagaggctcctctgcctgcccaggcccacggcggggcaccagacgttggcccccaaggtcacaccccagaaggagataggggctttgctcctgcacaaacatcccggtctcctccatataagccagagccacacggcccctcacagcagccaggatgcctggtctgctctctccgccggccctggtgctgtcggtgatgggggctctgctgatggccggggaccctggggaagaggtctccagcaccccggccctgcctggagggccagccacaggcaccgggggtctcatcttccacccggattgggactggcagcccccgggcagtccccaagaccccctgtgcctggtgacgctggacaggggtggtaacgggagcggctccccgcttcgggtggtgggggcgctgagaggctacgagcacgccttcctcgaggctgtgcggcgggcccgctggggtccccacggcctggccaccttcggagtttgcacccccagggacaggcaggccgccccgttctctctgcggcagctgcaggcgtggctgggggagcccggggggcggcggctggtggtgctgcacctggaggaaggtacgtagggagggggccacggcctgggggggtgccacgctgccaccactctttccagaccgggtcctgccggagcccaactctagacgcatcttggcctccggggaaggctgaggctgagggcccaggaagtgggggcccttcgtgtctgggggctgcaaaccccccgatgctttgcttccaagcccacccctcctgcagccctcctgggaggtgtttgccgccccccccaccccacgaaggagcagaagggagtccgggcagagccatgctccgcccacacccctcctccaagggtggttagtgtgcggcctgttcccggacgctgctggaggaaatggtttcccaggggaccctatgggctcccccgctaaggagagccccagacagagaccccatggggtgctccaggctcctcgcattggggcaaggccctgcacccgatgtctgggcgtcaagcctctcccggtacgggtgggggctctccctgcaggactgcaagacgggcttcggggaggtgctggggcctcggtgactcaggtgttgccccttccctatttgtccctcctggccacagtgacatgggagccgacaccctcactgaagttccaggagcccccgcctggaggggccggtcccctagagctggcgatgctggtgctgtaccccggccctggccccgaggtcacggtcacaggggctgggctgccaggcacccaggtaccggggtgttgaaggcggttagttctggggcctccaggagccctcccccaacagaggagggggaatggggttttttaactgtgctgaacagaaaggttccaagtccgctggttggaaccttgaaggggggtgtcaagggcaatgggcagagcagggctggtgtcctcgcccccccccccccacctggctaggctgagcccccgtctccacagagcctctgtcagtcccgggacacccgctacctggtgctggcggtggaccacccagaaggggcctggcgcagccccgggctcaccctgaccctgcaaccccgcagagacggtaggctctccaagagagggaccgggtaagggtgggcggcccgcggtcctccgcccccgctcagccaggcccccgtgctccgccacgcaggtgcgcccctgagcaccgcccagctgcaggagctgctgttcggccccgacccccgctgcttcacacgcatgaccccggcgctgctcctgctgccggggcccgcgcctgcaccgctgcccgcgcgtggcctgctggaccaagtgcctctcccgccgcccaggtgtgcgcaggcccacgttgggggtgtcgggaggggaaccctgcacctgcccctaccgcccaactccgccttccaggccctcccaggagcaggcgcctgaggagccacggtccagcgccgaccccttcctggagacgctcacgcgcctggtgcgcgcgctgcgggggcccccggcccaggcctcgccggcgcgcctggccctggaccccggcgcgctggccggcttcccgcagggcctggtcaacctgtcggaccccgcggcgcaggagcgcctgctcaacggcggcgacgagccgctgctgctgcttctgctgccacccgccacgcccaccgccgccgccgccgccgccgccggggaccccgcgccgccgcgcggcccggcgtccgcgccctgggccgccggcctagcgcgtcgcgtggccgccgagctgcaggccgcggccgccgagctgcgagggctcccggggctgccgccggccgccacgccgctgctggcgcgcctgctcgcgctgtgccccggggattcgggggactcgggggacccgggggccccccccggcggcccgggcggcccgctgcgcgcgctgctgctgctcaaggcgctgcagggtctgcgcgcggagtggcgcgggcgcgagcagggcggaccggcgcgggcacagcgcagcgcgggggccggggcggccgacgggccgtgcgcgctgcgcgagctgagcgtggacctgcgcgccgagcgctccgtgctcatcccggagacgtaccaggccaacaactgccagggcgcgtgcggctggccgcagtccgaccgcaacccgcgctacggcaaccacgtggtgctgctgctcaagatgcaggcccgcggcgccgccctggcgcgcccgccctgctgcgtgcccacggcctacgcgggcaagctcctcatcagcctgtcggaggagcgcatcagcgcgcaccacgtgcccaacatggtggccaccgagtgcggctgccggtgagccccgcaccgtgccccccgagtggcgtccccgcccgtatttattcggacccccatcatcgccccaataaagaccagcaagcacag

SEQ ID NO: 20 - protein, 586 aa, Artificial Sequence
  Exemplary LR-fcMIS-2 amino acid sequence
MKWVTFISLLLLFSSAYSGDPGEEVSSTPALPGGPATGTGGLIFHPDWDWQPPGSPQDPLCLVTLDRGGNGSGSPLRVVGALRGYEHAFLEAVRRARWGPHGLATFGVCTPRDRQAAPFSLRQLQAWLGEPGGRRLVVLHLEEVTWEPTPSLKFQEPPPGGAGPLELAMLVLYPGPGPEVTVTGAGLPGTQSLCQSRDTRYLVLAVDHPEGAWRSPGLTLTLQPRRDGAPLSTAQLQELLFGPDPRCFTRMTPALLLLPGPAPAPLPARGLLDQVPLPPPRPSQEQAPEEPRSSADPFLETLTRLVRALRGPPAQASPARLALDPGALAGFPQGLVNLSDPAAQERLLNGGDEPLLLLLLPPATPTAAAAAAAGDPAPPRGPASAPWAAGLARRVAAELQAAAAELRGLPGLPPAATPLLARLLALCPGDSGDSGDPGAPPGGPGGPLRALLLLKALQGLRAEWRGREQGGPARARRSAGAGAADGPCALRELSVDLRAERSVLIPETYQANNCQGACGWPQSDRNPRYGNHVVLLLKMQARGAALARPPCCVPTAYAGKLLISLSEERISAHHVPNMVATECGCR

(19) United States
(12) Patent Application Publication
(10) Pub. No.: US 2024/0218038 A1
(43) Pub. Date: Jul. 4, 2024

(54) COMPOSITIONS AND METHODS FOR PREVENTING OR DELAYING PUBERTY IN PREPUBESCENT NON-HUMAN ANIMALS AND HUMANS

(71) Applicant: The General Hospital Corporation, Boston, MA (US)

(72) Inventors: Patricia K. DONAHOE, Boston, MA (US); David PEPIN, Boston, MA (US)

(73) Assignee: The General Hospital Corporation, Boston, MA (US)

(21) Appl. No.: 18/283,556

(22) PCT Filed: Mar. 22, 2022
(86) PCT No.: PCT/US22/21312

§ 371 Date: Sep. 22, 2023

Related U.S. Application Data
(60) Provisional application No. 63/164,254, filed on Mar. 22, 2021.
(60) Provisional application No. 63/197,061, filed on Jun. 4, 2021.

(51) Int. Cl. C07K 14/575; A01K 67/0278; A61K 38/00; A61P 15/00; C12N 15/86; C12N 15/88; G01N 33/68

(52) U.S. Cl. CPC C07K 14/575; A01K 67/0278; A61P 15/00; C12N 15/86; C12N 15/88; G01N 33/6854; A01K 2217/072; A01K 2227/10; A01K 2267/01; A61K 38/00; C12N 2750/14143; G01N 2333/575

(57) ABSTRACT

The present invention relates to compositions and methods of reducing fertility and/or preventing puberty in prepubescent non-human subjects (e.g., kittens and puppies) by administering a composition comprising a vector comprising a nucleic acid encoding a Mullerian Inhibiting Substance (MIS) operatively linked to one or more regulatory elements. In some embodiments, administration is via a single or multiple injection. Other aspects relate to method and compositions for delaying puberty in a human female subject by administering a recombinant human MIS variant protein.

### CROSS-REFERENCE TO RELATED APPLICATIONS

This application claims the benefit of priority of U.S. Provisional Application No. 63/164,254, filed, Mar. 22, 2021, and U.S. Provisional Application No. 63/197,061, filed Jun. 4, 2021, the contents of which are incorporated by reference herein in their entireties for any purpose.

### SEQUENCE LISTING

The present application is being filed along with a Sequence Listing in electronic format. The Sequence Listing is provided as a file entitled 2022-03-03_01133-0001-OOPCT_Seq_List_ST25 created Mar. 4, 2022, which is 62 Kb in size. The information in the electronic format of the sequence listing is incorporated herein by reference in its entirety.

### FIELD

This application relates to compositions and methods of administering MIS proteins, and vectors comprising nucleic acids encoding MIS proteins for reducing fertility and/or preventing, or delaying puberty in prepubescent non-human subjects (e.g., kittens and puppies) and human subjects.

### BACKGROUND

Müllerian Inhibiting Substance (MIS) also known as anti-Müllerian hormone (AMH), is a 140-kDa disulfide-linked homodimer glycoprotein member of the large transforming growth factor-β (TGFβ) multigene family of glycoproteins. The proteins in this gene family are all produced as dimeric precursors and undergo posttranslational processing for activation, requiring cleavage and dissociation to release bioactive C-terminal fragments. Similarly, the 140 kilodalton (kDa) disulfide-linked homodimer of MIS is proteolytically cleaved to generate its active C-terminal fragments.

MIS is a reproductive hormone produced in fetal testes, which inhibits the development of female secondary sexual structures in males. Before sexual differentiation, the fetus is bipotential, and the developmental choice of male Wolffian ducts (i.e., prostate, vas deferens) over female Müllerian ducts (i.e., Fallopian tubes, uterus, vagina) in the male is controlled in part by MIS.

The human MIS gene is located on chromosome 19, and its expression is sexually dimorphic. In males, MIS expression begins at 9 weeks gestation in the fetal testes and continues at high levels until puberty, when expression levels fall dramatically. In females, MIS is produced only postnatally in granulosa cells from prepuberty through menopause at levels similar to adult males, after which expression ceases. In male fetuses MIS causes regression of the Müllerian ducts, the precursors to the Fallopian tubes, uterus, cervix, and upper third of the vagina.

Endogenously, MIS is produced by the granulosa cells and is an important gatekeeper of primordial follicle recruitment into the growing pool. In females, MIS is expressed by the granulosa cells of the ovary after sexual differentiation of the Müllerian duct. Because of the correlation between MIS production and the number of growing follicles, and its steady secretion throughout the ovarian cycle, MIS is used clinically to estimate the size of the ovarian reserve (the total pool of follicles) (Kalaiselvi et al., 2012). In males, overexpression of MIS inhibits steroidogenesis in Leydig cells, causing a marked drop in testosterone levels (Teixeira et al, 1999).

It was previously discovered that the human MIS protein arrests folliculogenesis at the initial stage of primordial follicle development. It was previously demonstrated that in a mouse model of fertility, administration of human MIS protein to sexually mature female mice, e.g., via gene transfer, can prevent follicle maturation and oocyte release, thus inhibiting ovulation. It was further demonstrated that administering human MIS protein to sexually mature female mice resulted in significantly reduced reproduction rates. (WO2015/089321.)

Currently, it is recommended that pet owners and shelters spay or neuter animals, including cats and dogs. Surgical spaying or neutering young animals is considered a responsible way to care for animals. In females, it typically involves abdominal surgery to remove one or both ovaries and/or uterus. In males, it typically involves surgical removal of the testes. Spaying and neutering is encouraged (and in some countries required for adopted animals) to prevent the births of unwanted litters, which contribute to the overpopulation of unwanted animals in the rescue system. The ASPCA (American Society for the Prevention of Cruelty to Animals©) has indicated the pet homelessness problem results in millions of healthy dogs and cats being euthanized in the United States each year. In addition, the ACPCA also indicates there are medical and behavioral benefits to spaying and neutering animals including: preventing certain infections or tumors (e.g., uterine infections and mammary tumors in females and testicular cancer and prostate problems in males); avoiding female pets going into heat; making it less likely for male pets to roam; and may lead to better behaved males. Spay and neutering are common surgeries, but there can be risks, for example with general anesthesia. Additionally, it takes time for the animals to heal after the surgery. Accordingly, bathing must be avoided for, e.g., at least ten days after surgery; the animal must refrain from running or jumping post-surgery; and the incision site must be monitored to avoid infection and proper healing.

Kittens and puppies are more desirable for adoption than adult cats and dogs. Animal shelters currently surgically spay or neuter kittens and puppies as early as 8 weeks of age, prior to adoption. Recommendations suggest that female kittens should be surgically spayed before they reach approximately 5 months of age and female puppies before they reach approximately 6 months of age. Accordingly, there is a need for reducing fertility and/or preventing puberty in prepubescent animals, including kittens and puppies, and a simple method to achieve long term infertility can serve as an alternative to spaying or neutering.

### SUMMARY OF THE INVENTION

In accordance with the present invention, one aspect of the technology disclosed herein relates to compositions comprising a vector comprising a nucleic acid encoding a Müllerian Inhibiting Substance (MIS) protein and methods of reducing fertility and/or preventing or delaying puberty in a pre-pubescent subject (e.g., a kitten or a puppy, or a human subject) comprising administering to the subject an effective amount of the composition are provided.

Accordingly, one aspect of the present invention provides a method of reducing fertility and/or preventing puberty in prepubescent animals, including kittens and puppies, by administration of MIS protein, e.g., via gene transfer. If prepubescent animals, including kittens and puppies, are administered MIS prior to puberty, the single treatment may prevent them from entering puberty or reduce their fertility. In prepubescent animals, including kittens and puppies, long term infertility can serve as an alternative to surgical spaying or neutering. Accordingly, one aspect of the present invention relates to compositions and methods of administering MIS proteins (e.g., by viral vectors encoding MIS proteins) for reducing fertility and/or preventing puberty in prepubescent non-human subjects, such as kittens and puppies.

Another aspect of the technology described herein relates to a method of reducing fertility in a prepubescent non-human subject comprising administering to the subject an effective amount of a composition comprising a recombinant feline or canine MIS protein as disclosed herein, or a vector comprising a nucleic acid encoding a recombinant feline or canine MIS protein operatively linked to one or more regulatory elements.

Another aspect of the technology described herein relates to a method of preventing puberty in a prepubescent non-human subject comprising administering to the subject an effective amount of a composition comprising a vector comprising a nucleic acid encoding a MIS protein operatively linked to one or more regulatory elements.

Another aspect of the technology described herein relates to a method of delaying puberty in a prepubescent human subject, e.g., a female human subject, comprising administering to the subject an effective amount of a composition comprising a recombinant human MIS (rhMIS) protein as disclosed herein. In some embodiments, the method is reversibly delaying puberty in a prepubescent human subject. In some embodiments, the recombinant human MIS protein is a mature protein produced from a pre-protein from the human MIS protein comprising a change in at least amino acid 450 of SEQ ID NO: 4 from a Q to a R, or a conservative amino acid of R (Q450R). In some embodiments, the conservative amino acid of R is K. Such a method for delaying puberty in a human female subject is useful, for example, when a subject is in need of delaying puberty, for example, in order to provide the subject more time before beginning a gender reassignment treatment and/or surgery, or before the female subject begins treatment to transition from a female to a male gender. In some embodiments, a method for delaying puberty in a human female subject is useful, for example, where the subject has a disease or disorder associated with reduce bone growth and where puberty will stop bones from growing. In some embodiments, a method for delaying puberty in a human female subject is useful, for example, where the subject has idiopathic precocious puberty. In some embodiments, a method for delaying puberty in a human female subject is useful, for example, where the subject has a difference in sexual development, and delaying puberty by administering a modified hMIS protein is useful to allow gender affirming treatments to be administered to the subject prior to, or before puberty changes secondary sexual characteristics.

In some embodiments, the disclosure also provides a composition comprising a modified feline MIS protein, wherein the modified feline MIS protein is a chimeric feline MIS protein comprising an amino acid sequence of SEQ ID NO: 3, or a recombinant feline MIS protein having at least 80%, at least 85%, at least 90%, at least 95%, at least 96%, at least 97%, at least 98%, or at least 99% sequence identity to amino acids 22-575 sequence of SEQ ID NO: 1.

In some embodiments, the disclosure also provides a composition comprising a nucleic acid encoding a chimeric feline MIS protein comprising an amino acid sequence of SEQ ID NO: 3, wherein the nucleic acid has at least 80%, at least 85%, at least 90%, at least 95%, at least 96%, at least 97%, at least 98%, or at least 99% sequence identity to the nucleic acid sequence of SEQ ID NO: 6.

In some embodiments, the disclosure also provides a composition comprising a nucleic acid encoding a modified canine MIS protein (clMIS) comprising an amino acid sequence of SEQ ID NO: 15, wherein the nucleic acid has at least 80%, at least 85%, at least 90%, at least 95%, at least 96%, at least 97%, at least 98%, or at least 99% sequence identity to the nucleic acid sequence of SEQ ID NO: 16.

In some embodiments, the prepubescent non-human subject is a kitten or a puppy.

In some embodiments, the prepubescent non-human subject is female. In some embodiments, the prepubescent non-human subject is male.

In some embodiments, the MIS protein comprises:
a) a wild-type feline MIS protein, the amino acid sequence of SEQ ID NO: 1 or SEQ ID NO: 18, or an amino acid sequence having at least 80%, at least 85%, at least 90%, at least 95%, at least 96%, at least 97%, at least 98%, or at least 99% sequence identity to the amino acid sequence of SEQ ID NO: 1 or SEQ ID NO: 18;
b) a wild-type canine MIS protein, the amino acid sequence of SEQ ID NO: 2, or an amino acid sequence having at least 80%, at least 85%, at least 90%, at least 95%, at least 96%, at least 97%, at least 98%, or at least 99% sequence identity to the amino acid sequence of SEQ ID NO: 2; or
c) a wild-type human MIS protein, the amino acid sequence of SEQ ID NO: 4, or an amino acid sequence having at least 80%, at least 85%, at least 90%, at least 95%, at least 96%, at least 97%, at least 98%, or at least 99% sequence identity to the amino acid sequence of SEQ ID NO: 4.

In some embodiments, the prepubescent non-human subject is a kitten that is 12 months old or less, 11 months old or less, 10 months old or less, 9 months old or less, 8 months old or less, 7 months old or less, 6 months old or less, 5 months old or less, 4 months old or less, 3 months old or less, or 2 months old or less. In some embodiments, the prepubescent non-human subject is a kitten weighing 2 kg or less.

In some embodiments, the prepubescent non-human subject is a puppy that is 24 months old or less, 22 months old or less, 20 months old or less, 18 months old or less, 16 months old or less, 14 months old or less, 12 months old or less, 11 months old or less, 10 months old or less, 9 months old or less, 8 months old or less, 7 months old or less, 6 months old or less, 5 months old or less, 4 months old or less, 3 months old or less, or 2 months old or less.

In some embodiments, the prepubescent non-human subject has been weaned. In some embodiments, the prepubescent non-human subject has not been weaned.

In some embodiments, the vector is a viral vector, a plasmid, a cosmid, or a phagemid. In some embodiments, the vector is a viral vector. In some embodiments, the composition further comprises a cell comprising the vector.

In some embodiments, the composition comprises a sterile, injectable solution. In some embodiments, the composition comprises an aqueous, sterile, injectable solution. In some embodiments, the composition comprises a lipid, lipid emulsion, liposome, nanoparticle, or exosomes.

In some embodiments, the vector is an adenoviral vector, an adeno-associated virus (AAV) vector, a poxvirus vector, or a lentiviral vector. In some embodiments, the vector is an AAV vector. In some embodiments, the vector is an AAV9 vector.

In some embodiments, the one or more regulatory elements comprise a promoter element. In some embodiments, the one or more regulatory elements comprise a promoter element and an enhancer element. In some embodiments, the one or more regulatory elements comprise a constitutively active promoter.

In some embodiments, the composition comprises a pharmaceutically acceptable carrier.

In some embodiments, the administering is via injection. In some embodiments, the administering is via intravenous, subcutaneous, or intramuscular administration. In some embodiments, the administering is via intramuscular administration. In some embodiments, the administering is via a single injection. In some embodiments, the administering is via a single one-time injection. In some embodiments, the administering is via a single dose split into multiple injections. In some embodiments, the administering is via a single dose split into two injections.

In some embodiments, the effective amount of the composition administered to the prepubescent non-human subject is 1×10¹³ vector genomes or less, 5×10¹² vector genomes or less, 1×10¹² vector genomes or less, 5×10¹¹ vector genomes or less, or 1×10¹¹ vector genomes or less per kilogram weight of the subject.

In some embodiments, the concentration of MIS protein in the serum of the prepubescent non-human subject at or after 6 months, at or after 9 months, at or after 12 months, at or after 15 months, or at or after 24 months following administration of the composition is greater than 250 ng/ml, greater than 300 ng/ml, greater than 400 ng/ml, greater than 500 ng/ml, greater than 600 ng/ml, greater than 700 ng/ml, greater than 800 ng/ml, greater than 900 ng/ml, greater than 1 μg/ml, greater than 1.5 μg/ml, greater than 2 μg/ml, greater than 3 μg/ml, greater than 4 μg/ml, greater than 5 μg/ml, greater than 6 μg/ml, greater than 7 μg/ml, greater than 8 g/ml, greater than 9 μg/ml, greater than 10 μg/ml, or greater than 11 μg/ml. In some embodiments, the MIS protein concentration is determined by ELISA.

In some embodiments, the prepubescent non-human subject is female and following administration of the composition, (a) does not develop follicles, (b) does not develop follicles with viable eggs, (c) does not experience puberty, (d) does not show signs of estrus, and/or (e) is infertile.

In some embodiments, the prepubescent non-human subject is male and following administration of the composition, (a) does not experience puberty, and/or (b) is infertile.

The disclosure also provides vectors comprising nucleic acids encoding feline or canine Müllerian Inhibiting Substance (fcMIS or clMIS). In some embodiments, the vector comprises a nucleic acid encoding a feline Müllerian Inhibiting Substance (MIS) protein operatively linked to one or more regulatory elements, (a) wherein the feline MIS protein comprises a wild-type feline MIS protein having an amino acid sequence of SEQ ID NO: 1 or SEQ ID NO: 18, or an amino acid sequence having at least 80%, at least 85%, at least 90%, at least 95%, at least 96%, at least 97%, at least 98%, or at least 99% sequence identity to amino acids 22-588 of SEQ ID NO: 1 or to amino acids 22-589 of SEQ ID NO: 18, or (b) wherein the feline MIS protein comprises a chimeric feline MIS protein having an amino acid sequence of SEQ ID NO: 3 or an amino acid sequence having at least 80%, at least 85%, at least 90%, at least 95%, at least 96%, at least 97%, at least 98%, or at least 99% sequence identity to amino acids 22-572 of SEQ ID NO: 3. In some embodiments, the nucleic acid of the vector encodes a feline MIS protein comprising a protein having at least 85% sequence identity to amino acids 22-588 of SEQ ID NO: 1 or to amino acids 22-589 of SEQ ID NO: 18, and wherein amino acid residue Q at position 478 of SEQ ID NO: 1 or position 479 of SEQ ID NO: 18 is changed from a Q to a R (arginine), or a conservative amino acid of R. In some embodiments, the nucleic acid of the vector encodes a feline MIS protein comprising a protein having at least 85% sequence identity to amino acids 22-572 of SEQ ID NO: 3, and wherein amino acid residue Q at position 465 of SEQ ID NO: 3 is changed from a Q to a R (arginine), or a conservative amino acid of R such as, a K (lysine). In some embodiments, the conservative amino acid of R is K.

In some embodiments, the vector comprises a nucleic acid encoding a canine Müllerian Inhibiting Substance (MIS) protein operatively linked to one or more regulatory elements, wherein the canine MIS protein comprises a wild-type canine MIS protein having an amino acid sequence of SEQ ID NO: 2 or an amino acid sequence having at least 80%, at least 85%, at least 90%, at least 95%, at least 96%, at least 97%, at least 98%, or at least 99% sequence identity to amino acids 22-588 of SEQ ID NO: 2. In some embodiments, the nucleic acid of the vector encodes a canine MIS protein comprising a protein having at least 85% sequence identity to amino acids 23-543 of SEQ ID NO: 2 and wherein amino acid residue Q at position 462 of SEQ ID NO: 2 is changed from a Q to a R (arginine), or a conservative amino acid of R. in some embodiments, the conservative amino acid of R is K.

In some embodiments, a modified feline Müllerian Inhibiting Substance (MIS) protein is produced from a feline MIS proprotein selected from: (a) a feline MIS protein comprising a non-MIS leader sequence in place of amino acids 1-21 of SEQ ID NO: 1 or of SEQ ID NO: 18, and an amino acid sequence having at least 80%, at least 85%, at least 90%, at least 95%, at least 96%, at least 97%, at least 98%, or at least 99% sequence identity to amino acids 22-588 of SEQ ID NO: 1 or to amino acids 22-589 of SEQ ID NO: 18, or (b) a chimeric feline MIS protein comprising a non-MIS leader sequence in place of amino acids 1-21 of SEQ ID NO: 3, and having an amino acid sequence having at least 80%, at least 85%, at least 90%, at least 95%, at least 96%, at least 97%, at least 98%, or at least 99% sequence identity to amino acids 22-572 of SEQ ID NO: 3, or (c) a modified feline MIS protein (LR-fcMIS) comprising amino acids of SEQ ID NO: 14 or SEQ ID NO: 20, and an amino acid sequence having at least 80%, at least 85%, at least 90%, at least 95%, at least 96%, at least 97%, at least 98%, or at least 99% sequence identity to amino acids 22-588 of SEQ ID NO: 14 or amino acids 22-589 of SEQ ID NO: 20. In some embodiments, the feline MIS protein comprises a protein having at least 85% sequence identity to amino acids 22-588 of SEQ ID NO: 1 or to amino acids 22-589 of SEQ ID NO: 18, and wherein amino acid residue Q at position 478 of SEQ ID NO: 1 or position 479 of SEQ ID NO: 18 is changed from a Q to a R (arginine), or a conservative amino acid of R. In some embodiments, the chimeric feline MIS protein comprises a protein having at least 85% sequence identity to amino acids 22-572 of SEQ ID NO: 3, and wherein amino acid residue Q at position 465 of SEQ ID NO: 3 is changed from a Q to a R (arginine), or a conservative amino acid of R such as, a K (lysine). In some embodiments, the non-leader sequence of the modified feline MIS protein is selected from any of SEQ ID NO: 9-13.

In some embodiments, a modified canine Müllerian Inhibiting Substance (MIS) protein is produced from a canine MIS proprotein, where the canine MIS proprotein comprises a non-MIS leader sequence in place of amino acids 1-21 of SEQ ID NO: 2, and an amino acid sequence having at least 80%, at least 85%, at least 90%, at least 95%, at least 96%, at least 97%, at least 98%, or at least 99% sequence identity to amino acids 22-588 of SEQ ID NO: 2, or an amino acid sequence of SEQ ID NO: 15 or an amino acid sequence having at least 80%, at least 85%, at least 90%, at least 95%, at least 96%, at least 97%, at least 98%, or at least 99% sequence identity to SEQ ID NO: 15. In some embodiments, the canine MIS comprises a protein having at least 85% sequence identity to amino acids 23-543 of SEQ ID NO: 2 and wherein amino acid residue Q at position 462 of SEQ ID NO: 2 is changed from a Q to a R (arginine), or a conservative amino acid of R. In some embodiments, the conservative amino acid of R is K.

The disclosure also provides pharmaceutical compositions comprising any modified feline MIS protein disclosed herein or any modified canine MIS protein disclosed herein.

The disclosure also provides methods of reversibly delaying puberty in a prepubescent human female subject comprising administering to the subject an effective amount of a composition comprising a recombinant human Müllerian Inhibiting Substance (rhMIS) protein, wherein the recombinant human MIS protein is comprises amino acid residues 25-560 of SEQ ID NO: 4, or a protein at least 85% sequence identity to SEQ ID NO: 4, and wherein the amino acid residue 450 of SEQ ID NO: 4 is changed from a Q to R or a conservative amino acid of R. In some embodiments, the rhMIS protein is produced from a rhMIS proprotein comprising a non-MIS leader sequence in place of amino acids 1-24 of SEQ ID NO: 4, and an amino acid sequence having at least 85% sequence identity to amino acids 25-560 of SEQ ID NO: 4 and wherein amino acid residue 450 of SEQ ID NO: 4 is changed from a Q to R a conservative amino acid of R. In some embodiments, the conservative amino acid of R is a K. In some embodiments, the rhMIS protein comprises a protein having an amino acid sequence of at least 19-554 of SEQ ID NO: 7, or a protein at least 85% sequence identity to SEQ ID NO: 7. In some embodiments, the rhMIS protein comprises a protein having an amino acid sequence of at least 19-554 of SEQ ID NO: 7, or a protein at least 85% sequence identity to SEQ ID NO: 7, and a non-MIS leader sequence selected from any of SEQ ID NO: 9-13, or a non-leader sequence having at least 85% sequence identity to any of SEQ ID NO: 9-13. In some embodiments, the recombinant human MIS protein is manufactured or produced by a nucleic acid disclosed in WO2015089321.

In some embodiments, the prepubescent human female subject is in need of delaying puberty. In some embodiments, the prepubescent human female subject in need of delaying puberty has one or more conditions selected from: gender dysphoria, intersex, atypical genitalia at birth, both male and female genitalia at birth, mosaic genetics, Klinefelter syndrome, central precocious puberty (CPP) or peripheral precocious puberty or congenital adrenal hyperplasia (CAH).

The disclosure also provides methods of detecting anti-fcMIS antibodies or anti-clMIS antibodies antibodies in non-human subjects administered a viral vector expressing fcMIS or clMIS comprising (a) obtaining a sample from a non-human subject administered a viral vector encoding fcMIS or clMIS, optionally wherein the fcMIS or clMIS comprises the amino acid sequence of SEQ ID NO: 1, SEQ ID NO: 2, SEQ ID NO: 3, SEQ ID NO: 14, SEQ ID NO: 15, SEQ ID NO: 18, or SEQ ID NO: 20; (b) optionally isolating recombinant fcMIS or clMIS, optionally wherein the recombinant fcMIS or clMIS comprises a FLAG tag; (c) adding fcMIS or clMIS to a substrate, optionally wherein the substrate is an ELISA plate; (d) adding a test sample to the substrate; (e) incubating the substrate with a detectable antibody, optionally wherein the detectable antibody is a goat anti-IgG HRP; and (f) performing an enzyme substrate reaction, optionally wherein the enzyme substrate is HRP. In some embodiments, the test sample is diluted with a blocking buffer before being added to the substrate. In some embodiments, the method further comprises incubating the substrate with Bovine Serum Albumin before the step of adding a test sample to the substrate. In some embodiments, the method further comprises one or more washing step to remove excess MIS or excess detectable antibody.

### DESCRIPTION OF CERTAIN SEQUENCES

Table 1 provides a listing of certain sequences referenced herein.

TABLE 1
Description of Certain Sequences

| SEQ ID | | |
|---|---|---|
| NO: | SEQUENCE | DESCRIPTION |
| 1 | MPGLLSPPALVLSVMGALLMAGDPGEEVSSTPALPGGPATGTG | Exemplary wild-type |
| | GLIFHPDWDWQPPGSPQDPLCLVTLDRGGNGSGSPLRVVGALR | felis catus MIS amino |
| | GYEHAFLEAVRRARWGPHGLATFGVCTPRDRQAAPFSLRQLQA | acid sequence (wt- |
| | WLGEPGGRRLVVLHLEEVTWEPTPSLKFQEPPPGGAGPLELAM | fcMIS or fcMISv2) |
| | LVLYPGPGPEVTVTGAGLPGTQSLCQSRDTRYLVLAVDHPEGA | (amino acids 1-21 |
| | WRSPGLTLTLQPRRDGAPLSTAQLQELLFGPDPRCFTRMTPAL | (underlined) is the |
| | LLLPGPAPAPLPARGLLDQVPLPPPRPSQEQAPEEPRSSADPF | endogenous fcMIS |
| | LETLTRLVRALRGPPAQASPARLALDPGALAGFPQGLVNLSDP | leader sequence, |
| | AAQERLINGGDEPLLLLLLPPATPTAAAAAAGDPAPPRGPASA | amino acids 22-588 is |
| | PWAAGLARRVAAELQAAAAELRGLPGLPPAATPLLARLLALCP | core fcMIS protein |
| | GDSGDSGDPGAPPGGPGGPLRALLLLKALQGLRAEWRGREQAG | sequence) |
| | PARAQRSAGAGAADGPCALRELSVDLRAERSVLIPETYQANNC | |
| | QGACGWPQSDRNPRYGNHVVLLLKMQARGAALARPPCCVPTAY | |
| | AGKLLISLSEERISAHHVPNMVATECGCR | |
| 2 | MGALALWPLALALSGMGPLLGAEAPGGEVSGTPASPGEPATGT | Exemplary wild-type |
| | GGLLFQPDWDWPPSAPQDPLCLVTLDKGGNGSSPPLRVAGALR | canis lupus MIS |
| | GYEHTFLEAVRRARWGPHDLATFGACAASDGRTTQLSLRQLQA | amino acid sequence |
| | WLGAPGGRRLVVLHLEEVTWEPALSLKFQEPPPGGASPLELAL | (wt-clMIS or clMIS) |
| | LVLYPGPGPEVAVTGAGLPGTQNLCRSRNTRYLVLALDHPVGA | (amino acids 1-22 |
| | WHSPRVTLTVHARGDGAPLSTPQLQELLEGPDARCFTRMTPAL | (underlined) is the |
| | LVLRLPGPTAVPARGLLDLVPFPPPRPSREPAEPPPSADPFLE | endogenous canine |
| | TLTRLVRALRGPPTPASPPRLALDPGALAGFPQGLLNLSDPAT | leader sequence, |
| | QERLLGGEEPLLLLLPPPTAAAGPPAPPPRPASAPWAAGLALR | amino acids 22-573 is |
| | VAAELRAAAAELRGLPGLPPAAAPLLERLLALCPGGSGGSGGS | core clMIS protein |
| | GDPLRALLLLKALQGLRAEWRGRERGGPPRAQRSAGAGAADGP | sequence) |
| | CALRELSVDLRAERSVLIPETYQANNCQGACGWPQSDRNPRYG | |
| | NHVVLLLKMQARGAALARPPCCVPTAYGGKLLISLSEERISAH | |
| | HVPNMVATECGCR | |
| 3 | MPGLLSPPALVLSVMGALLMAGDPGEEVSSTPALPGGPATGTG | Exemplary chimeric |
| | GLIFHPDWDWQPPGSPQDPLCLVTLDRGGNGSGSPLRVVGALR | felis catus MIS amino |
| | GYEHAFLEAVRRARWGPHGLATFGVCTPRDRQAAPFSLRQLQA | acid sequence |
| | WLGEPGGRRLVVLHLEEVTWEPTPSLKFQEPPPGGAGPLELAM | (fcMISv1) |
| | LVLYPGPGPEVTVTGAGLPGTQSLCQSRDTRYLVLAVDHPEGA | (amino acids 1-21 is |
| | WRSPGLTLTLQPRRDGAPLSTAQLQELLFGPDPRCFTRMTPAL | the endogenous fcMIS |
| | LLLPGPAPAPLPARGLLDQVPLPPPRPSQEQAPEEPRSSADPF | leader sequence, |
| | LETLTRLVRALRGPPAQASPARLALDPGALAGFPQGLVNLSDP | amino acids 22-572 is |
| | AAQERLINGGDEPLLLLLPPPTAAAGPPAPPPRPASAPWAAGL | core chimeric fcMIS |
| | ALRVAAELRAAAAELRGLPGLPPATAPLLERLLALCPGGSGGS | protein sequence) |
| | GGSGDPLRALLLLKALQGLRAEWRGRERGGPPRAQRSAGAGAA | |
| | DGPCALRELSVDLRAERSVLIPETYQANNCQGACGWPQSDRNP | |
| | RYGNHVVLLLKMQARGAALARPPCCVPTAYAGKLLISLSEERI | |
| | SAHHVPNMVATECGCR | |
| 4 | MRDLPLTSLALVLSALGALLGTEALRAEEPAVGTSGLIFREDL | Exemplary wild-type |
| | DWPPGSPQEPLCLVALGGDSNGSSSPLRVVGALSAYEQAFLGA | homo sapiens MIS |
| | VQRARWGPRDLATFGVCNTGDRQAALPSLRRLGAWLRDPGGQR | amino acid sequence |
| | LVVLHLEEVTWEPTPSLRFQEPPPGGAGPPELALLVLYPGPGP | (hsMIS) |
| | EVTVTRAGLPGAQSLCPSRDTRYLVLAVDRPAGAWRGSGLALT | (amino acids 1-24 is |
| | LQPRGEDSRLSTARLQALLFGDDHRCFTRMTPALLLLPRSEPA | the endogenous hsMIS |
| | PLPAHGOLDTVPFPPPRPSAELEESPPSADPFLETLTRLVRAL | leader sequence, |
| | RVPPARASAPRLALDPDALAGFPQGLVNLSDPAALERLLDGEE | amino acids 25-560 is |
| | PLLLLLRPTAATTGDPAPLHDPTSAPWATALARRVAAELQAAA | core hsMIS protein |
| | AELRSLPGLPPATAPLLARLLALCPGGPGGLGDPLRALLLLKA | sequence, with amino |
| | LQGLRVEWRGRDPRGPGRAQRSAGATAADGPCALRELSVDLRA | acid 450 is Q) |
| | ERSVLIPETYQANNCQGVCGWPQSDRNPRYGNHVVLLLKMQVR | |
| | GAALARPPCCVPTAYAGKLLISLSEERISAHHVPNMVATECGC | |
| | R | |
| 5 | ATGCCTGGCCTGCTGTCCCCTCCCGCCCTGGTGCTGTCCGTCA | Exemplary wild-type |
| | TGGGTGCCCTGCTGATGGCTGGAGACCCTGGTGAAGAAGTGTC | feline MIS (wt-fcMIS |
| | AAGCACCCCAGCCCTGCCTGGAGGACCTGCAACCGGCACCGGA | or fcMISv2) nucleic |
| | GGCCTGATCTTCCACCCAGACTGGGATTGGCAGCCACCTGGCT | acid sequence |
| | CTCCACAGGATCCACTGTGCCTGGTGACCCTGGACAGAGGAGG | |
| | AAACGGATCTGGCTCCCCACTGAGAGTGGTGGGCGCCCTGAGG | |
| | GGATACGAGCACGCCTTTCTGGAGGCCGTGAGGAGAGCAAGAT | |
| | GGGGACCTCACGGCCTGGCCACCTTCGGCGTGTGCACCCCTAG | |
| | GGATAGACAGGCCGCCCCATTTTCCCTGAGGCAGCTGCAGGCA | |
| | TGGCTGGGAGAGCCAGGAGGCCGGCGCCTGGTGGTGCTGCACC | |
| | TGGAGGAGGTGACCTGGGAGCCAACCCCCAGCCTGAAGTTCCA | |
| | GGAGCCACCACCTGGAGGAGCAGGACCCCTGGAGCTGGCCATG | |
| | CTGGTGCTGTACCCTGGACCAGGACCAGAGGTGACCGTGACCG | |
| | GAGCAGGCCTGCCTGGCACCCAGTCTCTGTGCCAGTCCAGGGA | |
| | TACCAGATACCTGGTGCTGGCCGTGGACCACCCAGAGGGAGCA | |
| | TGGCGCTCCCCTGGCCTGACCCTGACCCTGCAGCCTAGGAGAG | |
| | ATGGAGCACCACTGAGCACCGCACAGCTGCAGGAGCTGCTGTT | |
| | CGGACCTGACCCACGCTGTTTTACCAGGATGACCCCCGCCCTG | |
| | CTGCTGCTGCCAGGACCTGCACCAGCACCACTGCCTGCCAGAG | |
| | GCCTGCTGGATCAGGTGCCCCTGCCACCACCTAGACCTAGCCA | |
| | GGAGCAGGCACCTGAGGAGCCACGGTCCAGCGCCGACCCTTTC | |
| | CTGGAAACCCTGACCAGGCTGGTGCGCGCCCTGAGGGGACCAC | |
| | CAGCACAGGCCTCTCCAGCCAGACTGGCCCTGGATCCAGGCGC | |
| | CCTGGCCGGATTTCCTCAGGGCCTGGTGAACCTGTCCGATCCA | |
| | GCAGCACAGGAGCGGCTGCTGAATGGAGGCGACGAGCCACTGC | |
| | TGCTGCTGCTGCTGCCTCCAGCAACCCCAACCGCCGCCGCAGC | |
| | AGCAGCAGGCGACCCTGCCCCCCCTCGGGGCCCAGCCTCTGCC | |
| | CCCTGGGCCGCCGGCCTGGCCCGGCGCGTGGCCGCCGAGCTGC | |
| | AGGCCGCCGCCGCCGAGCTGAGAGGCCTGCCAGGCCTGCCACC | |
| | CGCCGCCACCCCCCTGCTGGCCCGGCTGCTGGCCCTGTGCCCT | |
| | GGCGACTCCGGCGATAGCGGCGACCCAGGAGCACCTCCAGGAG | |
| | GACCAGGAGGCCCTCTGCGCGCCCTGCTGCTGCTGAAGGCCCT | |
| | GCAGGGCCTGAGGGCCGAGTGGCGGGGCCGCGAGCAGGCCGGC | |
| | CCTGCCAGAGCACAGCGGTCTGCCGGAGCAGGAGCAGCAGATG | |
| | GCCCATGTGCACTGAGGGAGCTGAGCGTGGACCTGAGGGCAGA | |
| | GAGGTCTGTGCTGATCCCCGAAACCTACCAGGCCAACAATTGC | |
| | CAGGGAGCATGTGGATGGCCACAGTCCGACAGAAACCCCCGGT | |
| | ACGGCAATCACGTGGTGCTGCTGCTGAAGATGCAGGCAAGGGG | |
| | AGCCGCCCTGGCCAGGCCCCCTTGCTGCGTGCCAACCGCATAC | |
| | GCAGGCAAGCTGCTGATCAGCCTGTCTGAAGAAAGAATCTCTG | |
| | CTCATCACGTCCCCAATATGGTCGCCACTGAATGCGGTTGTCG | |
| | GTGA | |
| 6 | ATGCCTGGGCTGCTGTCTCCACCCGCTCTCGTCCTGTCCGTGA | Nucleic acid sequence |
| | TGGGGGCTCTCCTGATGGCTGGCGACCCTGGGGAAGAAGTGTC | encoding an |
| | CTCTACCCCCGCCCTGCCTGGAGGACCAGCAACCGGCACCGGA | exemplary chimeric |
| | GGCCTGATCTTCCACCCAGACTGGGATTGGCAGCCACCTGGCA | fcMIS (fcMISv1) |
| | GCCCCCAGGACCCTCTGTGCCTGGTGACCCTGGATAGGGGAGG | |
| | AAACGGATCTGGCAGCCCACTGAGGGTGGTGGGCGCCCTGAGA | |
| | GGATACGAGCACGCCTTCCTGGAGGCCGTGAGGAGAGCAAGAT | |
| | GGGGACCTCACGGCCTGGCCACCTTCGGCGTGTGCACCCCACG | |
| | GGACCGCCAGGCAGCACCTTTCTCCCTGAGACAGCTGCAGGCA | |
| | TGGCTGGGAGAGCCAGGAGGCCGGCGCCTGGTGGTGCTGCACC | |
| | TGGAGGAGGTGACCTGGGAGCCAACCCCTTCTCTGAAGTTCCA | |
| | GGAGCCACCACCTGGAGGAGCAGGACCCCTGGAGCTGGCAATG | |
| | CTGGTGCTGTACCCAGGACCAGGACCTGAGGTGACCGTGACCG | |
| | GAGCAGGCCTGCCTGGCACCCAGAGCCTGTGCCAGTCCAGGGA | |
| | CACCAGATACCTGGTGCTGGCAGTGGATCACCCAGAGGGAGCA | |
| | TGGAGATCCCCCGGCCTGACCCTGACCCTGCAGCCAAGGAGAG | |
| | ACGGAGCACCTCTGTCTACCGCACAGCTGCAGGAGCTGCTGTT | |
| | CGGACCAGATCCAAGGTGTTTCACCAGGATGACCCCCGCCCTG | |
| | CTGCTGCTGCCTGGACCAGCACCAGCACCTCTGCCAGCAAGGG | |
| | GCCTGCTGGACCAGGTGCCACTGCCACCACCTCGGCCCTCTCA | |
| | GGAGCAGGCACCAGAGGAGCCACGCAGCTCCGCCGATCCCTTC | |
| | CTGGAAACCCTGACCAGACTGGTGCGGGCCCTGAGGGGACCAC | |
| | CAGCACAGGCCAGCCCTGCCCGGCTGGCCCTGGACCCAGGCGC | |
| | CCTGGCCGGCTTCCCACAGGGCCTGGTGAACCTGTCCGACCCA | |
| | GCAGCACAGGAGCGCCTGCTGAACGGAGGCGATGAGCCACTGC | |
| | TGCTGCTGCTGCCTCCACCAACCGCAGCAGCAGGACCTCCAGC | |
| | ACCACCTCCAAGGCCTGCCAGCGCCCCATGGGCCGCCGGCCTG | |
| | GCCCTGCGGGTGGCCGCCGAGCTGCGCGCCGCCGCCGCCGAGC | |
| | TGCGGGGCCTGCCTGGCCTGCCCCCTGCCACCGCCCCACTGCT | |
| | GGAGCGCCTGCTGGCCCTGTGCCCCGGCGGCAGCGGCGGCTCC | |
| | GGCGGCTCTGGCGACCCTCTGAGGGCCCTGCTGCTGCTGAAGG | |
| | CCCTGCAGGGCCTGAGAGCAGAGTGGAGGGGAAGAGAGCGGGG | |
| | CGGCCCACCCAGGGCCCAGAGAAGCGCCGGAGCAGGAGCAGCA | |
| | GACGGACCTTGTGCCCTGAGAGAGCTGTCTGTGGATCTGAGGG | |
| | CAGAGCGCAGCGTGCTGATCCCAGAAACCTACCAGGCCAACAA | |
| | CTGCCAGGGAGCATGTGGATGGCCTCAGTCCGATAGGAACCCA | |
| | AGATACGGCAACCACGTGGTGCTGCTGCTGAAGATGCAGGCAA | |
| | GGGGAGCCGCCCTGGCCAGACCTCCATGCTGCGTGCCAACCGC | |
| | ATACGCAGGCAAGCTGCTGATCTCCCTGTCTGAGGAAAGAATC | |
| | TCTGCTCATCACGTCCCAAACATGGTCGCAACCGAGTGTGGCT | |
| | GTCGCTGA | |
| 7 | MKWVTFISLL FLFSSAYSLR AEEPAVGTSG LIFR | Exemplary |
| | EDLDWPPGSP QEPLCLVALG GDSNGSSSPL RVVGALSAYE | recombinant modified |
| | QAFLGAVORA RWGPRDLATF GVCNTGDRQA ALPSLRRLGA | homo sapiens MIS |
| | WIRDPGGQRL VVLHLEEVTW EPTPSLRFQE PPPGGAGPPE | (LRhsMIS) protein |
| | LALLVLYPGP GPEVTVTRAG LPGAQSLCPS RDTRYLVLAV | LRhsMIS (Q450R) |
| | DRPAGAWRGS GLALTLQPRG EDSRLSTARL QALLFGDDHR | (amino acids 1-18 is |
| | CFTRMTPALL LLPRSEPAPL PAHGOLDTVP FPPPRPSAEL | an exemplary non- |
| | EESPPSADPF LETLTRLVRA LRVPPARASA PRLALDPDAL | MIS leader sequence |
| | AGFPQGLVNL SDPAALERLL DGEEPLLLLL RPTAATTGDP | (human albumin |
| | APLHDPTSAP WATALARRVA AELQAAAAEL RSLPGLPPAT | sequence), amino |
| | APLLARLLAL CPGGPGGLGD PLRALLLLKA LQGLRVEWRG | acids 19-554 is core |
| | RDPRGPGRAR RSAGATAADG PCALRELSVD LRAERSVLIP | hsMIS protein |
| | ETYQANNCQG VCGWPQSDRN PRYGNHVVLL LKMQVRGAAL | sequence, where |
| | ARPPCCVPTA YAGKLLISLS EERISAHHVP NMVATECGCR | amino acid 444 is a R) |
| 8 | ATGAAGTGGGTGAGCTTCATCAGCCTGCTGTTCCTGTTCAGCA | Nucleic acid sequence |
| | GCGCTTACTCCCGCGGTGTGTTCCGCCGCAGAGCAGAGGAGCC | encoding an |
| | AGCTGTGGGCACCAGTGGCCTCATCTTCCGAGAAGACTTGGAC | exemplary |
| | TGGCCTCCAGGCAGCCCACAAGAGCCTCTGTGCCTGGTGGCAC | recombinant modified |
| | TGGGCGGGGACAGCAATGGCAGCAGCTCCCCCCTGCGGGTGGT | homo sapiens (hsMIS) |
| | GGGGGCTCTAAGCGCCTATGAGCAGGCCTTCCTGGGGGCCGTG | protein of SEQ ID |
| | CAGAGGGCCCGCTGGGGCCCCCGAGACCTGGCCACCTTCGGGG | NO: 7 |
| | TCTGCAACACCGGTGACAGGCAGGCTGCCTTGCCCTCTCTACG | LRhsMIS (Q450R) |
| | GCGGCTGGGGGCCTGGCTGCGGGACCCTGGGGGGCAGCGCCTG | |
| | GTGGTCCTACACCTGGAGGAAGTGACCTGGGAGCCAACACCCT | |
| | CGCTGAGGTTCCAGGAGCCCCCGCCTGGAGGAGCTGGCCCCCC | |
| | AGAGCTGGCGCTGCTGGTGCTGTACCCTGGGCCTGGCCCTGAG | |
| | GTCACTGTGACGAGGGCTGGGCTGCCGGGTGCCCAGAGCCTCT | |
| | GCCCCTCCCGAGACACCCGCTACCTGGTGTTAGCGGTGGACCG | |
| | CCCTGCGGGGGCCTGGCGCGGCTCCGGGCTGGCCTTGACCCTG | |
| | CAGCCCCGCGGAGAGGACTCCCGGCTGAGTACCGCCCGGCTGC | |
| | AGGCACTGCTGTTCGGCGACGACCACCGCTGCTTCACACGGAT | |
| | GACCCCGGCCCTGCTCCTGCTGCCGCGGTCCGAGCCCGCGCCG | |
| | CTGCCTGCGCACGGCCAGCTGGACACCGTGCCCTTCCCGCCGC | |
| | CCAGGCCATCCGCGGAACTCGAGGAGTCGCCACCCAGCGCAGA | |
| | CCCCTTCCTGGAGACGCTCACGCGCCTGGTGCGGGCGCTGCGG | |
| | GTCCCCCCGGCCCGGGCCTCCGCGCCGCGCCTGGCCCTGGATC | |
| | CGGACGCGCTGGCCGGCTTCCCGCAGGGCCTAGTCAACCTGTC | |
| | GGACCCCGCGGCGCTGGAGCGCCTACTCGACGGCGAGGAGCCG | |
| | CTGCTGCTGCTGCTGAGGCCCACTGCGGCCACCACCGGGGATC | |
| | CTGCGCCCCTGCACGACCCCACGTCGGCGCCGTGGGCCACGGC | |
| | CCTGGCGCGCCGCGTGGCTGCTGAACTGCAAGCGGCGGCTGCC | |
| | GAGCTGCGAAGCCTCCCGGGTCTGCCTCCGGCCACAGCCCCGC | |
| | TGCTGGCGCGCCTGCTCGCGCTCTGCCCAGGTGGCCCCGGCGG | |
| | CCTCGGCGATCCCCTGCGAGCGCTGCTGCTCCTGAAGGCGCTG | |
| | CAGGGCCTGCGCGTGGAGTGGCGCGGGCGGGATCCGCGCGGGC | |
| | CGGGTCGGGCACGGCGCAGCGCGGGGGCCACCGCCGCCGACGG | |
| | GCCGTGCGCGCTGCGCGAGCTCAGCGTAGACCTCCGCGCCGAG | |
| | CGCTCCGTACTCATCCCCGAGACCTACCAGGCCAACAATTGCC | |
| | AGGGCGTGTGCGGCTGGCCTCAGTCCGACCGCAACCCGCGCTA | |
| | CGGCAACCACGTGGTGCTGCTGCTGAAGATGCAGGCCCGTGGG | |
| | GCCGCCCTGGCGCGCCCACCCTGCTGCGTGCCCACCGCCTACG | |
| | CGGGCAAGCTGCTCATCAGCCTGTCGGAGGAGCGCATCAGCGC | |
| | GCACCACGTGCCCAACATGGTGGCCACCGAGTGTGGCTGCCGG | |
| | TGA | |
| 9 | MTRLTVLALLAGLLASSRA | Exemplary leader |
| | | sequence |
| 10 | MKWVTFISLLFLESSAYS | Exemplary leader |
| | | sequence |
| 11 | MKWVTFISLLFLFSSAYSRGVERR | Exemplary leader |
| | | sequence |
| 12 | MKWVTFISLLFLFSSAYSRGVERR | Exemplary leader |
| | | sequence |
| 13 | MKWVSFISLLFLFSSAYS | Exemplary leader |
| | | sequence |
| 14 | MKWVTFISLLLLFSSAYSGDPGEEVSSTPALPGGPATGTGGLI | Exemplary LR- |
| | FHPDWDWQPPGSPQDPLCLVTLDRGGNGSGSPLRVVGALRGYE | fcMISv2 amino acid |
| | HAFLEAVRRARWGPHGLATFGVCTPRDRQAAPFSLRQLQAWLG | sequence |
| | EPGGRRLVVLHLEEVTWEPTPSLKFQEPPPGGAGPLELAMLVL | (amino acids 1-18 is |
| | YPGPGPEVTVTGAGLPGTQSLCQSRDTRYLVLAVDHPEGAWRS | an exemplary non- |
| | PGLTLTLQPRRDGAPLSTAQLQELLFGPDPRCFTRMTPALLLL | MIS leader sequence, |
| | PGPAPAPLPARGLLDQVPLPPPRPSQEQAPEEPRSSADPFLET | amino acids 19-585 is |
| | LTRLVRALRGPPAQASPARLALDPGALAGFPQGLVNLSDPAAQ | core fcMIS protein |
| | ERLINGGDEPLLLLLLPPATPTAAAAAAGDPAPPRGPASAPWA | sequence, with Q475R |
| | AGLARRVAAELQAAAAELRGLPGLPPAATPLLARLLALCPGDS | modification, amino |
| | GDSGDPGAPPGGPGGPLRALLLLKALQGLRAEWRGREQAGPAR | acids RARRS in bold |
| | ARRSAGAGAADGPCALRELSVDLRAERSVLIPETYQANNCQGA | indicate enhanced |
| | CGWPQSDRNPRYGNHVVLLLKMQARGAALARPPCCVPTAYAGK | cleavage site) |
| | LLISLSEERISAHHVPNMVATECGCR | |
| 15 | MKWVTFISLFFLFSSAYSEAPGGEVSGTPASPGEPATGTGGLL | Exemplary LR-clMIS |
| | FQPDWDWPPSAPQDPLCLVTLDKGGNGSSPPLRVAGALRGYEH | amino acid sequence |
| | TFLEAVRRARWGPHDLATFGACAASDGRTTQLSLRQLQAWLGA | (amino acids 1-18 is |
| | PGGRRLVVLHLEEVTWEPALSLKFQEPPPGGASPLELALLVLY | an exemplary non- |
| | PGPGPEVAVTGAGLPGTQNLCRSRNTRYLVLALDHPVGAWHSP | MIS leader sequence, |
| | RVTLTVHARGDGAPLSTPQLQELLFGPDARCFTRMTPALLVLR | amino acids 19-565 is |
| | LPGPTAVPARGLLDLVPFPPPRPSREPAEPPPSADPFLETLTR | core clMIS protein |
| | LVRALRGPPTPASPPRLALDPGALAGFPQGLLNLSDPATQERL | sequence, with Q458R |
| | LGGEEPLLLLLPPPTAAAGPPAPPPRPASAPWAAGLALRVAAE | modification, amino |
| | LRAAAAELRGLPGLPPAAAPLLERLLALCPGGSGGSGGSGDPL | acids RARRS in bold |
| | RALLLLKALQGLRAEWRGRERGGPPRARRSAGAGAADGPCALR | indicate enhanced |
| | ELSVDLRAERSVLIPETYQANNCQGACGWPQSDRNPRYGNHVV | |
| | LLLKMQARGAALARPPCCVPTAYGGKLLISLSEERISAHHVPN | |
| | MVATECGCR | |
| 16 | ATGAAGTGGGTGACCTTCATCAGCCTATTCTTCCTGTTCAGCA | cleavage site) |
| | GCGCCTACAGCGAAGCACCTGGCGGCGAGGTTTCCGGAACACC | Exemplary nucleic |
| | CGCTTCACCTGGCGAACCAGCAACTGGAACAGGAGGCCTACTA | acid sequence |
| | TTCCAGCCAGACTGGGATTGGCCCCCTTCAGCCCCCCAAGACC | encoding LR-clMIS |
| | CTCTATGCCTAGTAACCCTAGATAAAGGAGGCAACGGCTCTTC | |
| | CCCACCCCTACGAGTGGCAGGAGCCCTACGAGGCTACGAACAC | |
| | ACATTCCTAGAGGCCGTACGACGAGCTCGATGGGGACCCCACG | |
| | ACCTAGCAACCTTTGGCGCCTGCGCCGCTTCTGATGGACGAAC | |
| | AACCCAGCTATCCCTACGACAGCTACAAGCTTGGCTAGGAGCA | |
| | CCTGGAGGCCGACGACTAGTGGTACTACACCTAGAAGAGGTGA | |
| | CATGGGAACCCGCCCTATCTCTAAAGTTTCAGGAGCCTCCACC | |
| | AGGAGGAGCCTCCCCTCTAGAACTAGCTCTACTAGTACTATAC | |
| | CCTGGACCAGGACCAGAGGTGGCAGTAACCGGAGCTGGCCTAC | |
| | CAGGCACTCAAAACCTATGCCGATCTCGAAACACCCGATACCT | |
| | CGTGCTCGCTCTAGACCACCCTGTAGGAGCATGGCACTCACCA | |
| | CGAGTGACCCTAACTGTACACGCACGAGGAGACGGCGCCCCAC | |
| | TATCTACCCCCCAGCTACAAGAGCTACTATTCGGCCCCGATGC | |
| | CCGATGCTTTACTCGAATGACACCTGCTCTACTAGTGCTACGA | |
| | CTACCTGGCCCAACTGCTGTACCTGCACGAGGACTACTAGACC | |
| | TAGTGCCATTCCCTCCACCCCGACCTTCCCGAGAACCAGCAGA | |
| | GCCTCCACCCTCAGCCGATCCATTTCTAGAAACACTAACCCGA | |
| | CTAGTGCGAGCCCTACGAGGACCTCCAACACCCGCTTCCCCCC | |
| | CTCGACTAGCACTAGACCCCGGAGCTCTAGCAGGCTTCCCTCA | |
| | GGGACTACTAAACCTATCAGATCCAGCTACTCAAGAGCGACTA | |
| | CTAGGAGGCGAAGAGCCCCTACTACTACTACTACCACCCCCTA | |
| | CAGCAGCCGCTGGACCACCAGCTCCTCCACCCCGACCCGCCTC | |
| | CGCTCCTTGGGCAGCCGGACTAGCACTACGAGTAGCTGCAGAA | |
| | CTACGAGCCGCTGCAGCCGAGCTACGAGGACTACCAGGCCTAC | |
| | CTCCAGCTGCAGCCCCCCTACTAGAACGACTACTAGCCCTATG | |
| | CCCTGGAGGATCCGGAGGATCAGGAGGATCTGGCGACCCACTA | |
| | CGAGCCCTACTACTACTAAAAGCTCTACAGGGCCTACGAGCAG | |
| | AATGGCGAGGACGAGAGAGAGGAGGACCACCTCGAGCACGGCG | |
| | ATCTGCAGGAGCAGGAGCTGCAGACGGACCTTGCGCACTACGA | |
| | GAGCTATCAGTGGATCTACGAGCCGAACGATCTGTACTAATCC | |
| | CAGAGACTTACCAGGCCAACAACTGCCAAGGAGCTTGCGGCTG | |
| | GCCACAGTCCGATCGAAACCCCCGATATGGCAACCACGTGGTA | |
| | CTACTACTAAAAATGCAAGCACGAGGAGCCGCTCTAGCCCGAC | |
| | CACCATGCTGCGTGCCTACCGCATATGGAGGCAAGCTACTAAT | |
| | TTCACTATCTGAAGAACGGATCTCCGCACATCACGTACCTAAT | |
| | ATGGTAGCTACTGAGTGTGGTTGTAGATGAGGTACC | |
| 17 | ATGGGCGCATTAGCACTTTGGCCTTTAGCCTTAGCACTATCAG | Nucleic acid sequence |
| | GAATGGGACCACTACTGGGAGCAGAAGCACCTGGCGGCGAGGT | encoding wt-clMIS |
| | TTCCGGAACACCCGCTTCACCTGGCGAACCAGCAACTGGAACA | |
| | GGAGGCCTACTATTCCAGCCAGACTGGGATTGGCCCCCTTCAG | |
| | CCCCCCAAGACCCTCTATGCCTAGTAACCCTAGATAAAGGAGG | |
| | CAACGGCTCTTCCCCACCCCTACGAGTGGCAGGAGCCCTACGA | |
| | GGCTACGAACACACATTCCTAGAGGCCGTACGACGAGCTCGAT | |
| | GGGGACCCCACGACCTAGCAACCTTTGGCGCCTGCGCCGCTTC | |
| | TGATGGACGAACAACCCAGCTATCCCTACGACAGCTACAAGCT | |
| | TGGCTAGGAGCACCTGGAGGCCGACGACTAGTGGTACTACACC | |
| | TAGAAGAGGTGACATGGGAACCCGCCCTATCTCTAAAGTTTCA | |
| | GGAGCCTCCACCAGGAGGAGCCTCCCCTCTAGAACTAGCTCTA | |
| | CTAGTACTATACCCTGGACCAGGACCAGAGGTGGCAGTAACCG | |
| | GAGCTGGCCTACCAGGCACTCAAAACCTATGCCGATCTCGAAA | |
| | CACCCGATACCTCGTGCTCGCTCTAGACCACCCTGTAGGAGCA | |
| | TGGCACTCACCACGAGTGACCCTAACTGTACACGCACGAGGAG | |
| | ACGGCGCCCCACTATCTACCCCCCAGCTACAAGAGCTACTATT | |
| | CGGCCCCGATGCCCGATGCTTTACTCGAATGACACCTGCTCTA | |
| | CTAGTGCTACGACTACCTGGCCCAACTGCTGTACCTGCACGAG | |
| | GACTACTAGACCTAGTGCCATTCCCTCCACCCCGACCTTCCCG | |
| | AGAACCAGCAGAGCCTCCACCCTCAGCCGATCCATTTCTAGAA | |
| | ACACTAACCCGACTAGTGCGAGCCCTACGAGGACCTCCAACAC | |
| | CCGCTTCCCCCCCTCGACTAGCACTAGACCCCGGAGCTCTAGC | |
| | AGGCTTCCCTCAGGGACTACTAAACCTATCAGATCCAGCTACT | |
| | CAAGAGCGACTACTAGGAGGCGAAGAGCCCCTACTACTACTAC | |
| | TACCACCCCCTACAGCAGCCGCTGGACCACCAGCTCCTCCACC | |
| | CCGACCCGCCTCCGCTCCTTGGGCAGCCGGACTAGCACTACGA | |
| | GTAGCTGCAGAACTACGAGCCGCTGCAGCCGAGCTACGAGGAC | |
| | TACCAGGCCTACCTCCAGCTGCAGCCCCCCTACTAGAACGACT | |
| | ACTAGCCCTATGCCCTGGAGGATCCGGAGGATCAGGAGGATCT | |
| | GGCGACCCACTACGAGCCCTACTACTACTAAAAGCTCTACAGG | |
| | GCCTACGAGCAGAATGGCGAGGACGAGAGAGAGGAGGACCACC | |
| | TCGAGCACAACGATCTGCAGGAGCAGGAGCTGCAGACGGACCT | |
| | TGCGCACTACGAGAGCTATCAGTGGATCTACGAGCCGAACGAT | |
| | CTGTACTAATCCCAGAGACTTACCAGGCCAACAACTGCCAAGG | |
| | AGCTTGCGGCTGGCCACAGTCCGATCGAAACCCCCGATATGGC | |
| | AACCACGTGGTACTACTACTAAAAATGCAAGCACGAGGAGCCG | |
| | CTCTAGCCCGACCACCATGCTGCGTGCCTACCGCATATGGAGG | |
| | CAAGCTACTAATTTCACTATCTGAAGAACGGATCTCCGCACAT | |
| | CACGTACCTAATATGGTAGCTACTGAGTGTGGTTGTAGATGAG | |
| | GTACC | |
| 18 | MPGLLSPPALVLSVMGALLMAGDPGEEVSSTPALPGGPATGTG | Exemplary wild-type |
| | GLIFHPDWDWQPPGSPQDPLCLVTLDRGGNGSGSPLRVVGALR | felis catus MIS amino |
| | GYEHAFLEAVRRARWGPHGLATFGVCTPRDRQAAPFSLRQLQA | acid sequence (wt- |
| | WLGEPGGRRLVVLHLEEVTWEPTPSLKFQEPPPGGAGPLELAM | fcMIS-2); based on |
| | LVLYPGPGPEVTVTGAGLPGTQSLCQSRDTRYLVLAVDHPEGA | NCBI Reference |
| | WRSPGLTLTLQPRRDGAPLSTAQLQELLFGPDPRCFTRMTPAL | Sequence: |
| | LLLPGPAPAPLPARGLLDQVPLPPPRPSQEQAPEEPRSSADPF | XP_011286375.3 |
| | LETLTRLVRALRGPPAQASPARLALDPGALAGFPQGLVNLSDP | |
| | AAQERLLNGGDEPLLLLLLPPATPTAAAAAAAGDPAPPRGPAS | |
| | APWAAGLARRVAAELQAAAAELRGLPGLPPAATPLLARLLALC | |
| | PGDSGDSGDPGAPPGGPGGPLRALLLLKALQGLRAEWRGREQG | |
| | GPARAQRSAGAGAADGPCALRELSVDLRAERSVLIPETYQANN | |
| | CQGACGWPQSDRNPRYGNHVVLLLKMQARGAALARPPCCVPTA | |
| | YAGKLLISLSEERISAHHVPNMVATECGCR | |
| 19 | CGGTGTCCCTGTGTCCTCCCAGGATGCCGGTGGGTGAAGGACA | Nucleic acid sequence |
| | GACCCAGGCAGTCAGCAGCGGGTCTGGGCTCTCTTCTGCGGCC | encoding an |
| | GCTCACCCTCCTTGGGGTCTCCAGCCAAATGGCTGTGCTTCTG | exemplary felis catus |
| | CAGCTCCAGGGTGCCAAGAGGCAGTGTGAGCGAGCACTTGGGG | MIS (wt-fcMIS-2) of |
| | AGCCCCAGCTTCGCTGGGAGACCACCCTCTGGCCAAGCCCACG | SEQ ID NO: 18; based |
| | TGCACCCAGCGGTCTGAACACCAAGATCTCCGGTCCCCATCAG | on NCBI Reference |
| | AGCCGGGTGCCGGAGGCCTCCAGGGCGCCTGCCCCCCTGCCCC | Sequence: |
| | CATTCCAGAGCTGTTGATCGCCGGTCTGGTTCCCACTCCCTCC | XP_011286375.3 |
| | TGCAGAGGGGGAAAACCTCATCAAGGACAGTCGTTGGACCAAC | |
| | TGGGCACGGGCGGCACTCTGTATCACGGGTAGGAAGATAGGCG | |
| | GTCAGGCTGGAACAGAAGAGGCTTTGAGAGGCTCCTCTGCCTG | |
| | CCCAGGCCCACGGCGGGGCACCAGACGTTGGCCCCCAAGGTCA | |
| | CACCCCAGAAGGAGATAGGGGCTTTGCTCCTGCACAAACATCC | |
| | CGGTCTCCTCCATATAAGCCAGAGCCACACGGCCCCTCACAGC | |
| | AGCCAGGATGCCTGGTCTGCTCTCTCCGCCGGCCCTGGTGCTG | |
| | TCGGTGATGGGGGCTCTGCTGATGGCCGGGGACCCTGGGGAAG | |
| | AGGTCTCCAGCACCCCGGCCCTGCCTGGAGGGCCAGCCACAGG | |
| | CACCGGGGGTCTCATCTTCCACCCGGATTGGGACTGGCAGCCC | |
| | CCGGGCAGTCCCCAAGACCCCCTGTGCCTGGTGACGCTGGACA | |
| | GGGGTGGTAACGGGAGCGGCTCCCCGCTTCGGGTGGTGGGGGC | |
| | GCTGAGAGGCTACGAGCACGCCTTCCTCGAGGCTGTGCGGCGG | |
| | GCCCGCTGGGGTCCCCACGGCCTGGCCACCTTCGGAGTTTGCA | |
| | CCCCCAGGGACAGGCAGGCCGCCCCGTTCTCTCTGCGGCAGCT | |
| | GCAGGCGTGGCTGGGGGAGCCCGGGGGGCGGCGGCTGGTGGTG | |
| | CTGCACCTGGAGGAAGGTACGTAGGGAGGGGGCCACGGCCTGG | |
| | GGGGGTGCCACGCTGCCACCACTCTTTCCAGACCGGGTCCTGC | |
| | CGGAGCCCAACTCTAGACGCATCTTGGCCTCCGGGGAAGGCTG | |
| | AGGCTGAGGGCCCAGGAAGTGGGGGCCCTTCGTGTCTGGGGGC | |
| | TGCAAACCCCCCGATGCTTTGCTTCCAAGCCCACCCCTCCTGC | |
| | AGCCCTCCTGGGAGGTGTTTGCCGCCCCCCCCACCCCACGAAG | |
| | GAGCAGAAGGGAGTCCGGGCAGAGCCATGCTCCGCCCACACCC | |
| | CTCCTCCAAGGGTGGTTAGTGTGCGGCCTGTTCCCGGACGCTG | |
| | CTGGAGGAAATGGTTTCCCAGGGGACCCTATGGGCTCCCCCGC | |
| | TAAGGAGAGCCCCAGACAGAGACCCCATGGGGTGCTCCAGGCT | |
| | CCTCGCATTGGGGCAAGGCCCTGCACCCGATGTCTGGGCGTCA | |
| | AGCCTCTCCCGGTACGGGTGGGGGCTCTCCCTGCAGGACTGCA | |
| | AGACGGGCTTCGGGGAGGTGCTGGGGCCTCGGTGACTCAGGTG | |
| | TTGCCCCTTCCCTATTTGTCCCTCCTGGCCACAGTGACATGGG | |
| | AGCCGACACCCTCACTGAAGTTCCAGGAGCCCCCGCCTGGAGG | |
| | GGCCGGTCCCCTAGAGCTGGCGATGCTGGTGCTGTACCCCGGC | |
| | CCTGGCCCCGAGGTCACGGTCACAGGGGCTGGGCTGCCAGGCA | |
| | CCCAGGTACCGGGGTGTTGAAGGCGGTTAGTTCTGGGGCCTCC | |
| | AGGAGCCCTCCCCCAACAGAGGAGGGGGAATGGGGTTTTTTAA | |
| | CTGTGCTGAACAGAAAGGTTCCAAGTCCGCTGGTTGGAACCTT | |
| | GAAGGGGGGTGTCAAGGGCAATGGGCAGAGCAGGGCTGGTGTC | |
| | CTCGCCCCCCCCCCCCCACCTGGCTAGGCTGAGCCCCCGTCTC | |
| | CACAGAGCCTCTGTCAGTCCCGGGACACCCGCTACCTGGTGCT | |
| | GGCGGTGGACCACCCAGAAGGGGCCTGGCGCAGCCCCGGGCTC | |
| | ACCCTGACCCTGCAACCCCGCAGAGACGGTAGGCTCTCCAAGA | |
| | GAGGGACCGGGTAAGGGTGGGCGGCCCGCGGTCCTCCGCCCCC | |
| | GCTCAGCCAGGCCCCCGTGCTCCGCCACGCAGGTGCGCCCCTG | |
| | AGCACCGCCCAGCTGCAGGAGCTGCTGTTCGGCCCCGACCCCC | |
| | GCTGCTTCACACGCATGACCCCGGCGCTGCTCCTGCTGCCGGG | |
| | GCCCGCGCCTGCACCGCTGCCCGCGCGTGGCCTGCTGGACCAA | |
| | GTGCCTCTCCCGCCGCCCAGGTGTGCGCAGGCCCACGTTGGGG | |
| | GTGTCGGGAGGGGAACCCTGCACCTGCCCCTACCGCCCAACTC | |
| | CGCCTTCCAGGCCCTCCCAGGAGCAGGCGCCTGAGGAGCCACG | |
| | GTCCAGCGCCGACCCCTTCCTGGAGACGCTCACGCGCCTGGTG | |
| | CGCGCGCTGCGGGGGCCCCCGGCCCAGGCCTCGCCGGCGCGCC | |
| | TGGCCCTGGACCCCGGCGCGCTGGCCGGCTTCCCGCAGGGCCT | |
| | GGTCAACCTGTCGGACCCCGCGGCGCAGGAGCGCCTGCTCAAC | |
| | GGCGGCGACGAGCCGCTGCTGCTGCTTCTGCTGCCACCCGCCA | |
| | CGCCCACCGCCGCCGCCGCCGCCGCCGCCGGGGACCCCGCGCC | |
| | GCCGCGCGGCCCGGCGTCCGCGCCCTGGGCCGCCGGCCTAGCG | |
| | CGTCGCGTGGCCGCCGAGCTGCAGGCCGCGGCCGCCGAGCTGC | |
| | GAGGGCTCCCGGGGCTGCCGCCGGCCGCCACGCCGCTGCTGGC | |
| | GCGCCTGCTCGCGCTGTGCCCCGGGGATTCGGGGGACTCGGGG | |
| | GACCCGGGGGCCCCCCCCGGCGGCCCGGGCGGCCCGCTGCGCG | |
| | CGCTGCTGCTGCTCAAGGCGCTGCAGGGTCTGCGCGCGGAGTG | |
| | GCGCGGGCGCGAGCAGGGCGGACCGGCGCGGGCACAGCGCAGC | |
| | GCGGGGGCCGGGGCGGCCGACGGGCCGTGCGCGCTGCGCGAGC | |
| | TGAGCGTGGACCTGCGCGCCGAGCGCTCCGTGCTCATCCCGGA | |
| | GACGTACCAGGCCAACAACTGCCAGGGCGCGTGCGGCTGGCCG | |
| | CAGTCCGACCGCAACCCGCGCTACGGCAACCACGTGGTGCTGC | |
| | TGCTCAAGATGCAGGCCCGCGGCGCCGCCCTGGCGCGCCCGCC | |
| | CTGCTGCGTGCCCACGGCCTACGCGGGCAAGCTCCTCATCAGC | |
| | CTGTCGGAGGAGCGCATCAGCGCGCACCACGTGCCCAACATGG | |
| | TGGCCACCGAGTGCGGCTGCCGGTGAGCCCCGCACCGTGCCCC | |
| | CCGAGTGGCGTCCCCGCCCGTATTTATTCGGACCCCCATCATC | |
| | GCCCCAATAAAGACCAGCAAGCACAG | |
| 20 | MKWVTFISLLLLFSSAYSGDPGEEVSSTPALPGGPATGTGGLI | Exemplary LR-fcMIS- |
| | FHPDWDWQPPGSPQDPLCLVTLDRGGNGSGSPLRVVGALRGYE | 2 amino acid |
| | HAFLEAVRRARWGPHGLATFGVCTPRDRQAAPFSLRQLQAWLG | sequence; based on |
| | EPGGRRLVVLHLEEVTWEPTPSLKFQEPPPGGAGPLELAMLVL | NCBI Reference |
| | YPGPGPEVTVTGAGLPGTQSLCQSRDTRYLVLAVDHPEGAWRS | Sequence: |
| | PGLTLTLQPRRDGAPLSTAQLQELLFGPDPRCFTRMTPALLLL | XP_011286375.3 |
| | PGPAPAPLPARGLLDQVPLPPPRPSQEQAPEEPRSSADPELET | (amino acids 1-18 is |
| | LTRLVRALRGPPAQASPARLALDPGALAGFPQGLVNLSDPAAQ | an exemplary non- |
| | ERLINGGDEPLLLLLLPPATPTAAAAAAAGDPAPPRGPASAPW | MIS leader sequence, |
| | AAGLARRVAAELQAAAAELRGLPGLPPAATPLLARLLALCPGD | amino acids 19-586 is |
| | SGDSGDPGAPPGGPGGPLRALLLLKALQGLRAEWRGREQGGPA | core wt-fcMIS-2 |
| | RARRSAGAGAADGPCALRELSVDLRAERSVLIPETYQANNCQG | protein sequence, with |
| | ACGWPQSDRNPRYGNHVVLLLKMQARGAALARPPCCVPTAYAG | Q475R modification |
| | KLLISLSEERISAHHVPNMVATECGCR | amino acids RARRS |
| | | in bold indicate |
| | | enhanced cleavage |
| | | site) |

### BRIEF DESCRIPTION OF THE DRAWINGS

FIGS. 1A-1H show a pilot study with fcMISv1 feline transgene in vitro, in mice and in cats. FIG. 1A shows a sequence alignment of divergent sections of the feline MIS protein from the domestic cat genome release 8.0 (fcMISv1; SEQ TD NO: 3 positions 361-467) and version 9.0 (fcMISv2; SEQ TD NO: 1, positions 361-480). FIG. 1B shows protein gel electrophoresis stained with colloidal blue of Flag-affinity purified Flag-fcMISv1 and Flag-fcMISv2 proteins (2 μg), with or without in vitro plasmin cleavage. Recombinant human LR-hsMIS and murine Flag-mmMIS (cleaved and uncleaved) are provided as controls. FIG. 1C shows representative western blot of tissue lysates from mice treated with 5e12 vg/kg of AAV9-fcMISv1 or 5e12 vp/kg of AAV9-empty negative control, and recombinant LR-hsMIS (100 ng) as a positive control. The blot was probed with an antibody to the C-terminus of MIS, or B-actin and GAPDH as loading controls. FIG. 1D shows representative fetal rat urogenital ridge sections stained in H&E following incubation with purified Flag-fcMISv1 (uncleaved or cleaved) protein or CHO cell clone conditioned media containing fcMISv1 adjusted to 5 μg/mL (untransfected CHO conditioned media negative control) of CHO feline MIS protein. Activity of MIS is scored from grade 0-5 representing the degree of Müllerian duct regression. (F=Wolffian duct; M=Müllerian duct.) FIG. 1E shows representative gross morphology of the uterine horn and ovary of a mouse treated with AAV9-fcMISv1 (5e12 vg/kg) or control AAV9-empty (5e12 vp/kg) at day 50 post-treatment. FIGS. 1F-1H show serum MIS and anti-fcMISv1 antibody titers in three cats following treatment with 5e12 vg/kg fcMISv1.

FIG. 2 shows histological analyses of uteri and ovaries of cats treated with fcMISv1. Three years after treatment with AAV9-fcMISv1, the three female cats were spayed, and the histology of the uterus and ovary were examined. Note that Subject 11WBL24, which developed rapid and potent immunity to the fcMISv1 transgene has multiple corpora lutea and few primordial follicles in the ovary, and cystic endometrial hyperplasia in the uterus. In contrast, Subject 11WBL25 which maintained MIS levels in the μg/ml range, has abundant primordial follicle populations in the ovary and normal corpora lutea and normal endometrium.

FIGS. 3A-3G show cloning of a feline MIS AAV9 vector and validation in mice. FIG. 3A shows design of codon-optimized feline MIS transgenes. FIG. 3B shows Western blot of conditioned media and MIS proteins (100 ng) purified by Flag affinity, cleaved in vitro or not with plasmin, from stable CHO clones overexpressing human, mouse, and cat transgenes. The blot was probed with an antibody to the C-terminus of MIS. FIG. 3C shows representative fetal rat urogenital ridge sections stained with H&E following incubation with purified protein or conditioned media adjusted to 5 μg/ml of human or feline MIS protein. Activity of MIS is scored from grade 0-5 representing the degree of Müllerian duct regression. (F=Wolffian duct, M=Müllerian duct). FIG. 3D shows representative western blot of tissue lysates from mice treated with 5e12 vg/kg of AAV9-fcMISv2 or 5e12 vp/kg of AAV9-empty negative control, and recombinant LR-hsMIS (100 ng) as a positive control. The Blot was probed with an antibody to the c-terminus of MIS, or B-actin and GAPDH as loading controls. FIG. 3E shows serum concentration of MIS measured by ELISA in mice following treatment with 5e12vg/kg or 1e13 vg/kg of AAV9-fcMISv2. FIG. 3F shows representative middle section of an ovary 4 weeks after treatment with 5e12 vg/kg or 1e13 vg/kg of AAV9-fcMISv2, or 5e12 vp/kg of AAV9-empty negative control. FIG. 3G) the respective total follicle counts in the whole ovaries (N=5 per group).

FIG. 4 shows the mean serum MIS concentration (g/ml) in mature cats from pilot study 1 treated with AAV9-chimeric feline MIS (5e12 vector particles (vp)/kg), and cats from pilot study 2 following injection with either low (5e12 vp/kg) or high (1e13 vp/kg) doses of AAV9-wt feline MIS, as described in Example 1.D.

FIGS. 5A-5C show individual profiles of serum MIS concentration (g/ml) (square) and circulating anti-MIS antibody concentration (circle) in each mature cat from pilot study 2 following injection with low dose of AAV9-wt feline MIS (5e12 vp/kg, n=3) (FIG. 5A), high dose of AAV9-wt feline MIS (1e13 vp/kg, n=3) (FIG. 5B), or control empty vector particles (5e12 vp/kg, n=3) (FIG. 5C), as described in Example 1.D.

FIGS. 6A-6G show evaluation of AAV9-fcMISv2 in domestic cats. FIG. 6A shows sexually mature female domestic cats were treated intramuscularly with 5e12 vg/kg (low MIS) or 1e13 vg/kg (high MIS) of AAV9-fcMISv2, or 5e12 vp/kg of AAV9-empty vector (control). Fertility was assessed during two mating studies concluding at the one-year and two-year post-treatment mark. Serum concentration of MIS (FIG. 6B), luteinizing hormone (LH; FIGS. 6C and 6E), and inhibin B (FIGS. 6D and 6E) were measured by ELISA in cats following treatment with AAV9-fcMISv2. FIG. 6F shows concentrations of estradiol (E2) and progesterone (P4) in dried fecal pellet collected from cats throughout the pre- and post-treatment periods. FIG. 6G shows assessment of estrus and luteal phase frequency based on fecal steroid profiles in pre- and post-treatment periods.

FIGS. 7A-7G show viral vector shedding assessment and individual serum fcMISv2 and anti-drug antibody profiles in domestic cats treated with AAV9-fcMISv2 or empty vector controls. FIGS. 7A-7D show viral genome quantification by qPCR in blood (FIG. 7A), stool (FIG. 7B), urine (FIG. 7C), and oral swab (FIG. 7D) samples following treatment. FIGS. 7E-7G show serum MIS and anti-fcMISv2 antibody titers in individual cats following treatment with AAV9-fcMISv2 (FIGS. 7E-7F) or empty vector control (FIG. 7G).

FIGS. 8A-8I show sex steroid and assessment of cyclicity in cats treated with AAV9-fcMISv2. The concentration of E2 and P4 was in dried fecal pellet collected from cats throughout the pre-treatment (left panels) and post-treatment (right panels) periods. Peak steroid concentration over baselines were used to estimate estrus and luteal phases.

FIGS. 9A-9D show evaluation of breeding behaviors during mating studies, and MIS levels during mating and in control kittens. FIGS. 9A-9B show control and AAV9-fcMISv2 female cats that were introduced to male breeders. Their behavior was recorded by video capture and assessed for successful and unsuccessful breeding attempts. FIG. 9C shows MIS levels at mating. FIG. 9D shows MIS levels in kittens born to control females.

FIG. 10 shows a western blot of transient transfection of cat and dog vectors in CHO cells.

FIG. 11 shows a western blot of transient transfection of cat and dog vectors in COS7 cells.

FIG. 12 shows qPCR of transient transfection of cat and dog vectors in COS7 cells.

FIGS. 13A-13B show concentrated media from CHO clones (FIG. 13A) and urogenital ridge regression bioassay (FIG. 13B).

FIG. 14 shows total follicle counts in mice 30 days after treatment with AAV9-fcMISv2 with a dose-response of vectors.

FIGS. 15A-15D show counts of primary follicles (FIG. 15A), secondary follicles (FIG. 15B), antral follicles (FIG. 15C), and corpus luteum (FIG. 15D) in mice 30 days after treatment with 1e13 vg/kg of AAV9-empty, AAV9-fcMISv2, or AAV9-LRclMIS.

FIG. 16 shows evaluation of circulating mis protein by ELISA (ANSH) during the 4 weeks post-treatment with 1e13 vg/kg.

FIGS. 17A-17B show qPCR quantification of viral genomes in the muscle (FIG. 17A) and liver (FIG. 17B) of mice at 30 days after treatment with 5e12 vg/kg.

FIG. 18 shows evaluation of MIS protein cleavage by western blot in liver lysates at 30 days after treatment with 1e13 vg/kg.

FIG. 19 shows evaluation of MIS protein expression by ELISA following treatment with AAV.MYO and AAV9-HR vectors at 5e12 vg/kg delivering fcMISv2 (SEQ ID NO: 1).

FIG. 20 show MIS and inhibin B profiles of kittens.

FIGS. 21A-21B show normalized inhibin B in female (FIG. 21A) and male kittens (FIG. 21B)

FIG. 22 shows anti-MIS neutralizing antibody profiles in kittens compared to the positive control Subject 11WBL24.

FIGS. 23A-23C show 23 fecal steroid profiles in Subjects M200586, M200667, and M200756.

FIGS. 24A-24B show uterine horn measurements performed by transabdominal ultrasound. Measurements were performed on cats treated with 5e12 vg/kg AAV9-fcMISv2 (low), 1e13 vg/kg AAV9-fcMISv2 (high), or 5e12 vp/kg of AAV9-empty vector (control). FIG. 24A shows measurements for all treated cats. In FIG. 24B, “treated” represents average data points for cats treated “low” and “high” doses.

FIGS. 25A-25B show uterine horn measurements performed by transabdominal ultrasound at 6-10 months after treatment. The measurements shown were corrected for the age of cats. Measurements were performed on cats treated with 5e12 vg/kg AAV9-fcMISv2 (low), 1e13 vg/kg AAV9-fcMISv2 (high), or 5e12 vp/kg of AAV9-empty vector (control). FIG. 25A shows measurements for all treated cats. In FIG. 25B, “treated” represents average data points for cats treated “low” and “high” doses.

### DETAILED DESCRIPTION

The present invention relates to compositions and methods of administering a nucleic acid encoding a Müllerian inhibiting substance (MIS) protein (e.g., administering a viral vector comprising a nucleic acid encoding a MIS protein) for reducing fertility and/or preventing puberty and/or preventing reproduction in prepubescent non-human subjects, such as kittens and puppies.

As set forth in detail herein, administering MIS via gene delivery to reduce fertility and/or preventing puberty is currently under clinical development in prepubescent non-human subjects, including male and female kittens and puppies.

A. MIS as an Agent for Long-Term Reduction of Fertility and/or Prevention of Puberty of Non-Human Subjects, e.g., Kittens and Puppies

As discussed herein, one aspect of the present invention relates to administering a nucleic acid encoding a MIS protein (i.e., by gene transfer) to a prepubescent non-human subject (e.g., kitten or puppy) as a method of long-term reduction of fertility and/or prevention of puberty, for example as an alternative to surgical spaying or neutering. Accordingly, a single injection of a vector (e.g., a viral vector) expressing a MIS protein may be a safe and effective alternative to surgical spaying or neutering in prepubescent kittens and puppies. The methods as disclosed herein can be used to reduce fertility in kittens and puppies and/or prevent them from reaching puberty.

As used herein, “prepubescent” refers to a subject that has not reached puberty and is considered sexually immature.

In some embodiments, subjects amenable to treatment include any non-human prepubescent subjects, for example, prepubescent subjects who would undergo surgical spaying or neutering. Surgical spaying or neutering is common in, e.g., kittens, cats, puppies, and dogs. In some embodiments, the prepubescent subject is a kitten, or puppy or any animal that has not undergone puberty. In some embodiments, subjects can be administered a nucleic acid encoding a MIS protein (e.g., by viral vector) as a single dose. In some embodiments, the dose can be administered as a single injection or split into multiple injections.

In some embodiments, the prepubescent subject is a kitten. In other embodiments, the prepubescent subject is a puppy.

In some embodiments, the prepubescent kitten is a female kitten. In some embodiments, the prepubescent kitten is a male kitten. In some embodiments, the prepubescent kitten is 12 months old or less, 11 months old or less, 10 months old or less, 9 months old or less, 8 months old or less, 7 months old or less, 6 months old or less, 5 months old or less, 4 months old or less, 3 months old or less, or 2 months old or less. In some embodiments, the prepubescent kitten weighs 2 kg or less.

In some embodiments, the prepubescent puppy is a female puppy. In some embodiments, the prepubescent puppy is a male puppy. In some embodiments, the prepubescent puppy is 24 months old or less, 22 months old or less, 20 months old or less, 18 months old or less, 16 months old or less, 14 months old or less, 12 months old or less, 11 months old or less, 10 months old or less, 9 months old or less, 8 months old or less, 7 months old or less, 6 months old or less, 5 months old or less, 4 months old or less, 3 months old or less, or 2 months old or less.

B. MIS as an Agent for Increasing Sperm Numbers and/or Sperm Concentrations

An aspect of the present disclosure relates to administering a MIS protein or a nucleic acid encoding a MIS protein (i.e., by gene transfer) to a non-human male subject (e.g., endangered or rare animal) as a method of increasing sperm numbers and/or sperm concentrations. It may be beneficial in some scenarios to increase sperm numbers and/or sperm concentrations in the subject to aid the collection and storage of sperm samples for future artificial insemination. Accordingly, a single injection of a vector (e.g., a viral vector) expressing a MIS protein may be a safe and effective method to increase sperm numbers and/or sperm concentrations in non-human male subjects. The methods as disclosed herein can be used to increase fertility in non-human male subjects.

In some embodiments, the non-human male subject is an adult or is sexually mature. Methods related to the determination of adulthood or reproductive maturity of the non-human male subject is known to one of ordinary skill in the art. Examples of these methods include (1) measurements of sex hormones (e.g., testosterone, progesterone, and estrogen) and, (2) analysis for morphological signs of reproductive maturity (i.e., puberty), such as enlargement of penis and testes, the presence of penile spines, and other morphological changes in reproductive organs.

### C. MIS Protein as an Agent for Delaying Puberty of Human Subjects

In some embodiments, the subject is a prepubescent human subject, e.g., a female prepubescent human subject, where the method comprises administering to the subject an effective amount of a composition comprising a recombinant human MIS protein as disclosed herein. In some embodiments, the subject is a human female subject in need of delaying puberty, for example, in order to provide the subject more time before beginning a gender reassignment treatment and/or surgery, or before the female subject begins treatment to transition from a female to a male gender, or to give the subject more time to fully understand the subject gender identity. By way of explanation only, the term a “human female subject” typically refers to a subject that is assigned as a being of female biological sex at birth (e.g., has XX chromosomes and/or appearance of female genitalia or appears of a female biological gender or presence of female external and/or internal reproductive anatomy). In some embodiments, a “human female subject” referred to herein can also include subjects designated as intersex at birth, or a subject that has atypical genitalia at birth, or has both male and female reproductive organs, or has only internal (but not external) female reproductive anatomy, or has only external (but not internal) female reproductive anatomy, or mosaic genetics (where some chromosomes are labeled XY and other XX), or Klinefelter syndrome (in which the individual has XXY chromosomes) or where the subject is referred to or designated as having a non-binary gender. By way of explanation only, where the method comprises administering to a human subject an effective amount of a composition comprising a recombinant human MIS protein as disclosed herein for delaying puberty, the delay of puberty is for the period that the human MIS is administered to the subject. In some embodiments, puberty is delayed about 6-months, or about 8 months, or about 12 months, or about 18 months or about 2 years, or about 3 years or about 4 years, or about 5 years, or longer than 5 years. In some embodiments, puberty in a human subject is not prevented, rather it is reversibly delayed for a period of time and once the subject stops being administered the recombinant human MIS protein, puberty will progress or resume at some point in the human subject. In some embodiments, if the subjects undergoes a gender reassignment, puberty of the reassigned gender will resume after treatment and stopping of the treatment with the recombinant human MIS protein.

In some embodiments, the human female subject has idiopathic precocious puberty, where precocious puberty is where the child's body begins to change into that of an adult too soon. Precocious puberty is premature development of body characteristics that normally occur during puberty (the period in life at which rapid physical and physiologic changes occur, including development of reproductive capability). Puberty normally occurs between 13 and 15 years old in boys and between 9 and 16 years old in girls. In some embodiments, the human female subject has premature puberty, or central precocious puberty (CPP) or peripheral precocious puberty, which is where puberty begins age 8 or before for girls. Precocious puberty signs and symptoms include development of at least one or more of the following before age 7 in girls and before age 9 in boys: breast growth, first period in girls (onset of menses), maturation of the external genitialia, pubic or underarm hair, rapid growth, acne and adult body odor. In some embodiments, the human female subject has gender atypical genitalia at birth and has both male and female reproductive organs. CPP may be caused by one or more of: a tumor in the brain or spinal cord (central nervous system), a defect in the brain present at birth, such as excess fluid buildup (hydrocephalus) or a noncancerous tumor (hamartoma), radiation to the brain or spinal cord, injury to the brain or spinal cord, McCune-Albright syndrome (a genetic disease that affects bones and skin color and causes hormonal problems), congenital adrenal hyperplasia (a group of genetic disorders involving abnormal hormone production by the adrenal glands) or hypothyroidism. Peripheral precocious puberty in girls may be associated with one or more of ovarian cysts or ovarian tumors, as well as a tumor in the adrenal glands or in the pituitary gland that releases estrogen or testosterone, McCune-Albright syndrome or exposure to external sources of estrogen or testosterone, such as creams or ointments.

In some embodiments, the human female subject has gender dysphoria. In some embodiments, the human female subject has congenital adrenal hyperplasia (CAH).

In some embodiments, the human female subject has a disease or disorder associated with reduced bone growth and where puberty will stop bones from growing.

### D. Müllerian Inhibiting Substance (MIS) Proteins

Without wishing to be bound by any theory, the Müllerian Inhibiting Substance (MIS) is a member of the TGFβmultigene family of glycoproteins. The proteins in this gene family are all produced as dimeric precursors and undergo posttranslational processing for activation, requiring cleavage and dissociation to release bioactive C-terminal fragments. MIS is a 140 kDa dimer which consists of identical 70 kDa disulfide-linked monomers, each composed of a 57 kDa N-terminal domain and a 12.5 kDa carboxyl-terminal (C-terminal). Thus, MIS comprises 2 identical monomers (and thus is termed a “homodimer”), each monomer comprising two domains, the N-terminal and C-terminal domain, which are held in non-covalent association. The purified C-terminal domain is the biologically active moiety and cleavage is required for activity. The N-terminal domain may assist with protein folding in vivo and facilitate delivery of the C-terminal peptide to its receptor, e.g., MISRI and MISRII. A non-cleavable mutant of MIS is biologically inactive.

The carboxy-terminal active domain shares amino acid homology with other TGFβ family members, such as TGF-B 1, 2, and 3, inhibin, activin, and bone morphogenetic proteins, as well as a number of Growth and Differentiation Factors (GDFs). The structure of the MIS carboxy-terminal domain is supported by seven cysteines involved both in intra- and intermolecular disulfide bridges that lead to its structural stability, as revealed by homology to the three-dimensional structure of TGFβ using molecular modeling (Lorenzo, Donahoe, et al., unpublished data).

Like other TGFβ family members, MIS can be cleaved by plasmin which generates its amino- and carboxy-terminal domains. This proteolytic process is required for physiological activity and occurs at a site in a position similar to the dibasic cleavage site found in the sequence of TGFβ. The resultant products are tightly associated in a non-covalent complex that dissociates at low pH; therefore, technically complex and time-demanding protocols with plasmin treatment and molecular size exclusion chromatography are required to enhance or complete the separation of the carboxy terminus from the amino terminus.

The cat MIS gene was previously cloned, but it was discovered that the older version of the cat genome may have had incomplete coverage of the MIS sequence. Previous efforts to clone the missing region were only partially successful likely because of high GC content and secondary structures leading to structural rearrangement artifacts. By piecing together partial sequences acquired by Sanger sequencing and relying on the high degree of homology of the C-terminus of MIS in Carnivora, the inventors obtained an updated chimeric feline MIS transgene produced synthetically with a synonymous codon usage diminishing the GC content enough to allow viral packaging was constructed. This chimeric feline MIS construct (SEQ ID NO: 3) was discovered to have introduced 17 amino acid substitutions and omitted two peptide motifs totaling 13 AA for a total of 30 amino acid mismatches (yet was still bioactive in UGR assay) when the more comprehensive updated (“version 9”) build of the cat genome was released (see from GenBank Accession No. GCA_000181335.4). A wild-type feline MIS construct (SEQ ID NO: 1) matching the cat V9.0 reference sequence and having optimized codon usage to reduce GC content was prepared (SEQ ID NOs: 1 and 5). An update to the cat V9.0 sequence has since been released (see GenBank Accession No. GCA_000181335.5). This updated cat V9.0 genome sequence information includes an updated cat MIS protein sequence (SEQ ID NO: 18). The updated cat v9.0 MIS sequence (SEQ ID NO: 18) differs from the original cat v9.0 MIS sequence (SEQ ID NO: 1) by having one additional alanine residue within an alanine-rich stretch at positions 370-376 of SEQ ID NO: 18 compared to 370-375 of SEQ ID NO: 1. The difference of one alanine residue at this stretch of feline MIS is not anticipated to affect its processing, activity, and/or antigencity.

In some embodiments, the MIS protein comprises a wild-type feline MIS (fMIS) protein. In some embodiments, the MIS protein comprises the amino acid sequence of SEQ ID NO: 1. In some embodiments, the MIS protein comprises an amino acid sequence having at least 80%, at least 85%, at least 90%, at least 95%, at least 96%, at least 97%, at least 98%, at least 99% sequence identity to the amino acid sequence of SEQ ID NO: 1. In some embodiments, the MIS protein comprises the amino acid sequence of SEQ ID NO: 18. In some embodiments, the MIS protein comprises an amino acid sequence having at least 80%, at least 85%, at least 90%, at least 95%, at least 96%, at least 97%, at least 98%, at least 99% sequence identity to the amino acid sequence of SEQ ID NO: 18. In some embodiments, the MIS protein comprises the amino acid sequence of SEQ ID NO: 3. In some embodiments, the MIS protein comprises an amino acid sequence having at least 80%, at least 85%, at least 90%, at least 95%, at least 96%, at least 97%, at least 98%, at least 99% sequence identity to the amino acid sequence of SEQ ID NO: 3.

In some embodiments, the disclosure comprises a composition comprising a chimeric feline MIS protein. In some embodiments, the chimeric feline MIS protein comprises the amino acid sequence of SEQ ID NO: 3. In some embodiments, the chimeric feline MIS protein comprises an amino acid sequence having at least 80%, at least 85%, at least 90%, at least 95%, at least 96%, at least 97%, at least 98%, at least 99% sequence identity to the amino acid sequence of SEQ ID NO: 3. In some embodiments, the disclosure comprises a composition comprising a chimeric feline MIS protein comprising at least amino acids 22-572 of SEQ ID NO: 3, or a chimeric feline MIS protein comprising a protein that has at least 85% sequence identity to amino acids 22-572 of SEQ ID NO: 3, and wherein the endogenous chimeric feline MIS protein leader sequence of residues 1-21 of SEQ ID NO: 3 is replaced with a non-MIS leader sequence disclosed herein.

In some embodiments, the MIS protein comprises a wild-type canine MIS protein. In some embodiments, the MIS protein comprises the amino acid sequence of SEQ ID NO: 2. In some embodiments, the MIS protein comprises an amino acid sequence having at least 80%, at least 85%, at least 90%, at least 95%, at least 96%, at least 97%, at least 98%, at least 99% sequence identity to the amino acid sequence of SEQ ID NO: 2.

In some embodiments, the disclosure comprises a composition comprising a wild-type canine MIS protein. In some embodiments, the MIS protein comprises the amino acid sequence of SEQ ID NO: 2. In some embodiments, the MIS protein comprises an amino acid sequence having at least 80%, at least 85%, at least 90%, at least 95%, at least 96%, at least 97%, at least 98%, at least 99% sequence identity to the amino acid sequence of SEQ ID NO: 2. In some embodiments, the disclosure comprises a composition comprising a canine MIS protein comprising at least amino acids 23-572 of SEQ ID NO: 2, or a canine MIS protein comprising a protein that has at least 85% sequence identity to amino acids 23-573 of SEQ ID NO: 2, and wherein the endogenous canine MIS leader sequence of residues 1-22 of SEQ ID NO: 2 is replaced with a non-MIS leader sequence disclosed herein.

As used herein, “percent sequence identity” in the context of two or polypeptide sequences, refer to two or more sequences or subsequences that are the same or have a specified percentage of amino acid residues or conservative substitutions thereof, that are the same, when compared and aligned for maximum correspondence, as measured using one of the following sequence comparison algorithms, or by visual inspection. By way of example, a first amino acid sequence can be considered similar to a second amino acid sequence when the first amino acid sequence is at least 50%, 60%, 70%, 75%, 80%, 90%, or even 95% identical, or conservatively substituted, to the second amino acid sequence when compared to an equal number of amino acids as the number contained in the first sequence, or when compared to an alignment of polypeptides that has been aligned by a computer similarity program known in the art, as discussed below.

It will be appreciated by those of skill that cloned genes readily can be manipulated to alter the amino acid sequence of a MIS protein. The cloned gene for a MIS protein can be manipulated by a variety of well-known techniques for in vitro mutagenesis, among others, to produce variants of the naturally occurring protein, which may be used in accordance with the methods and compositions described herein.

The variation in primary structure of a MIS protein useful in the invention, for instance, may include deletions, additions and substitutions. The substitutions may be conservative or non-conservative. The differences between the natural protein and variant generally conserve desired properties, mitigate or eliminate undesired properties and add desired or new properties.

The mature wild-type MIS protein is initially produced as a prohormone comprising a N-terminal leader sequence, which corresponds to amino acid residues 1-21 of wild-type feline MIS protein of SEQ ID NO: 1 or SEQ ID NO: 18, amino acid residues 1-22 of wild-type canine MIS protein of SEQ ID NO: 2, and amino acid residues 1-24 of wild-type human MIS protein of SEQ ID NO: 4. This leader sequence is cleaved off to render the mature MIS protein. Moreover, the mature protein is cleaved at RAQ/R furin cleavage site (at amino acid residue 476-479 of wild-type feline MIS of SEQ ID NO: 1; at amino residue 477-480 of wild-type feline MIS of SEQ ID NO: 18; or amino acid residues 463-466 of chimeric feline MIS protein of SEQ ID NO: 3) to result in a N-terminal and C-terminal domains. A N-terminal and C-terminal MIS domains homodimerize with another WT feline MIS protein comprising the N-terminal and C-terminal domains to form the mature protein. In some embodiments, the RAQ/R furin cleavage site of SEQ ID NO: 1, SEQ ID NO: 18, or SEQ ID NO: 3 can be modified. In some embodiments, the Q amino acid residue at position 478 of SEQ ID NO: 1, or at position 479 of SEQ ID NO: 18, or at position 465 of SEQ ID NO: 3 can be changed from a Q to a R (arginine), or a conservative amino acid of R such as, a K (lysine).

In all aspects of the invention, a MIS protein or a nucleic acid sequence encoding the same for use in the methods and compositions as disclosed herein can have a non-endogenous MIS leader sequence, where the native MIS leader sequence has been replaced with a different leader sequence, such as, for example, a human serum albumin (HSA) leader sequence. In some embodiments, a MIS protein or a nucleic acid sequence encoding the same for use in the methods and compositions as disclosed herein is a modified MIS protein where the primary RAQ/R cleavage site (corresponding to amino acid 476-479 of wild-type feline MIS of SEQ ID NO: 1, corresponding to amino acid 477-480 of wild-type feline MIS of SEQ ID NO: 18, corresponding to amino acid 460-463 of wild-type canine MIS of SEQ ID NO: 2, and corresponding to amino acid 448-451 of wild-type human MIS of SEQ ID NO: 4 is changed to RAR/R, and/or where the endogenous MIS leader sequence has been replaced with an albumin leader sequence. MIS proteins useful in the methods as disclosed herein can be wild-type MIS, or MIS variants, such as LR-MIS, LRF-MIS and the like as disclosed in WO2015089321, which is incorporated herein in its entirety.

In some embodiments, a non-endogenous leader sequence for use in the present invention is a functional fragment or variation of an HSA leader sequence disclosed in WO2015089321, U.S. Pat. No. 5,759,802, EP patent 2277889, each of which is incorporated by reference herein in its entirety. Other leader sequences are encompassed for use in a MIS protein as disclosed herein, e.g., to replace the endogenous leader sequence. Such leader sequences are well known in the art, and include the leader sequences comprising an immunoglobulin signal peptide fused to a tissue-type plasminogen activator propeptide (IgSP-tPA), as disclosed in US 2007/0141666, which is incorporated by reference herein in its entirety. Numerous other signal peptides are used for production of secreted proteins. One of them is a murine immunoglobulin signal peptide (IgSP, EMBL Accession No. M13331). IgSP was first identified in 1983 by Loh et al. (Cell. 33:85-93). IgSP is known to give a good expression in mammalian cells. For example, EP patent No. 0382762 discloses a method of producing horseradish peroxidase by constructing a fusion polypeptide between IgSP and horseradish peroxidase. Other leader sequences include, for example, but not limited to, the MPIF-1 signal sequence (e.g., amino acids 1-21 of GenBank Accession number AAB51134); the stanniocalcin signal sequence; the invertase signal sequence; the yeast mating factor alpha signal sequence (e.g., K. lactis killer toxin leader sequence); a hybrid signal sequence; an HSA/MFa-1 hybrid signal sequence (also known as HSA/kex2); a K. lactis killer/MFα-1 fusion leader sequence; the Immunoglobulin Ig signal sequence; the Fibulin B precursor signal sequence; the clusterin precursor signal sequence; and the insulin-like growth factor-binding protein 4 signal sequence, examples of which are disclosed in WO2015089321, which is incorporated by reference herein in its entirety.

In some embodiments, the non-endogenous leader sequence for use in the present invention is a functional fragment or variation of a azurodicin (Azuro or “A”) leader sequence comprising amino acids of MTRLTVLALLAGLLASSRA (SEQ ID NO: 9), of a variant or fragment of SEQ ID NO: 9 having at least 85%, or a at least 80%, at least 85%, at least 90%, at least 95%, at least 96%, at least 97%, at least 98%, at least 99% sequence identity to the amino acid sequence of SEQ ID NO: 9.

In some embodiments, the non-endogenous leader sequence for use in the present invention is a HSA sequence is a functional fragment of SEQ ID NO: 10, for example, or at least 23, or at least 22, or at least 21, or at least 20, or at least 19, or at least 18, or at least 17, or at least 16, or at least 15, or at least 14, or at least 13, or at least 12, or at least 11, or at least 10, or less than 10 consecutive or non-consecutive amino acids of SEQ ID NO: 10. Modified versions of HSA leader sequence are also encompassed for use in the present invention and are disclosed in U.S. Pat. No. 5,759,802 which is incorporated herein in its entirety by reference. In some embodiments, a HSA leader sequence is MKWVTFISLLFLFSSAYS (SEQ ID NO: 10) or MKWVTFISLLFLFSSAYSRGVFRR (SEQ ID NO: 11) or variations therefor, which are disclosed in EP patent EP2277889 which is incorporated herein in its entirety. Variants of the pre-pro region of the HSA signal sequence (e.g., MKWVTFISLLFLFSSAYSRGVFRR, SEQ ID NO: 12) include fragments, such as the pre region of the HSA signal sequence (e.g., MKWVTFISLLFLFSSAYS, SEQ ID NO:10) or variants thereof, such as, for example, MKWVSFISLLFLFSSAYS, (SEQ ID NO:13).

Certain homologues and functional derivatives and functional fragments of feline, canine, and human MIS protein are known in the art or can be identified by a person of ordinary skill in the art by expression of MIS from an expression library.

### E. Delivery of MIS Proteins Via Gene Transfer

Accordingly, in one aspect, the present invention relates to a method of reducing fertility and/or preventing puberty in a prepubescent non-human subject, including kittens and puppies and other animals, the method comprising administering to the subject a composition comprising a vector comprising a nucleic acid encoding a MIS protein (e.g., wild-type MIS protein or a variant MIS protein).

In some embodiments, a nucleic acid encoding a MIS protein can be effectively used to reduce fertility and/or prevent puberty in a prepubescent non-human subject via gene transfer. The general principle is to introduce the nucleic acid into a target cell within a subject (in vivo) or into a target cell outside the subject and transfer the cell into the subject (ex vivo), and where the nucleic acid is transcribed into a MIS protein.

Accordingly, in some embodiments, the method described herein can reduce fertility and/or prevent puberty in prepubescent non-human subjects after a single injection of a composition comprising a vector comprising a nucleic acid encoding a MIS protein, wherein the composition administered to the subject can sustain the expression of MIS equal to or above a threshold level. The threshold level is the minimal level of MIS that may be needed to reduce fertility and/or prevent puberty.

It should be noted that the threshold level can depend on the subject, the species of the subject, and/or the age or maturity of the subject. There are a variety of practical situations where infertility is desired, for example, in veterinary applications.

Entry into the cell can be facilitated by suitable techniques known in the art such as providing the nucleic acid in the form of a suitable vector, or encapsulation of the nucleic acid in a liposome.

A desired mode of gene transfer is to provide the nucleic acid in such a way that it will replicate inside the cell, enhancing and prolonging the desired effect. Thus, the nucleic acid is operably linked to a suitable regulatory element, such as a promoter, e.g., the natural promoter of the corresponding gene, a heterologous promoter that is intrinsically active in liver, neuronal, bone, muscle, skin, joint, or cartilage cells, or a heterologous promoter that can be induced by a suitable agent.

Accordingly, in some embodiments, a vector (e.g., a viral vector) comprises a nucleic acid encoding a wild-type MIS protein comprising, for example, the amino acid sequence of SEQ ID NO: 1, SEQ ID NO: 18, SEQ ID NO: 2, or SEQ ID NO: 4. In some embodiments, the vector comprises a nucleic acid encoding a feline MIS protein of SEQ ID NO: 1 or SEQ ID NO: 18. In some embodiments, the vector comprises a nucleic acid encoding a canine MIS protein of SEQ ID NO: 2. In some embodiments, the vector comprises a nucleic acid encoding a human MIS protein of SEQ ID NO: 4.

In some embodiments, the vector comprises a nucleic acid that encodes a protein which has an amino acid sequence that has at least 90%, at least 91%, at least 92%, at least 93%, at least 94%, at least 95%, at least 96%, at least 97%, at least 98%, at least 99% sequence identity to the amino acid sequence of the MIS protein natively produced in the subject. In some embodiments, the vector comprises a nucleic acid that encodes a MIS protein comprising an amino acid sequence that has at least 90%, at least 91%, at least 92%, at least 93%, at least 94%, at least 95%, at least 96%, at least 97%, at least 98%, or at least 99% sequence identity to the amino acid sequence of SEQ ID NO: 1, SEQ ID NO: 18, SEQ ID NO: 2, or SEQ ID NO: 4, or a functional fragment thereof.

In some embodiments, the vector comprises a nucleic acid encoding a MIS protein of SEQ ID NO: 3. In some embodiments, the vector comprises a nucleic acid that encodes a MIS protein comprising an amino acid sequence that has at least 90%, at least 91%, at least 92%, at least 93%, at least 94%, at least 95%, at least 96%, at least 97%, at least 98%, or at least 99% sequence identity to the amino acid sequence of SEQ ID NO: 3.

In some embodiments, the vector comprises a nucleic acid sequence encoding a MIS protein, wherein the nucleic acid comprises the nucleotide sequence of SEQ ID NO: 5 or a nucleotide sequence having at least 95%, at least 96%, at least 97%, at least 98%, or at least 99% sequence identity to the nucleic acid sequence of SEQ ID NO: 5.

In some embodiments, the vector comprises a nucleic acid sequence encoding a MIS protein, wherein the nucleic acid comprises the nucleotide sequence of SEQ ID NO: 6 or a nucleotide sequence having at least 95%, at least 96%, at least 97%, at least 98%, or at least 99% sequence identity to the nucleic acid sequence of SEQ ID NO: 6.

A variety of vectors that comprise a nucleic acid encoding a MIS protein can be encompassed for use in the methods of the present invention. In some embodiments, the vector is an expression vector. Expression vectors compatible with eukaryotic cells, preferably those compatible with vertebrate cells, can be used, for example, to produce recombinant constructs for production of viral vectors harboring nucleic acids encoding a MIS protein as disclosed herein, for recombinant expression of such a MIS protein. Eukaryotic cell expression vectors are well known in the art and are available from several commercial sources.

A nucleic acid can be introduced into a target cell by any suitable method. For example, a nucleic acid encoding a MIS protein can be introduced into a cell by transfection (e.g., calcium phosphate or DEAE-dextran mediated transfection), lipofection, electroporation, microinjection (e.g., by direct injection of naked DNA), biolistics, infection with a viral vector containing the nucleic acid, cell fusion, chromosome-mediated gene transfer, microcell-mediated gene transfer, nuclear transfer, and the like.

Alternatively, in some embodiments, a plasmid expression vector can be used. Plasmid expression vectors include, but are not limited to, pcDNA3.1, pET vectors (Novagen®), pGEX vectors (GE Life Sciences), and pMAL vectors (New England labs. Inc.) for protein expression in E. coli host cell such as BL21, BL21(DE3) and AD494(DE3)pLysS, Rosetta (DE3), and Origami(DE3) (Novagen®); the strong CMV promoter-based pcDNA3.1 (Invitrogen™ Inc.) and pCIneo vectors (Promega) for expression in mammalian cell lines such as CHO, COS, HEK-293, Jurkat, and MCF-7; replication incompetent adenoviral vector vectors pAdeno X, pAd5F35, pLP-Adeno-X-CMV (Clontech®), pAd/CMV/V5-DEST, pAd-DEST vector (Invitrogen™ Inc.) for adenovirus-mediated gene transfer and expression in mammalian cells; pLNCX2, pLXSN, and pLAPSN retrovirus vectors for use with the Retro-X™ system from Clontech for retroviral-mediated gene transfer and expression in mammalian cells; pLenti4/V5-DEST™, pLenti6/V5-DEST™, and pLenti6.2/V5-GW/lacZ (INVITROGEN™ Inc.) for lentivirus-mediated gene transfer and expression in mammalian cells; adenovirus-associated virus expression vectors such as pAAV-MCS, pAAV-IRES-hrGFP, and pAAV-RC vector (Stratagene®) for adeno-associated virus-mediated gene transfer and expression in mammalian cells.

In some embodiments, a nucleic acid (e.g., DNA, modRNA, or RNAa) encoding a MIS protein as disclosed herein, can be suitably administered as a vector, e.g., a viral vector. In some embodiments, the vector is a viral vector.

Viral vector systems which can be utilized in the present invention include, but are not limited to, (a) adenovirus vectors; (b) retrovirus vectors, e.g., lentivirus vectors, murine moloney leukemia virus, etc.; (c) adeno-associated virus vectors; (d) herpes simplex virus vectors; (e) SV40 vectors; (f) polyoma virus vectors; (g) papilloma virus vectors; (h) picornavirus vectors; (i) pox virus vectors such as an orthopox, e.g., vaccinia virus vectors or avipox, e.g., canary pox or fowl pox; and (j) a helper-dependent or gutless adenovirus. Replication-defective viruses can also be advantageous. In a preferred embodiment, the vector is an adeno-associated virus vector.

In some embodiments, a viral vector such as an adeno-associated virus (AAV) vector is used. AAVs, which normally infect mammals, including humans, but are non-pathogenic, have been developed and employed as gene therapy vectors in clinical trials in the United States and Europe (Daya and Berns, Clinical Microbiology Reviews 2008, 21, 583-593). AAV vectors may be prepared using any one of a number of methods available to those of ordinary skill in the art. Exemplary AAV vectors are disclosed in Walsh et al., Proc. Soc. Exp. Biol. Med. 204:289-300 (1993); U.S. Pat. No. 5,436,146 which is incorporated herein by reference; Gao et al., Gene Therapy 2005, 5, 285-297; Vandenberghe et al., Gene Therapy 2009, 16, 311-319; Gao et al., PNAS 2002, 99, 11854-11859; Gao et al., PNAS 2003, 100, 6081-6086; Gao et al., J. of Virology 2004, 78, 6381-6388.

It should be noted that the selection of a particular type of AAV vectors can depend on the target tissue. In some embodiments, the vector is an adeno-associated virus (AAV) vector. In some embodiments, the AAV vector is an AAV1, AAV2, AAV4, AAV5, AAV6, AAV7, AAV8, AAV9, AAV9.HR, AAVrh.10, AAVMYO, or AAV2.5. In some embodiments, the AAV is AAV9. In some embodiments, a AAV vector for expressing a MIS protein is AAV9, as disclosed herein in the Examples.

Adenoviruses are other viral vectors that can be used in gene transfer methods. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995).

A retroviral vector can also be used (see Miller et al., Meth. Enzymol. 217:581-599 (1993)). These retroviral vectors contain the components necessary for the correct packaging of the viral genome and integration into the host cell DNA. A nucleic acid encoding a MIS protein is cloned into one or more vectors, which facilitate delivery of the gene into a subject.

In another embodiment, the vector is a pox virus such as a vaccinia virus, for example an attenuated vaccinia such as Modified Virus Ankara (MVA) or NYVAC, an avipox such as fowl pox or canary pox.

In another embodiment, lentiviral vectors are used, such as the HIV based vectors described in U.S. Pat. Nos. 6,143,520; 5,665,557; and 5,981,276, which are herein incorporated by reference.

The vector may or may not be incorporated into the genome of a cell. The constructs may include viral sequences for transfection, if desired. Alternatively, the construct may be incorporated into vectors capable of episomal replication, e.g., EPV and EBV vectors.

Constructs for the expression of a nucleic acid encoding a MIS protein as disclosed herein, e.g., DNA, modRNA or RNAa, can generally be operatively linked to regulatory elements, e.g., promoters, enhancers, etc., to ensure the expression of the construct in target cells. Other specifics for vectors and constructs are described in further detail below.

As used herein, the term “tissue-specific promoter” means a nucleic acid sequence that serves as a promoter, i.e., regulates expression of a selected nucleic acid sequence operably linked to the promoter, and which selectively affects expression of the selected nucleic acid sequence in specific cells of a tissue, such as cells of ovarian origin.

The term “constitutively active promoter” refers to a promoter of a gene which is expressed at all times within a given cell. Exemplary promoters for use in mammalian cells include cytomegalovirus (CMV), CMV early enhancer/chicken R actin (CBA) promoter, and the like.

The term “inducible promoter” refers to a promoter of a gene which can be expressed in response to a given signal, for example addition or reduction of an agent. Non-limiting examples of an inducible promoter are “tet-on” and “tet-off” promoters, or promoters that are regulated in a specific tissue type.

In some embodiments, the regulatory element comprises a constitutively active promoter. In some embodiments, the regulatory element comprises the CMV early enhancer/chicken R actin (CBA) promoter.

In some embodiments, compositions being administered comprise an inducible vector. Use of inducible vectors to regulate gene expression or protein synthesis is known in the art, see for example, in WO1993022431, US20110301228, U.S. Pat. No. 6,500,647, WO2005053750, or U.S. Pat. No. 6,784,340, which are herein incorporated by reference.

In some embodiments, the MIS protein is expressed by an inducible vector, which can comprise one or more regulatory elements, e.g., promoters, enhancers, etc., which are operatively linked to the polynucleotide encoding a MIS protein, whereby the regulatory elements can control the expression level of MIS.

Typical regulatory elements include, but are not limited to, transcriptional promoters, inducible promoters and transcriptional elements, an optional operate sequence to control transcription, a sequence encoding suitable mRNA ribosomal binding sites, and sequences to control the termination of transcription and/or translation. Included in the term “regulatory elements” are nucleic acid sequences such as initiation signals, enhancers, and promoters, which induce or control transcription of protein coding sequences with which they are operatively linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein. In some instances, the promoter sequence is recognized by the synthetic machinery of the cell, or introduced synthetic machinery, required for initiating transcription of a specific gene.

Regulatory sequences can be a single regulatory sequence or multiple regulatory sequences, or modified regulatory sequences or fragments thereof. Modified regulatory sequences are regulatory sequences where the nucleic acid sequence has been changed or modified by some means, for example, but not limited to, mutation, methylation etc.

Regulatory sequences useful in the methods as disclosed herein are promoter elements which are sufficient to render promoter-dependent gene expression controllable for cell type-specific, tissue-specific or inducible by external signals or agents (e.g., enhancers or repressors); such elements may be located in the 5′ or 3′ regions of the native gene, or within an intron.

In some embodiments, when a MIS protein encoded by a viral vector is expressed endogenously in a subject, the expression level of the MIS protein disclosed herein can be constant over a desired period of time, for example, at least 1 week, at least 2 weeks, at least 3 weeks, at least 1 month, at least 2 months, at least 3 months, at least 6 months, at least 1 year, at least 5 years, or over the lifetime of the subject. In some embodiments, the expression of the MIS protein disclosed herein can be sustained at or above a therapeutically effective dosage level over a desired period of time.

Other expression vectors can be used in some embodiments. For example, plasmids, episomes, bacterial artificial chromosomes, yeast artificial chromosomes, bacteriophages, and such vectors can integrate into the host's genome or replicate autonomously in the particular cell. Other forms of expression vectors known by those skilled in the art which serve equivalent functions can also be used.

Another gene transfer approach is ex vivo gene transfer, which involves transferring a nucleic acid to cells in tissue culture and delivering the transduced cells to a subject. The nucleic acid may be transferred to cells in culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred nucleic acid. Those cells are then delivered to a subject.

In certain embodiments, a nucleic acid encoding a MIS protein can be introduced into cells by electroporation (see, e.g., Wong and Neumann, Biochem. Biophys. Res. Commun. 107:584-87 (1982)) and biolistics (e.g., a gene gun; Johnston and Tang, Methods Cell Biol. 43 Pt A:353-65 (1994); Fynan et al., Proc. Natl. Acad. Sci. USA 90:11478-82 (1993)).

In certain embodiments, a nucleic acid sequence encoding a MIS protein can be introduced into target cells by transfection or lipofection. Suitable agents for transfection or lipofection include, for example, calcium phosphate, DEAE dextran, lipofectin, lipfectamine, DIMRIE C, Superfect, and Effectin (Qiagen), unifectin, maxifectin, DOTMA, DOGS (Transfectam; dioctadecylamidoglycylspermine), DOPE (1,2-dioleoyl-sn-glycero-3-phosphoethanolamine), DOTAP (1,2-dioleoyl-3-trimethylammonium propane), DDAB (dimethyl dioctadecylammonium bromide), DHDEAB (N,N-di-n-hexadecyl-N,N-dihydroxyethyl ammonium bromide), HDEAB (N-n-hexadecyl-N,N-dihydroxyethylammonium bromide), polybrene, poly(ethylenimine) (PEI), and the like. (See, e.g., Banerjee et al., Med. Chem. 42:4292-99 (1999); Godbey et al., Gene Ther. 6:1380-88 (1999); Kichler et al., Gene Ther. 5:855-60 (1998); Birchaa et al., J. Pharm. 183:195-207 (1999)).

Various delivery systems are known and can be used to directly administer therapeutic agents, e.g., encapsulation of a vector comprising a nucleic acid encoding a MIS protein in liposomes, microparticles, microcapsules, recombinant cells capable of expressing the compound, and receptor-mediated endocytosis (see, e.g., Wu and Wu, 1987, J. Biol. Chem. 262:4429-4432). U.S. Pat. No. 5,676,954 (which is herein incorporated by reference) reports on the injection of genetic material, complexed with cationic liposome carriers, into mice. U.S. Pat. Nos. 4,897,355, 4,946,787, 5,049,386, 5,459,127, 5,589,466, 5,693,622, 5,580,859, 5,703,055, and international publication NO: WO 94/9469 (which are herein incorporated by reference) provide cationic lipids for use in transfecting DNA into cells and mammals. U.S. Pat. Nos. 5,589,466, 5,693,622, 5,580,859, 5,703,055, and international publication NO: WO 94/9469 (which are herein incorporated by reference) provide methods for delivering DNA-cationic lipid complexes to mammals. Such cationic lipid complexes or nanoparticles can be used to administer therapeutic agents, e.g., a vector comprising a nucleic acid encoding a MIS protein and can also be used to deliver protein, e.g., a MIS protein.

### F. Detection of Antibodies Against MIS

An aspect of the present disclosure relates to detecting antibodies against feline MIS (fcMIS, e.g., SEQ ID NO: 1, SEQ ID NO: 3, SEQ ID NO: 14, SEQ ID NO: 18, SEQ ID NO: 20) or canine MIS (cMIS, e.g., SEQ ID NO: 3 or SEQ ID NO: 15). In some embodiments, it may be beneficial to determine if a subject has an immunological response against an administered a vector composition, e.g., an AAV vector, poxivirus vector, or a lentivirus vector expressing fcMIS or clMIS. In some embodiments, determination of the immunological response comprises detection of IgG antibodies against fcMIS or clMIS in the subject. Test samples may be collected from the subject for use in the detection of antibodies against fcMISv1 or clMIS. In some embodiments, the test sample comprises blood or serum.

In some embodiments, a method of detecting antibodies against fcMIS or clMIS in serum comprises (a) optionally isolating recombinant fcMIS or clMIS, such as FLAG-tagged fcMIS or FLAG-tagged clMIS; (b) adding isolated fcMIS or clMIS to a substrate, optionally wherein the substrate is an ELISA plate; (c) adding a test sample to the substrate; (d) incubating the substrate with a detectable antibody, optionally wherein the detectable antibody is goat anti-feline IgG (H+L) HRP; and (e) performing an enzyme substrate reaction, optionally wherein the enzyme substrate is HRP.

In some embodiments, the isolated fcMIS or clMIS can be isolated according to materials and methods known to one skilled in the art. In some embodiments, isolated fcMIS is any exemplary fcMIS protein disclosed herein, such as SEQ ID NO: 1, SEQ ID NO: 3, SEQ ID NO: 14, SEQ ID NO: 18, or SEQ ID NO: 20. In some embodiments, the isolated clMIS is any clMIS protein disclosed herein, such as SEQ ID NO: 2 or SEQ ID NO: 15. In some embodiments, the recombinant fcMIS or clMIS has a purification tag, such as a His-tag or a FLAG-tag. In some embodiments, the recombinant fcMIS is FLAG-tagged fcMISv1 or FLAG-tagged fcMISv2 or recombinant FLAG-tagged clMIS. In some embodiments, the isolated fcMIS or clMIS is purified from conditioned cell culture media.

In some embodiments, the method of detecting antibodies against fcMIS or clMIS comprises adhering isolated fcMIS or clMIS to the surface of a substrate, such as an ELISA plate. In some embodiments, excess amounts of the isolated fcMIS or clMIS are added to the substrate to ensure full coverage of the surface of the substrate with fcMIS or clMIS. In some embodiments, the isolated MIS and the substrate are incubated for about 30 minutes, 12 hours, 24 hours, or overnight. In some embodiments, the isolated fcMIS or clMIS and the substrate are incubated at about room temperature, 4° C., 20° C., 25° C., 30° C., or 37° C. After this incubation step, in some embodiments, excess fcMIS or clMIS that do not adhere to the substrate are washed away with appropriate buffers or wash solutions.

In some embodiments, a blocking step is performed after the substrate is coated with isolated fcMIS or clMIS. This blocking step is performed before test samples are added to the substrate. In some embodiments, the blocking step comprises incubating the fcMIS or clMIS-coated substrate with a blocking solution. In some embodiments, the blocking solution comprises Bovine Serum Albumin, goat serum, and/or a phosphate buffer saline solution with a low concentration of Tween 20 detergent (PBST).

Test samples from subjects (e.g., subjects administered a vector, e.g., viral vector expressing fcMIS or clMIS) may be added to the fcMIS- or clMIS-coated substrate. In some embodiments, the test samples are diluted with an appropriate buffer before being added to the substrate. In some embodiments, the test samples are diluted in the blocking buffer described above. In some embodiments, the test samples are diluted by a factor or 10, 20, 50, 100, 200, 500, or 1000. In some embodiments, the blocking buffer or an appropriate coating buffer is used to represent blanks.

In some embodiments, an anti-fcMIS antibody or anti-clMIS antibody is used as a positive control. In some embodiments, the anti-fcMIS antibody or clMIS antibody is diluted with an appropriate buffer before being added to the substrate. In some embodiments, the anti-fcMIS or anti-clMIS antibody is diluted in the blocking buffer described above. In some embodiments, the anti-fcMIS antibody or anti-clMIS antibody is diluted by a factor or 10, 20, 50, 100, 200, 500, or 1000.

In some embodiments, whole molecule IgG antibodies, such as cat or dog IgG antibodies, are used as antibody standards for detection by ELISA. In some embodiments, the whole molecule IgG antibodies are diluted with an appropriate buffer before being added to the substrate. In some embodiments, the whole molecule IgG antibodies are diluted in the blocking buffer described above. In some embodiments, the whole molecule IgG antibodies are diluted by a factor or 10, 20, 50, 100, 200, 500, or 1000.

After the test samples, anti-fcMIS antibody or anti-clMIS antibody, whole molecule IgG antibody standards, and blanks are added to the fcMIS-coated or clMIS-coated substrate, the substrate is incubated at an appropriate temperature for a certain amount of time. In some embodiments, the substrate is incubated for about 30 minutes, 1 hour, 12 hours, 24 hours, or overnight. In some embodiments, the substrate is incubated at about room temperature, 4° C., 20° C., 25° C., 30° C., or 37° C. After the incubation step, the substrate is washed with 3, 5, or more wash steps using an appropriate buffer or wash solution.

In some embodiments, after the wash steps, the substrate is incubated with a detectable antibody, such as goat anti-IgG horseradish peroxidase (HRP). The goat anti-IgG HRP is used according to manufacturer recommendations. In some embodiments, the substrate is incubated for about 30 minutes, 1 hour, 12 hours, 24 hours, or overnight. In some embodiments, the substrate is incubated at about room temperature, 4° C., 20° C., 25° C., 30° C., or 37° C. In some embodiments, the substrate is incubated in the dark. For detection of anti-MIS antibodies in test samples, an enzyme substrate reaction, such as an HRP enzyme substrate reaction, is performed according to manufacturer recommendations.

### G. Administration of a Composition

In some embodiments, the amount of composition administered is “effective” when it is sufficient to reduce fertility and/or prevent puberty in a non-human animal, or delay puberty in a human subject. In some embodiments, the amount of a composition comprising a vector (e.g., a viral vector) comprising a nucleic acid encoding a MIS protein is administered to a subject (e.g., as a one-time administration) such that the concentration of MIS protein in the serum of the prepubescent non-human subject (e.g., kitten or puppy) at or after 6 months, at or after 9 months, at or after 12 months, at or after 15 months, or at or after 24 months following administration of the composition is greater than 250 ng/ml, greater than 300 ng/ml, greater than 400 ng/ml, greater than 500 ng/ml, greater than 600 ng/ml, greater than 700 ng/ml, greater than 800 ng/ml, greater than 900 ng/ml, greater than 1 μg/ml, greater than 1.5 μg/ml, greater than 2 μg/ml, greater than 3 μg/ml, greater than 4 μg/ml, greater than 5 μg/ml, greater than 6 μg/ml, greater than 7 μg/ml, greater than 8 μg/ml, greater than 9 g/ml, greater than 10 μg/ml, or greater than 11 μg/ml.

MIS protein concentration may be determined by any number of protein quantification methods understood in the field, including antibody-based assays. For example, enzyme-linked immunosorbent assay (ELISA) methods can be utilized to quantify MIS protein concentration, such as commercially available kits, including the AMH Gen II ELISA (Beckman Coulter, Cat No. A73818). Anti-human MIS/AMH IgG antibodies may cross-react with and bind to cat and dog MIS protein. In some embodiments, MIS protein concentration is determined by ELISA.

In some embodiments, a composition comprising a vector (e.g., a viral vector) comprising a nucleic acid encoding a MIS protein can be administered at one time. In some embodiments, the composition can be divided into subdoses, e.g., 2-4 subdoses and administered over a period of time, e.g., at appropriate intervals through a day or other appropriate schedule. In some embodiments, administration can be chronic, e.g., one or more doses and/or treatments daily over a period of weeks or months. The dosage should not be so large as to cause adverse side effects. In some embodiments, the administration comprises a single dose. In some embodiments, the administration comprises dividing the single dose into two or multiple doses.

In some embodiments, the composition comprises a MIS protein that is a natural (i.e., wild-type) feline MIS protein that corresponds to SEQ ID NO: 1 or SEQ ID NO: 18. In some embodiments, the MIS protein is a natural (i.e., wild-type) canine MIS protein that corresponds to SEQ ID NO: 2.

In some embodiments, the composition comprises a modified feline MIS protein that corresponds to SEQ ID NO: 1, SEQ ID NO: 18, or SEQ ID NO: 3, or a protein having at least 85% sequence identity to at least amino acids 22-588 of SEQ ID NO: 1, a protein having at least 85% sequence identity to at least amino acids 22-589 of SEQ ID NO: 18, or a protein having at least 85% sequence identity to 22-572 of SEQ ID NO: 3, where the RAQ/R furin cleavage site of SEQ ID NO: 1, SEQ ID NO: 18, or SEQ ID NO: 3 is modified. In some embodiments, residue 478 of SEQ ID NO: 1 or residue 479 of SEQ ID NO: 18 is changed from a Q to R (Q478R) or K (Q478K). In some embodiments, residue 465 of SEQ ID NO: 3 is changed from a Q to R (Q465R) or K (Q465K), and optionally, the endogenous leader sequence of 1-21 of SEQ ID NO: 1, SEQ ID NO: 18, or SEQ ID NO: 3 is replaced with a non-MIS leader sequence as disclosed herein.

In some embodiments, the MIS protein is a natural (i.e., wild-type) canine MIS protein that corresponds to SEQ ID NO: 2. In some embodiments, the composition comprises a modified canine MIS protein that corresponds to SEQ ID NO:2, or a protein having at least 85% sequence identity to at least amino acids 23-573 of SEQ ID NO: 2, where the RAQ/R furin cleavage site of SEQ ID NO: 2 is modified. In some embodiments, residue 462 of SEQ ID NO: 2 is changed from a Q to R (Q462R) or K (Q462K), and optionally, the endogenous leader sequence of 1-22 of SEQ ID NO: 2 is replaced with a non-MIS leader sequence as disclosed herein.

In some embodiments, the MIS protein is a natural (i.e., wild-type) human MIS protein that corresponds to SEQ ID NO: 4. In some embodiments, the recombinant hMIS protein comprises a combination of a non-MIS leader sequence or a functional fragment thereof in place of the MIS leader sequence of amino acids 1-25 of SEQ ID NO: 4, and a modification of at least one amino acid between residues 448-452 of SEQ ID NO: 4 to increase cleavage as compared to in the absence of a modification, wherein the recombinant MIS protein has increased cleavage and increased yield of production in vitro as compared to wild-type MIS protein corresponding to amino acid residues of SEQ ID NO: 4. In some embodiments, the recombinant hMIS protein for use in the method and compositions herein lacks a leader sequence (i.e., the leader sequence has been cleaved off), but is produced from the processing of a hMIS preprotein that comprises a non-MIS leader sequence in place of the endogenous MIS leader sequence of amino acid residues 1-25 of SEQ ID NO: 4. That is, in some embodiments, the recombinant hMIS protein for use in the methods disclosed herein can be produced from a pre-proprotein comprising a non-MIS leader sequence or a functional fragment thereof in place of the MIS leader sequence of amino acids 1-25 of SEQ ID NO: 4, wherein the leader sequence is cleaved off during production. In some embodiments, the MIS protein is a recombinant modified human MIS protein that corresponds to SEQ ID NO: 7. In some embodiments, the composition comprises a modified human MIS protein that has at least 85% sequence identity to at least amino acids 25-560 of SEQ ID NO: 4, where the RAQ/R furin cleavage site of SEQ ID NO: 4 is modified. In some embodiments, residue 450 of SEQ ID NO: 4 is changed from a Q to R (Q450R) or K (Q450K), and optionally, the endogenous leader sequence of 1-24 of SEQ ID NO: 4 is replaced with a non-MIS leader sequence as disclosed herein, such as, for example, but not limited a leader sequence selected from any of SEQ ID: 9-13. In some embodiments, the recombinant MIS protein, such as human MIS protein does not comprise a FLAG tag or other tag.

In some embodiments, a human recombinant MIS protein disclosed herein is administered to a human subject to delay puberty, e.g., to reversibly delay puberty in a human female subject. In some embodiments, the MIS protein (e.g., a protein comprising amino acids at least 85% sequence identity to 25-560 of SEQ ID NO: 4 where residue 450 of SEQ ID NO: 4 is changed from a Q to R (Q450R) or K (Q450K), and optionally, the endogenous leader sequence of 1-24 of SEQ ID NO: 4 is replaced with a non-MIS leader sequence) is administered to a female subject concurrent with, or before, or after a treatment for central precocious puberty, such as, but not limited to any one or more of the following treatments selected from: GnRH (Gonadotropin-releasing hormone), GnRH agonist, GnRH analogues, metformin, progestin, or superagonist treatment. GnRH analogues include, but are not limited to: leuprolide acetate (Lupron Depot), or triptorelin (TRELSTAR™, Triptodur Kit). Other treatments for delaying puberty or for Central precocious puberty or precocious puberty include, SUPPRELIN LA™ (Histrelin), LUPRON DEPOT-PED™, TAMOXIFEN™, LEUPROLIDE™ SOLTAMOX™, VANTAS™, HISTRELIN™, SYNAREL™, TRIPTORELIN™, TRIPTODUR™, NAFARELIN™, FENSOLVI™. In some embodiments, administration of a rhMIS variant protein as disclosed herein to a female subject to delay puberty is administered to a subject for a period of between 1-3 months, 3 to 6-months, 6 to 12-months, 12 to 18 months, 18 to 24 months, or 2-3 years, 3-4 years, or 4-5 years, or 5-6 years, or longer than 6 years. Treatment can be daily, e.g., via use of a pump, implant or transdermal skin patch, or can be weekly. In some embodiments, administration of a rhMIS variant protein as disclosed herein to a female subject to delay puberty is administered to a female subject with central or peripheral precocious puberty at the age as early as 5 years, and the subject can be within the age range of 6-16 years if the subject has gender dysphoria and is need of delaying puberty to provide to give the subject with more time to fully understand the subject gender identity, or before a gender reassignment treatment and/or surgery, etc. Methods to administer a rhMIS protein are disclosed in US Application US20200071376 or US20160039898, which is incorporated herein in its entirety by reference.

In some embodiments, the composition comprises a chimeric feline MIS protein that corresponds to SEQ ID NO: 3. In some embodiments, the MIS protein is a recombinant protein or a functional fragment or derivative or variant thereof.

In some embodiments, administration of a composition comprising a vector (e.g., a viral vector) comprising a nucleic acid encoding a MIS protein, as disclosed herein can be a one-time administration. In some embodiments, administration of a composition as disclosed herein is by pulsed administration. In some embodiments, the effective dose of a composition can be administered as two, three, four, five, six or more sub-doses administered separately at appropriate intervals throughout the day, optionally, in unit dosage forms. In some embodiments, administration is repeated to maintain a desired level of an active ingredient in the body.

In some embodiments, an effective amount of a composition comprising a viral vector encoding a MIS protein can be provided at a dose of 1×10¹³ vector genomes, at a dose of 1×10¹³ vector genomes or less, at a dose of 5×10¹² vector genomes, at a dose of 5×10¹² vector genomes or less, at a dose of 1×10¹² vector genomes or less, at a dose of 5×10¹¹ vector genomes or less, or at a dose of 1×10¹¹ vector genomes or less per kilogram weight of the prepubescent subject (e.g., kitten or puppy). In some embodiments, an effective amount of a composition comprising a viral vector encoding a MIS protein can be provided at a dose of from 1×10¹¹ vector genomes to 1×10¹³ vector genomes per kilogram weight of the prepubescent subject (e.g., kitten or puppy). In some embodiments, an effective amount of a composition comprising a viral vector encoding a MIS protein can be provided at a dose of from 1×10¹¹ vector genomes to 1×10¹² vector genomes per kilogram weight of the prepubescent subject (e.g., kitten or puppy).

Vector genome (vg) and vector particle (vp) concentration may be determined by genome copy quantification methods understood in the field, such as by PCR (e.g., droplet digital PCR (ddPCR) technology) and comparison to a reference standard. Viral particle (vp) concentration may be determined by quantification methods understood in the field, such as by SDS-PAGE followed by Coomassie or silver stain and comparison to a reference standard.

In determining the effective amount of a composition comprising a vector (e.g., a viral vector) comprising a nucleic acid encoding a MIS protein, to be administered, circulating plasma levels of MIS proteins, formulation toxicities, and fertility status is evaluated. Such an effective dose, including the lowest dose effective to produce a therapeutic effect, will generally depend upon the factors described above. The selected dosage level will also depend upon a variety of factors including the activity of the particular compound of the present invention employed, the route of administration, the time of administration, the rate of excretion of the particular compound being employed, the duration of the treatment, other drugs, compounds and/or materials used in combination with the particular compound employed, the age, sex, weight, condition, general health and prior medical history of the subject being treated, and like factors well known in the medical arts.

For example, an effective amount can be estimated in an animal model to achieve a desirable concentration range and route of administration. Such information can then be used to determine useful doses and routes for administration in other subjects. Generally, the therapeutically effective amount is dependent on the desired therapeutic effect.

In some embodiments, a composition comprising a vector (e.g., a viral vector) comprising a nucleic acid encoding a MIS protein as disclosed herein can be administered to a prepubescent subject by any suitable and/or conventional injectable route of administration (e.g., intramuscular, intravenous, subcutaneous, or intraarterial).

When administering a composition comprising a vector (e.g., a viral vector) comprising a nucleic acid encoding a MIS protein as disclosed herein, it will generally be formulated in a dosage unit injectable form (e.g., solution, suspension, or emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (e.g., glycerol, propylene glycol, liquid polyethylene glycol), suitable mixtures thereof, and/or vegetable oils.

The term “dosage unit” form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the vector (e.g., viral vector) as disclosed herein and the particular biological effect to be achieved, and/or (b) the route of administration.

### H. Pharmaceutical Compositions

In some embodiments, a vector (e.g., a viral vector) comprising a nucleic acid encoding a MIS protein as disclosed herein can be formulated as a pharmaceutical composition by any suitable means, e.g., as a sterile injectable solution, e.g., which can be prepared by incorporating the composition in the required amount of the appropriate solvent with various of the other ingredients, as desired.

In certain embodiments, a composition comprising a vector (e.g., a viral vector) comprising a nucleic acid encoding a MIS protein as disclosed herein can be administered to a subject as a pharmaceutical composition with a pharmaceutically acceptable carrier. In certain embodiments, these pharmaceutical compositions optionally further comprise one or more additional therapeutic agents.

Pharmaceutically acceptable carriers are familiar to those skilled in the art. For compositions formulated as liquid solutions, acceptable carriers include saline and sterile water, and may optionally include antioxidants, buffers, bacteriostats and other common additives. One skilled in this art may further formulate the compounds of this invention in an appropriate manner, and in accordance with accepted practices, such as those disclosed in Reming' on's Pharmaceutical Sciences, Gennaro, Ed., Mack Publishing Co., Easton, Pa. 1990.

A pharmacological formulation of a composition comprising a vector (e.g., a viral vector) comprising a nucleic acid encoding a MIS protein as disclosed herein can be administered to the subject in an injectable formulation containing any pharmaceutically acceptable carrier, such as various vehicles, adjuvants, additives, and diluents.

Proper fluidity can be maintained, for example, by the use of a coating such as lecithin, by the maintenance of the required particle size in the case of a dispersion and by the use of surfactants. Non-aqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, e.g., parabens, chlorobutanol, phenol and sorbic acid. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

In another embodiment, a composition comprising a vector (e.g., a viral vector) comprising a nucleic acid encoding a MIS protein as disclosed herein can comprise lipid-based formulations. Any of the known lipid-based drug delivery systems can be used in the practice of the invention. For instance, multivesicular liposomes, multilamellar liposomes and unilamellar liposomes can all be used so long as a sustained release rate of the encapsulated active compound can be established. Methods of making controlled release multivesicular liposome drug delivery systems are described in PCT Application Publication Nos: WO 9703652, WO 9513796, and WO 9423697, the contents of which are incorporated herein by reference.

The composition of the synthetic membrane vesicle is usually a combination of phospholipids, usually in combination with steroids, especially cholesterol. Other phospholipids or other lipids may also be used. Examples of lipids useful in synthetic membrane vesicle production include phosphatidylglycerols, phosphatidylcholines, phosphatidylserines, phosphatidylethanolamines, sphingolipids, cerebrosides, and gangliosides, with preferable embodiments including egg phosphatidylcholine, dipalmitoylphosphatidylcholine, distearoylphosphatidyleholine, dioleoylphosphatidylcholine, dipalmitoylphosphatidylglycerol, and dioleoylphosphatidylglycerol.

In preparing lipid-based vesicles containing a nucleic acid encoding MIS protein, such variables as the efficiency of encapsulation, labiality of the vector and/or nucleic acid, homogeneity and size of the resulting population of vesicles, compound-to-lipid ratio, permeability, instability of the preparation, and pharmaceutical acceptability of the formulation should be considered.

The compounds utilized in the present invention, such as a nucleic acid encoding a MIS protein as disclosed herein can be administered parenterally to the subject in the form of slow-release subcutaneous implants or targeted delivery systems such as polymer matrices, liposomes, and microspheres. Other such implants, delivery systems, and modules are well known to those skilled in the art.

Prior to introduction, a composition comprising a vector (e.g., a viral vector) comprising a nucleic acid encoding the same as disclosed herein can be sterilized, by any of the numerous available techniques of the art, including filtration.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of ordinary skill in the art.

### Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are useful to an embodiment, yet open to the inclusion of unspecified elements, whether useful or not.

The singular terms “a,” “an,” and “the” include plural referents unless context clearly indicates otherwise. Similarly, the word “or” is intended to include “and” unless the context clearly indicates otherwise.

Other than in the operating examples, or where otherwise indicated, all numbers expressing quantities of ingredients or reaction conditions used herein should be understood as modified in all instances by the term “about.” The term “about” when used in connection with percentages may mean±5% of the value being referred to. For example, about 100 means from 95 to 105.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

As used herein, the term “polypeptide” refers to a polymer of amino acids and its equivalent and does not refer to a specific length of the product; thus, peptides, oligopeptides and proteins are included within the definition of a polypeptide. A derivative is a polypeptide having conservative amino acid substitutions, as compared with another sequence. Derivatives further include other modifications of proteins, including, for example, modifications such as glycosylations, acetylations, phosphorylations, and the like.

The term “Müllerian Inhibiting Substance” and “MIS” are used interchangeably herein and also refer to anti-Müllerian hormone or AMH, as well as to compounds and materials which are structurally similar to MIS or a fragment thereof. By “MIS” or “Müllerian Inhibiting Substance” is meant a polypeptide having an amino acid sequence at least 60%, or at least 70%, or at least 80%, or at least 90%, or at least 95%, or at least 96%, or at least 97%, or at least 98%, or at least 99% identical to amino acid residues of SEQ ID NO: 1, SEQ ID NO 18, SEQ ID NO: 2, or SEQ ID NO: 4 and fragments thereof. The present invention is intended to include variant forms of MIS which have substantially the same, or greater biological activity as a wild-type MIS. Examples of such variant MIS molecules include a deletion, insertion, or alteration in the amino acid sequence of wild-type MIS. Additionally, MIS proteins can be obtained using recombinant DNA technology, or from chemical synthesis of the MIS protein.

The term “wild-type” refers to the naturally-occurring polynucleotide sequence encoding a protein, or a portion thereof, or protein sequence, or portion thereof, respectively, as it normally exists in vivo.

Accordingly, as disclosed herein, a wild-type amino acid sequence for the pre-proprotein of feline MIS corresponds to SEQ ID NO: 1 or SEQ ID NO: 18 (where amino acid residues 1-21 correspond to the leader sequence); a wild-type amino acid sequence for the pre-proprotein of canine MIS corresponds to SEQ ID NO: 2 (where amino acid residues 1-22 correspond to the leader sequence); and a wild-type amino acid sequence for the pre-proprotein of human MIS corresponds to SEQ ID NO: 4 (where amino acid residues 1-24 correspond to the leader sequence). A wild-type amino acid sequence for the proprotein of feline MIS comprises amino acid residues 22-588 of SEQ ID NO: 1 or amino acid residues 22-589 of SEQ ID NO: 18; a wild-type amino acid sequence for the proprotein of canine MIS comprises amino acid residues 23-572 of SEQ ID NO: 2; a wild-type amino acid sequence for the proprotein of human MIS comprises amino acid residues 25-560 of SEQ ID NO: 4. The proprotein is then post-translationally processed by cleavage as discussed herein to form a bioactive MIS homodimer.

A “nucleic acid encoding MIS” is meant to include a nucleic acid encoding a polypeptide of SEQ ID NO: 1, SEQ ID NO: 18, SEQ ID NO: 2, SEQ ID NO: 3, or SEQ ID NO: 4, or encoding a polypeptide comprising an amino acid sequence having at least 60%, or at least 70%, or at least 80%, or at least 90%, or at least 95%, or at least 96%, or at least 97%, or at least 98%, or at least 99% sequence identity to the amino acid sequence of SEQ ID NO: 1, SEQ ID NO: 18, SEQ ID NO: 2, SEQ ID NO: 4, amino acid residues 22-588 of SEQ ID NO: 1, amino acid residues 22-589 of SEQ ID NO: 18, amino acid residues 23-572 of SEQ ID NO: 2, or amino acid residues 26-560 of SEQ ID NO: 4.

The term “variant” refers to any change in the genetic material of an organism, in particular a change (i.e., deletion, substitution, addition, or alteration) in a wild-type polynucleotide sequence or any change in a wild-type protein sequence. The term “mutant” is used interchangeably with “variant.” Although it is often assumed that a change in the genetic material results in a change of the function of the protein, the terms “variant” and “mutant” refer to a change in the sequence of a wild-type protein regardless of whether that change alters the function of the protein (e.g., increases, decreases, imparts a new function), or whether that change has no effect on the function of the protein (e.g., the mutation or variation is silent). The term mutation is used interchangeably herein with polymorphism in this application. Variants can be naturally-occurring, synthetic, recombinant, or chemically modified polynucleotides or polypeptides isolated or generated using methods well known in the art. Variants can include conservative or non-conservative amino acid changes, as described below. Polynucleotide changes can result in amino acid substitutions, additions, deletions, fusions and truncations in the polypeptide encoded by the reference sequence. Variants can also include insertions, deletions or substitutions of amino acids, including insertions and substitutions of amino acids and other molecules that do not normally occur in the peptide sequence that is the basis of the variant, for example but not limited to insertion of ornithine, which do not normally occur in human proteins.

The term “nucleic acid” is well known in the art. A “nucleic acid” as used herein will generally refer to a molecule (i.e., strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T,” or a cytosine “C”) or RNA (e.g., an A, a G, a uracil “U,” or a C). The term “nucleic acid” encompasses the terms “oligonucleotide” and “polynucleotide,” each as a subgenus of the term “nucleic acid.” The term “oligonucleotide” refers to a molecule of between 3 and 100 nucleobases in length. The term “polynucleotide” refers to at least one molecule of greater than 100 nucleobases in length. The term “nucleic acid” also refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. The terms “polynucleotide sequence” and “nucleotide sequence” are also used interchangeably herein.

As used herein, the term “gene” refers to a nucleic acid comprising an open reading frame encoding a polypeptide, including both exon and (optionally) intron sequences. A “gene” refers to coding sequence of a gene product, as well as non-coding regions of the gene product, including 5′UTR and 3′UTR regions, introns and the promoter of the gene product. These definitions generally refer to a single-stranded molecule, but in specific embodiments will also encompass an additional strand that is partially, substantially or fully complementary to the single-stranded molecule. Thus, a nucleic acid may encompass a double-stranded molecule or a double-stranded molecule that comprises one or more complementary strand(s) or “complement(s)” of a particular sequence comprising a molecule. As used herein, a single stranded nucleic acid may be denoted by the prefix “ss,” a double stranded nucleic acid by the prefix “ds,” and a triple stranded nucleic acid by the prefix “ts.” The term “gene” refers to the segment of DNA involved in producing a polypeptide chain; it includes regions preceding and following the coding region as well as intervening sequences (introns) between individual coding segments (exons).

The term “regulatory sequences” is used interchangeably with “regulatory elements” herein to refer to a segment of nucleic acid, typically but not limited to DNA or RNA or analogues thereof, that modulates the transcription of the nucleic acid sequence to which it is operatively linked, and thus act as transcriptional modulators. Regulatory sequences modulate the expression of gene and/or nucleic acid sequence to which they are operatively linked. Regulatory sequences often comprise “regulatory elements,” which are nucleic acid sequences that are transcription binding domains and are recognized by the nucleic acid-binding domains of transcriptional proteins and/or transcription factors, repressors or enhancers, etc. Typical regulatory sequences include, but are not limited to, transcriptional promoters, inducible promoters and transcriptional elements, an optional operate sequence to control transcription, a sequence encoding suitable mRNA ribosomal binding sites, and sequences to control the termination of transcription and/or translation. Regulatory sequences can be a single regulatory sequence or multiple regulatory sequences, or modified regulatory sequences or fragments thereof. Modified regulatory sequences are regulatory sequences where the nucleic acid sequence has been changed or modified by some means, for example, but not limited to, by mutation, methylation, etc.

The term “operatively linked” as used herein refers to the functional relationship of the nucleic acid sequences with regulatory sequences of nucleotides, such as promoters, enhancers, transcriptional and translational stop sites, and other signal sequences. For example, operative linkage of nucleic acid sequences, typically DNA, to a regulatory sequence or promoter region refers to the physical and functional relationship between the DNA and the regulatory sequence or promoter such that the transcription of such DNA is initiated from the regulatory sequence or promoter, by an RNA polymerase that specifically recognizes, binds and transcribes the DNA. In order to optimize expression and/or in vitro transcription, it may be necessary to modify the regulatory sequence for the expression of the nucleic acid or DNA in the cell type for which it is expressed. The desirability of, or need of, such modification may be empirically determined. Enhancers need not be located in close proximity to the coding sequences whose transcription they enhance. Furthermore, a gene transcribed from a promoter regulated in trans by a factor transcribed by a second promoter may be said to be operatively linked to the second promoter. In such a case, transcription of the first gene is said to be operatively linked to the first promoter and is also said to be operatively linked to the second promoter.

A “promoter” is a region of a nucleic acid sequence at which initiation and rate of transcription are controlled. It may contain elements at which regulatory proteins and molecules may bind, such as RNA polymerase and other transcription factors, to initiate the specific transcription of a nucleic acid sequence.

The term “enhancer” refers to a cis-acting regulatory sequence involved in the transcriptional activation of a nucleic acid sequence. An enhancer can function in either orientation and may be upstream or downstream of the promoter.

The term “functional” when used in conjunction with “variant” or “fragment” refers to a polypeptide which possesses a biological activity (either functional or structural) that is substantially similar to a biological activity of the polypeptide which it is a functional derivative, variant or functional fragment thereof. The term functional derivative is intended to include the fragments, analogues or chemical derivatives of a molecule.

The term “substantially similar” in this context means that the biological activity, e.g., activation of receptor MISRII is at 25% or at least 35%, or at least 50% as active as a reference polypeptide, e.g., a corresponding wild-type MIS polypeptide, and preferably at least 60% as active, 70% as active, 80% as active, 90% as active, 95% as active, 100% as active or even higher (i.e., the variant or derivative has greater activity than the wild-type), e.g., 110% as active, 120% as active, or more. Stated another way, a “substantially similar” functional fragment of a MIS protein in this context is meant that at least 25%, at least 35%, at least 50% of the relevant or desired biological activity of a corresponding reference MIS protein (e.g., wild-type MIS protein) is retained. In the instance of a functional fragment or peptide of a MIS protein as disclosed herein (e.g., SEQ ID NO: 1, 18, 2, or 4), a functional fragment would be a protein or peptide comprising a portion that retains an activity to activate MISRII.

The term “biologically active variant” or “biologically active fragment” are used interchangeably, and refers to a compound which possesses a biological activity (either functional or structural) that is substantially similar to a biological activity of the entity or molecule it is a functional derivative of (e.g., a wild-type MIS protein).

The term “conservative substitution,” when describing a polypeptide, refers to a change in the amino acid composition of the polypeptide that does not substantially alter the polypeptide's activity. For example, a conservative substitution refers to substituting an amino acid residue for a different amino acid residue that has similar chemical properties. Conservative amino acid substitutions include replacement of a leucine with an isoleucine or valine, an aspartate with a glutamate, or a threonine with a serine. “Conservative amino acid substitutions” result from replacing one amino acid with another having similar structural and/or chemical properties, such as the replacement of a leucine with an isoleucine or valine, an aspartate with a glutamate, or a threonine with a serine. Thus, a “conservative substitution” of a particular amino acid sequence refers to substitution of those amino acids that are not critical for polypeptide activity or substitution of amino acids with other amino acids having similar properties (e.g., acidic, basic, positively or negatively charged, polar or non-polar, etc.) such that the substitution of even critical amino acids does not reduce the activity of the peptide.

Conservative substitution tables providing functionally similar amino acids are well known in the art. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). (See also Creighton, Proteins, W. H. Freeman and Company (1984).)

As used herein, the term “nonconservative substitution” refers to a change in an amino acid residue for a different amino acid residue that has different chemical properties. The nonconservative substitutions include, but are not limited to aspartic acid (D) being replaced with glycine (G); asparagine (N) being replaced with lysine (K); or alanine (A) being replaced with arginine (R).

In some embodiments, individual substitutions, deletions or additions that alter, add or delete a single amino acid or a small percentage of amino acids can also be considered “conservative substitutions” if the change does not reduce the activity of the MIS protein. Insertions or deletions are typically in the range of 1 to 5 amino acids. The choice of conservative amino acids may be selected based on the location of the amino acid to be substituted in the peptide, for example if the amino acid is on the exterior of the peptide and exposed to solvents, or on the interior and not exposed to solvents.

The terms “homology,” “identity,” and “similarity” refer to the degree of sequence similarity between two peptides or between two optimally aligned nucleic acid molecules. Homology and identity can each be determined by comparing a position in each sequence which can be aligned for purposes of comparison. For example, it is based upon using a standard homology software in the default position, such as BLAST. When an equivalent position in the compared sequences is occupied by the same base or amino acid, then the molecules are identical at that position; when the equivalent site occupied by similar amino acid residues (e.g., similar in steric and/or electronic nature such as, for example conservative amino acid substitutions), then the molecules can be referred to as homologous (similar) at that position. Expression as a percentage of homology/similarity or identity refers to a function of the number of similar or identical amino acids at positions shared by the compared sequences, respectfully. A sequence which is “unrelated” or “non-homologous” shares less than 40% identity, though preferably less than 25% identity with the sequences as disclosed herein.

As used herein, the term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T. C, G. U. or I) or residue occurs in both sequences to yield the number of matched positions, dividing the number of matched positions by the total number of positions in the comparison window (i.e., the window size), and multiplying the result by 100 to yield the percentage of sequence identity.

The terms “substantial identity” as used herein denotes a characteristic of a polynucleotide or amino acid sequence, wherein the polynucleotide or amino acid comprises a sequence that has at least 85% sequence identity, preferably at least 90% to 95% sequence identity, more usually at least 99% sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which can include deletions or additions which total 20 percent or less of the reference sequence over the comparison window. The reference sequence can be a subset of a larger sequence. The term “similarity,” when used to describe a polypeptide, is determined by comparing the amino acid sequence and the conserved amino acid substitutes of one polypeptide to the sequence of a second polypeptide.

As used herein, the terms “homologous” or “homologues” are used interchangeably, and when used to describe a polynucleotide or polypeptide, indicates that two polynucleotides or polypeptides, or designated sequences thereof, when optimally aligned and compared, for example using BLAST with default parameters for an alignment are identical, with appropriate nucleotide insertions or deletions or amino-acid insertions or deletions, in at least 70% of the nucleotides, usually from 75% to 99%, and more preferably at least 98 to 99% of the nucleotides. The term “homolog” or “homologous” as used herein also refers to homology with respect to structure and/or function. With respect to sequence homology, sequences are homologs if they are at least 50%, at least 60 at least 70%, at least 80%, at least 90%, at least 95% identical, at least 97% identical, or at least 99% identical. Determination of homologs of the genes or peptides of the present invention can be easily ascertained by the skilled artisan.

The term “substantially homologous” refers to sequences that are at least 90%, at least 95% identical, at least 96%, identical at least 97% identical, at least 98% identical or at least 99% identical. Homologous sequences can be the same functional gene in different species. Determination of homologs of the genes or peptides of the present invention can be easily ascertained by the skilled artisan.

A molecule is said to be “substantially similar” to another molecule if both molecules have substantially similar structures or if both molecules possess a similar biological activity, for example if two MIS molecules are able to activate MISRII or inhibit ovarian follicle maturation. Thus, provided that two molecules possess a similar activity, are considered variants and are encompassed for use as disclosed herein, even if the structure of one of the molecules not found in the other, or if the sequence of amino acid residues is not identical. Thus, provided that two molecules possess a similar biological activity, they are considered variants as that term is used herein even if the structure of one of the molecules not found in the other, or if the sequence of amino acid residues is not identical. As such, nucleic acid and amino acid sequences having lesser degrees of similarity but comparable biological activity to a MIS protein are considered to be equivalents. In determining polynucleotide sequences, all subject polynucleotide sequences capable of encoding substantially similar amino acid sequences are considered to be substantially similar to a reference polynucleotide sequence, regardless of differences in codon sequence.

By “enhanced proteolytic stability” is meant a reduction of in the rate or extent of proteolysis of a peptide sequence by at least 2%, at least 5%, at least 10%, at least 20%, at least 30%, at least 40%, at least 50%, at least 60%, at least 70%, at least 80%, at least 85%, at least 90%, at least 95%, at least 98%, or at least 99% as compared to a control sequence under the same conditions (e.g., in vivo or in an in vitro system such as in a cell or cell lysate). A peptide with enhanced proteolytic stability may contain any modification, for example, insertions, deletions, or point mutations which reduce or eliminate a site subject to proteolytic cleavage at a particular site. Sites of proteolytic cleavage may be identified based on known target sequences or using computer software (e.g., software described by Gasteiger et al., Protein Identification and Analysis Tools on the ExPASy Server. In John M. Walker, ed. The Proteomics Protocols Handbook, Humana Press (2005)). Alternatively, proteolytic sites can be determined experimentally, for example, by Western blot for the protein following expression or incubation in a cellular system or cellular lysate, followed by sequencing of the identified fragments to determine cleavage sites.

The term “recombinant” as used herein to describe a nucleic acid molecule, means a polynucleotide of genomic, cDNA, viral, semisynthetic, and/or synthetic origin, which, by virtue of its origin or manipulation, is not associated with all or a portion of the polynucleotide with which it is associated in nature. The term recombinant as used with respect to a protein or polypeptide, means a polypeptide produced by expression of a recombinant polynucleotide. The term recombinant as used with respect to a host cell means a host cell into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

The term “subject” can refer to a non-human subject or a human subject. A “non-human subject” refers to the animal (e.g., kitten or puppy) to whom the composition according to the present invention, is provided. In certain embodiments, the animal is a vertebrate such as, but not limited to a mammal, cat, dog, primate, rodent, domestic animal or game animal. In certain embodiments of the aspects described herein, the subject is a cat or a dog. A subject can be male or female. Additionally, a subject can be an adult or can be prepubescent (e.g., kitten or puppy).

As used herein, the terms “administering,” and “introducing” are used interchangeably herein and refer to the placement of a composition comprising a vector comprising a nucleic acid encoding a MIS protein as disclosed herein into a subject by a method or route which results in at least partial localization of the composition at a desired site. The compounds of the present invention can be administered by any appropriate route which results in reduce fertility and/or preventing puberty, or delaying puberty in the subject.

The term “effective amount” or “therapeutically effective amount” are used herein interchangeably to refer to a sufficient amount of pharmacological composition to provide the desired effect. For any given case, as set forth in detail herein, an appropriate “effective amount” can be determined by one of ordinary skill in the art and can be judged by an ordinarily skilled practitioner.

As used herein, the terms “prevent,” “preventing” and “prevention” refer to avoidance or delay in manifestation of a biological condition, symptom, or marker, e.g., puberty, estrus, or fertility. The terms “prevent,” “preventing,” and “prevention” include the avoidance or delay in manifestation of a biological condition, symptom, or marker (e.g., puberty, estrus, or fertility) relative to the condition, symptom, or marker of a control, an untreated subject, or a treated subject at a reference point. The terms “prevent,” “preventing,” and “prevention” include not only the avoidance or delay, but also a reduced severity or degree of any biological condition, symptom, or marker.

The terms “delay” or “delaying” as used herein with respect to delaying puberty refers to a postponement, or suspension or pause in puberty in the subject for a specific period of time, with puberty reoccurring after the treatment is stopped.

As used herein, the terms “reduce,” “reducing,” and “reduction” refer to a reduced severity or degree of a biological condition, symptom, or marker. The terms “reduce,” “reducing,” and “reduction” include the reduced severity or degree of a biological condition, symptom, or marker (e.g., puberty, estrus, or fertility) relative to that of a control, an untreated subject, or a treated subject at a reference point.

For example, by “reduce,” “reducing,” and “reduction” is meant a decrease by a statistically significant amount and can include a decrease by at least a 10% in the severity or degree of a condition, symptom, or measurable marker, relative to a control or reference, e.g., at least 15%, 20%, 30%, 40%, 50%, 60%, 70%, 80%, 90%, 95%, 99% 90% to 95%, 90% to 99%, 10% to 95%, 10% to 99%, or even 100% (i.e., no symptoms or measurable markers).

A “composition” or “pharmaceutical composition” are used interchangeably herein to refer to a composition that usually contains an excipient, such as a pharmaceutically acceptable carrier that is conventional in the art and that is suitable for administration to cells. Exemplary compositions and pharmaceutical compositions are described in detail herein.

“Pharmaceutically” or “pharmaceutically acceptable” refers to molecular entities and compositions that do not produce an adverse, allergic or other untoward reaction when administered to a mammal, as appropriate.

The phrase “pharmaceutically acceptable carrier” as used herein means a pharmaceutically acceptable material, composition or vehicle, such as a liquid or solid filler, diluent, excipient, solvent or encapsulating material, involved in maintaining the activity of or carrying or transporting the subject agents from one organ, or portion of the body, to another organ, or portion of the body. As set forth in detail herein, a pharmaceutically acceptable carrier or excipient refers to a non-toxic solid, semi-solid or liquid filler, diluent, encapsulating material or formulation auxiliary of any type. In addition to being “pharmaceutically acceptable” as that term is defined herein, each carrier must also be “acceptable” in the sense of being compatible with the other ingredients of the formulation.

The term “vector” refers to a nucleic acid molecule capable of transporting another nucleic acid to which it has been linked. A plasmid is a species of the genus encompassed by “vector.” A viral vector is a species of the genus encompassed by “vector.”

The term “viral vector” refers to the use of viruses, or virus-associated vectors as carriers of the nucleic acid construct into the cell. Constructs may be integrated and packaged into non-replicating, defective viral genomes like adenovirus, adeno-associated virus (AAV), or Herpes simplex virus (HSV) or others, including retroviral and lentiviral vectors, for infection or transduction into cells. The vector may or may not be incorporated into the cells' genome. The constructs may include viral sequences for transfection, if desired. Alternatively, the construct may be incorporated into vectors capable of episomal replication, e.g., EPV and EBV vectors.

The term “inducible vector” refers to a vector whose gene expression can be controlled. For example, the level of gene expression can be increased, decreased, or reduced to zero. In some embodiments, the inducible vector can comprise a switch that controls gene expression.

The term “statistically significant” or “significantly” refers to statistical significance and generally means a two standard deviation (2SD) or greater difference.

Definitions of common terms in cell biology and molecular biology can be found in “The Merck Manual of Diagnosis and ^{Th}erapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9). Definitions of common terms in molecular biology can also be found in Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

### EXAMPLES

### Example 1—Pilot Studies of AAV9-MIS Gene Transfer as a Long-Term Non-Surgical Contraceptive in Adult Female Cats

### A. In Vitro Studies, Mouse Studies, and an Adult Cat Pilot Study with a First-Generation, Chimeric Feline MIS (fcMISv1) Transgene

Gene delivery of an AAV9 viral vector containing a human MIS transgene to sexually mature female mice has been shown to induce lifetime contraception with no detectable adverse effects. (WO2015/089321.)

### Materials and Methods

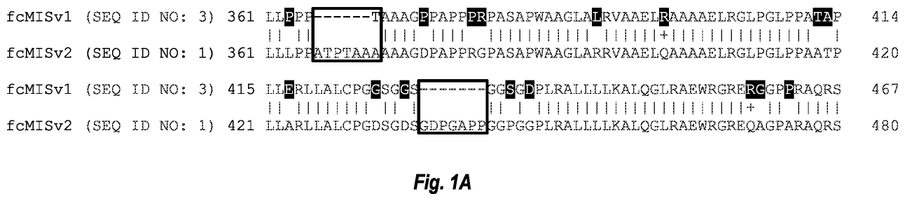

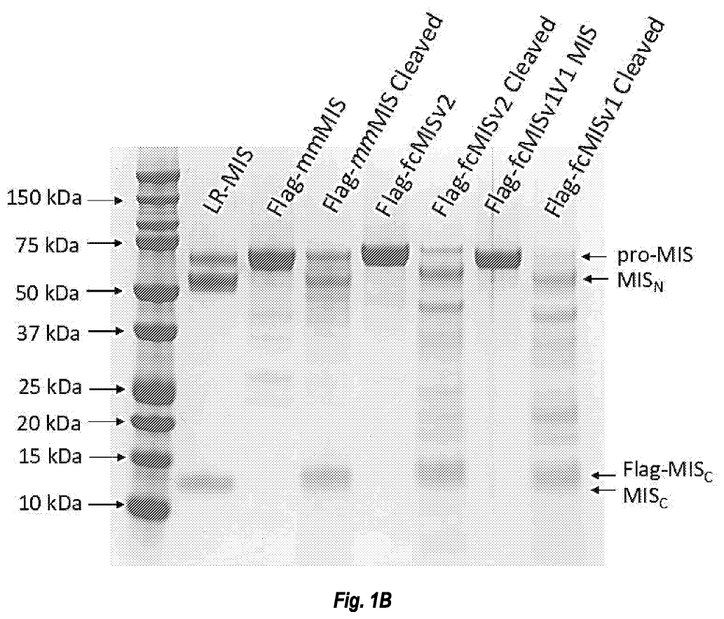

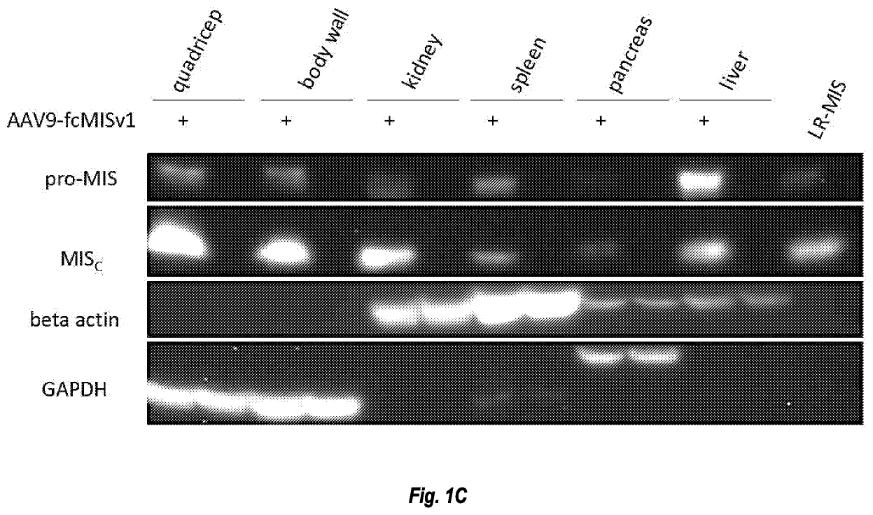

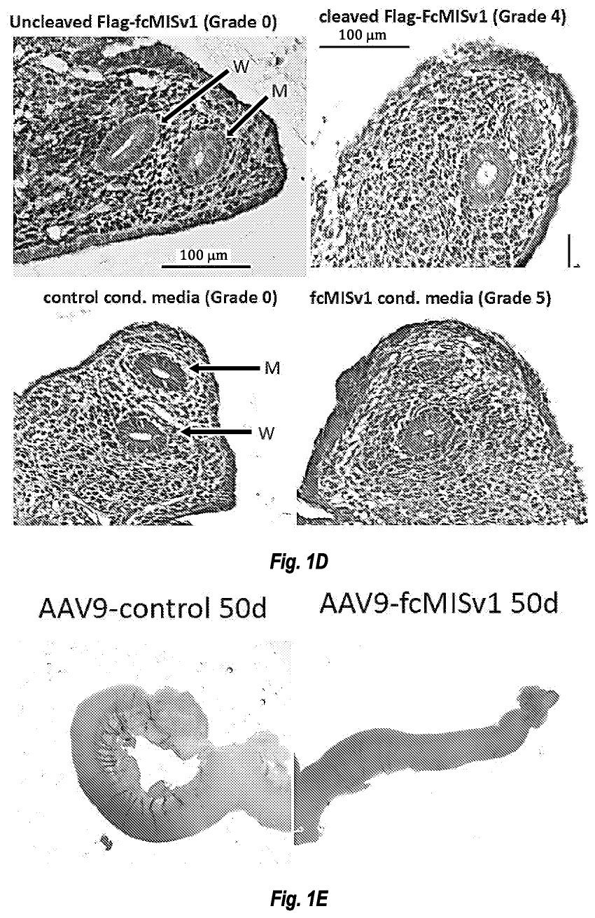

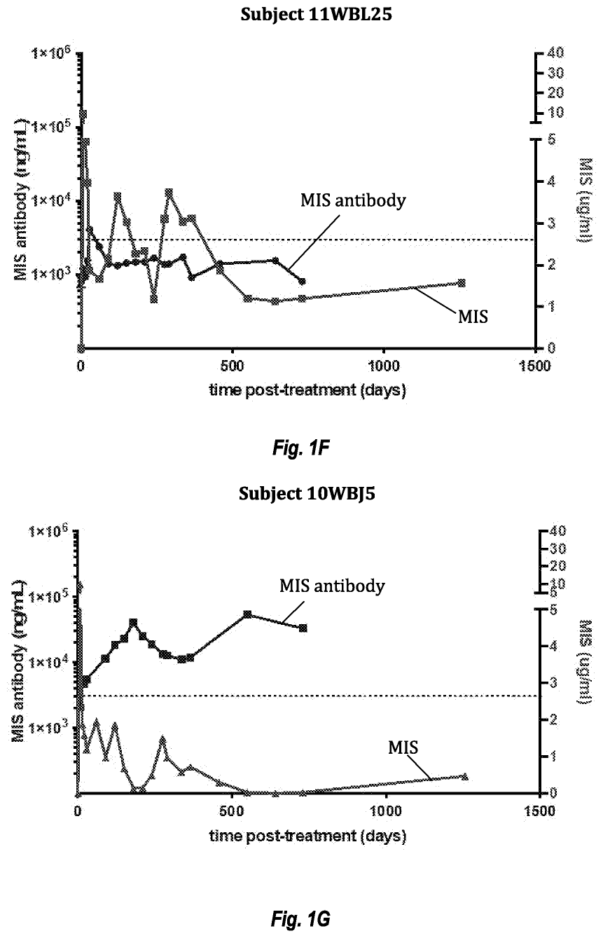

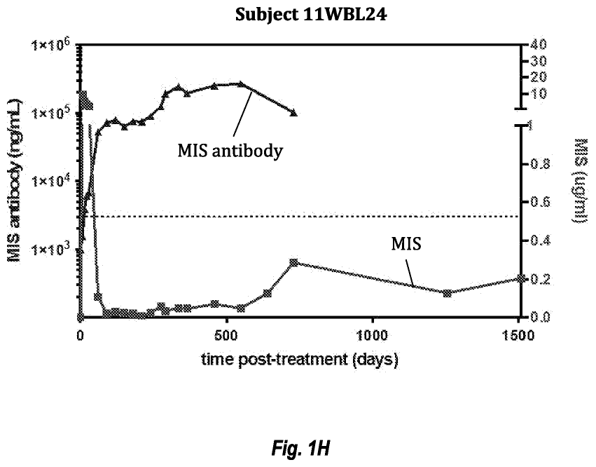

A first-generation, chimeric felis catus MIS transgene, fcMISv1 (SEQ ID NO: 3), was synthesized based on the partial wildtype MIS sequence of cat genome version 8.0 which was completed with consensus carnivora sequence. The partial MIS sequence was found to contain 30 amino acid differences when compared to the cat genome version 9.0, corresponding to the GC-rich region encoding amino acids 361-466 in the C-terminus of the MIS pro-domain (FIG. 1A), which does not participate in receptor binding. In this study, a viral vector was designed containing the chimeric felis catus MIS transgene (AAV9-fcMISv1).

The recombinant fcMISv1 (SEQ ID NO: 3) protein and a Flag-tagged variant were produced in CHO cells and subsequently purified. CHO-K1 (ATCC; #CCL-61) cells were kept in culture in DMEM culture medium with 5% FBS and 1% PenStrep. CHO-K1 cells were plated in 6-well plates for transfection. Once 80% confluent, CHO-K1 cells were transfected with cat MIS plasmids (Genscript) that express fcMISv1 or fcMISv2. In total, 5.1 μg of plasmid were transfected with 15.3 μl of Fugene6 (Promega, #E2691) (mass/volume ratio of 1:6) in each well. The transfection efficiency was confirmed to be greater than 80% by transfecting an identical plate with a GFP expression plasmid (PCDNA3-eGFP-N1). Conditioned culture medium was collected after 72 h and used for urogenital ridge regression assay, ELISA or Western Blot.

Experiments in mice were conducted with 6 week old Nu/Nu nude mice (Gnotobiotic Mouse Cox7 Core, Boston) approved by the National Institute of Health and Harvard Medical School Institutional Animal Care and Use Committee, in accordance with the Massachusetts General Hospital approved experimental protocol 2014N000275. The mice were housed in 12 hours light/12 hours night conditions with food and water provided ad libitum. Each mouse received a single intraperitoneal (i.p.) injection of AAV9-fcMISv1 or empty vector at 5e12 vg/kg, 1e13 vg/kg, or 5e12 vg/kg. Blood was collected from the mice cheeks prior to vector injection and weekly afterwards. Mice were euthanized one month after vector delivery, their ovaries harvested and fixed in formalin overnight before being mounted in formalin.

For Western blots, fresh dissected mouse tissues or transfected cells were homogenized in 250 μL RIPA Lysis Buffer System with protease inhibitor cocktail and PMSF (Santa Cruz Biotechnology, Santa Cruz, CA), sonicated twice for 15 seconds at 11% amplitude and centrifuged 20,800 g for 15 minutes at 4° C. Supernatant was collected and protein content measured using Pierce BCA Protein Assay (ThermoFisher Scientific, Rockford, IL). Samples were prepared with either 50 or 100 μg protein extract, 4× sample buffer and RIPA buffer to a final volume of 25 μL and electrophoresis in Nupage 4-12% Bis Tris 1.5 mm gels (ThermoFisher Scientific, Rockford, IL). Proteins were transferred to a Nu-PAGE PVDF membrane and blocked with 5% milk for 1 h. Overnight incubation in primary, goat anti-MIS C-terminus antibody, MIS C-20 (Santa Cruz, Santa Cruz, CA) 1:200 in 5% milk was followed by a 1.5-2 h incubation in donkey, anti-goat IgG HRP 1:2,000. ProSignal Dura ECL reagent was applied for 1-2 minutes, and membrane was exposed for 4 minutes. Membranes were then stripped, blocked in milk for 30 minutes and incubated in goat, anti-beta actin (Santa Cruz, Santa Cruz, CA) for 2 h, donkey anti-goat 1:2,000 for 1.5-2 h and imaged as before. Membranes were stripped again and incubated anti-GAPDH antibody.

Female cats were treated with AAV9-fcMISv1 vectors. On Day 0 of the study, cats were anesthetized using a ketamine/dexmedetomidine combination with partial reversal using atipamezole. A single injection (0.51-1.30 ml total volume) was administered in the right caudal thigh muscles of each individual. Cats were housed singly for seven days under Biosafety Level 2 (BSL-2) containment before returning to group-housing.

Three adult female cats were injected intra-muscularly with 5e12 vector particles (vp)/kg of AAV9-chimeric felis catus MIS (AAV9-fcMISv1). Table 2 provides the weights and age of the subjects at the time of injection. Viral shedding was assessed by viral genome qPCR in stools, urine, rectal swabs, and oral swabs.

TABLE 2
Subjects AAV9-chimeric feline MIS

| | Treatment | | |
|---|---|---|---|
| | (vector genomes/kg | Age at time of | Weight at time of |
| Subject | body weight) | injection | injection (kg) |
| 11WBL25 | 5e12 | 5 yr, 7 mo, 14 d | 2.66 |
| 10WBJ5 | 5e12 | 6 yr, 11 mo, 13 d | 2.84 |
| 11WBL24 | 5e12 | 5 yr, 7 mo 14 d | 3.76 |

In some instances, in situ hybridization was performed using RNAscope 2.5 HD Reagent Kit (RED, ACD Bio, #322350) as previously described (Saatcioglu et al, 2019). Ovarian tissue sections from cats were hybridized with probes designed to identify AMH and AMIHR2, following the manufacturer's instructions. Briefly, following xylene deparaffinization and heat-induced epitope retrieval. The tissues were then hybridized for 2 hours and processed for standard signal amplification steps, and chromogen development. Slides were finally counterstained with hematoxylin, air-dried and cover slipped with EcoMount.

### Results

Both chimeric felis catus MIS protein (fcMISv1; SEQ ID NO: 3) and the flag-tagged version (flag-fcMISv1) were biologically active. Flag-fcMISv1 was expressed and purified from CHO cells (FIG. 1B) and was incubated with fetal rat urogenital ridge sections. As shown in FIG. 1D, flag-fcMISv1 induced regression of the Müllerian duct in fetal rat urogenital ridge in vitro. In nude mice, the proteins were delivered using AAV9 vectors as follows: (1) 5e12 vg/kg of AAV9-fcMISv1, or (2) 5e12 vp/kg of AAV9-empty negative control. The expression of fcMISv1 (which includes both cleaved and uncleaved versions; MISc and pro-MIS, respectively, in FIG. 1C) from the AAV9-fcMISv1 vector was confirmed in the quadricep, body wall, kidney, spleen, pancreas, and liver of the treated mice (FIG. 1C). As shown in FIG. 1E, fcMISv1 was also biologically active in vivo as it demonstrated induction of hypotrophy of the ovaries by day 50 (FIG. 1E).

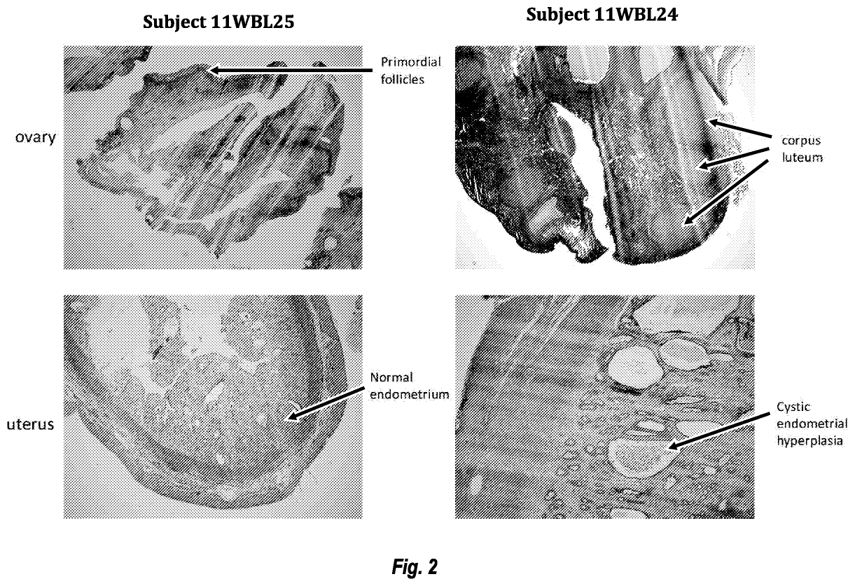

However, in female cats treated with 5e12 vg/kg of AAV9-fcMISv1, the chimeric feline MIS protein was rapidly eliminated within 7 days. Accordingly, levels of the inflammatory marker, C-reactive protein, peaked on day 7, but returned to baseline by day 14. All three cats displayed initial high circulating chimeric feline MIS protein (day 7: 6.44 μg/ml+/−0.03) in the blood. But expression was progressively lost in two of the three cats over the course of two years. Those two cats failed to maintain serum MIS levels in the μg/ml range. Further, high titers of anti-MIS antibodies could be detected within 14 days of treatment (FIGS. 1F-1H), likely because immunogenicity of the introduced sequence mismatches, while the cat which continued to produce MIS had evidence of ovarian suppression (FIG. 2).

### B. In Vitro Activity of Wild-Type Feline MIS Protein (fcMISv2) Expressed from a Codon Optimized Transgene

### Materials and Methods

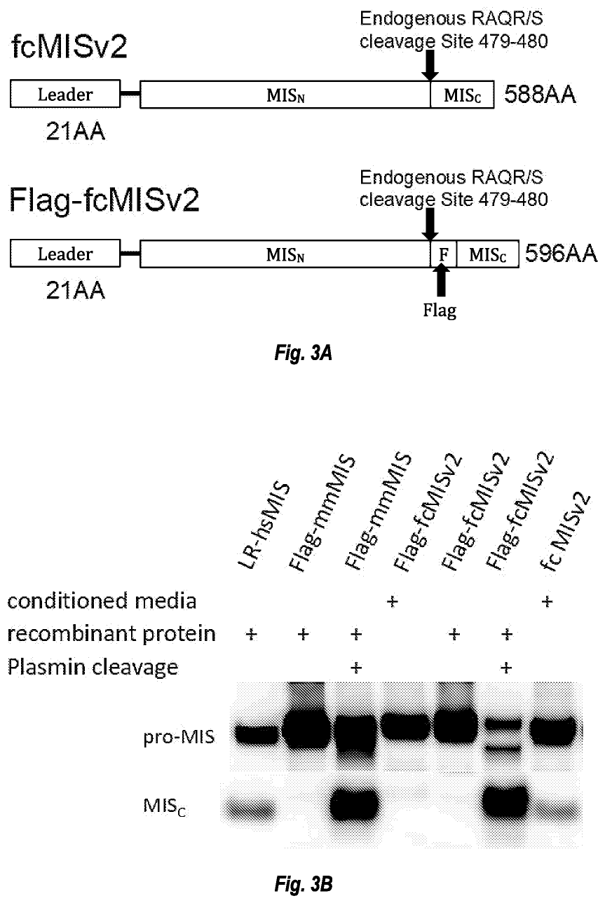

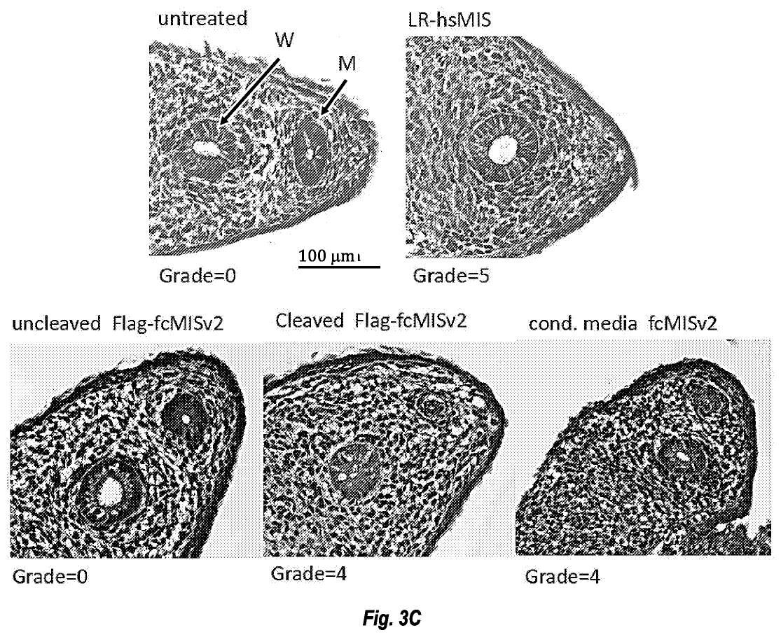

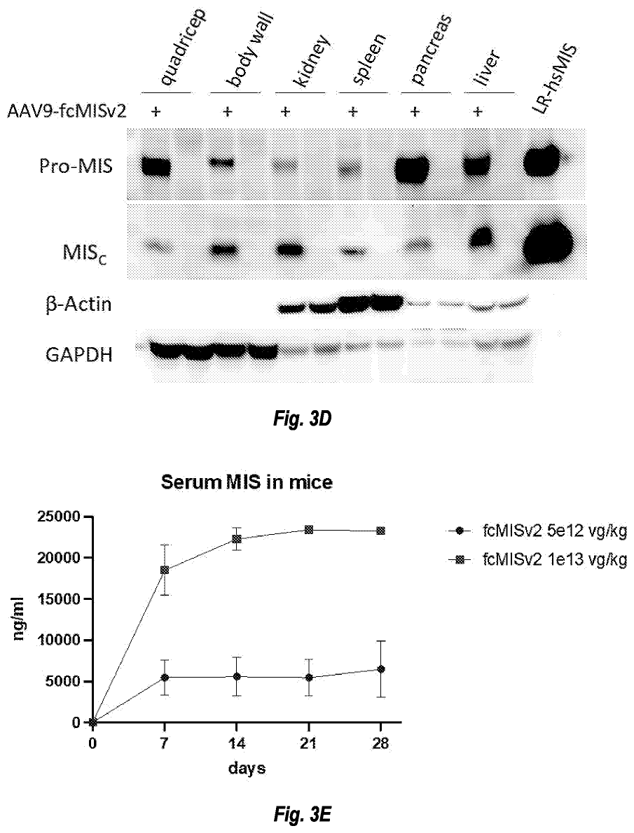

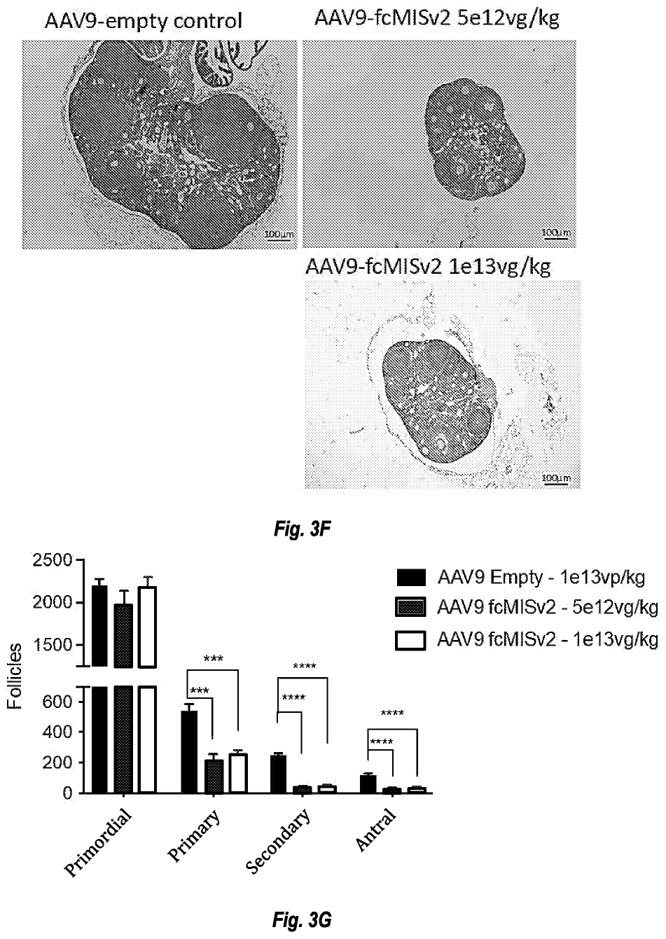

An AAV9 vector expressing wild-type felis catus MIS (wt-fcMIS or fcMISv2; SEQ ID NO: 1) was designed based on the domestic cat genome version 9.0 (FIG. 3A). A codon optimized transgene (SEQ ID NO: 5) was designed for feline translation and a reduced GC content to allow efficient viral packaging. A Flag-tagged variant was also designed (Flag-fcMISv2) for production and purification in CHO cells. Flag-fcMISv2 inhibited endogenous activating cleavage of pro-MIS, but could be processed with plasmin in vitro to produce MISN+C dimers (FIG. 3B).

Materials and methods related to CHO cells and Western blots were as described in Example 1.A. above.

Urogenital ridge regression assay was performed as previously described (Pépin 2013). Briefly, E14.5 female rat embryos urogenital ridges were dissected and set in culture on agar coated steel grids at the media/air interface and treated with conditioned media with human or feline MIS at 5 μg/ml, or with mock as negative control, for 72 h in humidified 5% CO₂ at 37° C. After incubation, the samples were fixed in Zamboni buffer, dehydrated in several steps overnight in a tissue processor and paraffin embedded. Ridge sections (8 m) were stained with hematoxylin and eosin. Scores from 0 (no regression) to 5 (complete regression of the Müllerian duct) were then attributed by two independent individuals.

### Results

Both the purified and enzymatically cleaved Flag-fcMISv2 induced regression of the Müllerian duct in the fetal rat urogenital ridge bioassay to a grade of 4 (out of 5) when adjusted to 5 μg/ml (FIG. 3C). The concentrated conditioned media from CHO cells that have stable overexpression of fcMISv2 also induced regression of the Müllerian duct in the fetal rat urogenital ridge (FIG. 3C).

### C. MIS Levels and Activity in Mice Following Treatment with AAV9-fcMISv2

The AAV9-fcMISv2 vector was also evaluated by intra-peritoneal injection using nude mice to avoid potential transgene immunogenicity.

### Materials and Methods

The fcMISv2 vector construct was further optimized to enhance transcription by using the CMV enhancer, the ubiquitous chicken 3-actin promoter, a synthetic intron, and a rabbit β-globin polyadenylation signal for terminating the 3′UTR (Gao et al, 2002), and packaged into AAV9 viral vectors (AAV9-fcMISv2). Given the efficient transduction of muscle tissues by the AAV9 serotype, and the relatively low abundance of AAV9 pre-existing antibodies in cats (Adachi et al., 2020; Li et al., 2019), this vector was used to deliver fcMISv2 (SEQ ID NO: 1) into this species.

Each mouse received a single intraperitoneal (i.p.) injection of AAV9-fcMISv2 at 5e12 vg/kg or 1e13 vg/kg, or 5e12 vg/kg of empty vector. Blood was collected from the mice cheeks prior to vector injection and weekly afterwards. Mice were euthanized one month after vector delivery, their ovaries harvested and fixed in formalin overnight before being mounted in formalin.

For follicle count analysis in mice, formalin fixed paraffin embedded mice ovaries were serially cut at 5 microns, and one cut every five was kept for follicles quantification as previously described. Following hematoxylin/eosin staining, slides were individually photographed and follicles with a nucleated oocyte were quantified. Follicles with one layer of squamous granulosa cells were qualified as primordial, one layer of cuboidal granulosa cells are primary, several layers of cuboidal granulosa cells are secondary and finally the ones presenting an antrum, tertiary or antral. A factor five was applied to obtain the final number of follicles. One ovary from 4 to 5 mice per group was quantified.

### Results

At one month following treatment, the fcMISv2 protein (SEQ ID NO: 1) was expressed in several tissues, including quadricep and body wall muscles, and peritoneal organs such as kidney, spleen, pancreas, and liver, and efficiently cleaved by endogenous proteases (FIG. 3D). Following treatment with either 5e12 vg/kg or 1e13 vg/kg of AAV9-fcMISv2, transduced tissues secreted MIS protein into the circulation, which, in turn, maintained levels of MIS above 0.5 μg/ml, which is above the 0.25 μg/ml target level necessary to ensure contraception in mice (Kano, 2017) (FIG. 3E). One month after treatment with either 5e12vg/kg or 1e13vg/kg of AAV9-fcMISv2, the ovaries of nude mice already displayed visible hypotrophy, (FIG. 3F), and displayed a significant reduction in follicle counts from all stages of follicle maturation (FIG. 3G).

### D. Pilot Study of Adult Female Cats Treated with AAV9-fcMISv2

### Summary of Pilot Study and Results

The effect of the AAV9-fcMISv2 vector on feline reproduction was studied in domestic cats (Felis silvestris catus). Adult female cats were injected with the AAV9-wt felis catus MIS (AAV9-fcMISv2) or empty vector control as follows: (1) High dose AAV9-fcMISv2 (1e13 vg/kg; n=3); (2) Low dose AAV9-fcMISv2 (5e12 vg/kg; n=3); and (3) Control empty vector (5e12 vp/kg; n=3).

Estradiol (E2) and progesterone (P4) metabolites were quantified from fecal samples collected 3×/week, beginning six months before treatment. Cats were also monitored for behavioral signs of estrus. One cat in the high dose group exhibited transient injection site edema; no other injection site reactions were observed. Physical exams, blood work, and well-being assessments conducted throughout the study were otherwise unremarkable. Serum MIS concentration and anti-transgene antibody titers were monitored by ELISA during the 8-month post-injection observational study. Following the initial 8-month observation period, the cats were monitored over a 4-month breeding trial, and underwent ongoing observation to determine if pregnancies had been averted and to monitor the levels of steroidogenesis and ovarian proteins that persist.

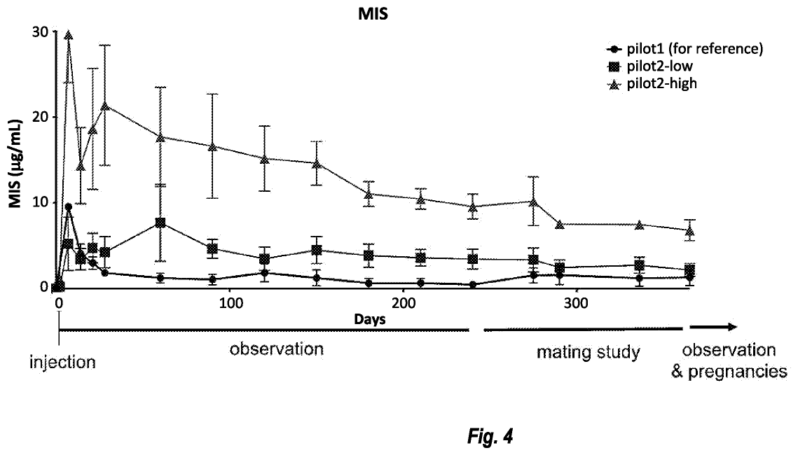

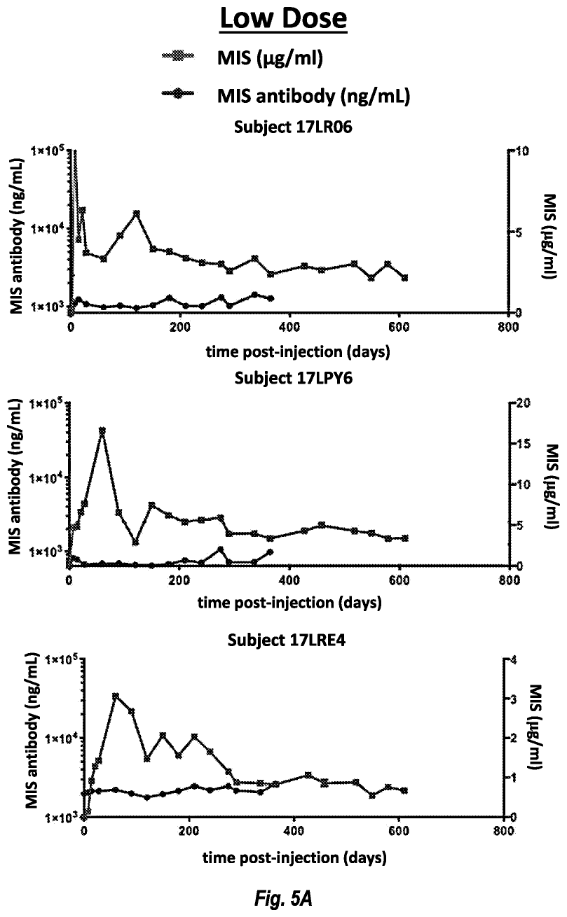

All cats treated with AAV9-fcMISv2 displayed circulating MIS level throughout the study, including the mating period (FIG. 4). Overall, MIS levels initially declined and appeared to reach a stable level of MIS expression over the year of observation for both treatment groups, reflecting characteristic stabilization of expression from the persisting viral genomes following AAV gene transfer. Serum concentration of MIS remained stable during the study period, averaging 2.88 μg/ml+/−2.32 in the low group, 11.78 μg/ml+/−2.51 in the high group, while remaining basal (5 ng/ml+/−1) in controls at 6 months. No anti-MIS antibodies were detected during this period in any of the cats treated with AAV9-fcMISv2. In contrast with these results, the two cats with loss of MIS gene expression in the first pilot study had antibody titers of 58 μg/ml±25, and MIS levels of 0.07 μg/ml±0.07 at the 6-month timepoint.

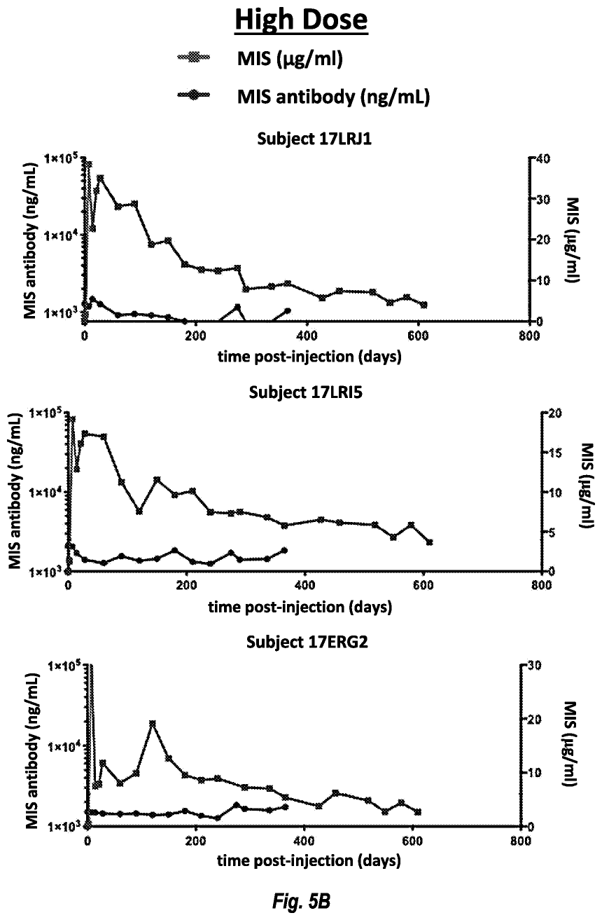

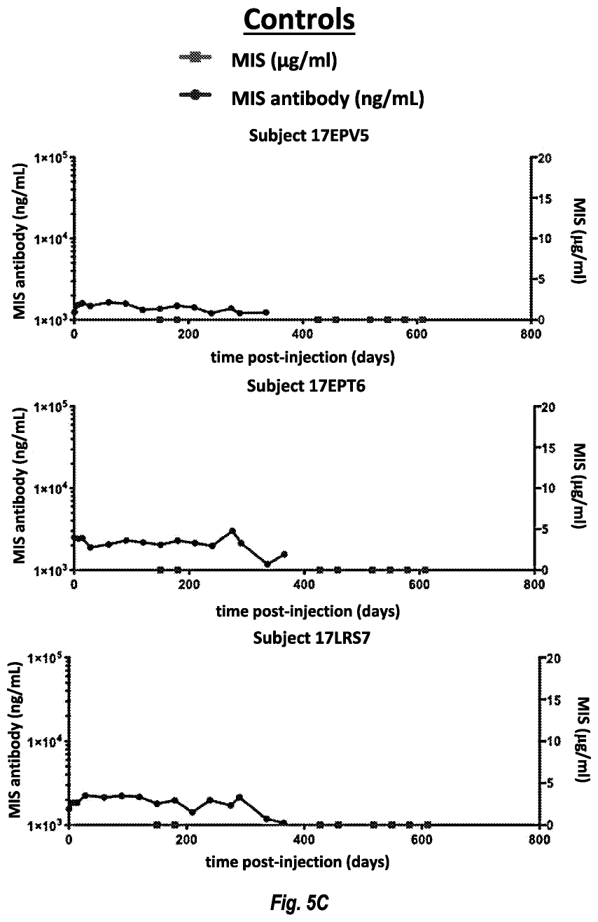

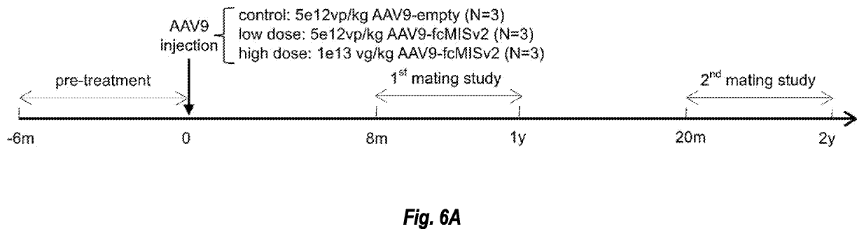

Five of the six treated cats maintained a circulating MIS concentration of above 1 μg/ml over the first year of observation. (FIG. 5A and FIG. 5B). At the one-year timepoint, in the low dose (5e12 vp/kg) group, Subject 17LPY6 had a circulating MIS concentration of 3.34 μg/ml, Subject 17LR06 had a circulating MIS concentration of 2.38 μg/ml, and Subject 17LRE4 had a circulating concentration MIS of 0.82 μg/ml (ranging from 1.65 to 0.82 μg/ml during the mating period). In the high dose (1e13 vp/kg) group, all cats had high circulating MIS concentrations at the one-year timepoint with Subject 17LRI5 at 5.78 μg/ml, Subject 17LRJ1 at 9.28 μg/ml, and Subject 17ERG2 at 5.36 μg/ml.

FIGS. 5A, 5B, and 5C show individual profiles of serum MIS concentration (g/ml) (square) and circulating anti-MIS antibody concentration (circle) over time in each mature cat following injection with low dose of AAV9-wt feline MIS (5e12 vp/kg, n=3) (FIG. 5A), high dose of AAV9-wt feline MIS (1e13 vp/kg, n=3) (FIG. 5B), or control empty vector particles (5e12 vp/kg, n=3) (FIG. 5C).

In contrast to the first pilot study with AAV9-chimeric feline MIS (AAV9-fcMISv1), AAV9-wild-type feline MIS (AAV9-fcMISv2) did not elicit detectable antibodies against fcMISv2 in any of the treated cats, as tested by ELISA.

Evidence of ovarian suppression was observed, particularly in the high dose cohort as demonstrated by reduced E2/P4 levels and peaks. Pooling values from the low and high-dose groups, mean fecal E2 metabolite concentrations were compared between two time periods: 6 months pre-injection versus 6 months post-injection. Analyses revealed a significant E2 reduction (P=0.048) in treated cats (145.4 ng/gm dried feces) relative to controls (198.4 ng/gm) during the post-injection period. Mean E2 did not differ between pre-injection groups (P=0.262). Number of monthly estrus phases (defined as 2+ consecutive fecal samples with E2>1.5× E2 baseline) did not differ between groups either pre- or post-injection (P=0.802, P=0.201 respectively). However, treated cats tended to have fewer estrus phases post- vs. pre-injection (pre: 0.89; post: 0.53; P=0.081).

Based on these results, a four-month breeding trial was initiated using a proven-breeder male that was group-housed (8 hours/day, 5 days/week) with the nine females and continually monitored to record all breeding interactions. See Example 1.E. below.

### Materials and Methods

The cats were maintained in a research colony at the Cincinnati Zoo and Botanical Garden's Center for Conservation and Research of Endangered Wildlife (CREW). All procedures were approved by their Institutional Animal Care and Use Committee (Identification Number 18-132) and the Cincinnati Children's Hospital Medical Center Institutional Biosafety Committee (IBC 2018-0066). All cats treated in the study (n=9) were sexually intact females from different dams, which provided genetic diversity among the study population. Upon arrival at CREW, the cats were boarded for several months for acclimatization to the surrounds and keepers; to collect blood, fecal, and urine samples to establish baseline levels of hormones of interest, and to ensure the cats were sexually mature at the time of injection. Cats were group-housed, fed a commercial dry cat food, and provided access to fresh water ad libitum. Two proven breeder male cats (2 years old and 13 years old) used during the breeding trials were singly housed in a separate room, but otherwise kept under the same conditions. Physical exams and blood work (complete blood count (CBC) and serum biochemistry panel) were performed prior to study enrollment.

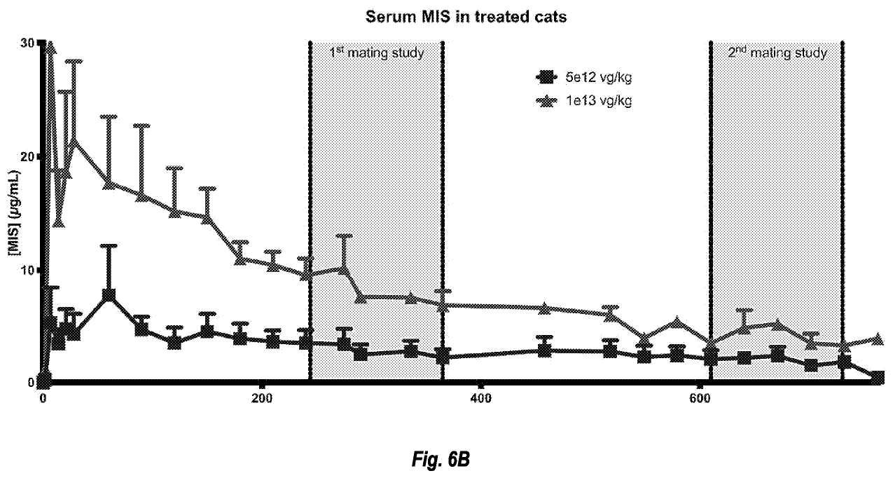

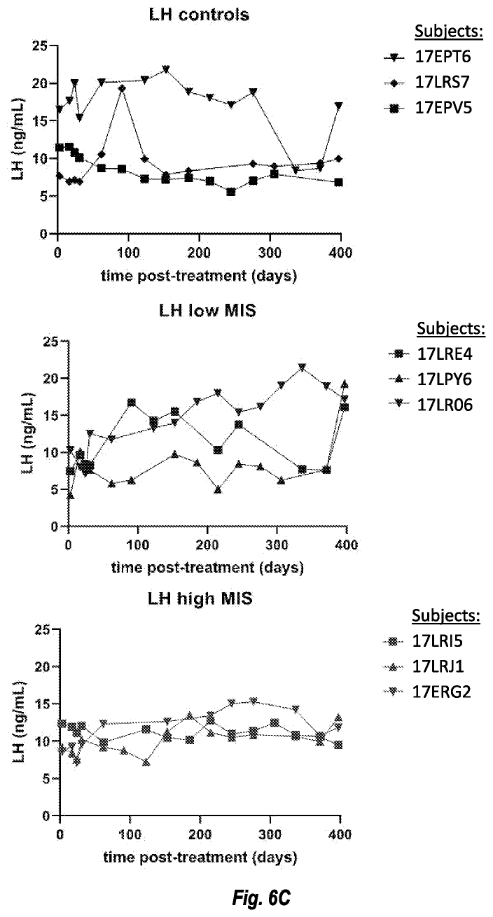

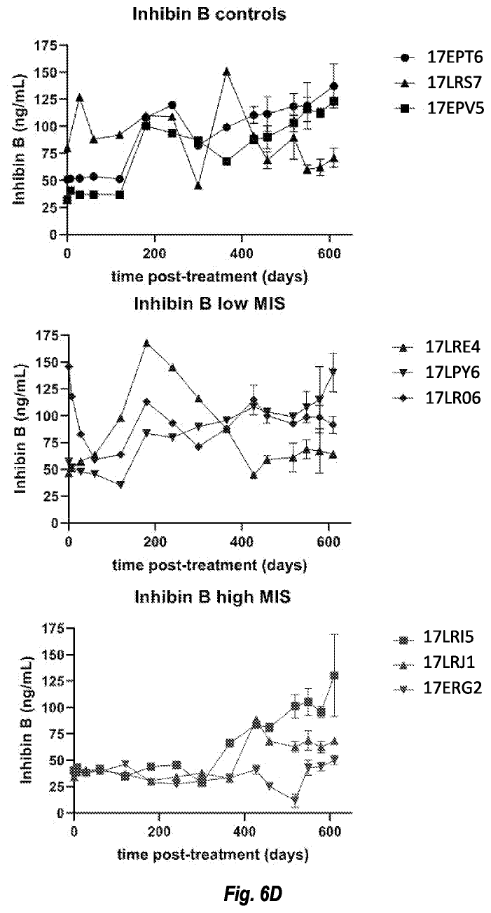

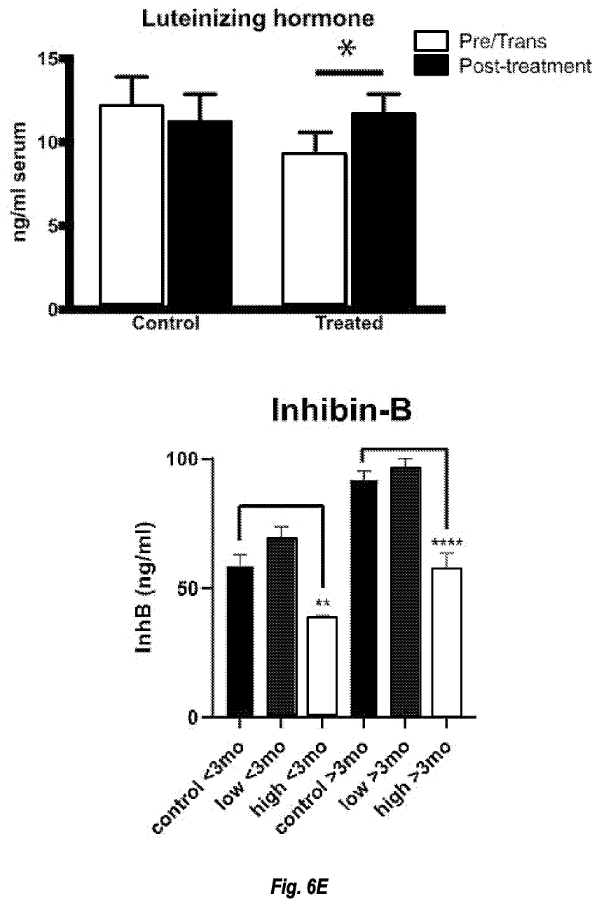

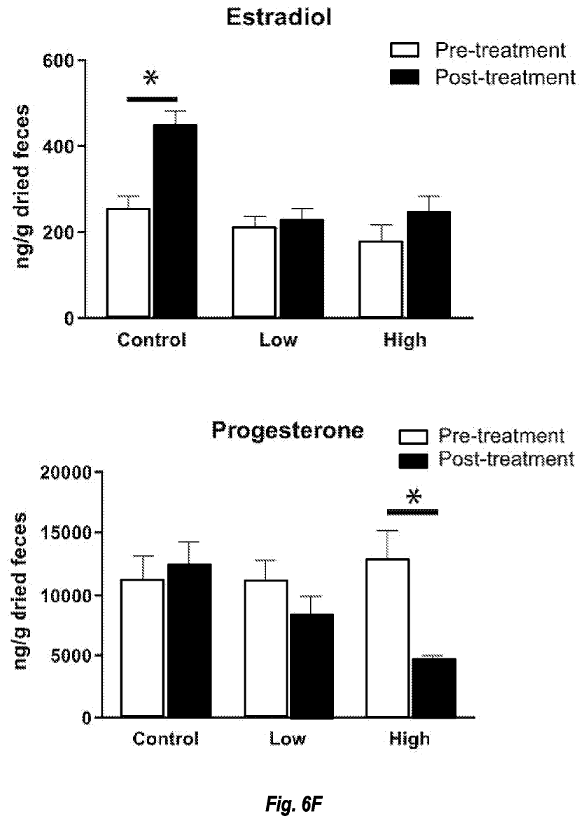

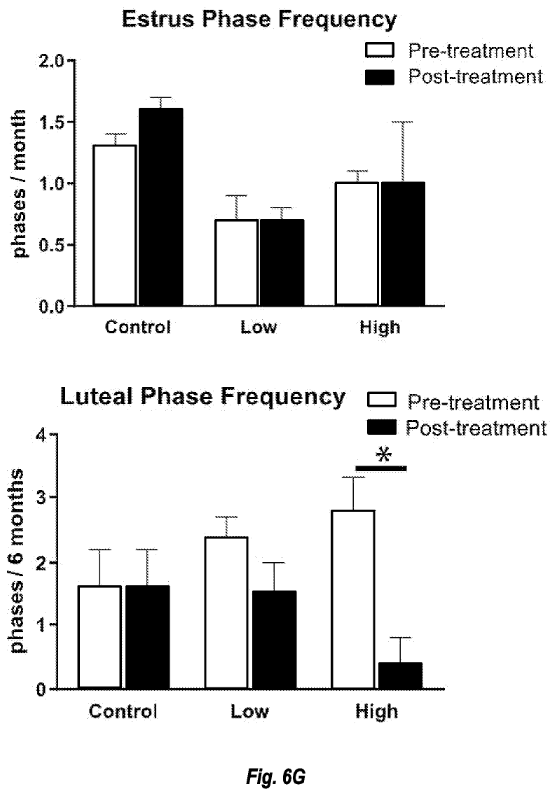

The adult female cats were randomly assigned to one of these three treatment groups using the random number generator function in Microsoft Excel. FIG. 6A is a schematic of the study design and Table 3 provides the age and weights of the subjects at the time of injection.

TABLE 3
Subjects AAV9-wt feline MIS

| | Treatment | Age at time of | Weight at time of |
|---|---|---|---|
| Subject | Group | injection | injection (kg) |
| 17LR06 | Low dose | 1 yr, 2 mo, 10 d | 4.2 |
| 17LRE4 | Low dose | 1 yr, 2 mo, 16 d | 2.54 |
| 17LPY6 | Low dose | 1 yr, 2 mo, 30 d | 2.67 |
| 17ERG2 | High dose | 1 yr, 2 mo, 12 d | 2.77 |
| 17LRJ1 | High dose | 1 yr, 2 mo, 14 d | 3.24 |
| 17LRI5 | High dose | 1 yr, 2 mo, 15 d | 2.03 |
| 17EPV5 | Control | 1 yr, 3 mo, 2 d | 3.36 |
| 17LRS7 | Control | 1 yr, 2 mo, 9 d | 3.2 |
| 17EPT6 | Control | 1 yr, 3 mo, 2 d | 2.61 |

On Day 0 of the study, cats were anesthetized using a ketamine/dexmedetomidine combination with partial reversal using atipamezole. A single injection (0.51-1.30 ml total volume) was administered in the right caudal thigh muscle of each cat. Cats were housed singly for seven days under Biosafety Level 2 (BSL-2) containment before returning to group-housing.

Female cats were assessed daily for general wellbeing during the first two weeks and no adverse events were observed. Physical exams and blood work were performed two weeks prior to treatment, at day 0 (before MIS treatment), repeated every three months through Year 1 of the study, and every six months thereafter; all results were unremarkable. Injection sites were examined daily for 14 days, weekly for the next two weeks, and then monthly thereafter. One cat in the group administered with 1e13 vg/kg of AAV9-fcMISv2 (i.e., the high dose group) demonstrated slight edema at the injection site on Days 3 and 4. No evidence of pain stimulus behaviors, change in temperature, or tissue mass formation were observed, and the edema resolved by Day 5. No other injection site reactions were observed.

Viral shedding in feces and bodily fluids was measured by quantitative PCR performed at the University of Florida Powell Gene Therapy Center Toxicology Core. Fecal and urine samples were collected daily from individual cats during the 7-day post-treatment period in biolevel safety-2 housing. Oral swabs were obtained at Days 0 (pre-treatment), 2, 7, and 14. Whole blood (jugular vein, cephalic vein, or lateral saphenous vein) was collected in microtainer EDTA tubes at Days 0 (pre-treatment), 2, 21, 28, and monthly thereafter through Month 6. Fecal samples were sealed in plastic bags. Urine, oral swabs, and blood samples were transferred into 1.8 ml cryovials. All samples were stored at −20° C. until analysis.

For feline MIS protein analysis, venous blood samples were collected at Days 0 (pre-treatment), 2, 7, 14, 21, 28, and monthly thereafter through Year 2. Blood was collected into serum separator tubes, allowed to clot for approximately 15 minutes, and centrifuged for 10 minutes at 1534 g. The recovered serum was transferred into 1.8 ml cryovials and stored at −80° C. until analysis. MIS levels were measured using the AMH Gen II ELISA Ruo (Beckman Coulter, Miami, FL). Briefly, cat sera were diluted in diluent as follows: All control, day zero low and day zero high dose cats 1:10; subsequent low dose cats 1:1000 to 1:500 with Subject 17LRE4 and exception 1:100; high dose cats 1:2000 to 1:1000. Manufacturer's instructions were followed for the remainder of the ELISA.

An ELISA assay was developed to measure anti-fcMISv1/v2 IgG in cat serum without the use of an antibody sandwich. In this ELISA, recombinant FLAG-tagged fcMISv1 (FLAG-fcMISv1) or FLAG-tagged fcMISv2 (FLAG-fcMISv2) was purified from conditioned media. FLAG-fcMISv1 or v2 protein (i.e., the capture protein) was added directly to the ELISA plate (Immulon HB2 ELISA plate, Thermo Fisher Scientific, Rochester NY cat. 3455) at 5 μg/ml. The ELISA plate was directly coated with the FLAG-tagged wt feline MIS protein rather than being immobilized with a rabbit anti-FLAG antibody, which may be a source of high background due to cross-reactivity with the developing antibody. Standard wells were coated with whole molecule cat IgG (Rockland Antibodies and Assays, Limerick, PA cat. 002-0102-0002), in eight 3-fold dilutions in coating buffer (CB) starting with 900 ng/mL. Control and blank wells received just coating buffer. (Rows of capture protein alternated with rows of controls). The plate was then incubated 30 minutes at RT and overnight at 4° C. Two rinses were performed, and the plate was blocked for 2.5 h with 200 μL/well 1% Bovine Serum Albumin (Jackson Laboratories cat. 001-000-162)+7.5% normal goat serum (Abcam cat. Ab7481) in PBST. Samples were diluted by a factor of 100 in blocking buffer, added, and the plate incubated for 1 h at RT. After 5 more washes, the plate was incubated for 1 hour in the dark at 4° C. with goat anti-feline IgG (H+L) HRP (Novus Biologicals cat. NBP73347) 1:10,000 in PBST. The plate was rinsed 5 times and the enzyme substrate reaction performed. For hormone metabolite analysis, fecal samples were collected on three non-consecutive days per week beginning six months prior to MIS treatment and concluding two years post-treatment. To facilitate identification of fecal samples from group-housed females, a unique combination of commercially available food-grade dye (Wilton Industries, Woodridge, IL, USA) and/or glitter (Dixon Ticonderoga Company, Appleton, WI, USA) was fed to each cat in a small amount of canned food each day preceding a sample collection day. Fecal samples collected could then be identified to each individual cat by the presence of the dye color or glitter. Samples were sealed in plastic bags, labeled with name and date collected, and stored at −20° C. until processing.

For fecal E2 and P4 analysis, fecal samples were lyophilized via a freeze dryer (Labconoco Corp., Kansas City, MO, USA) in their plastic bags, pulverized into a fine powder, and then weighed (250±5 mg) into labeled 15 ml polypropylene conical tubes. Each of the samples was then extracted by adding 2.5 ml of 90% ethanol (or a 1:10 w:v) overnight on a mechanical rocker (>12 h). Extracted samples were then centrifuged at 1000 g for 15 minutes, supernatants were pipetted off, and samples stored in 2.0 ml cryovials at −20° C. until analysis. Procedures for all enzyme immunoassays (EIAs) were modified from previously published methods. A polyclonal antibody produced against 170-estradiol (R0008) was used in conjunction with a horseradish peroxidase (HRP) ligand to determine estrogens (E2), whereas the Arbor Assays P4 mini-kit (ISWE003, Arbor Assays, Ann Arbor, MI, USA) was used to quantify progestogens (P4). This kit included both antibody and HRP. Both assays have been previously used in this lab and validated for use in domestic cats. For both assays, samples and standards were analyzed in duplicate.

For statistical analysis of fecal hormone metabolites, hormone baseline values were calculated for each female based on the six-month pre-treatment sampling period. Data were analyzed using the R statistical package hormLong. For each individual, baseline E2 and P4 values were calculated using an iterative process excluding all points greater than the mean plus 1.5 standard deviations. An estrus phase was defined as two or more consecutive fecal samples with an E2 value greater than 1.5 times baseline. A luteal phase was defined as six or more consecutive fecal samples with a P4 value greater than 1.5 times baseline. Number of estrus phases, number of luteal phases, and mean fecal metabolite concentrations were compared between two time periods: six months pre-treatment versus 22 months post-treatment, after a two-month post-treatment transition phase. Periods of pregnancy and lactation were excluded from analyses. To account for the different lengths of time for each sampling period, estrus phases are reported as the number of estrus phases in a one-month period (total number of estrus phases/number of days in sampling period×30 days) and luteal phases are reported as the number of luteal phases in a six-month period (total number of luteal phases/number of days in sampling period×180 days). Data were analyzed as a randomized complete block design, using an ANOVA, where Period and Treatment Group (and their interaction) are fixed effects, and the individual animals are included as a random effect (blocks). The Tukey's multiple mean comparison test was used for pairwise comparisons. Analyses were performed using SAS© Studio software (Release: 3.8, Enterprise Edition, SAS Institute Inc., Cary, NC, USA).

For serum LH analysis, diluted serum samples (1:5) were analyzed utilizing a double antibody EIA adapted from Graham et al., Zoo Biol, 20:227-236 (2001). NIH-bovine LH standards, controls, and samples were added to plate wells in duplicate. After overnight incubation, biotinylated NIH-ovine LH was added to all wells and allowed to compete for 4-hour at room temperature. After competition, plates were then incubated with streptavidin-peroxidase. The EIA was validated for cat serum by demonstrating parallelism between dilutions of pooled serum and the standard curve. Furthermore, bovine LH (used for standards) was added to cat serum samples and a dose-response curve was generated. Intra- and inter-assay coefficients of variation were 3.5% and 8%.

For statistical analysis of serum LH, serum LH samples were divided into three groups: pre-treatment/transition (immediately before treatment through two-month transition period; Pre/Trans), early post-treatment (Months 3-8, Early post), and late post-treatment (Months 15-20, Late post). These groups were selected to avoid sampling periods during the breeding trials, or subsequent pregnancy and lactation. Analyses were conducted using SAS® Studio software, Release: 3.8. The change in serum LH across time (samples) in treated vs. non-treated cats was evaluated within the MIXED procedure using a generalized linear mixed effect model, with treatment, time (3 levels: Pre/Trans, Early post, and Late post), and the treatment×time interaction as fixed effects, and cat (individuals) as random blocks, using the Satterthwaite adjustment for degrees of freedom. Tukey's multiple mean comparison test was used to compare the levels of time within each treatment group. Significant differences were declared when P<0.05.

For inhibin B analysis in blood, inhibin B was measured using the Cat INHB-B ELISA Kit (Mybiosource.com, San Diego, CA). Undiluted cat serum samples were assayed using the manufacturer's protocol.

### Results

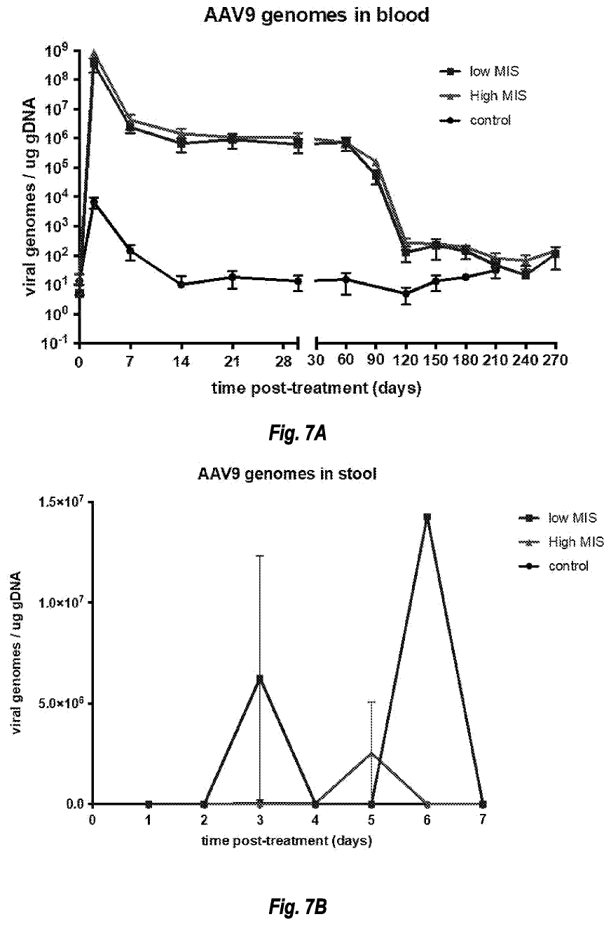

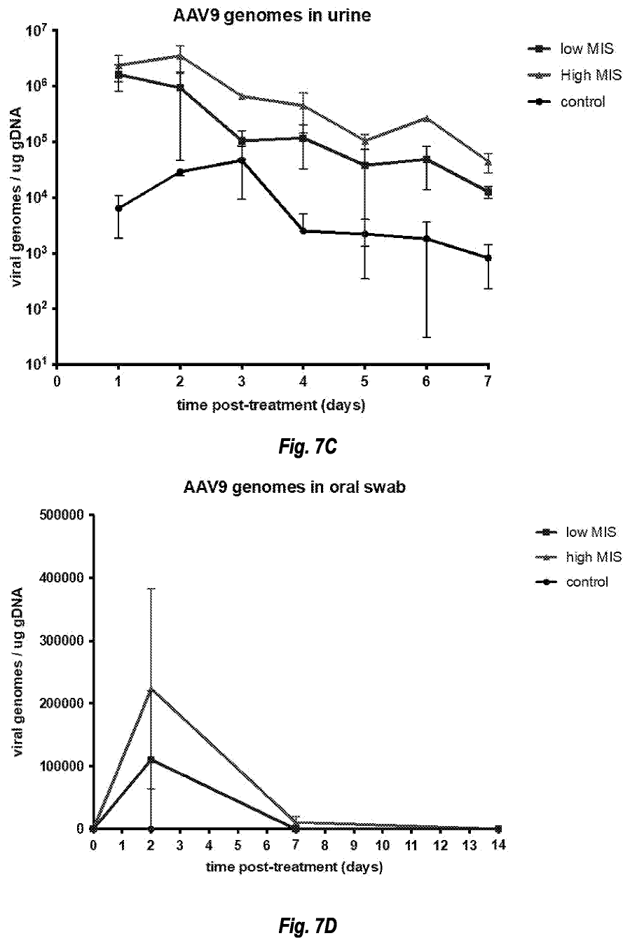

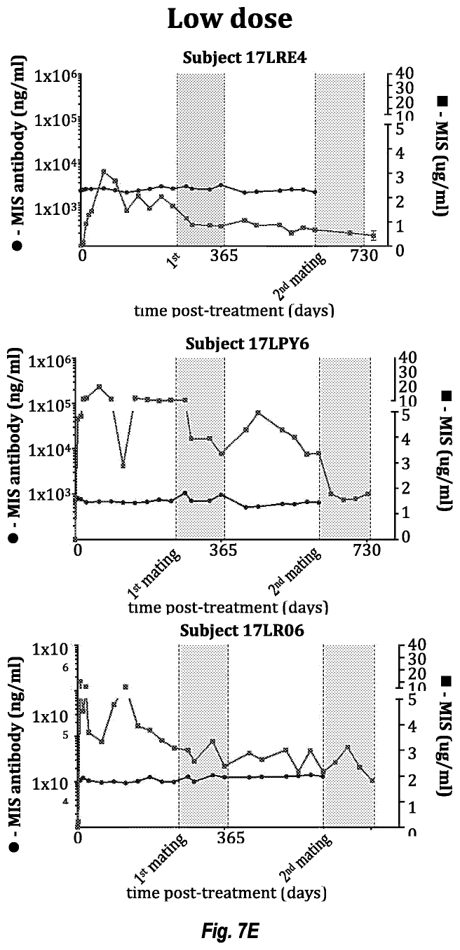

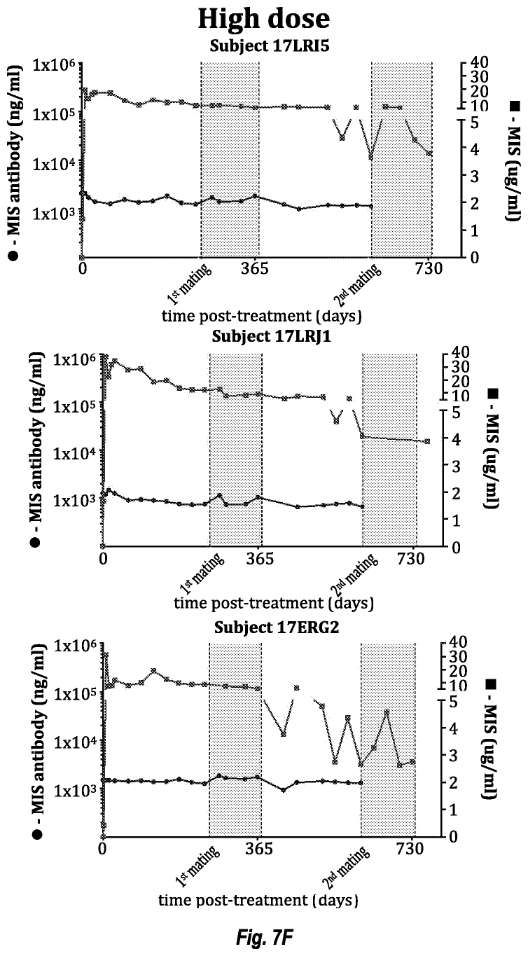

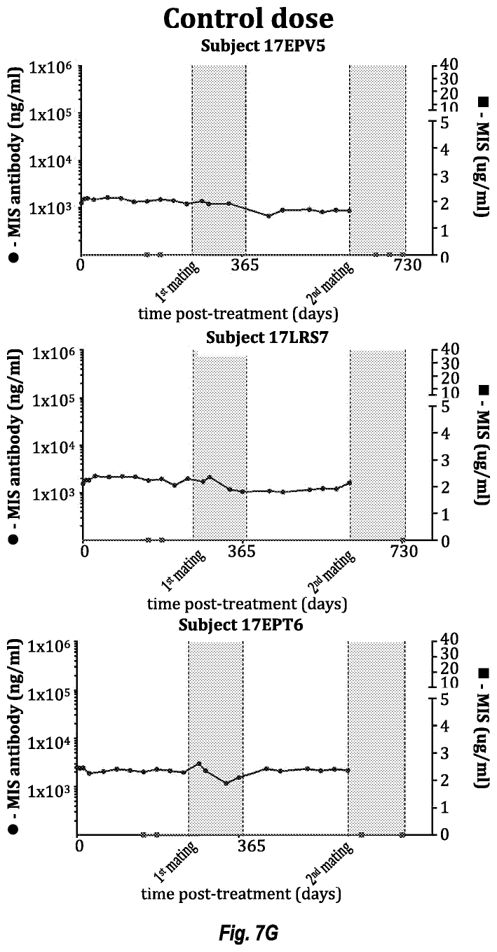

Viral genomes were detected in the blood on day 2 and remained elevated for 2 months before rapidly decreasing at the 3-4-month period, likely reflecting infected cell turnover (such as liver cells) releasing cell free DNA (FIG. 7A). Viral shedding was detected in the urine on day 1 and steadily decreased over 7 days, while measurements in oral swabs and stool samples showed variability across individuals and groups (FIGS. 7B-7D).

Circulating MIS levels were initially robust but gradually decreased during the first year, eventually reaching a relative plateau in the second year, which remained above 0.5 μg/ml (FIGS. 7E-7G). One treated cat, Subject 17LRE4, in the group treated with 5e12 vg/kg of AAV9-fcMISv2 (i.e., the low dose group) had the lowest circulating MIS levels, averaging 0.93 μg/ml and 0.61 μg/ml during the first and second mating trials respectively (FIG. 7E). Intriguingly, Subject 17LRE4, also had viral genomes two orders of magnitude lower at day 2 compared to the other cats in her group, suggesting misinjection or pre-existing vector immunity (FIG. 7E).

Importantly, unlike the robust anti-transgene antibody response observed with fcMISv1 (FIG. 1F-1H), no anti-fcMISv2 antibodies were detected over background in any of the cats given AAV9-fcMISv2 using the direct capture ELISA (FIGS. 7E-G).

As previously observed, there is a delay between AAV9-MIS treatment and complete ovarian suppression of approximately 1 month in mice, likely corresponding to the time required for the cohort of pre-existing growing follicles to complete maturation and be cleared from the ovary (Kano et al., 2019, 2017). Cat reproductive hormones were measured and compared during the first 3 months of treatment and the remainder of the post-injection period. The hormones measured were luteinizing hormone (LH) (FIG. 6C), a pituitary feedback hormone, and inhibin B (FIG. 6D), a marker of growing follicles.

Average serum LH was calculated over three time periods: (1) immediately before treatment through two-month transition period (Pre/Trans), (2) early post-treatment (3-8 months; Early post), and late post-treatment (15-20 months; Late post). Analysis revealed there were no differences in LH for any period in the control cats. However, in the AAV9-MIS-treated cats, LH was elevated in both post-treatment periods compared to Pre/Trans (P=0.0008, Early post; P=0.0036 Late post) (Table 4; FIG. 6E). There was no difference between Early post and Late post periods in treated cats.

TABLE 4
Serum LH

| | Serum LH (ng/ml) | | |
|---|---|---|---|
| Treatment | Pre/Trans | Early post | Late post |
| Control | 12.1 (1.8) | 11.2 (1.7) | 0.6955 |
| Treated | 9.3 (1.3) | 11.7 (1.2) | 0.0001 |

Data presented as mean (SEM)

Conversely, inhibin B was significantly reduced in the high dose group during all periods (FIGS. 6D-6E). These profiles suggest hypergonadotropic hypogonadism, particularly at the high dose.

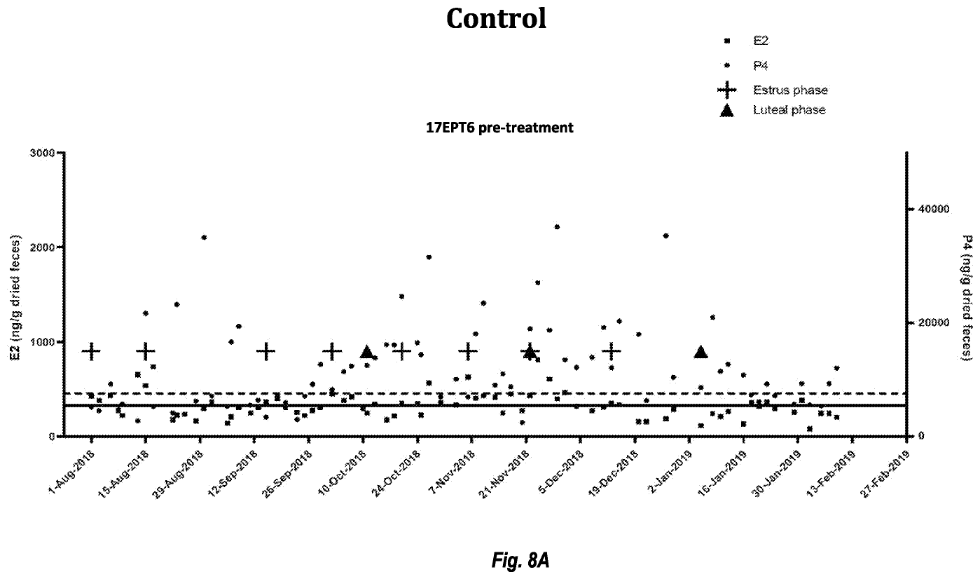

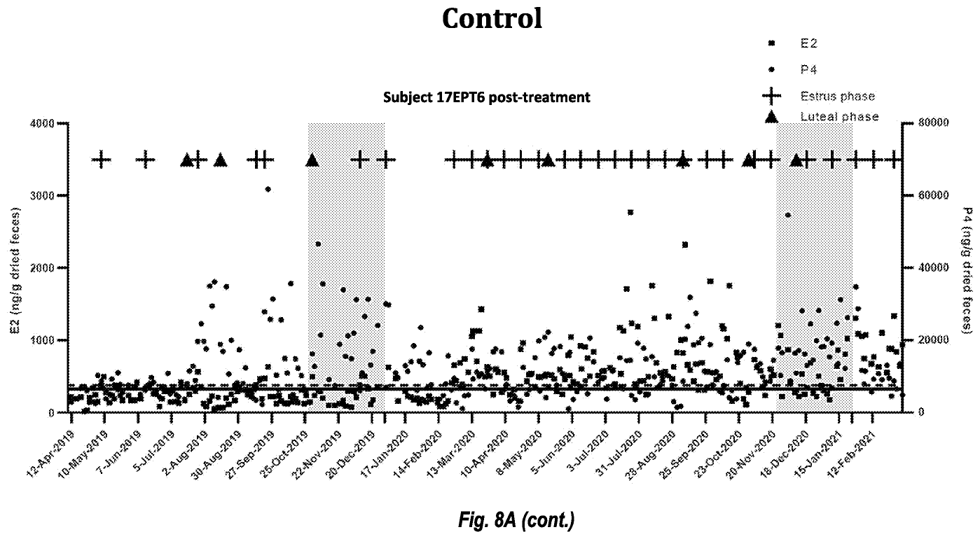

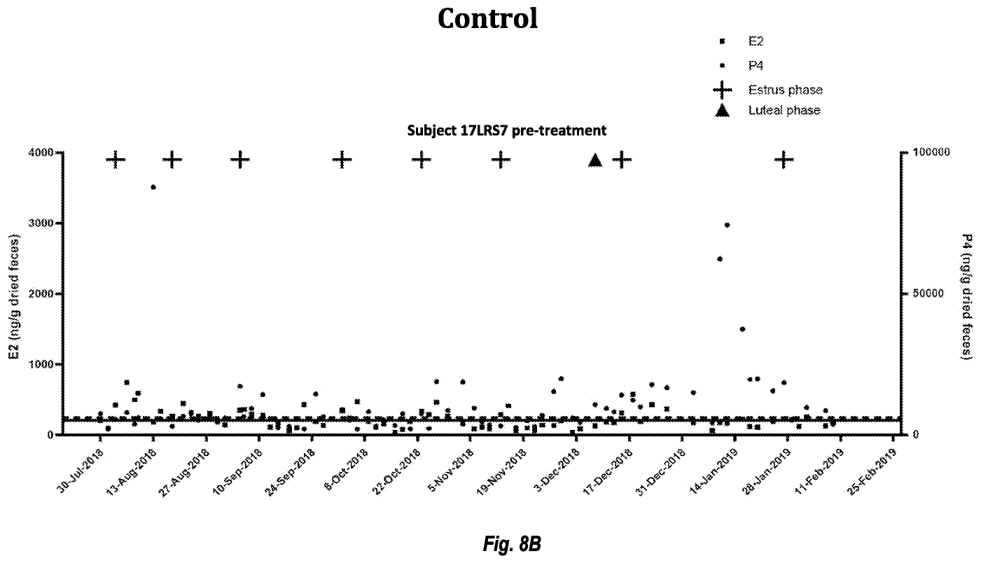

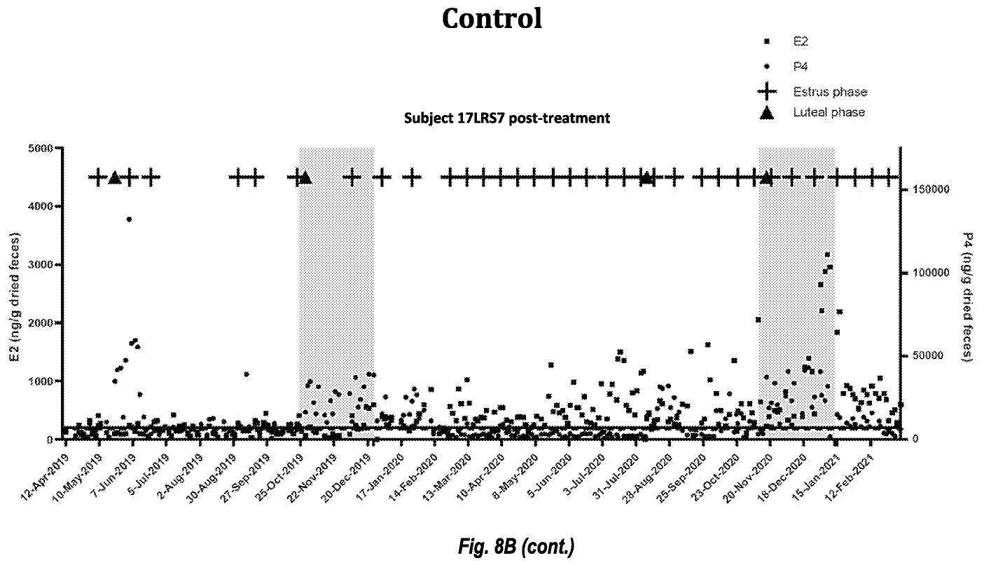

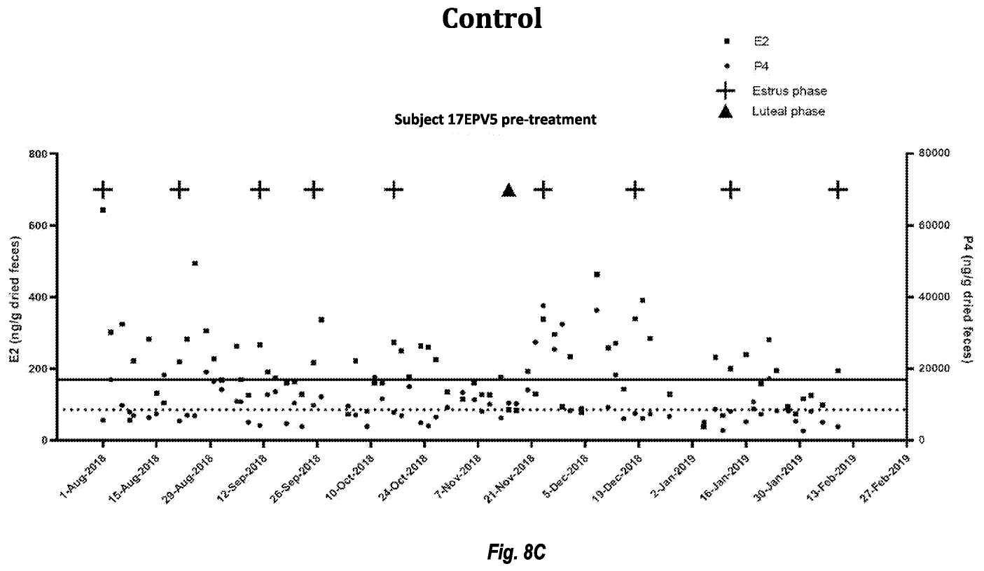

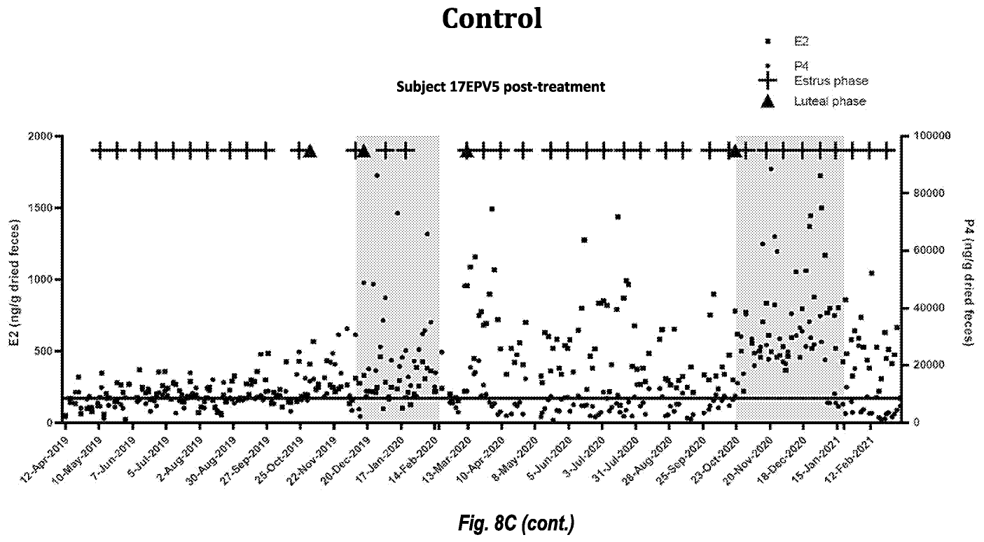

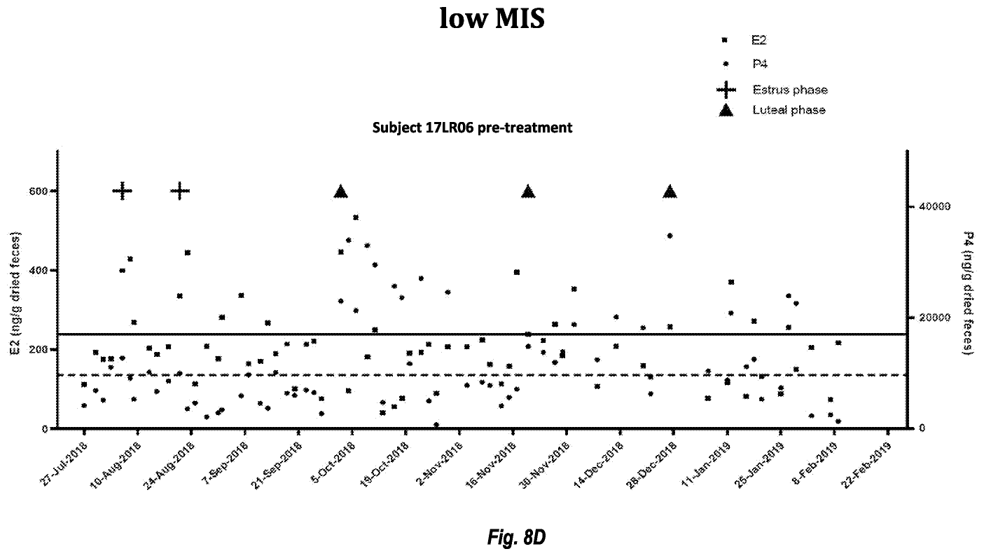

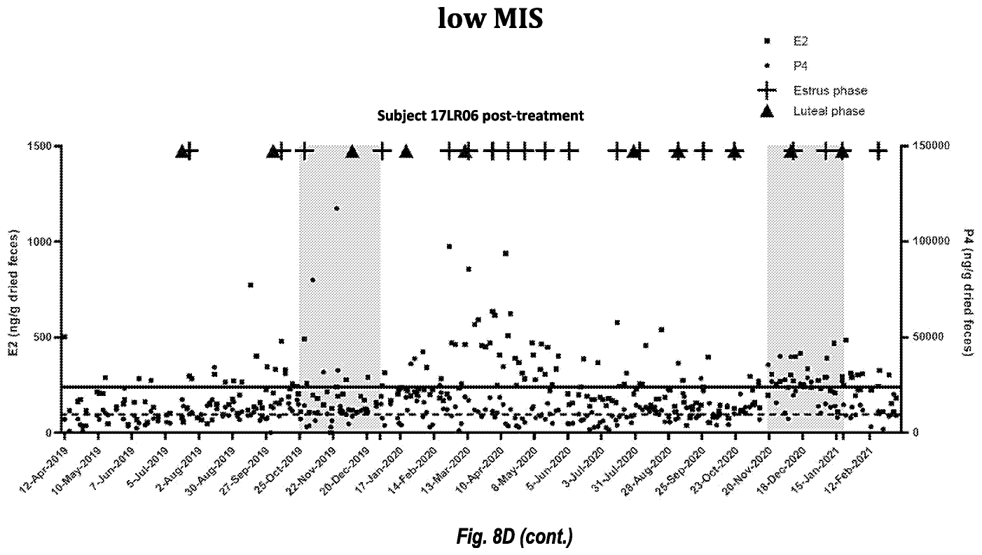

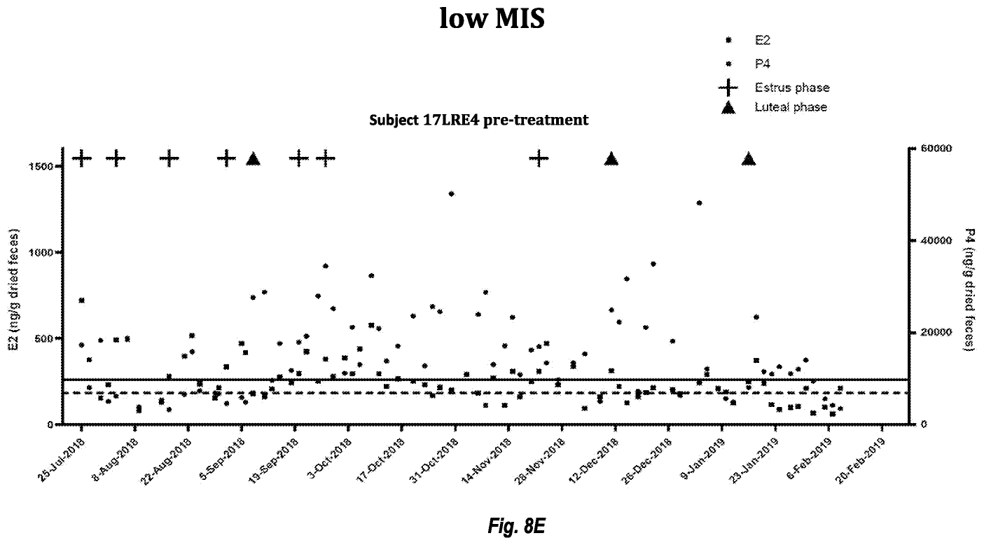

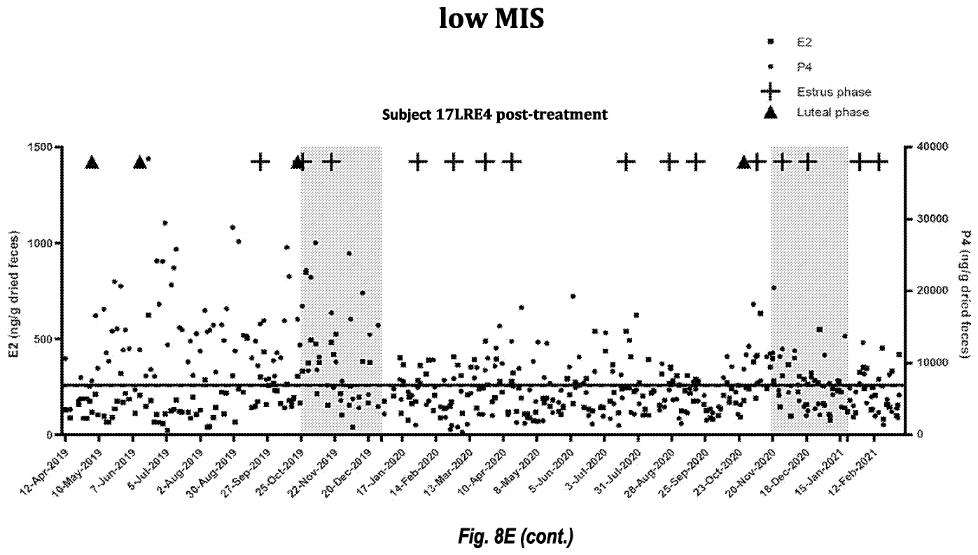

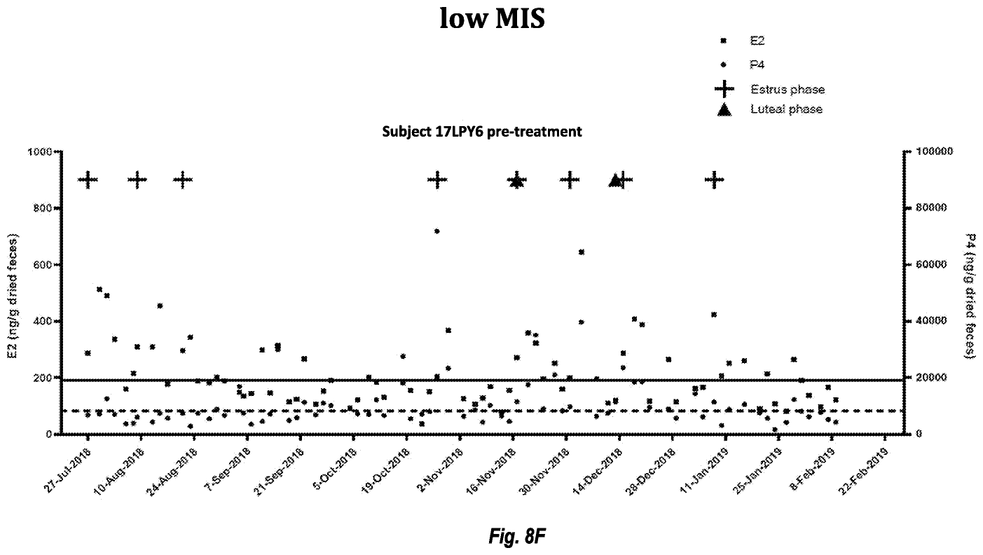

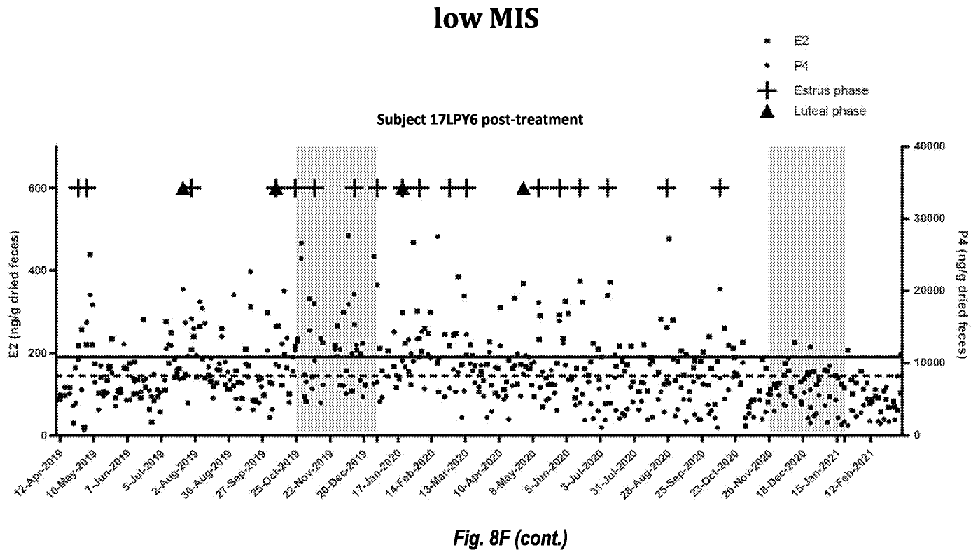

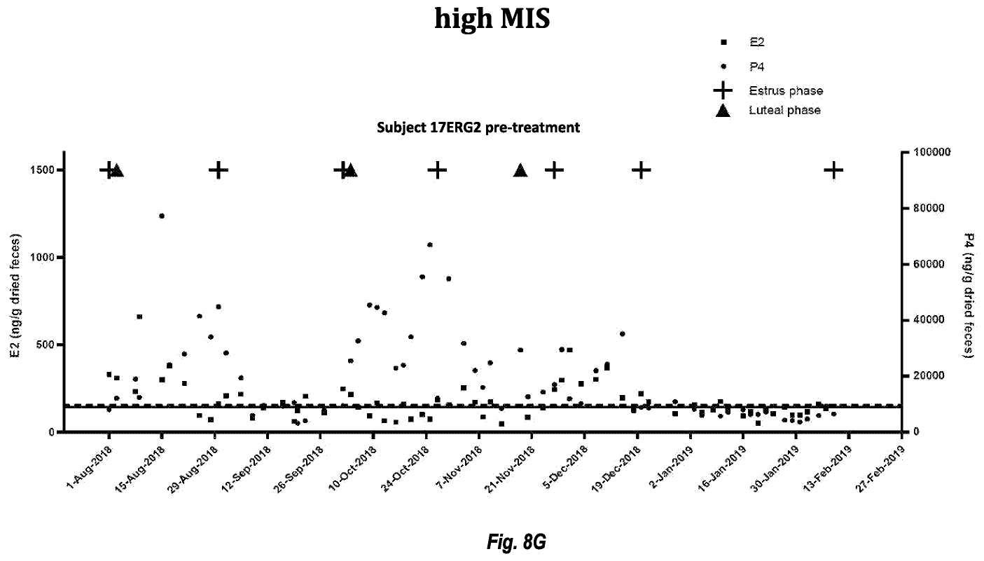

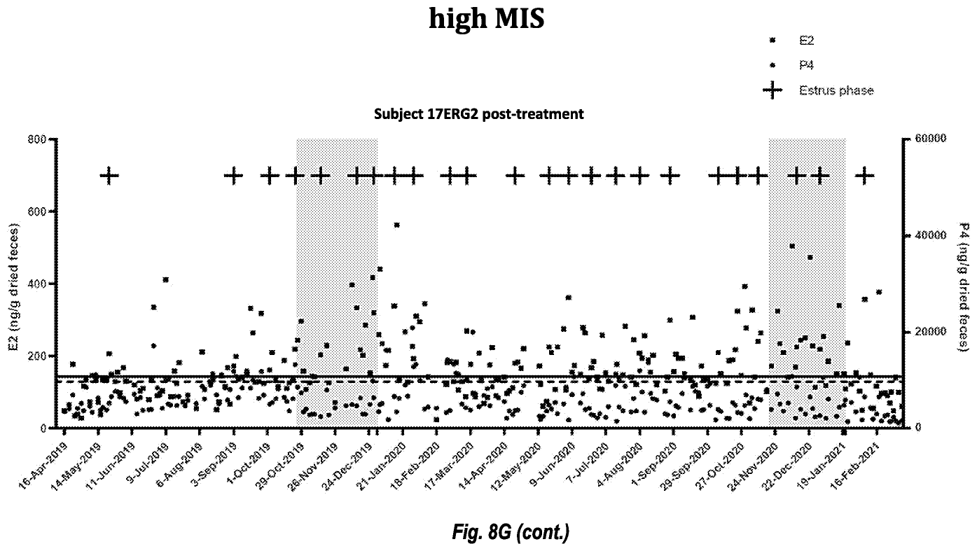

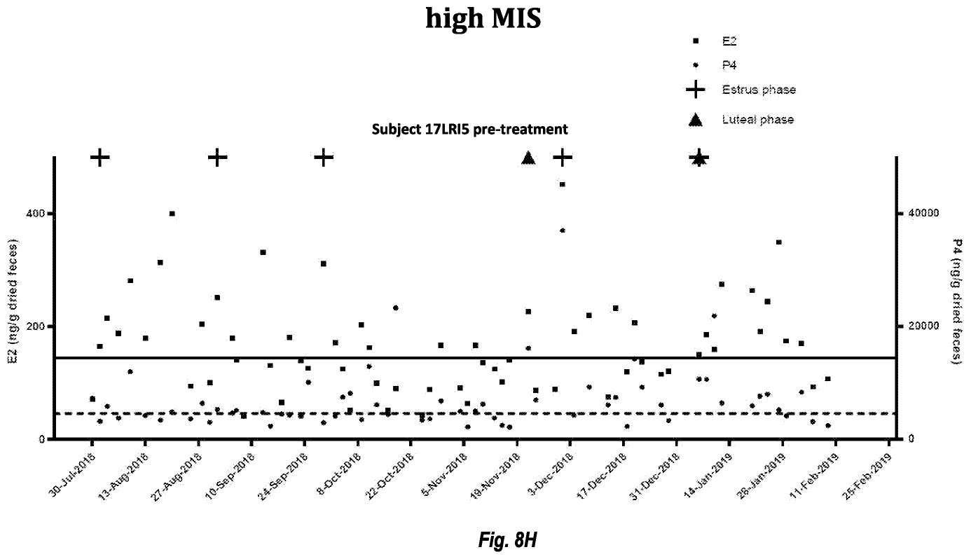

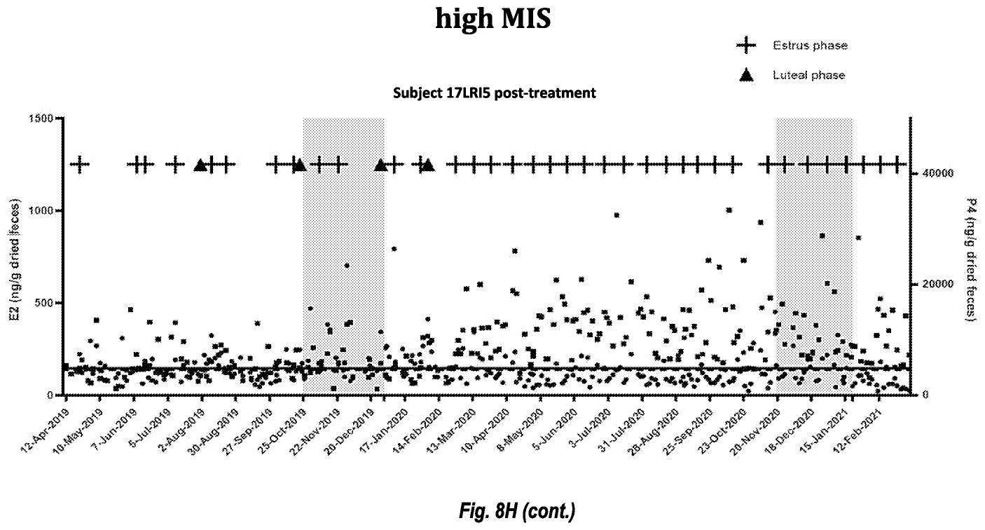

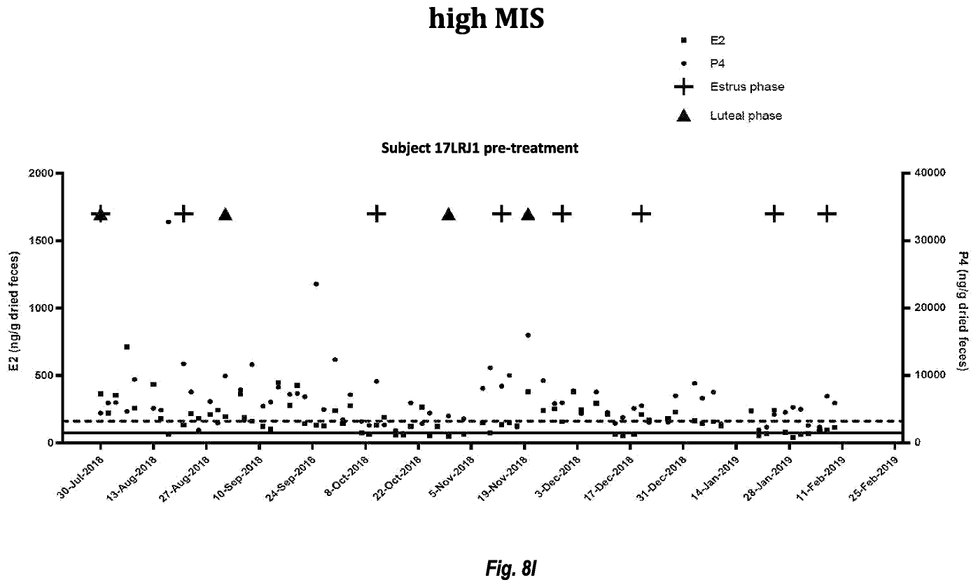

Fecal estradiol (E2) and progesterone (P4) were measured in thrice weekly fecal samples from 6 month prior to injection to the end of the study (2 years) (FIGS. 8A-8I and Table 5). Reproductive cyclicity was confirmed in all females prior to treatment. In mice, there is a delay between AAV9-fcMISv2 treatment and complete ovarian suppression of approximately 1 month, likely corresponding to the time required for already developing follicles to completely mature and be cleared from the ovary (Kano et al., 2019, 2017; Meinsohn et al., 2021). While the timing of ovarian suppression by MIS in cats is uncertain, a two-month delay between MIS treatment and effect was estimated. Therefore, mean fecal E2 and P4 metabolite concentrations were compared 6 months pre-treatment versus 22 months post-treatment, after a 2-month transition phase (FIG. 6F). A significant increase in E2 was observed in the controls during the post-treatment observation and breeding periods (P=0.0008). No differences were observed between time periods in the low or high dose AAV9-fcMISv2 groups. Mean P4 metabolite concentration was reduced in the high dose group following treatment (P=0.0029). The low dose group trended downward (P=0.0986) whereas the control group did not differ between time points.

TABLE 5
Fecal E2 and P4 profiles

| | E2 metabolites | | | P4 metabolites | | |
|---|---|---|---|---|---|---|
| | (ng/g dried feces) | | | (ng/g dried feces) | | |
| | Pre | Post | P- | Pre | Post | P- |
| Treatment | Tx | Tx | value | Tx | Tx | value |
| Control | 253.0 | 450.2 | 0.0008 | 11,291 | 12,509 | 0.5012 |
| | (30.6) | (32.0) | | (1827.9) | (1725.4) | |
| Low | 210.7 | 228.7 | 0.6327 | 11,187 | 8,447 | 0.0986 |
| | (26.6) | (25.5) | | (1588.1) | (1495.0) | |
| High | 179.0 | 247.5 | 0.2146 | 12,870 | 4,768 | 0.0029** |
| | (37.6) | (36.0) | | (2246.5) | (2115.0) | |

Data presented as mean (SEM),
**indicates P < 0.01.

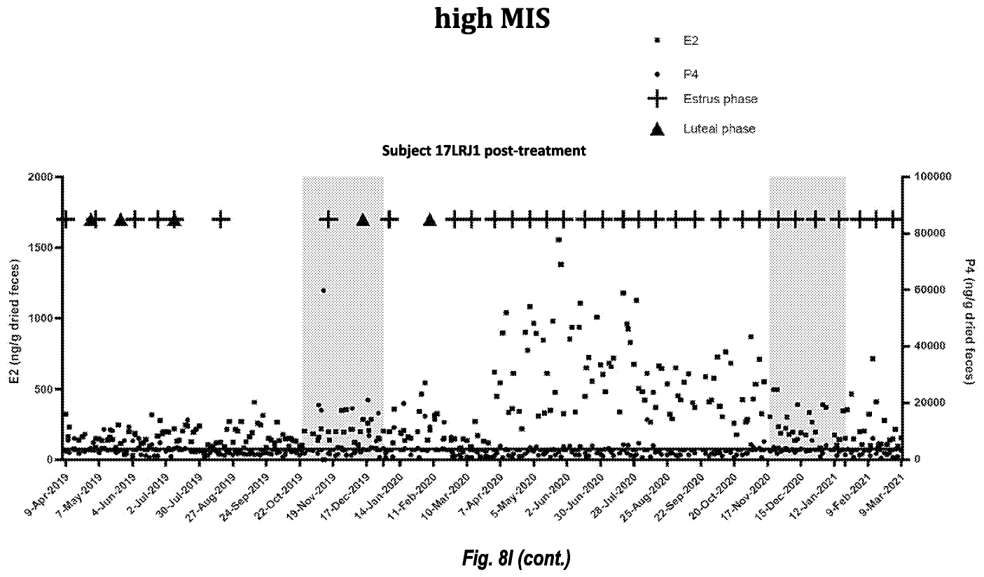

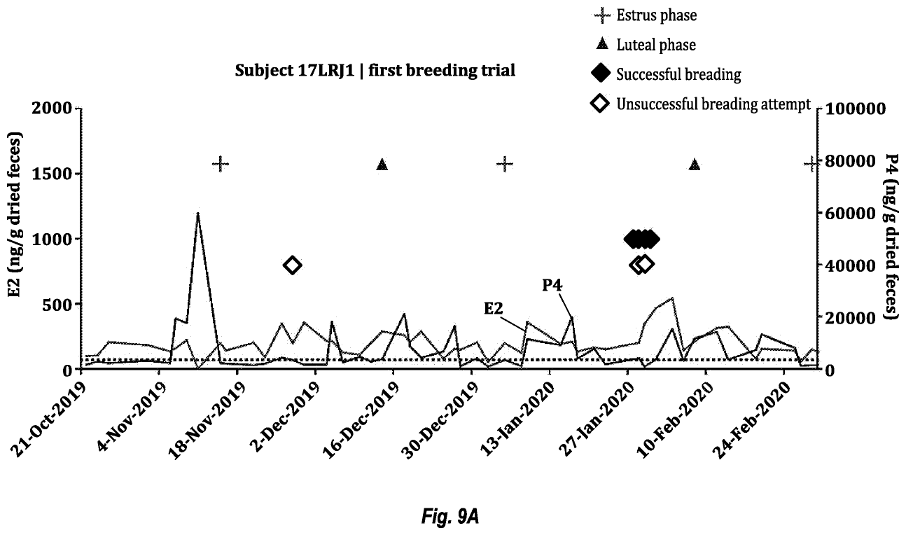

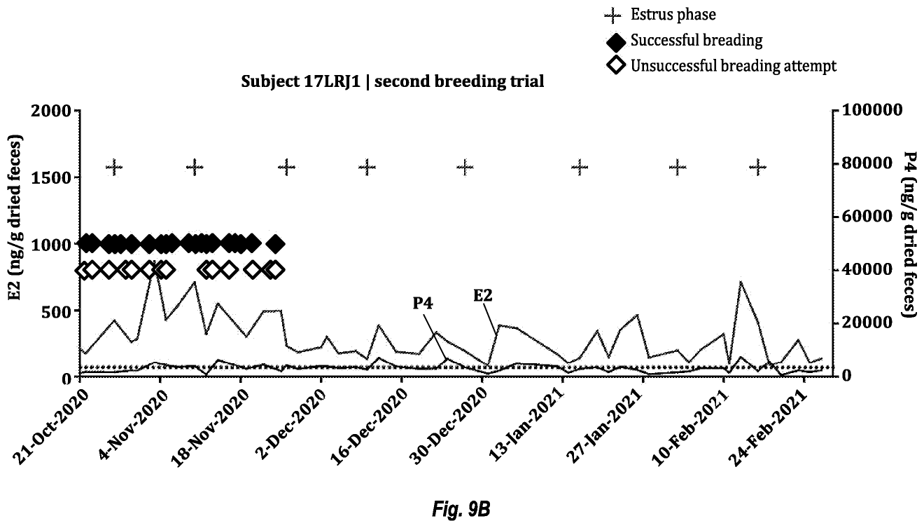

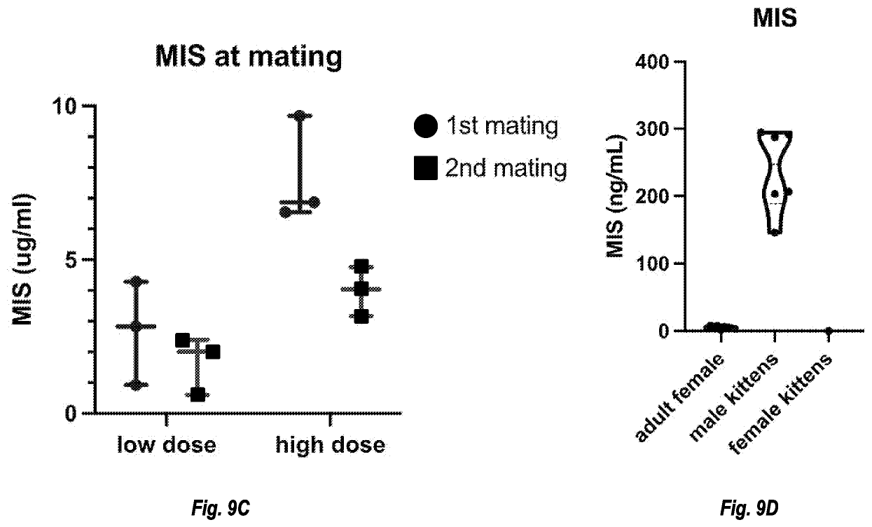

Fecal E2 and P4 levels were used to infer the timing of estrous and luteal phases (FIGS. 6F, 9A and 9B, Table 6). The frequency of estrous phases per month and luteal phases per six months were compared between 6 months pre-treatment and 24 months post-treatment (excluding the 2-month transition phase). No differences were observed in estrus phase frequency for any group (FIG. 6G). Luteal phase frequency was reduced in the high dose AAV9-fcMISv2 group (P=0.0263), but not in the low dose group or controls (FIG. 6G).

Average serum LH was calculated at two time periods: samples taken prior to MIS treatment and during the two-month transition period (Pre/Trans) and samples taken post-treatment (excluding breeding trial, pregnancy, and lactation periods, Post-Tx). Data from the two treatment groups were pooled and analysis revealed there were no differences in LH for the control cats (FIG. 6E). However, Post-Tx LH was significantly higher than Pre/Trans LH in MIS-treated cats (P=0.0001).

TABLE 6
Estimation of estrous and luteal phases

| | Estrus phases per month | | | Luteal phases per six months | | |
|---|---|---|---|---|---|---|
| | Pre | Post | P- | Pre | Post | P- |
| Treatment | Tx | Tx | value | Tx | Tx | value |
| Control | 1.3 (0.1) | 1.6 (0.1) | 0.4565 | 1.6 (0.6) | 1.6 (0.6) | 1.0000 |
| Low | 0.7 (0.2) | 0.7 (0.1) | 0.9999 | 2.4 (0.3) | 1.5 (0.5) | 0.4291 |
| High | 1.0 (0.1) | 1.3 (0.1) | 0.6833 | 2.8 (0.5) | 0.4 (0.4) | 0.0263* |

Data presented as mean (SEM),
*indicates p > 0.05.

### E. Repeat Breeding Extension

### Materials and Methods

In a repeat breeding study, a proven breeder male cat was transferred into the group-housing room containing the nine AAV-MIS study females (3 controls, 6 AAV-MIS treated). The male received previous controlled exposure (housed in a cat carrier) to the females within the room to facilitate integration. The male was housed with the females for 8 hours each day for 5 days each week for 4 consecutive months. Breeding activity was documented through a combination of direct observation and remote audio/video monitoring. Each female was assessed by abdominal palpation and ultrasound exam weekly to determine pregnancy status (i.e., presence and viability of fetuses). All females received prior operant conditioning to voluntarily accept these procedures with minimal restraint or disturbance. The pregnant females were reassessed via ultrasonography every three weeks to monitor fetal development and viability. Females remained in group housing until ˜Day 50 of pregnancy and then were transferred into the maternity room with individual caging for subsequent natural parturition (typically at ˜Day 63-65 post-breeding). Pregnant females were monitored in person each day by keepers and remotely via an internet-accessible video camera linkage through the expected time of parturition.

Two four-month breeding trials were initiated at the 8-month and 20-month timepoints after treatment with vectors (FIG. 6A), using a different proven-breeder male for each trial. The male was group-housed with the 9 females for 8 hours/day, 5 days/week, and continually video monitored to record all breeding interactions. Weekly transabdominal ultrasonography was performed to assess pregnancy status. Interactions were assessed by video review and scored as a successful breeding (defined by intromission and appropriate response from female) or breeding attempt (defined by male attempting to mount female or successful mount without intromission). The identity of each female for an interaction was determined by a member of the animal keeper staff.

In this example, a “breeding bout” is defined as a successive repetitive breeding behavior period. A “breeding bout” consists of a time period (a single day or a number of consecutive days) in which a queen successfully breeds with a male. A “breeding bout” typically represents the duration of one estrous phase in which the female is receptive to the male, although rarely there may be a prolonged estrous phase (i.e., beyond 8 days) or multiple overlapping estrous phases that results in a really long breeding bout. In Subject 17LRJ1, for example, an atypical breeding bout was observed consisting of about 125 confirmed breeding behaviors over 33 days.

For statistical analysis of breeding trials, the number of breeding females, total breeding bouts, number of luteal phases post-breeding, number of pregnant females, and total kittens produced were compared between treatments within each breeding trial using a Chi-square test of homogeneity, where the null hypothesis was a uniform distribution across treatments. Analyses were performed using SAS© Studio software (Release: 3.8, Enterprise Edition, SAS Institute Inc., Cary, NC, USA).

### Results

For both trials, all control females conceived following their first breeding bout (Tables 7 and 9). The controls gave birth to 2-4 healthy kittens in each litter. In contrast, no AAV9-MIS-treated females gave birth during either trial and no gestational sacs or fetuses were observed at weekly ultrasound exams.

TABLE 7
Breeding activity, ovulation, and pregnancy occurrence
in AAV9-fcMIS treated cats versus controls

| | Trial 1 | | | Trial 2 | | |
|---|---|---|---|---|---|---|
| | Control | Low | High | Control | Low | High |
| | n = 3 | n = 3 | n = 3 | n = 3 | n = 3 | n = 3 |
| Number of females that | 3 | 1 | 1 | 3 | 1 | 1 |
| allowed breeding | | | | | | |
| Total number of | 3 | 6 | 1 | 3 | 3 | 1 |
| breeding bouts | | | | | | |
| Number of luteal | 3 | 0 | 0 | 3 | 0 | 0 |
| phases that followed | | | | | | |
| a breeding bout* | | | | | | |
| Number of pregnant | 3 | 0 | 0 | 3 | 0 | 0 |
| females* | | | | | | |
| Total kittens produced* | 10 | 0 | 0 | 11 | 0 | 0 |

*Differed between controls and treated within each breeding trial (P < 0.05).

Two of the AAV9-MIS-treated cats did exhibit breeding activity during the trials (Tables 7 and 10). Subject 17LRJ1 (high dose AAV9-fcMISv2) allowed one breeding bout during each trial. Subject 17LRE4 (low dose AAV9-fcMISv2) allowed 6 breeding bouts during the first trial and 1 breeding bout during the second. No luteal phases were detected in fecal hormone analyses following any of the bouts (Table 7). No breeding behavior was observed from the other AAV9-MIS-treated females.

Using a chi-square test of homogeneity, no difference was seen between the number of females that allowed breeding or total breeding bouts. There were significant differences in number of luteal phases that followed a breeding bout (P=0.0498), number of females that became pregnant (P=0.0498), and total kittens produced (P<0.0001; Table 7).

To determine if the introduction of males had an effect on E2 levels during estrous, E2 peaks in fecal pellets were compared during the mating period to those during the pre-treatment period when females were not co-habiting with males. A significant increase of E2 peaks only was found in the control cats following introduction of the males (Table 8).

TABLE 8
Estradiol peaks during mating study.

| | Mean peak E2 (ng/g dried feces) | | |
|---|---|---|---|
| Treatment | Pre Tx | Post Tx | P-value |
| Control | 361.3 (36.2) | 590.2 (36.7) | <0.0001 |
| Low | 345.6 (36.6) | 335.5 (38.7) | 0.9968 |
| High | 227.5 (35.9) | 311.3 (36.7) | <0.0001 |

Data presented as mean (SEM)

Since no kittens were born from treated females, maternal-fetal transmission of MIS was not assessed. However, reference values in male kittens born from control females were established at 254 ng/ml±72 (FIG. 9D).

TABLE 9
First Breeding Trial

| | | | Description | | | Estrus | Luteal | |
|---|---|---|---|---|---|---|---|---|
| Treatment | Cat | Dates | of breeding | S | A | phase? | phase? | Comments |
| Control | 17EPV5 | Dec. 11, 2019- | Multiple successes | 12 | 2 | Yes | Yes | Resulted in pregnancy |
| | | Dec. 13, 2019 | and few attempts | | | | | |
| | 17LRS7 | Oct. 22, 2019- | Multiple successes | 20 | 5 | Yes | Yes | Resulted in pregnancy |
| | | Oct. 25, 2019 | and few attempts | | | | | |
| | 17EPT6 | Oct. 28, 2019- | Multiple successes | 18 | 4 | No | Yes | Resulted in pregnancy |
| | | Oct. 31, 2019 | and few attempts | | | | | |
| Low | 17LR06 | | | | | | | No breeding behavior observed |
| | 17LRE4 | Oct. 30, 2019- | Multiple successes | 8 | 6 | Yes | No | Luteal phase was ongoing |
| | | Nov. 1, 2019 | and few attempts | | | | | from Oct. 22, 2019 |
| | | Nov. 21, 2019- | Two successes | 2 | 0 | Yes | No | Luteal phase was ongoing |
| | | Nov. 22, 2019 | | | | | | from Oct. 22, 2019 |
| | | Dec. 16, 2019- | Multiple successes | 4 | 4 | No | No | Intermittent elevated P seen |
| | | Dec. 17, 2019 | and few attempts | | | | | |
| | | Jan. 2, 2020- | Multiple successes | 10 | 13 | No | No | Intermittent elevated P seen |
| | | Jan. 10, 2020 | and few attempts | | | | | |
| | | Jan. 23, 2020- | Mostly attempts with | 4 | 17 | Yes | No | Estrus observed at end of |
| | | Jan. 31, 2020 | some success at end | | | | | breeding bout |
| | | Feb. 7, 2020 | Mostly successes | 6 | 1 | Yes | No | Same estrus phase as last bout |
| | | | Total | 34 | 41 | | | |
| | 17LPY6 | Nov. 6, 2019 | No attempt at breeding | 0 | 0 | Yes | No | |
| | | | but male displayed | | | | | |
| | | | interest in female | | | | | |
| High | 17ERG2 | | | 0 | 0 | | | No breeding behavior observed |
| | 17LRJ1 | Nov. 28, 2019 | Single attempt | 0 | 1 | No | No | |
| | | Jan. 28, 2020- | Multiple successes | 12 | 5 | No | No | Intermittent high P4 before and |
| | | Jan. 31, 2020 | and attempts | | | | | after breeding was present |
| | | | Total | 12 | 6 | | | |
| | 17LRI5 | Jan. 2, 2020 | Multiple attempts, | 0 | 4 | No | No | |
| | | | no success | | | | | |

S: Successful breeding,
A: Attempted breeding

TABLE 10
Second Breeding Trial

| | | | Description | | | Estrus | Luteal | |
|---|---|---|---|---|---|---|---|---|
| Treatment | Cat | Dates | of breeding | S | A | phase? | phase? | Comments |
| Control | 17EPV5 | Oct. 22, 2020- | Multiple successes | 11 | 0 | Yes | Yes | Resulted in pregnancy |
| | | Oct. 27, 2020 | | | | | | |
| | 17LRS7 | Nov. 6, 2020- | Multiple successes | 44 | 0 | Yes | Yes | Resulted in pregnancy |
| | | Nov. 11, 2020 | | | | | | |
| | 17EPT6 | Nov. 23, 2020- | Multiple successes | 19 | 2 | Yes | Yes | Resulted in pregnancy |
| | | Jan. 26, 2020* | and few attempts | | | | | |
| Low | 17LR06 | | | 0 | 0 | | | No breeding behavior observed |
| | 17LRE4 | Nov. 5, 2020- | Multiple successes | 4 | 5 | Yes | No | Luteal phase was ongoing |
| | | Nov. 9, 2020 | and attempts | | | | | from Oct. 27, 2020 |
| | | Nov. 26, 2020- | Multiple successes | 11 | 8 | Yes | No | Luteal phase was ongoing |
| | | Dec. 2, 2020 | and attempts | | | | | from Oct. 27, 2020 |
| | | Dec. 7, 2021- | Mostly attempts | 1 | 4 | No | No | |
| | | Jan. 15, 2021 | | | | | | |
| | | | Total | 16 | 17 | | | |
| | 17LPY6 | | | 0 | 0 | | | No breeding behavior observed |
| High | 17ERG2 | | | 0 | 0 | | | No breeding behavior observed |
| | 17LRJ1 | Oct. 22, 2020- | Multiple successes | 125 | 26 | Yes (3) | No | Intermittent high P4 before and |
| | | Nov. 24, 2020 | and attempts | | | | | after breeding was present |
| | 17LRI5 | | | | | | | No breeding behavior observed |

S: Successful breeding
A: Attempted breeding
*Successful breeding also seen on Jan. 4, 2021 (1), Jan. 13, 2021 (1) and Jan. 14, 2021 (2). Estrus peak detected on Jan. 9, 2021 (but excluded from peak analysis because occurring during gestation).

### F. Discussion

Intraperitoneal administration of AAV9 to mice primarily transduces skeletal muscle and liver cells, which in turn act as in vivo bioreactors secreting MIS for systemic delivery to the ovaries (Kano et al., 2017). In the cats the MIS levels during the initial period of sampling followed an evolution of sharply decreasing concentration over time until a relative plateau was reached. This sharp drop may be due to turnover of shorter-lived transduced cells (e.g., liver cells), which may also be the source of viral genomes observed in the blood over the first 3 months, which was counterbalanced by an increased proportional secretion from long-lived muscle fibers that maintain long-term expression of the fcMISv2 transgene. It was hypothesized that muscle-tropic viral vectors may therefore be beneficial to ensure lifetime production of transgene at contraceptive levels.

In AAV9-fcMISv2-treated cats, a significant decrease was observed in P4 levels and luteal phases, which strongly suggests an absence of ovulation. However, a relatively modest reduction in inhibin B was observed while there were no differences in E2 levels. These data are consistent with the inhibition of maturation of preantral follicles previously demonstrated in mice treated with AAV9-MIS which caused a significant decrease in circulating inhibin B, inhibited cycling yet maintained levels of E2 at approximately half of baseline (Kano et al., 2017). Subsequent studies demonstrated a stall in preantral follicle maturation due to the inhibition of granulosa cell differentiation by MIS (Meinsohn et al., 2021), thus suggesting a pool of immature follicles, incapable of ovulation, is maintained in treated animals and continues to produce E2. This has important implications for the use of recombinant MIS or MISR2 agonists as contraceptives in humans since it implies that estrogen levels may not be suppressed by these therapies and, therefore, hormonal health could be maintained in the absence of cycling.

Although the timing of the onset of the contraceptive effect was not directly tested in this Example, contraception was observed at 8 months post-treatment. The hormonal profile of the AAV9-fcMISv2 treated cats indicates that most changes in reproductive hormones are stable by 3 months, suggesting a potential upper limit for the contraceptive effect of MIS. Similarly in mice, adult females treated with AAV9-LRMIS and paired with proven male breeders were fertile for the first 30 days, but remained infertile afterwards (Kano et al., 2019, 2017).

Interestingly, the control females demonstrated a significant rise in E2 during the post-treatment period (FIG. 6E). This time period included 8 months of cohabitation with an intact male. In sheep, the ram effect has been well described, whereas the sociosexual stimulation of introducing an intact male to seasonally anestrual females can induce an LH surge and ovulation (Martin et al., 1986). More recently, it has been shown that the LH surge results from increased E2 secretion (Fabre-Nys et al., 2015). The Vandenbergh effect, whereas the presence of a male surges the females' serum E2 and accelerates puberty, is best described in mice, but occurs in other species, including several other rodents as well as pigs and cattle (reviewed by (deCatanzaro, 2015)). A similar phenomenon has been documented in cats, in that the non-copulatory presence of a male can increase the rate of spontaneous ovulation (Gudermuth et al., 1997). Hence, the increase in E2 observed in control females may be attributed to a male proximity effect. Both treatment groups also showed a slight but non-significant increase in E2 (FIG. 6E), suggesting AAV9-fcMISv2-treatment may have diminished that response.

In female cats that have been previously ovariectomized, LH increased due to a lack of negative feedback from ovarian-derived E2 (Bateman et al., 2017; Concannon et al., 1989; Johnson and Gay, 1981; Rohlertz et al., 2012). Although E2 did not appear to be suppressed based on fecal hormone data, the rise of LH in MIS-treated cats (FIG. 6C) suggests some level of ovarian functional impairment. When estrus frequency was determined by assessing consecutive E2 elevations in fecal samples, no difference was observed in MIS-treated cats compared to controls (FIG. 6F). However, when estrus was defined behaviorally by the female permitting mounting and coitus, an effect of treatment can clearly be observed. All 3 control females mated repeatedly with both males, whereas 4 of the 6 MIS-treated females rebuffed every mating attempt by the breeder males during both breeding trials (Tables 7 and 10). Mate choice among females may have influenced breeding activity, but use of two different breeder males (of greatly different phenotypes and ages) and the consistent receptiveness of randomly assigned control females to both males negate that as a primary factor.

Because AAV9-fcMISv2 vectored contraception has been proposed as an alternative to ovariohysterectomy, the long-term health of retained reproductive organs is of key consideration. Cystic endometrial hyperplasia-pyometra complex is a clinically relevant and potentially life-threatening disease in intact female cats. Ovarian hormones contribute to the pathogenesis, with P4 playing a primary role. The disease is characterized by hyperplasia of the endometrium, cystic dilation of endometrial glands, uterine inflammation, and purulent discharge (Agudelo, 2005). The overall incidence is unknown. However, a study assessing the histopathology of 106 reproductive tracts from clinically healthy cats presenting for elective ovariohysterectomy detected cystic endometrial hyperplasia in 21 (˜20%) and pyometra in 2 (˜2%) (Binder et al., 2019).

The 6 treated females in this study received their AAV9-fcMISv2 injections approximately 3 years ago (March 2019). Physical exams and transabdominal ultrasounds performed every 3 months and bloodwork assessed every 6 months have found no evidence of cystic endometrial hyperplasia-pyometra or aberrations in systemic blood parameters in any female. Because these females are still intact, there are no uterine histopathological data available to evaluate. However, the 3 females treated with AAV9-fcMISv1 during the pilot study were spayed at 40 months post-treatment (at 9-10 years of age) and examined histologically. One female (Subject 11WBL25) produced minimal antibodies against the fcMISv1 protein (SEQ ID NO: 3) and retained a serum MIS protein level above the target level (0.50 μg/ml) (FIGS. 1F-1H). Her reproductive tract displayed normal uterine endometrium and a quiescent ovary containing only primordial follicles (FIG. 2). In contrast, histological analysis of the female with the highest anti-fcMISv1 antibody level and lowest serum MIS level (Subject 11WBL24) displayed multiple corpora lutea (indicative of spontaneous ovulation as this female was never housed with a male) and cystic endometrial hyperplasia (FIGS. 1F-1H and 2). These data suggest a possible protective effect of elevated MIS on uterine health in intact females by prevention of spontaneous ovulation.

One key aspect of that MIS-induced protection is likely the reduction in prolonged P4 exposure, as occurs during non-pregnant luteal phases. In the high dose group, both mean P4 levels and spontaneous ovulation rate were significantly reduced following treatment (FIGS. 6E-6F). Spontaneous ovulation may occur at high rates (>80%) in group-housed female cats with no direct contact with males (Gudermuth et al., 1997) and can contribute to cystic endometrial hyperplasia-pyometra complex through extended periods of P4 influence on the endometrium. In one case study, 45% of cats evaluated for inflammatory uterine disease or infertility had active corpora lutea due to spontaneous ovulation at the time of investigation (Lawler et al., 1991).

Similarly, high MIS levels appear to inhibit the occurrence of luteal phases induced by natural breeding as demonstrated in the two breeding trials (Tables 9-10).

Although serum MIS levels reflect a AAV9-fcMISv2 dose response relationship (with values ˜2× greater in the high dose than low dose cats) (FIG. 9C), inhibition of breeding-induced ovulation was observed in both dose groups. The one female in the low group that allowed breeding (Subject 17LRE4) had the lowest MIS level during each trial (0.92 and 0.61 μg/ml for trials 1 and 2, respectively). However, both MIS values remained above 0.50 μg/ml, which is double the 0.25 μg/ml threshold necessary for complete contraception in mice (Kano et al., 2017). In contrast, the one female in the high treatment group that allowed breeding (Subject 17LRJ1) had consistently elevated serum MIS (FIG. 9C). During the second breeding trial, her mean MIS level of 4.0 μg/ml coincided with an atypical 33-day breeding bout comprised of 125 documented copulations (Table 10). For comparison, the three control females had a mean (SE) of 24.7 (9.9) successful breedings in bouts lasting 4.3 (0.7) days during the same trial.

These findings document an overall decrease in fecal P4 concentrations, a reduction in spontaneous ovulation, and a complete inhibition of coitus-induced ovulation following AAV9-fcMISv2 treatment. It remains unknown if these reproductive alterations are caused by failure of ovarian follicles to complete maturation and ovulate in response to an LH surge or an impairment of the LH surge itself. Ovariohysterectomized cats (with elevated basal LH) still can generate an LH surge following gonadotropin releasing hormone (GnRH) treatment, suggesting that the pituitary remains responsive to breeding-induced hormonal stimuli. However, other research has shown that breeding early in estrus (Day 1) is less likely to induce an LH surge (˜43% ovulation rate), primarily due to lower concentrations of E2, as might occur with elevated MIS. Although timing of behavioral estrus can be variable relative to final stages of follicular development, the E2 threshold necessary for behavioral estrus appears to be lower than that produced by fully mature follicles (Shille et al., 1979). Thus, sustained E2 elevation is a key prerequisite to prime the hypothalamus and/or pituitary to respond to coitus with an ovulation reflex (Banks and Stabenfeldt, 1982), and may be impaired by high MIS concentration.

Taken together, these data support the use of AAV9-fcMISv2 as a durable contraceptive in adult cats. Continued long-term expression of the transgene needs to be investigated further but remains at or above the contraceptive range at the two-year timepoint in the low dose (5e12 vg/kg) group and (1e13 vg/kg) high dose group. Furthermore, no toxicities were noted in adult cats treated with MIS (Example 1.D. above). MIS concentrations demonstrated in gene therapy for contraception in adult cats (FIGS. 7E-7F) and MIS concentrations in male newborn kittens (0.5 μg/ml; FIG. 9D) fall within the same order of magnitude. This range of endogenous MIS concentrations have also been observed in other mammalian species, including baby boys (Lee et al., 1997).

Interestingly, others have shown that MIS may regulate pituitary gonadotropins, increasing the LH/FSH ratio (Cimino et al., 2016; Garrel et al., 2016; Tata et al., 2018). AAV9-fcMISv2 treated cats exhibited an initial decrease of LH, followed by an elevated baseline level (FIGS. 6C and 6E) consistent with hypergonadotropic hypogonadism. However, it is unclear how modulation of gonadotropins may contribute to contraception, given superphysiological MIS primarily inhibits follicles at the gonadotropin-independent primordial and early preantral stages in mice. Overall, the observed reduction in luteal phases, P4 levels, mating behavior and lack of conception, strongly support the hypothesis that MIS prevents ovulation by blocking the maturation of follicles to the ovulatory stage but maintains a pool of follicles capable of producing ovarian hormones. Furthermore, this hormonal data suggests that total ovarian suppression by MIS is not necessary for contraception given that animals in the low-dose group with milder suppression (according to E2, and inhibin B) and recorded estrus and mating events were still infertile.

### G. References

Adachi, K., Dissen, G. A., Lomniczi, A., Xie, Q., Ojeda, S. R., Nakai, H., 2020. Adeno-associated virus-binding antibodies detected in cats living in the Northeastern United States lack neutralizing activity. Sci Rep 10, 10073. https://doi.org/10.1038/s41598-020-66596-4
Agudelo, C. F., 2005. Cystic endometrial hyperplasia-pyometra complex in cats. A review. Vet Q 27, 173-182.
Banks, D. H., Stabenfeldt, G., 1982. Luteinizing hormone release in the cat in response to coitus on consecutive days of estrus. Biol Reprod 26, 603-611. https://doi.org/10.1095/biolreprod26.4.603
Bateman, H. L., Vansandt, L. M., Newsom, J., Swanson, W. F., 2017. Temporal changes in serum luteinizing hormone following ovariohysterectomy and gonadotropin-releasing hormone vaccination in domestic cats. Reprod Domest Anim 52 Suppl 2, 332-335. https://doi.org/10.1111/rda.12840
Behringer, R. R., Finegold, M. J., Cate, R. L., 1994. Müllerian-inhibiting substance function during mammalian sexual development. Cell 79, 415-425.
Binder, C., Aurich, C., Reifinger, M., Aurich, J., 2019. Spontaneous ovulation in cats-Uterine findings and correlations with animal weight and age. Anim Reprod Sci 209, 106167. https://doi.org/10.1016/j.anireprosci.2019.106167
Cate, R. L., Mattaliano, R. J., Hession, C., Tizard, R., Farber, N. M., Cheung, A., Ninfa, E. G., Frey, A. Z., Gash, D. J., Chow, E. P., 1986. Isolation of the bovine and human genes for Müllerian inhibiting substance and expression of the human gene in animal cells. Cell 45, 685-698.
Cimino, I., Casoni, F., Liu, X., Messina, A., Parkash, J., Jamin, S. P., Catteau-Jonard, S., Collier, F., Baroncini, M., Dewailly, D., Pigny, P., Prescott, M., Campbell, R., Herbison, A. E., Prevot, V., Giacobini, P., 2016. Novel role for anti-Müllerian hormone in the regulation of GnRH neuron excitability and hormone secretion. Nat Commun 7, 10055. https://doi.org/10.1038/ncomms10055
Concannon, P. W., Lein, D. H., Hodgson, B. G., 1989. Self-limiting reflex luteinizing hormone release and sexual behavior during extended periods of unrestricted copulatory activity in estrous domestic cats. Biol Reprod 40, 1179-1187. https://doi.org/10.1095/biolreprod40.6.1179
deCatanzaro, D., 2015. Sex steroids as pheromones in mammals: the exceptional role of estradiol. Horm Behav 68, 103-116. https://doi.org/10.1016/j.yhbeh.2014.08.003
di Clemente, N., Wilson, C., Faure, E., Boussin, L., Carmillo, P., Tizard, R., Picard, J. Y., Vigier, B., Josso, N., Cate, R., 1994. Cloning, expression, and alternative splicing of the receptor for anti-Müllerian hormone. Mol. Endocrinol. 8, 1006-1020. https://doi.org/10.1210/mend.8.8.7997230
Fabre-Nys, C., Chanvallon, A., Debus, N., Frangois, D., Bouvier, F., Dupont, J., Lardic, L., Lomet, D., Ramé, C., Scaramuzzi, R. J., 2015. Plasma and ovarian oestradiol and the variability in the LH surge induced in ewes by the ram effect. Reproduction 149, 511-521. https://doi.org/10.1530/REP-14-0587
Garrel, G., Racine, C., L'Hôte, D., Denoyelle, C., Guigon, C. J., di Clemente, N., Cohen-Tannoudji, J., 2016. Anti-Müllerian hormone: a new actor of sexual dimorphism in pituitary gonadotrope activity before puberty. Sci Rep 6, 23790. https://doi.org/10.1038/srep23790
Gudermuth, D. F., Newton, L., Daels, P., Concannon, P., 1997. Incidence of spontaneous ovulation in young, group-housed cats based on serum and faecal concentrations of progesterone. J Reprod Fertil Suppl 51, 177-184.
Johnson, L. M., Gay, V. L., 1981. Luteinizing hormone in the cat. I. Tonic secretion. Endocrinology 109, 240-246. https://doi.org/10.1210/endo-109-1-240
Kano, M., Hsu, J. Y., Saatcioglu, H. D., Nagykery, N., Zhang, L., Sabatini, M. E., Donahoe, P. K., Pépin, D., 2019. Neoadjuvant treatment with Müllerian Inhibiting Substance synchronizes follicles and enhances superovulation yield. J. Endocr. Soc. https://doi.org/10.1210/js.2019-00190
Kano, M., Sosulski, A. E., Zhang, L., Saatcioglu, H. D., Wang, D., Nagykery, N., Sabatini, M. E., Gao, G., Donahoe, P. K., Pépin, D., 2017. AMH/MIS as a contraceptive that protects the ovarian reserve during chemotherapy. Proc. Natl. Acad. Sci. U.S.A. 114, E1688-E1697. https://doi.org/10.1073/pnas.1620729114
Lawler, D. F., Evans, R. H., Reimers, T. J., Colby, E. D., Monti, K. L., 1991. Histopathologic features, environmental factors, and serum estrogen, progesterone, and prolactin values associated with ovarian phase and inflammatory uterine disease in cats. Am J Vet Res 52, 1747-1753.
Lawler, D. F., Johnston, S. D., Hegstad, R. L., Keltner, D. G., Owens, S. F., 1993. Ovulation without cervical stimulation in domestic cats. J Reprod Fertil Suppl 47, 57-61.
Lee, M. M., Donahoe, P. K., Silverman, B. L., Hasegawa, T., Hasegawa, Y., Gustafson, M. L., Chang, Y. C., MacLaughlin, D. T., 1997. Measurements of serum mullerian inhibiting substance in the evaluation of children with nonpalpable gonads. N. Engl. J. Med. 336, 1480-1486. https://doi.org/10.1056/NEJM199705223362102
Li, P., Boenzli, E., Hofmann-Lehmann, R., Helfer-Hungerbuehler, A. K., 2019. Pre-existing antibodies to candidate gene therapy vectors (adeno-associated vector serotypes) in domestic cats. PLoS One 14, e0212811. https://doi.org/10.1371/journal.pone.0212811
Martin, G., Oldham, C., Cognié, Y., Pearce, D. T., 1986. The physiological responses of anovulatory ewes to the introduction of rams—a review. https://doi.org/10.1016/0301-6226(86)90031-X
Meinsohn, M.-C., Saatcioglu, H. D., Wei, L., Li, Y., Horn, H., Chauvin, M., Kano, M., Nguyen, N. M. P., Nagykery, N., Kashiwagi, A., Samore, W. R., Wang, D., Oliva, E., Gao, G., Morris, M. E., Donahoe, P. K., Pépin, D., 2021. Single-cell sequencing reveals suppressive transcriptional programs regulated by MIS/AMH in neonatal ovaries. Proc Natl Acad Sci USA 118, e2100920118. https://doi.org/10.1073/pnas.2100920118
Paape, S. R., Shille, V. M., Seto, H., Stabenfeldt, G. H., 1975. Luteal activity in the pseudopregnant cat. Biol Reprod 13, 470-474. https://doi.org/10.1095/biolreprod13.4.470
Rohlertz, M., Ström Holst, B., Axner, E., 2012. Comparison of the GnRH-stimulation test and a semiquantitative quick test for LH to diagnose presence of ovaries in the female domestic cat. Theriogenology 78, 1901-1906. https://doi.org/10.1016/j.theriogenology.2012.06.027
Schmidt, P. M., Chakraborty, P. K., Wildt, D. E., 1983. Ovarian activity, circulating hormones and sexual behavior in the cat. II. Relationships during pregnancy, parturition, lactation and the postpartum estrus. Biol Reprod 28, 657-671. https://doi.org/10.1095/biolreprod28.3.657
Shille, V. M., Lundström, K. E., Stabenfeldt, G. H., 1979. Follicular function in the domestic cat as determined by estradiol-17 beta concentrations in plasma: relation to estrous behavior and cornification of exfoliated vaginal epithelium. Biol Reprod 21, 953-963. https://doi.org/10.1095/biolreprod2l.4.953
Takahashi, M., Hayashi, M., Manganaro, T. F., Donahoe, P. K., 1986a. The ontogeny of mullerian inhibiting substance in granulosa cells of the bovine ovarian follicle. Biol. Reprod. 35, 447-453.
Takahashi, M., Hayashi, M., Manganaro, T. F., Donahoe, P. K., 1986b. The ontogeny of mullerian inhibiting substance in granulosa cells of the bovine ovarian follicle. Biol. Reprod. 35, 447-453. https://doi.org/10.1095/biolreprod35.2.447
Tata, B., Mimouni, N. E. H., Barbotin, A.-L., Malone, S. A., Loyens, A., Pigny, P., Dewailly, D., Catteau-Jonard, S., Sundstrom-Poromaa, I., Piltonen, T. T., Dal Bello, F., Medana, C., Prevot, V., Clasadonte, J., Giacobini, P., 2018. Elevated prenatal anti-Müllerian hormone reprograms the fetus and induces polycystic ovary syndrome in adulthood. Nat. Med. 24, 834-846. https://doi.org/10.1038/s41591-018-0035-5
Teixeira, J., He, W. W., Shah, P. C., Morikawa, N., Lee, M. M., Catlin, E. A., Hudson, P. L., Wing, J., Maclaughlin, D. T., Donahoe, P. K., 1996. Developmental expression of a candidate mullerian inhibiting substance type II receptor. Endocrinology 137, 160-165. https://doi.org/10.1210/endo.137.1.8536608
Verhage, H. G., Beamer, N. B., Brenner, R. M., 1976. Plasma levels of estradiol and progesterone in the cat during polyestrus, pregnancy and pseudopregnancy. Biol Reprod 14, 579-585. https://doi.org/10.1095/biolreprod14.5.579
Vigier, B., Picard, J. Y., Tran, D., Legeai, L., Josso, N., 1984a. Production of anti-Müllerian hormone: another homology between Sertoli and granulosa cells. Endocrinology 114, 1315-1320. https://doi.org/10.1210/endo-114-4-1315
Vigier, B., Picard, J. Y., Tran, D., Legeai, L., Josso, N., 1984b. Production of anti-Müllerian hormone: another homology between Sertoli and granulosa cells. Endocrinology 114, 1315-1320. https://doi.org/10.1210/endo-114-4-1315
Weenen, C., Laven, J. S. E., Von Bergh, A. R. M., Cranfield, M., Groome, N. P., Visser, J. A., Kramer, P., Fauser, B. C. J. M., Themmen, A. P. N., 2004. Anti-Müllerian hormone expression pattern in the human ovary: potential implications for initial and cyclic follicle recruitment. Mol. Hum. Reprod. 10, 77-83. https://doi.org/10.1093/molehr/gah015
Wildt, D. E., Chan, S. Y., Seager, S. W., Chakraborty, P. K., 1981. Ovarian activity, circulating hormones, and sexual behavior in the cat. I. Relationships during the coitus-induced luteal phase and the estrous period without mating. Biol Reprod 25, 15-28. https://doi.org/10.1095/biolreprod25.1.15

### Example 2—Vector Design for Delivery of MIS

This Example describe efforts in designing a vector for optimal expression of MIS in cats and dogs. The fcMISv2 protein (SEQ ID NO: 1) and clMIS protein (SEQ ID NO: 2) were modified as follows: (1) the leader sequence was replaced with the human albumin leader sequence, and (2) a glutamine (Q) in the MIS cleavage site was replaced with an arginine (R). Such leader and cleavage site (“LR”) modified fcMISv2 and LR modified clMIS transgenes were engineered and cloned into both pcDNA3.1 and pAAV vectors. The protein sequence for the LR-fcMISv2 transgene is SEQ ID NO: 14 and the protein sequence for the LR-clMIS transgene is SEQ ID NO: 15.

### A. The Effect of LR Vector Modifications in CHO Cells and COS7 Cells

### Materials and Methods

Materials and methods relating to CHO cells are as described in Example 1.A. above.

COS7 cells (African green monkey kidney cell line; American Type Culture Collection, Manassas, VA) were cultured in DMEM supplemented with 10% fetal bovine serum (FBS), 2 mM L-glutamine, 100 U/ml penicillin, and 100 μg/ml streptomycin in a humidified 5% CO₂ incubator at 37° C. COS7 cells were plated in 6 well culture dishes (Corning Life Sciences) in DMEM with 1% female fetal bovine serum (FFBS) (to reduce bovine MIS) and transfected with pcDNA3.1 expression plasmids containing fcMISv2, LR-fcMISv2, clMIS, or LR-clMIS using Fugene6 (Promega) (mass/volume ratio of 1:6) according to manufacturer's instructions. The transfection efficiency was confirmed to be greater than 80% by transfecting an identical plate with a GFP expression plasmid (PCDNA3-eGFP-N1). Conditioned media was collected 72 h later and used for western blot analysis.

CHO cells or COS7 cells were transfected with pcDNA3.1 or pAAV vectors containing fcMISv2 or clMIS transgenes. Transient and stable CHO clones were used to compare transcript level, protein level, and cleavage ratio of fcMISv2 (SEQ ID NO: 1), LR-fcMISv2 (SEQ ID NO: 14), cMIS (SEQ ID NO: 2), and LR-clMIS (SEQ ID NO: 15). Materials and methods related to cells, transfection, and Western blots were as described in Examples 1.A. above. Materials and methods related to ELISA were as described in Example 1.D. above.

### Results

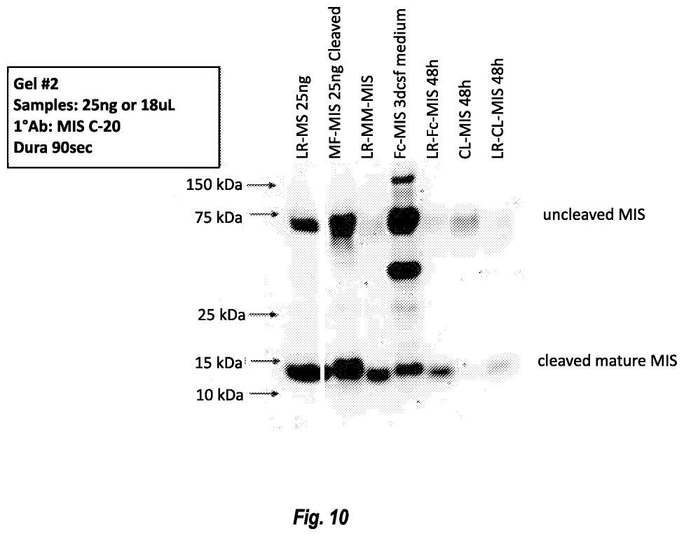

In transiently transfected CHO cells, CHO media conditioned for 48 h with LR-fcMISv2 had very high levels of cleavage with a strong cleaved mature MIS band (FIG. 10; LR-Fc-MIS 48 h) compared to media from CHO clones producing fcMISv2 (FIG. 10; Fc-MIS 3dcsf medium). This cleavage ratio can be compared across controls present on the western blot including purified human recombinant protein (FIG. 10; LR-MIS 25 ng), Flag-tagged mouse MIS or F-mmMIS (FIG. 10; MF-MIS 25 ng cleaved), LR-mmMIS conditioned media (FIG. 10; LR-MM-MIS), and fcMISv2 producing CHO clone serum free media prior to purification (FIG. 10; Fc-MIS 3dcsf medium), and CHO cells media conditioned for 48 h following transient transfection with the pCDNA3.1 plasmid encoding pCDNA-LR-fcMISv2 (FIG. 10; LR-Fc-MIS 48 h), pCDNA-clMIS (FIG. 10; CL-MIS 48 h), and pCDNA-LR-clMIS (FIG. 10; LR-CL-MIS 48 h). This recapitulates effects observed in human and murine transgene shown here as controls. Similarly, clMIS was mostly intact while LR-clMIS had almost complete cleavage. These data support the hypothesis (1) that LR modifications facilitate protein cleavage and activation across species, and that (2) unmodified transgenes, particularly the dog clMIS, are poorly cleaved by CHO cells.

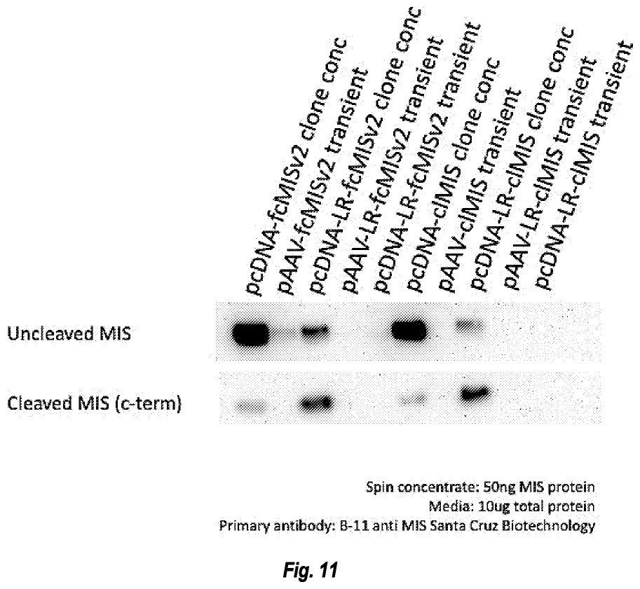

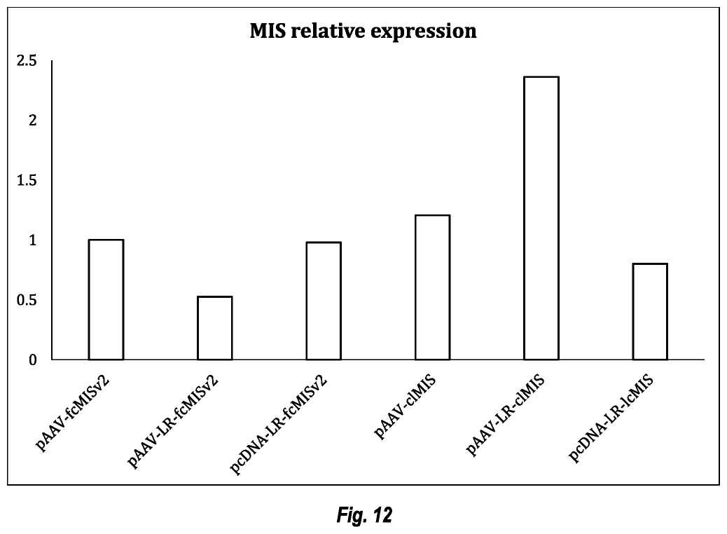

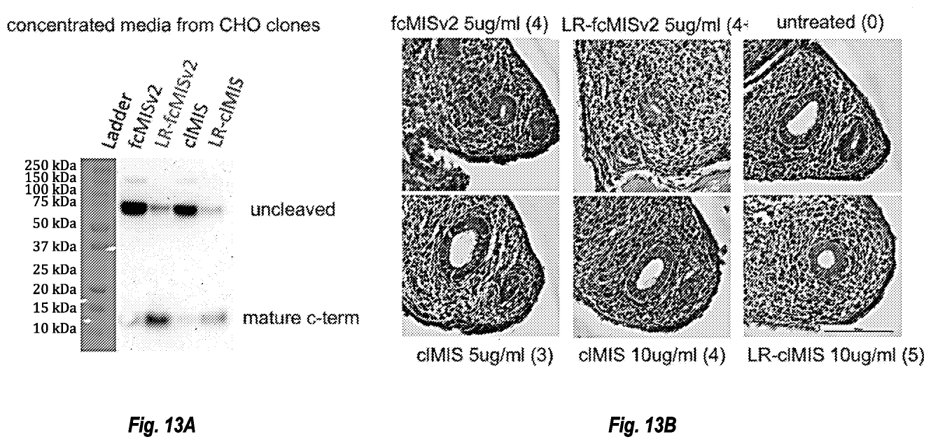

To ensure the variable cleavage ratios were not an artifact of CHO cells or the vector (pcDNA3.1 versus pAAV-CB6-PI; FIG. 10), the experiment was repeated using COS7 cells (FIG. 13). Again, in COS7 cells, strong expression of MIS from pcDNA based vectors was observed (FIG. 13). Also, cleavage of MIS from LR-based constructs were compared to non-LR variants, and enhanced cleavage was observed in the LR-based constructs (FIG. 11). These data strongly support the use of LR modifications to enhance activating cleavage. The cleavage effect observed appears to be fully post-translational as the various constructs were transcribed to relatively similar levels when compared by RT-qPCR (FIG. 12).

Finally, recombinant protein was evaluated in the rat urogenital ridge bioassay to compare biological activity. Using stably transfected CHO clones, the clones were screened for MIS protein expression with ELISA. Clones that demonstrated high protein expression were used in production cultures. Media from production cultures were collected and concentrated to a total protein concentration of 5 μg/ml. Concentrations were adjusted based on ELISA values. Because ELISA may, in some instances, underestimate the total amount of proteins in non-LR variants due to a possible detection bias (discussed in Example 2.E below), the observed increase in potency, which is achieved by the LR modifications and increasing cleavage, may be underestimated as well. The data demonstrate that stable high expressing CHO clones recapitulated enhanced cleavage of LR variants of fcMISv2 and clMIS (FIG. 13A). This translated to increased activity in the rat urogenital ridge assay, where regression of the Müllerian duct is scored from 0 to 5 (FIG. 13B). Overall, these data demonstrate that all the transgenes discussed in this Example were biologically active.

### B. Capsid Serotypes and Transgene Modifications in Mice

A benchmark for vector activity was established using AAV9-fcMISv2 as the reference vector. The benchmark allows one skilled in the art to make comparisons across myriad vectors, and to extrapolate results to those observed in cat with the same viral batch.

### Materials and Methods

Materials and methods related to CHO cells and experiments in mice were as described in Example 1.A. above. Materials and methods related follicle counts in mice were as described in Example 1.C. above. In this Example, vectors were administered by injection to mice and mouse experiments were carried out, generally, as described in Example 1.A. Mouse ovaries were recovered at 30 days after administration. Full sectioning and total follicle counts were carried out on those ovarian sections with at least 2 independent blinded observers.

### Results

The initial two AAV9-fcMISv2 concentrations administered to the mice were 5e12 vg/kg and 1e13 vg/kg, were the same as the concentrations used in cats. At these concentrations, folliculogenesis was suppressed in a dose-independent manner, possibly due to a saturating effect at high doses of MIS (FIG. 3G).

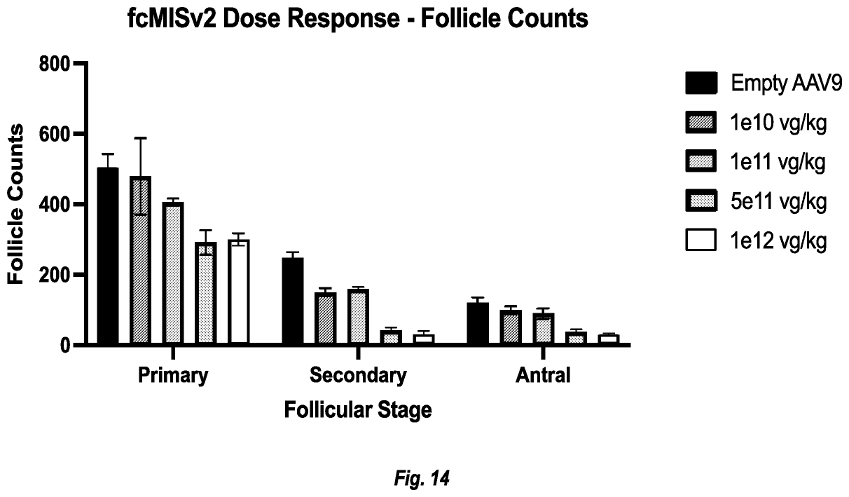

In order to compare activity across capsids and transgenes, a non-saturating dose of vector was used in the next round of experiments. A dose-response experiment was carried out with AAV9-fcMISv2 so that improvements to the suppressive effect of the vectors could be quantified. The AAV9-fcMISv2 vector was administered at 1E10, 1E11, 5e11, and 1e12 vg/kg. A dose-response effect was observed in the suppression of growing follicles (FIG. 14), with 1e11 vg/kg as a potential dose with partial but strong suppression. This 1e11 vg/kg dose may be used in future virus tests for translation into high MIS concentrations in the blood.

### C. AAV9-LR-c1MIS Activity in Mice

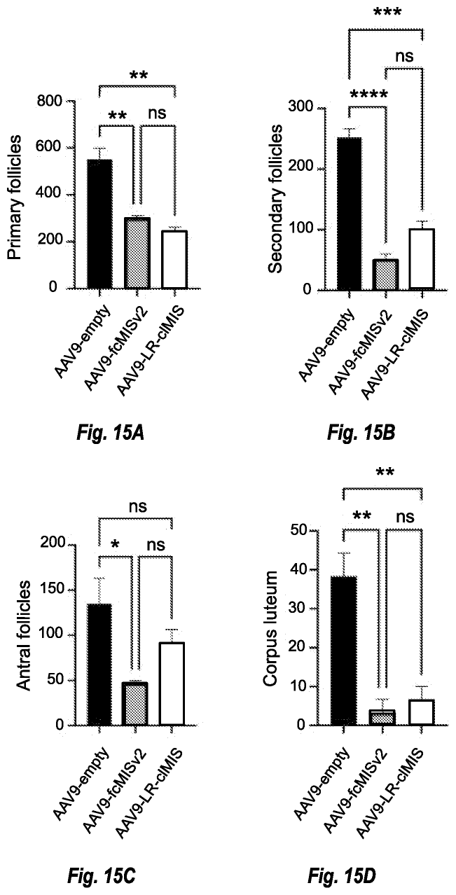

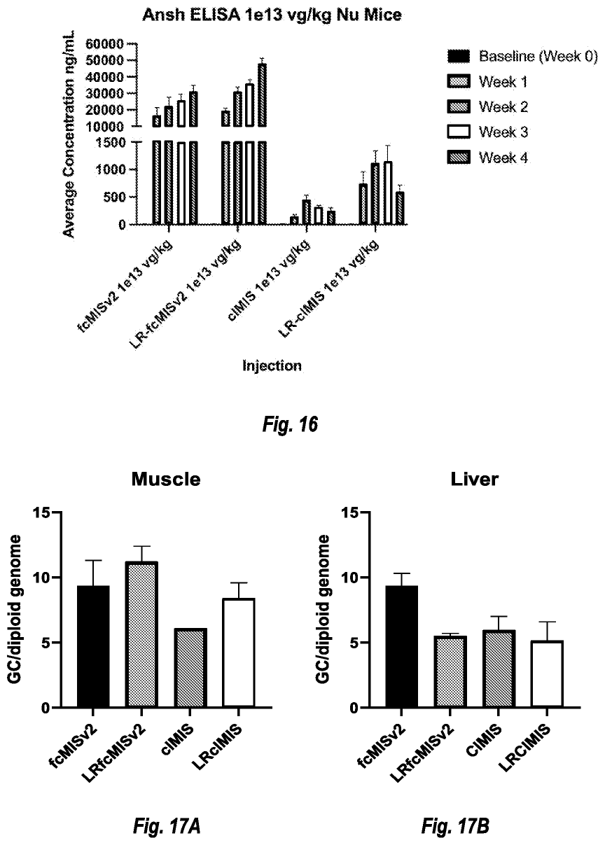

In anticipation of treating dogs, AAV9-clMIS or AAV9-LR-clMIS were tested for activity in mice. Materials and methods for this Example were as described in Examples 2.A. and 2.B. above. Both vectors were capable of ovarian suppression at the high dose (1e13 vg/kg) in mice. Despite lower expression detected in the blood (FIG. 16; Example 2.D. below), LR-clMIS demonstrated a strong suppressive effect at this dose which was comparable to that of AAV9-fcMISv2 (FIGS. 15A-15D). 1e13 vg/kg may be used as a high vector dose in dogs to possibly provide sufficiently high levels of MIS to saturate the suppressive effect.

### D. Cat MIS and Dog MIS Levels in Mice

### Materials and Methods

Materials and methods related to mouse experiments were as described in Example 2.B. above. The ELISA method was carried out as described in Example 1.D. above with the following exception—the Ansh Labs AMH ELISA kit (Ansh Labs catalog number AL-105) was used instead of the Beckman AMH Gen II ELISA kit.

### Results

The AAV9-clMIS and AAV9-LR-clMIS vectors at 5e12 vg/kg gave MIS concentrations that were lower than expected, i.e., in the 100-200 ng/ml range. The experiment was repeated using a higher vector concentration of 1e13 vg/kg. Again, for AAV9-clMIS, the MIS levels in mice were lower than expected (FIG. 16). However, for AAV9-LR-clMIS, the concentration of MIS was about twice as high as the unmodified AAV9-clMIS. According to the data, translational and post-translational processing of the dog transgene appears to be less efficient than processing of the cat transgene. However, the processing of MIS was improved with the introduction of the LR modification, which brings the circulating MIS within the presumed contraceptive range in cats and dogs.

Further, because the Ansh Labs ELISA could detect both cat and dog MIS, and the results were generally comparable to the Beckman ELISA (described above at Example 1.D. above), the Ansh Labs ELISA was used in subsequent analyses of MIS levels in the blood.

### E. Viral Infectivity Across Tissue Types

### Materials and Methods

Materials and methods related to cells, mice, transfections, and western blots were as described in Example 2.A. above.

### Results

To ensure that variable detection of MIS in the serum across the various vectors was not due to differences in viral batch quality (which could affect viral infectivity), the presence of vector DNA in different tissues were compared. Tissues from mouse quadriceps (“Muscle” in FIG. 17A) and liver were analyzed at 30 days following treatment with vectors expressing fcMISv2 (SEQ ID NO: 1), LR-fcMISv2 (SEQ ID NO: 14), cMIS (SEQ ID NO: 2), and LR-clMIS (SEQ ID NO: 15). In general, equivalent amounts of vector DNA were present in the tissues for all vectors. Although an excess of AAV9-fcMISv2 vector DNA was found in the liver compared to the other vectors (FIGS. 17A-17B), the amount of DNA is on an order of magnitude that is too small to account for the differences observed in circulating MIS as measured by ELISA. It was therefore noted that the quality of vector and/or virus preparations may influence MIS levels in an animal.

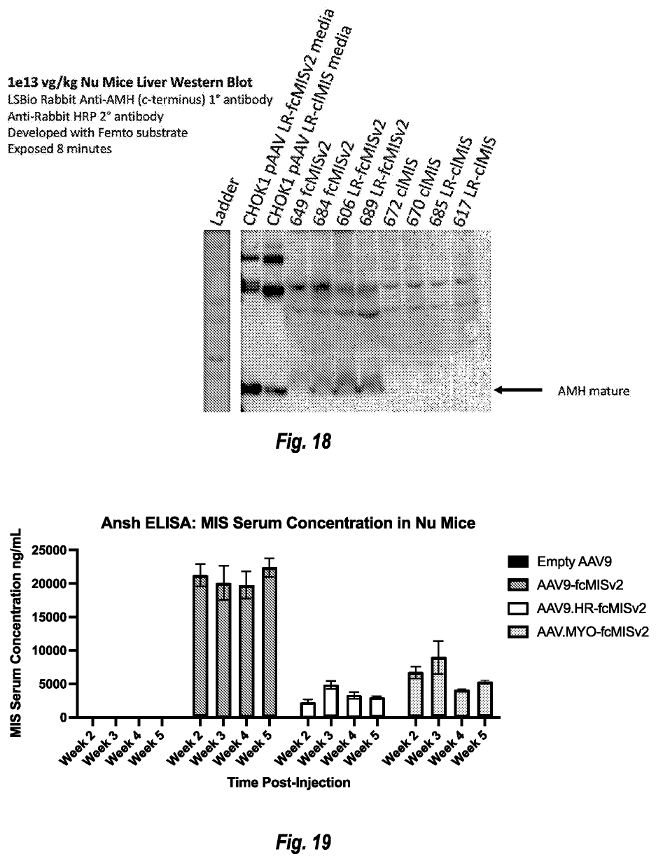

the secretion and cleavage of MIS in target tissues (liver and muscle tissues) from mice treated with AAV9-fcMISv2, AAV9-LR-fcMISv2, AAV9-clMIS, or AAV9-LR-clMIS was evaluated. The two vector concentrations used were 5e12 vg/kg and 1e13 vg/kg. Several antibodies were evaluated for their compatibility with murine lysates in western blots. One antibody, the LSBio rabbit anti-AMH, was able to reliably detect MIS in western blots but only in conditions with high concentrations of vector in tissues, i.e., the liver, where AAV9-fcMISv2 or AAV9-LR-fcMISv2 was present at high doses. The western blot is consistent with both fcMISv2 (SEQ ID NO: 1) and LR-fcMISv2 (SEQ ID NO: 14) being produced at a higher level than clMIS and LR-clMIS in the liver. Furthermore, in the liver, cleavage of the LR-fcMISv2 variant (SEQ ID NO: 14) was greater than cleavage of fcMISv2 (SEQ ID NO: 1) (FIG. 18).

The cleavage ratio of cat MIS and dog MIS may be quantified in the blood by ELISA. Cleavage ratio analysis may provide information regarding the benefits of LR modifications in vivo, and the ability of muscle cells to efficiently cleave LR-modified transgenes, which may be of particular importance in muscle tropic vectors described herein.

### F. Evaluation of Muscle Tropic Vectors

This example describes a study to determine if infection of a cell type with lower cell turnover rate promotes long-term production of the MIS transgenes. For example, muscle cells, in general, have a lower turnover rate than liver cells.

### Materials and Methods

AAV.MYO and AAV9.HR muscle-tropic vectors were used to deliver fcMISv2. The vectors were administered to nude mice 5e12 vg/kg by i.p. injection. Materials and methods related to mouse experiments were as described in Example 1.A.

### Results

Both AAV.MYO-fcMISv2 and AAV9.HR-fcMISv2 muscle-tropic vectors produced lower serum MIS levels than the AAV9-fcMISv2 (FIG. 19).

### Example 3—Effectiveness of AAV-Wt Feline MIS Treatment to Prevent Kittens from Entering Puberty, and to Provide Long-Term Sterility

### G. Preventing Puberty in Cats (Kittens)

A number of animal shelters surgically spay or neuter kittens as early as 8 weeks of age, prior to adoption, and recommendations suggest that kittens should be surgically spayed or neutered before they reach approximately 5 months of age. However, the long-term developmental effects of increasing MIS levels in prepubescent kittens (and puppies) are unknown. Nonetheless, the ability of MIS treatment via gene delivery to prevent puberty in cats (kittens) as well as any other associated developmental effects were assessed. Furthermore, parameters for using AAV-wt feline MIS when given as a single injection for long term reduction of fertility in kittens and/or for preventing kittens from entering puberty were assessed. By way of example, this study also serves as a non-human animal model to assess MIS treatment via gene delivery or delivery of a MIS protein for the delay of puberty in human subjects.

### Materials and methods

The kittens and virus preparations used in this Example were generated in the breeding study as described in Examples 1.D. and 1.E. above.

Timing for AAV-wt feline MIS (AAV9-fcMISv2) injection of kittens was dependent on the timing of parturition and weaning. Healthy kittens were weaned by 8 weeks of age. At approximately 3 months of age (e.g., 10-12 weeks of age), the healthy kittens (e.g., 9 females, 3 males) were randomized into a control and a treated group. The kittens were injected intramuscularly into caudal thigh muscles with the AAV9-fcMISv2 or empty vector control as follows: (1) High dose AAV9-fcMISv2 (1e13 vg/kg, e.g., 3 females and 1 male); (2) Low dose AAV9-fcMISv2 (5e12 vg/kg, e.g., 4 females and 1 male); and (3) Control empty vector (5e12 vp/kg, e.g., 2 female and 1 male). Table 11 provides a timetable for sampling and monitoring of the kittens.

TABLE 11
Kitten monitoring timetable

| | | | d | d | d | d | d | d | d | d | d | d | m | m | m | m | m | m | m | m | m |
|---|---|---|---|---|---|---|---|---|---|---|---|---|---|---|---|---|---|---|---|---|---|
| time | birth | pre | 0 | 1 | 2 | 3 | 4 | 5 | 6 | 7 | 14 | 21 | 1 | 2 | 3 | 4 | 5 | 6 | 7 | 8 | 9 |
| Blood | x | | x | | | | | | | x | x | x | x | x | x | x | x | x | x | x | x |
| MIS | | | | | | | | | | | | | | | | | | | | | |
| Blood a- | x | | x | | | | | | | x | x | x | x | x | x | x | x | x | x | x | x |
| MIS Ab | | | | | | | | | | | | | | | | | | | | | |
| Blood | | | x | | | | | | | x | x | x | x | x | x | x | x | x | x | x | x |
| Inh8 | | | | | | | | | | | | | | | | | | | | | |
| Blood | | | x | | | | | | | x | x | x | x | x | x | x | x | x | x | x | x |
| LH | | | | | | | | | | | | | | | | | | | | | |
| Fecal | | 3x/ | | | | | | | | x | x | x | 3x/ | 3x/ | 3x/ | 3x/ | 3x/ | 3x/ | 3x/ | 3x/ | 3x/ |
| E2/P4/T | | wk | | | | | | | | | | | wk | wk | wk | wk | wk | wk | wk | wk | wk |
| Blood VG | | | x | | | | | | | x | x | x | x | x | x | x | x | x | x | x | x |
| Urine VG | | | x | x | x | x | x | x | x | x | | | | | | | | | | | |
| Stool VG | | | x | x | x | x | x | x | x | x | | | | | | | | | | | |
| Oral VG | | | x | x | x | x | x | x | x | x | | | | | | | | | | | |
| Blood | | x | | | | | | | | | | | | | x | | | x | | | x |
| CBC | | | | | | | | | | | | | | | | | | | | | |
| semen | | | | | | | | | | | | | | | x | | | x | | | x |

Kittens were housed in cages (2-3 kittens from same treatment group/cage) for 5 days and then transferred to a single group enclosure for further monitoring. During this time, pooled fecal and urine samples and individual oral swabs were collected daily from group-housed kittens for assessment of viral shedding. To analyze viral shedding, qPCR of viral genomes in individual blood, mixed urine, mixed feces, and individual oral swabs were monitored. Blood was sampled on day 7 following injection, weekly the remainder of the month, and monthly after the first month. Urine, feces, and oral swabs were collected daily for the first week.

Food dye was mixed in with cat food and fed separately to each individual kitten (three times per week) beginning two weeks prior to injection and continuing for nine months post-injection (˜1 year of age). As kittens from the same treatment group were housed together, fecal samples were assessed for estrogen and progesterone metabolites (females) or testosterone metabolites (males) to allow determination of onset of puberty and reproductive maturity. As described above in Example 1.D., color/cat-specific food dyes and/or glitter was mixed with cat food and fed separately to individual cats to facilitate identification of cat-specific feces. Kittens were weighed weekly, beginning two weeks prior to injection until one year of age.

Whole blood samples (1 ml, minimum) were collected (1×/month) from each kitten for assessment of serum MIS, inhibin B, anti-MIS antibodies, and LH concentrations, beginning just prior to injection. Additional blood samples were collected at days 7, 14, 21, and 28-days post-injection for both treated and control kittens.

Safety monitoring included daily assessment of general health and regular evaluation of injection sites (daily for 14 days, weekly through month 2, and then monthly thereafter. Physical exams and CBC/biochemistry assessments were conducted just prior to injection and then every three months until one year of age.

Postnatal health of all kittens was monitored throughout their development to puberty. Kittens were assessed for signs of behavioral estrus using video, and direct observation. As untreated kittens may enter puberty between 4 and 6 months, the study ran for up to 10 months. If there were no signs of cats having estrus cycles by 10 months of age, a follow up breeding study was considered.

Sex steroids, E2 and P4, were measured in fecal samples. Fecal samples were lyophilized and processed for E2 and P4 extraction. Fecal hormone analysis of females provided evidence of puberty by displaying ovarian cyclicity, based on a gradual increase over time in basal estrogens and the occurrence of estrogen spikes concurrent with estrus. Young females do not typically ovulate spontaneously, therefore fecal progesterone may not be as informative, but female kittens may appear to gain the capacity to spontaneously ovulate as they age so there is the possibility to pick up luteal phases toward the end of the one-year period.

For females, transabdominal ultrasound was performed monthly and uterine horn measurements were recorded. Measurements were taken on each uterine horn, as close to the bifurcation as possible. The widest and narrowest diameter were measured and divided by 2 to determine the major and minor axes. Area was calculated as an ellipse (π×major axis×minor axis) and reported as mean area for one uterine horn (mm²).

For males, elevation of fecal testosterone was an indicator of puberty and was substantiated by presence of penile spines and sperm production. Every three months post-treatment, male kittens were anesthetized using a ketamine-dexmedetomidine combination for attempted semen collection using a standardized electroejaculation protocol. Recovered samples were assessed for presence of sperm, fluid volume, sperm concentration, motility and morphology. Testicular dimensions and penile morphology (i.e., presence of spines) were also assessed.

Because male and female kittens are group-housed until one year of age, some breeding activity may occur during latter months, especially between control cats. Beginning at 6 months of age, females were assessed weekly via abdominal ultrasonography for determination of pregnancy status. Any observed breeding activity among cats will be documented.

### Results

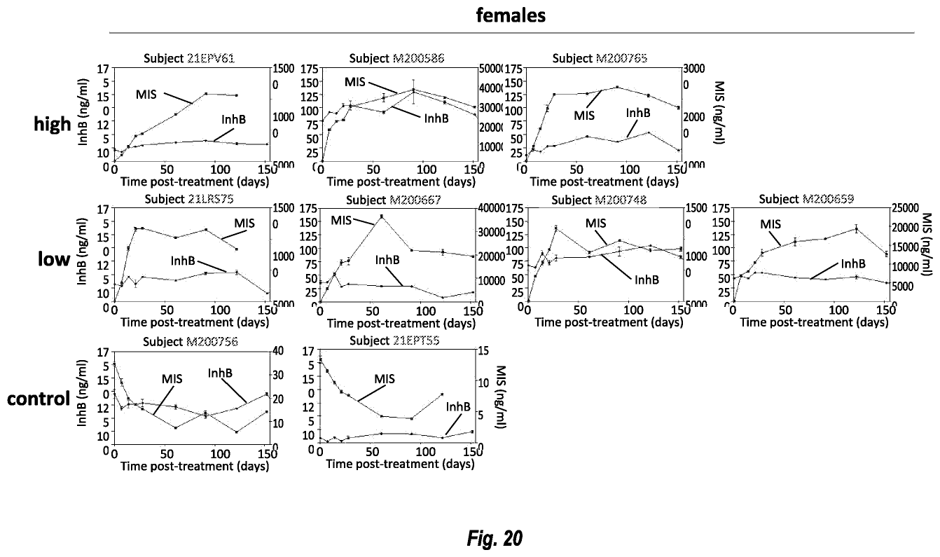

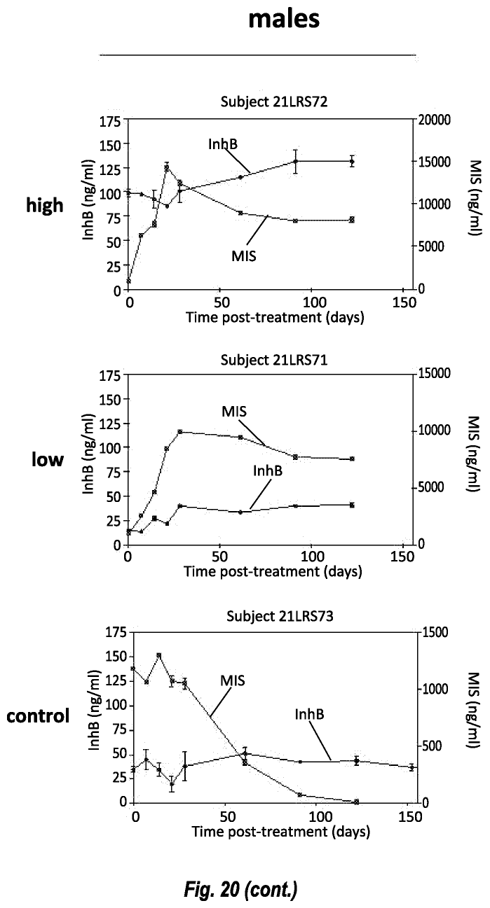

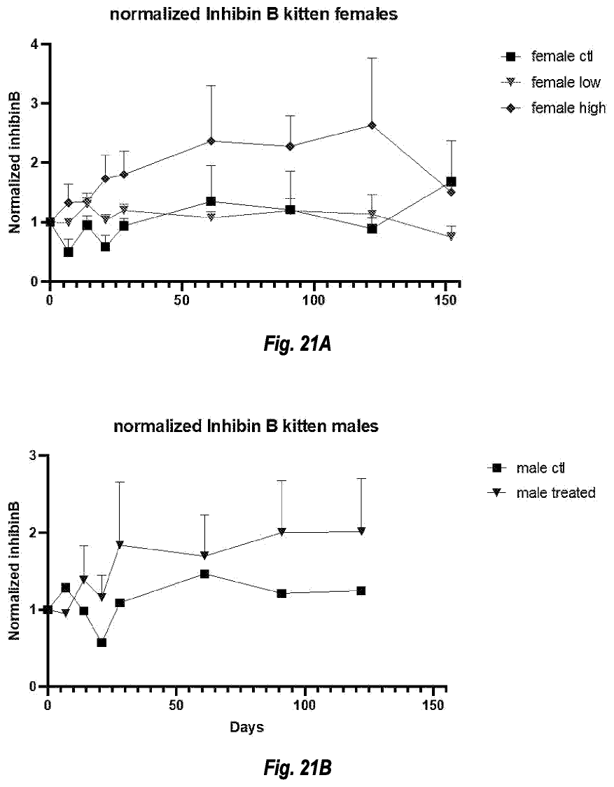

The prepared viruses used in this study (which were previously prepared as described in Example 1.E.) were confirmed effective. The average circulating concentration of MIS in kittens was measured at the 4-month timepoint. In the low dose group, i.e., 5e12 vg/kg, the circulating MIS concentration was 12.93 μg/ml. In the high dose group, i.e., 1e13 vg/kg, the circulating MIS concentration was 18.49 μg/ml. In the control group, the circulating MIS concentration was 8.36 ng/ml. These values were higher than what was measured at the same timepoint in adult cats, which averaged 3.48 μg/ml, and 15.17 μg/ml for the low and high dose respectively. The robust and stable expression of the transgene may be attributed to sustained and proportionally increasing contributions of muscle cells during the growth of the kittens (FIG. 20). In the controls, as expected, the endogenous MIS was high in young kittens, particularly males, and slowly declined as the males mature sexually to the low ng/ml levels. Inhibin B levels in young kittens were expected to mirror this pattern of high MIS expression, followed by a decline. However, in contrast to MIS, a significant increase of inhibin B was observed. This increase was associated with puberty, and with those inhibin B levels were maintained in the adults. Initial analysis suggested a small decline in inhibin B could be observed during the first month after treatment (˜7 months of age), particularly in control animals followed by a modest increase to 4 months post-treatment (˜10 months of age), when kittens are expected to approach puberty (FIGS. 21A-21B). Ongoing monitoring during puberty will be informative of the effect of MIS on hormones, particularly for inhibin B in the female high dose group that appeared to have a premature relative induction compared to controls or low dose (FIG. 21A).

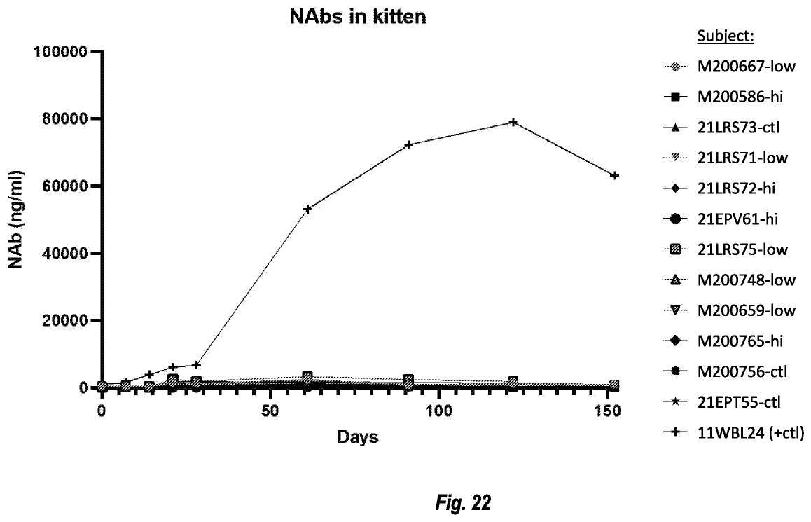

Further, no significant production of anti-MIS antibodies was observed in blood samples from kittens that received the vectors expressing fcMISv2 (FIG. 22). In contrast, Subject 11WBL24, which received AAV9-fcMISv1 during the first pilot study, demonstrated strong induction of anti-MIS antibody over the same timeframe (FIG. 22).

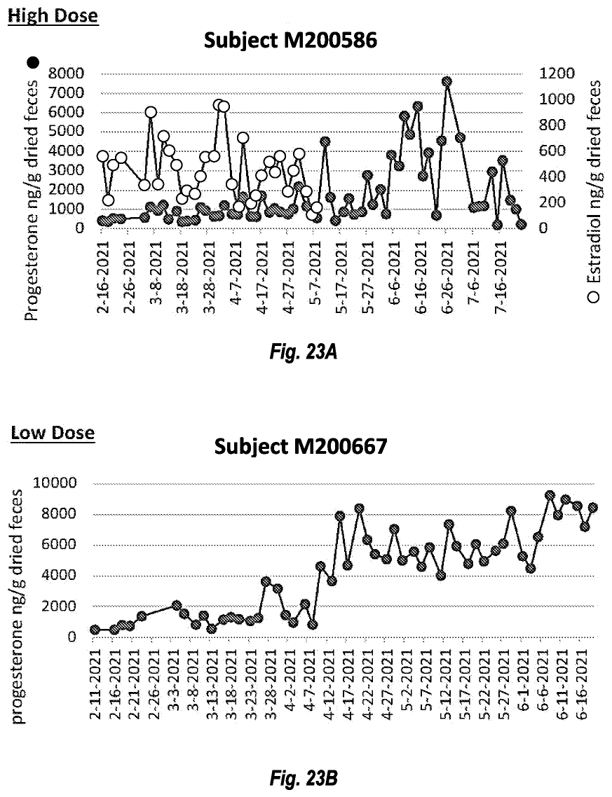

The effect of fcMISv2 on female kittens was studied. Fecal P4 levels from female kittens increased gradually over time (FIGS. 23A-C). This increase in P4 may be informative regarding reproductive maturation. All three kittens in FIGS. 23A-C, Subjects M200586, M200667, and M200756, were littermates and were expected to mature relatively synchronously. P4 values in prepubertal cats were much lower than what as observed with ovulation and the duration was much longer than a normal luteal phase. No previous studies have documented P4 changes in cats going through puberty. This rise in P4 may be indicative of the onset of puberty. Further characterization of fecal estrogen peaks (indicative of estrus) for these same three cats (Subjects M200586, M200667, and M200756,) may inform if at least some of these cats were showing reproductive cyclicity or not. These results, and the profile of the rest of the female cohort are pending.

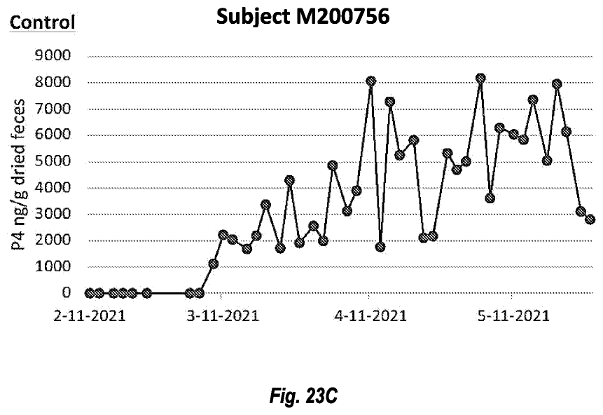

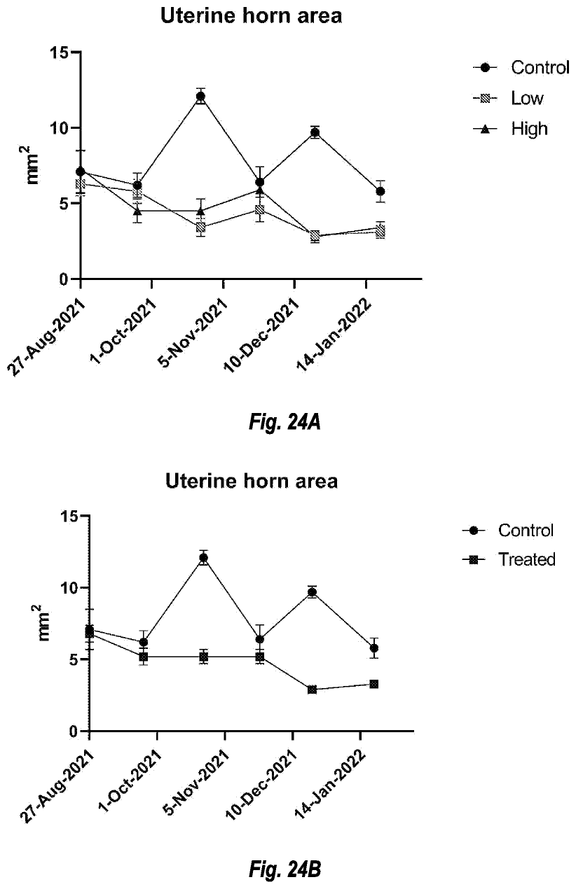

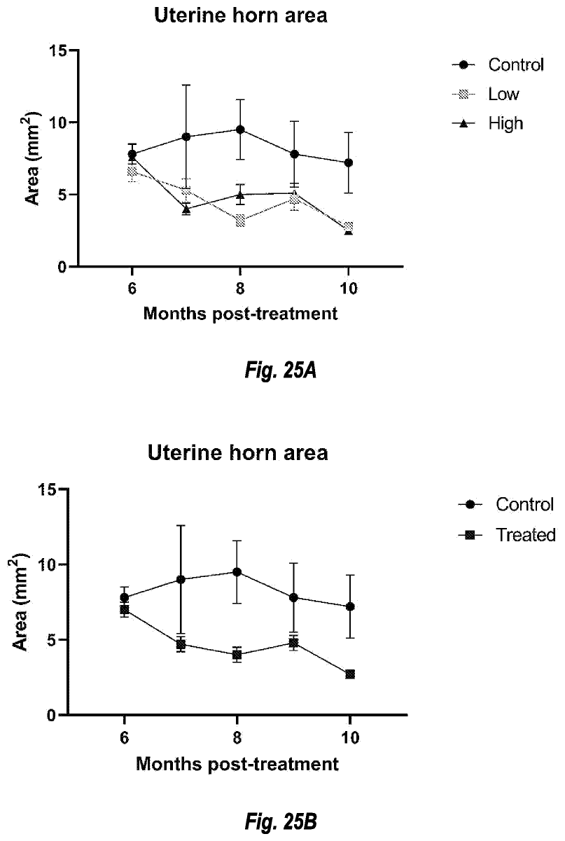

Following treatment with 5e12 vg/kg AAV9-fcMISv2 (low), 1e13 vg/kg AAV9-fcMISv2 (high), or 5e12 vp/kg of AAV9-empty vector (control), uterine horn measurements were performed in treated female cats. A reduction in uterine horn area was observed in cats treated with the high or low dose of AAV9-fcMISv2 (FIGS. 24A-25B), which may indicate contraception in cats over the time frame of the study. At 6 months post-treatment (i.e., when the cat is at about 9 months of age), there was no difference in uterine horn area between control cats and treated cats. Over time, however, there appeared to be a slight increase of uterine area in the control cats whereas the treated cats showed a slight decline. These results may indicate that the control females were actively cycling and possibly ovulating, leading to uterine glandular proliferation and regression, whereas the treated cats were not showing these cyclic changes (suggesting ovarian suppression). The large SEMs for the control mean values indicate that those uterine areas vary quite a bit over time while the treated females show minimal variation in area among individuals. The fecal hormone assays (when completed) should provide more insight into the relationship between changes in uterine areas and ovarian hormonal profiles.

The effect of fcMISv2 on male kittens was also studied. Both low and high doses of AAV9-fcMISv2 (i.e., 5e12 vg/kg and 1e13 vg/kg, respectively) in the male kittens, Subjects 21LRS71 and 21LRS72, respectively, did not significantly reduce sperm numbers by 8 months of age and those parameters continued to improve as they completed sexual maturation (Table 12). Furthermore, their sperm was fully functional as assessed in an IVM/IVF trial with domestic cat oocytes (Table 13). In contrast, the control male cat (Subject 21LRS73) appeared to be maturing properly by 5 months of age with the largest testes of the three males along with a fully formed preputial cavity and distinct penile spines (indicative of testosterone production) but he did not produce meaningful numbers of sperm. Insufficient amounts of motile sperm were collected from Subject 21LRS73 to assess his fertility in vitro. Although the cause of Subject 21LRS73's infertility was unknown, it was not presumed to be the result of AAV9 treatment.

In Subjects 21LRS71 and 21LRS72, sperm numbers were not negatively impacted by treatment with AAV9-fcMISv2. In fact, sperm numbers and sperm concentrations were higher than those observed in the control Subject 21LRS73 (Table 13). These increases in the AAV9-fcMIMSv2-treated cats were unexpected. The data disclosed in this example supports the use of AAV9-fcMISv2 in scenarios where increased sperm numbers and/or sperm concentrations may be desirable, such as in methods of sperm collection for artificial insemination of endangered or rare animals.

TABLE 12
Reproductive maturation of male kittens following prepubertal treatment with AAV9-fcMISv2 gene therapy

| | | | | | | | | | Sperm | Rate of | Normal |
|---|---|---|---|---|---|---|---|---|---|---|---|
| Male | Semen | | | | | | Sperm | Total | progress- | progress- | sperm |
| subject | collec- | | Body | Penile | Testes | Semen | concen- | sperm | sive | sive | mor- |
| (Trt | tion | Age | weight | mor- | volume | volume | tration | count | motility | motility | phology |
| group) | method | (mo) | (kg) | phology | (cm³) | (ml) | (×10⁶/ml) | (×10⁶) | (%) | (0-5) | (%) |
| Subject | EEJ | 5 | 2.74 | Complete | 1.36 | 0.13 | aspermic | N/A | N/A | N/A | N/A |
| 21LRS71 | | | | prepuce, | | | | | | | |
| (1e13 | | | | distinct | | | | | | | |
| vg/kg; | | | | spines | | | | | | | |
| high | EEJ | 8 | 3.72 | same | 2.78 | 0.10 | 75 | 7.35 | 70 | 3.5 | 20 |
| dose) | EEJ | 11 | 3.64 | same | 3.28 | 0.18 | 153 | 26.91 | 80 | 3.5 | 46 |
| | gamete | 12 | 3.48 | same | 3.28 | N/A | N/A | 32.17 | 60 | 3.5 | N/A |
| | rescue* | | | | | | | | | | |
| Subject | EEJ | 5 | 2.94 | Penile | 1.65 | 0.11 | aspermic | N/A | N/A | N/A | N/A |
| 21LRS72 | | | | frenulum, | | | | | | | |
| (5e12 | | | | distinct | | | | | | | |
| vg/kg; | | | | spines | | | | | | | |
| low | EEJ | 8 | 3.76 | Complete | 2.91 | 0.21 | 263 | 55.23 | 80 | 4.0 | 24 |
| dose) | | | | prepuce | | | | | | | |
| | EEJ | 11 | 3.62 | same | 3.09 | 0.21 | 384 | 79.19 | 80 | 4.0 | 49 |
| | gamete | 12 | 3.53 | same | 3.02 | N/A | N/A | 22.01 | 70 | 3.5 | N/A |
| | rescue* | | | | | | | | | | |
| Subject | EEJ | 5 | 3.36 | Complete | 2.32 | 0.21 | aspermic | N/A | N/A | N/A | N/A |
| 21LRS73 | | | | prepuce, | | | | | | | |
| (5e12 | | | | distinct | | | | | | | |
| vp/kg; | | | | spines | | | | | | | |
| control) | EEJ | 8 | 4.03 | same | 2.60 | 0.33 | 0.0035 | 0.0012 | N/A | N/A | N/A |
| | EEJ | 11 | 3.89 | same | 2.32 | 0.36 | 0.0209 | 0.0074 | N/A | N/A | 3 |
| | gamete | 12 | 3.84 | same | 2.54 | N/A | N/A | 0.117 | N/A | N/A | N/A |
| | rescue* | | | | | | | | | | |

TABLE 13
In vitro oocyte maturation/fertilization for evaluation of sperm
function in male kittens treated prepubertally with AAV9-fcMISv2

| | | Sperm motility (% | | Oocyte | |
|---|---|---|---|---|---|
| | AAV9- | progressive/rate) | Sperm motility (% | cleavage | Fertilization |
| Male | fcMISv2 | 0 h post- | progressive/rate) | (%) | (%)* |
| subject | treatment | insemination | 18 hpi | 48 hpi | 48 hpi |
| Subject | High dose | 90%/4.5 | 40%/2.5 | 18/37 | 17/35 |
| 21LRS71 | | | | (49) | (49%) |
| Subject | Low dose | 80%/4.0 | 50%/2.5 | 13/36 | 11/35 |
| 21LRS72 | | | | (36%) | (31%) |
| Subject | Control | N/A | N/A | N/A | N/A |
| 21LRS73 | | | | | |

### H. Follow-Up Breeding Study

### Materials and methods

One or multiple follow-up breeding studies were considered. For example, if there were no signs of cats having estrus cycles by 10 months of age, and/or if no females bred and/or conceived during the time period set forth above in this Example, a subsequent breeding trial may be warranted using a proven breeder male.

In general, materials and methods for this breeding study were as described in Example 1.E. above, with the following modifications. In this breeding study, a proven breeder male cat, Subject 18IDG51, was transferred into the group-housing room containing the six AAV9-fcMISv2 study females (1 control, 5 AAV-MIS treated). The cats were obtained from Marshall BioResources. A second proven breeder male, Subject 17CCW45, was transferred into a second room containing the three AAV9-fcMISv2 females (1 control, 2 treated) that were born at CREW (sired by Subject 18IDG51). In the weeks leading up to the start of the breeding trial, both males received controlled exposure (housed in a cat carrier) to the females within each room to facilitate integration. Each male was then housed with their respective females for 8 hours each day (from 8 am to 4 pm) for 5 days each week (Monday to Friday) for 4 consecutive months.

Following initial introduction of cats on the first day, the cat keeper remained in the room for 10 minutes to monitor interactions of the cats and ensure that excessive aggression does not occur between the male and females. After the keeper leaves the room, additional monitoring (about 5 minutes) occurred through the cat room window, and then periodically (every 30-60 minutes) throughout the first day and the remainder of the week. Some aggression was expected among cats during the initial direct exposure, including minor physical trauma (e.g., scratches) to the male or females. Secure retreat spaces (e.g., doorless cat carriers, cat condos, and shelves) were available in the room to allow cats to escape from any aggressor. Serious and/or continuous fighting required intervention and physical separation of cats by the keeper. Keepers assessed cats each day for any evidence of trauma and veterinary staff provided medical treatment as needed. Remote baby monitors were used by the cat keeper to hear vocalizations (related to breeding activity or aggression) from the cat room throughout the day when working in other areas of the cat colony. Two video cameras in each cat room recorded animal interactions continually throughout the day (from 8 am to 4 pm) and all video footage was reviewed by CREW volunteers to identify any possible breeding activity. These observations helped to document the occurrence of breeding activity that failed to result in ovulation and/or conception, and to determine the expected data of parturition for any pregnant cats. Any breeding activity observed by the cat keeper was recorded in the daily log, noting the identity of the female and the time of day to allow subsequent video review.

Each female was assessed by abdominal palpation and ultrasound exam weekly to determine pregnancy status (i.e., presence and viability of fetuses). All females received prior operant conditioning to voluntarily accept these procedures with minimal restraint or disturbance.

Any pregnant females were reassessed via ultrasonography every three weeks to monitor fetal development and viability. Females remained in group housing until ˜Day 50 of pregnancy and were then transferred into the maternity room with individual caging for subsequent natural parturition (typically at ˜Day 63-65 post-breeding). Pregnant females were monitored in person each day and remotely via an internet-accessible video camera linkage through the expected time of parturition.

Pregnant females were monitored in person each day (i.e., 8 am to 4 pm) by keepers and then continually (i.e., 4 pm to 8 pm) by CREW volunteer observers via an internet-accessible video camera linkage through the expected time of parturition. Keepers and veterinary staff were notified when any female went into labor. If dystocia occurred, kittens were delivered by C-section at the discretion of the attending veterinarian. Kittens received initial physical exams within 24 hours of birth, were weighed daily to monitor growth (through the first month post-partum, and then weekly), and provided with supportive care as necessary.

Complete necropsies were conducted on any still-born kittens or deceased neonates, including assessment of gender and anatomy of the reproductive tract. Entire reproductive tracts were recovered and fixed for later histological evaluation to assess possible MIS effects on in utero reproductive tract development.

Viable kittens were raised preferentially by the queens through weaning (typically at about 8 weeks of age). Kittens were hand-raised or fostered by other queens as necessary in certain situations, e.g., abandonment, aggression, or overgrooming by the dam.

Post-weaning, kittens may be adopted out as companion animals or retained in the CREW colony for future research. In the event viable female kittens were born to AAV9-fcMISv2-treated females, these female kittens were assessed for fertility through the time of puberty, i.e., at 7-8 months of age. blood samples were collected from kittens born to treated females within 12 hours of birth to assess serum MIS levels. If AAV9-fcMISV2K-treated females did not become pregnant from natural breeding, their fertility status were assessed to determine if these treated females were effectively contracepted.

During the breeding trial, voided fecal samples were collected three times a week from each female for hormone monitoring. As described above in Example 1.D. and Example 3.A., color/cat-specific food dyes and/or glitter was mixed with cat food and fed separately to individual cats to facilitate identification of cat-specific feces. Also, blood samples (each a minimum volume of 1 ml) were collected once a month from each female for assessment of serum MIS concentrations. To minimize handling distress, blood samples (>1 ml) were collected via medial saphenous or cephalic venipuncture following sedation using a low-dose combination of ketamine, dexmedetomidine, and/or butorphanol combination, with partial reversal with atipamezole. For recovery of smaller blood volumes (<1 ml) and/or collection from pregnant females, blood samples were collected via venipuncture using manual restraint only (i.e., securing cats in a nylon holding bag). This procedure was halted when the cat appeared stressed by the procedure.

### Example 4—Effectiveness of AAV-Wt Canine MIS Treatment to Prevent Puppies from Entering Puberty, and to Provide Long-Term Sterility

### I. Preventing Puberty in Dogs (Puppies)

The ability of MIS treatment via gene delivery to prevent puberty in dogs (puppies) as well as any other associated developmental effects are assessed. Furthermore, parameters for using canine MIS when given as a single injection (e.g., via gene delivery) for long term reduction of fertility in puppies and/or for preventing puppies from entering puberty are assessed. By way of example, this also serves as a non-human animal model to assess MIS treatment via gene delivery or delivery of a MIS protein for the delay of puberty in human subjects.

Timing for AAV-wt canine MIS injection of puppies is dependent on the timing of parturition and weaning. Healthy puppies will be weaned by 8 weeks of age. At approximately 3 months of age (e.g., 10-12 weeks of age), the healthy puppies will be randomized into a control and a treated group. The puppies will be injected i.m. into caudal thigh muscles with the AAV-wt canine MIS or empty vector control as follows: (1) High dose AAV-wt canine MIS (1e13 vg/kg); (2) Low dose AAV-wt canine MIS (5e12 vg/kg); and (3) Control empty vector (5e12 vp/kg). Table 14 provides a timetable for sampling and monitoring of the puppies.

TABLE 14
Puppy monitoring timetable

| | | | d | d | d | d | d | d | d | d | d | d | m | m | m | m | m | m | m | m | m |
|---|---|---|---|---|---|---|---|---|---|---|---|---|---|---|---|---|---|---|---|---|---|
| time | birth | pre | 0 | 1 | 2 | 3 | 4 | 5 | 6 | 7 | 14 | 21 | 1 | 2 | 3 | 4 | 5 | 6 | 7 | 8 | 9 |
| Blood | x | | x | | | | | | | x | x | x | x | x | x | x | x | x | x | x | x |
| MIS | | | | | | | | | | | | | | | | | | | | | |
| Blood a- | x | | x | | | | | | | x | x | x | x | x | x | x | x | x | x | x | x |
| MIS Ab | | | | | | | | | | | | | | | | | | | | | |
| Blood | | | x | | | | | | | x | x | x | x | x | x | x | x | x | x | x | x |
| Inh8 | | | | | | | | | | | | | | | | | | | | | |
| Blood | | | x | | | | | | | x | x | x | x | x | x | x | x | x | x | x | x |
| LH | | | | | | | | | | | | | | | | | | | | | |
| Fecal | | 3x/ | | | | | | | | x | x | x | 3x/ | 3x/ | 3x/ | 3x/ | 3x/ | 3x/ | 3x/ | 3x/ | 3x/ |
| E2/P4/T | | wk | | | | | | | | | | | wk | wk | wk | wk | wk | wk | wk | wk | wk |
| Blood VG | | | x | | | | | | | x | x | x | x | x | x | x | x | x | x | x | x |
| Urine VG | | | x | x | x | x | x | x | x | x | | | | | | | | | | | |
| Stool VG | | | x | x | x | x | x | x | x | x | | | | | | | | | | | |
| Oral VG | | | x | x | x | x | x | x | x | x | | | | | | | | | | | |
| Blood | | x | | | | | | | | | | | | | x | | | x | | | x |
| CBC | | | | | | | | | | | | | | | | | | | | | |
| semen | | | | | | | | | | | | | | | x | | | x | | | x |

Puppies will be housed in cages (2-3 puppies from same treatment group/cage) for 5 days and then transferred to a single group enclosure for further monitoring. During this time, pooled fecal and urine samples and individual oral swabs will be collected daily from group-housed puppies for assessment of viral shedding. To analyze viral shedding, qPCR of viral genomes in individual blood, mixed urine, mixed feces, and individual oral swabs will be monitored. Blood will be sampled on day 7 following injection, weekly the remainder of the month, and monthly after the first month. Urine, feces, and oral swabs will be collected daily for the first week.

Food dye will be mixed in with dog food and fed separately to each individual puppies (three times per week) beginning two weeks prior to injection and continuing for example, for nine months post-injection (e.g., ˜1 year of age). As puppies from the same treatment group may be housed together, fecal samples will be assessed for estrogen and progesterone metabolites (females) or testosterone metabolites (males) to allow determination of onset of puberty and reproductive maturity. Puppies will be weighed weekly, beginning two weeks prior to injection until the end of the study period.

Whole blood samples (1 ml, minimum) will be collected (1×/month) from each puppy for assessment of serum MIS, inhibin B, anti-MIS antibodies, and LH concentrations, beginning just prior to injection. Additional blood samples will be collected at days 7, 14, 21, and 28-days post-injection for both treated and control puppies.

Safety monitoring will include daily assessment of general health and regular evaluation of injection sites (daily for 14 days, weekly through month 2, and then monthly thereafter. Physical exams and CBC/biochemistry assessments will be conducted just prior to injection and then every three months until end of the study period.

Puppies will be assessed for signs of behavioral estrus using video, and direct observation. If there are no signs of dogs having estrus cycles, a follow up breeding study may be considered.

Fecal hormone analysis of females provides evidence of puberty by displaying ovarian cyclicity, based on a gradual increase over time in basal estrogens and the occurrence of estrogen spikes concurrent with estrus. Young females do not typically ovulate spontaneously, therefore fecal progesterone may not be as informative, but female puppies may appear to gain the capacity to spontaneously ovulate as they age so there is the possibility to pick up luteal phases toward the end of the study period.

For males, elevation of fecal testosterone is an indicator of puberty and is substantiated by presence of penile spines and sperm production. Every three months post-treatment, male puppies will be anesthetized using a ketamine-dexmedetomidine combination for attempted semen collection using a standardized electroejaculation protocol. Recovered samples will be assessed for presence of sperm, fluid volume, sperm concentration, motility and morphology. Testicular dimensions and penile morphology (i.e., presence of spines) also will be assessed.

Because male and female puppies will be group-housed until the end of the study period, some breeding activity may occur during latter months, especially between control dogs. During those latter months, females will be assessed weekly via abdominal ultrasonography for determination of pregnancy status. Any observed breeding activity among dogs will be documented.

### J. Follow-Up Breeding Study

One or multiple follow-up breeding studies may be considered. For example, if there are no signs of dogs having estrus cycles, and/or if no females breed and/or conceive during the study period set forth above in this Example, a subsequent breeding trial may be warranted using a proven breeder male.

In such a follow-up breeding study, a proven breeder male dog will be transferred into the group-housing room containing the AAV-wt canine MIS study females. The male will have received previous controlled exposure (housed in a dog carrier) to the females within the room to facilitate integration. The male will be housed with the females for 8 hours each day for 5 days each week for 4 consecutive months. Breeding activity will be documented through a combination of direct observation and remote audio/video monitoring. Each female will be assessed by abdominal palpation and ultrasound exam weekly to determine pregnancy status (i.e., presence and viability of fetuses). All females have received prior operant conditioning to voluntarily accept these procedures with minimal restraint or disturbance.

Any pregnant females will be reassessed via ultrasonography every three weeks to monitor fetal development and viability. Females will remain in group housing until appropriate to be transferred into the maternity room with individual caging for subsequent natural parturition. Pregnant females will be monitored in person each day and remotely via an internet-accessible video camera linkage through the expected time of parturition.

### Example 5—Effectiveness of Recombinant hMIS Protein Treatment to Delay Human Subjects from Entering Puberty

By way of example, delay of puberty was assessed in cats (kittens) and is assessed dogs (kittens) in Examples 2 and 3 as exemplary non-human animals to demonstrate that MIS treatment can delay puberty.

In Example 4, the ability of recombinant human MIS protein produced from the proprotein of SEQ ID NO: 7 for a reversible delay of puberty in human female subjects is assessed. That is, the administration of LR-MIS protein in delaying puberty in the female subject was assessed and determined to be reversible. hMIS protein can be administered to a prepubescent human female subject at the age when puberty has not started, or at around 8-16 years.

A human rhMIS protein, e.g., LR-MIS of SEQ ID NO: 7 was produced according to the methods disclosed in in US Application US20200071376, which is incorporated herein in its entirety. The availability of a biologically active rhMIS protein that can be produced and purified to high yields using CHO cells and allowed for higher and longer dosing in-vivo, which had previously been impractical with the poorly cleaved wild-type protein, or impossible using commercial C-terminal recombinant MIS protein, which was found to be devoid of activity. For example, the inventors previously demonstrated that incubation of fetal (E14.5) female rat urogenital ridges with 5 μg/ml of rhMIS for 72 h in ex vivo cultures resulted in near complete regression of the Mullerian duct, whereas the R&D Systems (Minneapolis, Minn.) c-terminal MIS has no observable activity on the Mullerian duct bioassay; this assay is the gold standard to test potency and specificity of the hormone.

Treatment of mice with rhMIS Protein Results in Reversible Ovarian Quiescence.

The rhMIS protein can be administered subcutaneously (s.c.), intravenously (i.v.), intraperitonealy (i.p.), each resulting in a half-life of approximately 4 h and reaching peak concentrations (Cmax) at 4 hours, 30 mins, and 2 hours respectively. The preferred route of delivery for rhMIS protein was subcutaneously, since its absorption kinetics where most favorable; however, when osmotic pumps were employed, intraperitoneal implantation was found to be optimal, producing steady delivery of up to one week (see, e.g., FIG. 1F in US2020/0071376). rhMIS activity was remarkably stable, with the material recovered from pumps implanted in mice for one week conserving full biological activity in the rat urogenital ridge bioassay (data not shown).

Administration of rhMIS to the female subject can by via any means disclosed in in US Application US20200071376, which is incorporated herein in its entirety. In some instances, administration can be via a pump (e.g., osmotic pump) or transdermal patch or the like.

To confirm that rhMIS protein can result in inhibition of primordial follicles to elucidate the kinetics of ovarian re-awakening, the effect of transient treatment with rhMIS protein is measured in prepubescent female mice, which is a representative animal model for prepubescent female humans. The rhMIS protein will be administered s.c. twice daily (every 12 h), at 1.5 mg/kg which in silico pharmacokinetic modeling predicted would maintain circulating levels of rhMIS above the target level of 0.25 μg/ml. Actual circulating levels of rhMIS can be measured by ELISA 12 h after injection, representing the trough, were maintained above the target threshold of 0.25 μg/ml, albeit with diminishing concentration during the 35 days of treatment. Ovaries from treated mice can be assessed, and a markedly reduction in size is assessed by analysis of a representative middle section following 35 days of treatment and if it contains fewer primary and no secondary or antral follicles, it demonstrates inhibition of follicle development. Assessment of the ovaries following cessation of treatment with rhMIS protein is assessed to determine the ovaries are released from quiescence and that folliculogenesis resumed. Assessment of ovarian volume in the mice can be assessed over 15 days, and an increase in size indicates that the size of primary follicles gradually increased from day 3 to day 10, and secondary follicles began to appear day 5 and increased to levels similar to control by day 15, at which time some antral follicles can be observed.

To test the efficacy of rhMIS protein for reversibly delaying puberty, the use of osmotic pumps implanted i.p. in C57BL/6N female mice was elected, which allows for very precise delivery of rhMIS (see FIG. 1F in US20200071376). In this model osmotic pumps loaded with 100 μl of a 1200 μg/ml solution of rhMIS diluted in saline, or saline loaded control pumps were implanted; the pumps were replaced every 7 or 5 days (see, e.g., FIG. 5E in US20200071376). After 2 weeks, mice were sacrificed, ovaries were retrieved, serially sectioned, and follicle counts performed. Significantly higher ovarian reserves were observed in mice implanted with rhMIS-eluting pumps compared to controls with saline pumps. Treatment with hrMIS alone did not significantly affect either primordial follicles or growing follicles within this short timespan; however, there was a trend towards lower numbers of growing follicles compared to saline only controls.

One could envision a lower dose rhMIS usage where primordial follicle activation could be slowed down but not completely arrested, thus reversibly delaying the onset of puberty.

In conclusion, it is demonstrated herein in Examples 1-3 the effectiveness of AAV-wt feline MIS or AAV-wt canine MIS at high doses can irreversibly prevent puberty in cats and dogs, respectively and that in Example 4, administration of rhMIS can reversibly delay the onset of puberty in a mouse animal model, demonstrating that rhMIS is a potent inhibitor of primordial follicle activation.

1. A method of reducing fertility in a prepubescent non-human subject comprising administering to the subject an effective amount of a composition comprising a vector comprising a nucleic acid encoding a Mullerian Inhibiting Substance (MIS) protein operatively linked to one or more regulatory elements.

2. (canceled)

3. The method of claim 1, wherein the prepubescent non-human subject is a kitten or a puppy.

4. The method of claim 1, wherein the prepubescent non-human subject is female.

5. The method of claim 1, wherein the MIS protein comprises:
a) a wild-type feline MIS protein, the amino acid sequence of SEQ ID NO: 1 or SEQ ID NO: 18, or an amino acid sequence having at least 80%, at least 85%, at least 90%, at least 95%, at least 96%, at least 97%, at least 98%, or at least 99% sequence identity to the amino acid sequence of SEQ ID NO: 1 or SEQ ID NO: 18;
b) a wild-type canine MIS protein, the amino acid sequence of SEQ ID NO: 2, or an amino acid sequence having at least 80%, at least 85% at least 90%, at least 95%, at least 96%, at least 97%, at least 98%, or at least 99% sequence identity to the amino acid sequence of SEQ ID NO: 2;
c) a wild-type human MIS protein, the amino acid sequence of SEQ ID NO: 4, or an amino acid sequence having at least 80%, at least 85%, at least 90%, at least 95%, at least 96%, at least 97%, at least 98%, or at least 99% sequence identity to the amino acid sequence of SEQ ID NO: 4;
d) a feline MIS protein comprising a non-MIS leader sequence in place of amino acids 1-21 of SEQ ID NO: 1 or of SEQ ID NO: 18, and an amino acid sequence having at least 80%, at least 85%, at least 90%, at least 95%, at least 96%, at least 97%, at least 98%, or at least 99% sequence identity to amino acids 22-588 of SEQ ID NO: 1 or to amino acids 22-589 of SEQ ID NO: 18, optionally wherein the non-MIS leader sequence comprises amino acids 1-18 of SEQ ID NO: 14; or
e) a modified feline MIS protein (LR-fcMIS) comprising amino acids of SEQ ID NO: 14 or SEQ ID NO: 20, and an amino acid sequence having at least 80%, at least 85%, at least 90%, at least 95%, at least 96%, at least 97%, at least 98%, or at least 99% sequence identity to amino acids 22-588 of SEQ ID NO: 14 or amino acids 22-589 of SEQ ID NO: 20.

6. The method of claim 1, wherein the prepubescent non-human subject is a kitten:
a) that is 12 months old or less, 11 months old or less, 10 months old or less, 9 months old or less, 8 months old or less, 7 months old or less, 6 months old or less, 5 months old or less, 4 months old or less, 3 months old or less, or 2 months old or less;
b) weighing 2 kg or less; and/or
c) that has been weaned.

7. (canceled)

8. The method of claim 1, wherein the prepubescent non-human subject is a puppy:
a) that is 24 months old or less, 22 months old or less, 20 months old or less, 18 months old or less, 16 months old or less, 14 months old or less, 12 months old or less, 11 months old or less, 10 months old or less, 9 months old or less, 8 months old or less, 7 months old or less, 6 months old or less, 5 months old or less, 4 months old or less, 3 months old or less, or 2 months old or less; and/or
b) that has been weaned.

9. (canceled)

10. (canceled)

11. The method of claim 1, wherein the vector is a viral vector, a plasmid, a cosmid, or a phagemid.

12. (canceled)

13. (canceled)

14. The method of claim 1, wherein the composition comprises a sterile, injectable solution.

15. (canceled)

16. The method of claim 1, wherein the composition comprises a lipid, lipid emulsion, liposome, nanoparticle, or exosomes.

17. The method of claim 1, wherein the vector is an adenoviral vector, an adeno-associated virus (AAV) vector, a poxvirus vector, or a lentiviral vector.

18. The method of claim 1, wherein the vector is an AAV vector.

19. (canceled)

20. The method of claim 1, wherein the one or more regulatory elements comprise:
a) a promoter element;
b) a promoter element and an enhancer element; and/or
c) a constitutively active promoter.

21. (canceled)

22. (canceled)

23. The method of claim 1, wherein the composition comprises a pharmaceutically acceptable carrier.

24. The method of claim 1, wherein the administering is via injection.

25. The method of claim 1, wherein the administering is via intravenous, subcutaneous, or intramuscular administration.

26. (canceled)

27. The method of am claim 1, wherein the administering is via a single injection.

28. The method of claim 1, wherein the administering is via a single one-time injection.

29. The method of claim 1, wherein the administering is via a single dose split into multiple injections.

30. (canceled)

31. The method of claim 1, wherein the effective amount of the composition administered to the prepubescent non-human subject is 1×10¹³ vector genomes or less, 5×10¹² vector genomes or less, 1×10¹² vector genomes or less, 5×10¹¹ vector genomes or less, or 1×10¹¹ vector genomes or less per kilogram weight of the subject.

32. The method of claim 1, wherein the concentration of MIS protein in the serum of the prepubescent non-human subject at or after 6 months, at or after 9 months, at or after 12 months, at or after 15 months, or at or after 24 months following administration of the composition is greater than 250 ng/ml, greater than 300 ng/ml, greater than 400 ng/ml, greater than 500 ng/ml, greater than 600 ng/ml, greater than 700 ng/ml, greater than 800 ng/ml, greater than 900 ng/ml, greater than 1 μg/ml, greater than 1.5 μg/ml, greater than 2 μg/ml, greater than 3 μg/ml, greater than 4 μg/ml, greater than 5 μg/ml, greater than 6 μg/ml, greater than 7 μg/ml, greater than 8 μg/ml, greater than 9 μg/ml, greater than 10 μg/ml, or greater than 11 μg/ml.

33. (canceled)

34. The method of claim 1, wherein the prepubescent non-human subject is female and following administration of the composition,
a) does not develop follicles,
b) does not develop follicles with viable eggs,
c) does not experience puberty,
d) does not show signs of estrus, and/or
e) is infertile.

35. A vector comprising a nucleic acid encoding a feline Mullerian Inhibiting Substance (MIS) protein operatively linked to one or more regulatory elements,
wherein the feline MIS protein comprises a wild-type feline MIS protein having an amino acid sequence of SEQ ID NO: 1 or SEQ ID NO: 18, or an amino acid sequence having at least 80%, at least 85%, at least 90%, at least 95%, at least 96%, at least 97%, at least 98%, or at least 99% sequence identity to amino acids 22-588 of SEQ ID NO: 1 or to amino acids 22-589 of SEQ ID NO: 18, or
wherein the feline MIS protein comprises a chimeric feline MIS protein having an amino acid sequence of SEQ ID NO: 3 or an amino acid sequence having at least 80%, at least 85%, at least 90%, at least 95%, at least 96%, at least 97%, at least 98%, or at least 99% sequence identity to amino acids 22-572 of SEQ ID NO: 3.

36. (canceled)

37. (canceled)

38. (canceled)

39. A vector comprising a nucleic acid encoding a canine Mullerian Inhibiting Substance (MIS) protein operatively linked to one or more regulatory elements, wherein the canine MIS protein comprises a wild-type canine MIS protein having an amino acid sequence of SEQ ID NO: 2 or an amino acid sequence having at least 80%, at least 85%, at least 90%, at least 95%, at least 96%, at least 97%, at least 98%, or at least 99% sequence identity to amino acids 22-588 of SEQ ID NO: 2.

40. (canceled)

41. (canceled)

42. A modified feline Mullerian Inhibiting Substance (MIS) protein which is produced from a feline MIS proprotein selected from:
a feline MIS protein comprising a non-MIS leader sequence in place of amino acids 1-21 of SEQ ID NO: 1 or of SEQ ID NO: 18, and an amino acid sequence having at least 80%, at least 85%, at least 90%, at least 95%, at least 96%, at least 97%, at least 98%, or at least 99% sequence identity to amino acids 22-588 of SEQ ID NO: 1 or to amino acids 22-589 of SEQ ID NO: 18, or
a chimeric feline MIS protein comprising a non-MIS leader sequence in place of amino acids 1-21 of SEQ ID NO: 3, and having an amino acid sequence having at least 80%, at least 85%, at least 90%, at least 95%, at least 96%, at least 97%, at least 98%, or at least 99% sequence identity to amino acids 22-572 of SEQ ID NO: 3, or
a modified feline MIS protein (LR-fcMIS) comprising amino acids of SEQ ID NO: 14 or SEQ ID NO: 20, and an amino acid sequence having at least 80%, at least 85%, at least 90%, at least 95%, at least 96%, at least 97%, at least 98%, or at least 99% sequence identity to amino acids 22-588 of SEQ ID NO:
14 or amino acids 22-589 of SEQ ID NO: 20.

43. The modified feline MIS protein of claim 42:
a) wherein the feline MIS protein comprises a protein having at least 85% sequence identity to amino acids 22-588 of SEQ ID NO: 1 or to amino acids 22-589 of SEQ ID NO: 18, and wherein amino acid residue Q at position 478 of SEQ ID NO: 1 or position 479 of SEQ ID NO: 18 is changed from a Q to a R (arginine), or a conservative amino acid of R;
b) wherein the chimeric feline MIS protein comprises a protein having at least 85% sequence identity to amino acids 22-572 of SEQ ID NO: 3, and wherein amino acid residue Q at position 465 of SEQ ID NO: 3 is changed from a Q to a R (arginine), or a conservative amino acid of R such as, a K (lysine); or
c) wherein the non-leader sequence is selected from any of SEQ ID NO: 9-13.

44. (canceled)

45. (canceled)

46. A modified canine Mullerian Inhibiting Substance (MIS) protein which is produced from a canine MIS proprotein, where the canine MIS proprotein comprises a non-MIS leader sequence in place of amino acids 1-21 of SEQ ID NO: 2, and an amino acid sequence having at least 80%, at least 85%, at least 90%, at least 95%, at least 96%, at least 97%, at least 98%, or at least 99% sequence identity to amino acids 22-588 of SEQ ID NO: 2, or an amino acid sequence of SEQ ID NO: 15 or an amino acid sequence having at least 80%, at least 85%, at least 90%, at least 95%, at least 96%, at least 97%, at least 98%, or at least 99% sequence identity to SEQ ID NO: 15.

47. The modified canine MIS protein of claim 46, wherein canine MIS comprises a protein having at least 85% sequence identity to amino acids 23-543 of SEQ ID NO: 2 and wherein amino acid residue Q at position 462 of SEQ ID NO: 2 is changed from a Q to a R (arginine), or a conservative amino acid of R: optionally wherein the conservative amino acid of R is K.

48. (canceled)

49. A pharmaceutical composition comprising the modified feline MIS protein of claim 43.

50. A method of reversibly delaying puberty in a prepubescent human female subject comprising administering to the subject an effective amount of a composition comprising a recombinant human Mullerian Inhibiting Substance (rhMIS) protein, wherein the recombinant human MIS protein is comprises amino acid residues 25-560 of SEQ ID NO: 4, or a protein at least 85% sequence identity to SEQ ID NO: 4, and wherein the amino acid residue 450 of SEQ ID NO: 4 is changed from a Q to R or a conservative amino acid of R.

51. (canceled)

52. (canceled)

53. (canceled)

54. (canceled)

55. (canceled)

56. (canceled)

57. (canceled)

58. A method of detecting anti-fcMIS antibodies or anti-clMIS antibodies in non-human subjects administered a viral vector expressing fcMIS or clMIS comprising
a) obtaining a sample from a non-human subject administered a viral vector encoding fcMIS or clMIS, optionally wherein the fcMIS or clMIS comprises the amino acid sequence of SEQ ID NO: 1, SEQ ID NO: 2, SEQ ID NO: 3, SEQ ID NO: 14, SEQ ID NO: 15, SEQ ID NO:18, or SEQ ID NO: 20;
b) optionally isolating recombinant fcMIS or clMIS, optionally wherein the recombinant fcMIS or clMIS comprises a FLAG tag;
c) adding fcMIS or clMIS to a substrate, optionally wherein the substrate is an ELISA plate;
d) adding a test sample to the substrate;
e) incubating the substrate with a detectable antibody, optionally wherein the detectable antibody is a goat anti-IgG HRP; and
f) performing an enzyme substrate reaction, optionally wherein the enzyme substrate is HRP.

59. (canceled)

60. (canceled)

61. (canceled)

62. A method of providing long term sterility in a prepubescent non-human subject comprising administering to the subject an effective amount of a composition comprising a vector comprising a nucleic acid encoding a Mullerian Inhibiting Substance (MIS) protein operatively linked to one or more regulatory elements.
